(19) United States
(12) Patent Application Publication
(10) Pub. No.: US 2023/0201335 A1
(43) Pub. Date: Jun. 29, 2023

(54) Antigen Presenting Polypeptide Complexes and Methods of Use Thereof

(71) Applicant: Cue Biopharma, Inc., Boston, MA (US)

(72) Inventors: Ronald D. SEIDEL, III, Natick, MA (US); Rodolfo J. CHAPARRO, Cambridge, MA (US); John F. ROSS, Arlington, MA (US); Chee Meng LOW, Boston, MA (US); Raymond J. MONIZ, Boston, MA (US); Gurpanna SAGGU, Westwood, MA (US)

(21) Appl. No.: 17/927,477

(22) PCT Filed: May 26, 2021
(86) PCT No.: PCT/US2021/034371

§ 371 Date: Nov. 23, 2022

Related U.S. Application Data
(60) Provisional application No. 63/120,671, filed on Dec. 2, 2020.
(60) Provisional application No. 63/030,243, filed on May 26, 2020.

(51) Int. Cl. A61K 39/29; C07K 14/74; C07K 14/55; C07K 14/47; A61K 39/00; A61K 39/12

(52) U.S. Cl. CPC A61K 39/292; C07K 14/70539; C07K 14/55; C07K 14/4748; A61K 39/001181; A61K 39/001153; A61K 39/12; A61K 2039/6056; A61K 2039/605

(57) ABSTRACT

The present disclosure provides Multimeric Antigen Presenting Polypeptides (MAPPs) for the presentation of antigens in the context of a class I MHC receptor. The present disclosure provides nucleic acids comprising nucleotide sequences encoding those MAPPs, as well as cells genetically modified with the nucleic acids. MAPPs of the present disclosure are useful for selectively modulating activity of T cells having T cell receptors that recognize the antigens. Thus, the present disclosure provides compositions and methods for modulating the activity of T cells, as well as compositions and methods for treating persons who have diseases and/or disorders including cancers, autoimmune diseases and/or allergies.

This application claims the benefit of U.S. Provisional Patent Appln. No. 63/120,671 filed Dec. 2, 2020, and the benefit of U.S. Provisional Patent Appln. No. 63/030,243 filed May 26, 2020.

This application contains a sequence listing submitted electronically via EFS-web, which serves as both the paper copy and the computer readable form (CRF) and consists of a file entitled “123640-8015.WO00_seqlist.txt”, which was created on May 26, 2021, which is 502,390 bytes in size, and which is herein incorporated by reference in its entirety.

### I. INTRODUCTION

An adaptive immune response involves the engagement of the T cell receptor (TCR), present on the surface of a T cell, with a small antigenic molecule (e.g., a peptide antigen) non-covalently presented on the surface of an antigen presenting cell (APC) by a major histocompatibility complex (MHC; also referred to in humans as a human leukocyte antigen (“HLA”) complex). This engagement represents the immune system's targeting mechanism and is a requisite molecular interaction for T cell modulation (activation or inhibition) and effector function. In addition to epitope-specific cell targeting, T cells may be targeted by immunomodulatory proteins found in, for example, APCs, that affect various functions of the target cells (e.g., activation or inhibition of various T cell functions) through of their costimulatory proteins found, for example, on the APC with counterpart costimulatory proteins (e.g., receptors) on the T cells. Both signals—epitope/TCR binding and engagement of APC costimulatory proteins with T cell costimulatory proteins—are required to drive T cell specificity and activation or inhibition. The TCR is specific for a given epitope; however, costimulatory proteins are not epitope specific, and instead are generally expressed on all T cells or on subsets of cells.

APCs generally serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived (infectious agent-derived), tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors. Conversely, the broad, non-specific activation of overly active T cell responses against self-antigens or shared antigens can give rise to T cells that inappropriately attack and destroy healthy tissues or cells.

Human MHC proteins are referred to as human leukocyte antigens (HLA) in humans. HLA proteins are divided into two major classes, Class I and Class II proteins, which are encoded by separate loci. Unless expressly stated otherwise, for the purpose of this disclosure, references to MHC or HLA Class I proteins. HLA Class I proteins each comprise a heavy chain (sometimes denoted “MHC-H”) encoded by gene loci including the classical HLA-A, HLA-B, HLA-C, and non-classical HLA-E, HLA-F and HLA-G loci. HLA Class I proteins also include a light chain protein, the β-2 microglobulin (β2M) polypeptide.

### II. SUMMARY

The present disclosure provides multimeric antigen-presenting polypeptide complexes (“MAPP” singular and “MAPPs” plural) that are at least heterodimeric and include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (See FIG. 1A). Framework polypeptides also comprise a multimerization sequence(s) that permits two or more framework polypeptides to associate, thereby forming a higher order structure (e.g., a duplex of the two or more heterodimers, a “duplex MAPP” see, e.g., FIGS. 1A and 1B). Neither the dimerization sequence nor the multimerization sequence of the framework polypeptide (or the counterpart dimerization sequence) comprises an MHC-H polypeptide sequence or a β2M polypeptide sequence; and as such, interaction brought about by those sequences are not consider dimerization or multimerization of framework and/or dimerization peptides. Accordingly, the framework polypeptides provide a structure upon which other polypeptides (e.g., immunomodulatory and/or MHC polypeptides) can be organized by interactions at the dimerization sequences, and which can interact with other framework polypeptides by way of multimerization sequences.

The framework and dimerization peptide containing MAPPs, duplex MAPPs, and MAPPs of higher order (e.g., triplex MAPPs) described herein provide a means by which epitope-presenting peptides (“peptide epitopes” or simply “epitopes”) may be presented in the context of an MHC (e.g., HLA) to a target T cell displaying a TCR specific for the epitope, while at the same time permitting for the flexible presentation of one or more immunomodulatory polypeptides (“MODs”). The MAPPs, duplex MAPPs, and higher order MAPPs thereby permit delivery of one or more MODs in an epitope selective (e.g., dependent/specific) manner that permits (i) formation of an active immune synapse with a target T cell selective for the epitope, and (ii) modulation (e.g., control/regulation) of the target T cell's response to the epitope.

The presentation by a MAPP of a peptide epitope to a target T cell is accomplished via a moiety that comprises MHC Class I polypeptides and the peptide epitope. Such moieties may be either (i) a single polypeptide chain, or (ii) a complex comprising two or more polypeptide chains.

Where the peptide epitope, an MHC-H and β2M polypeptide sequences, and optionally one or more MODs are provided in one polypeptide, it is termed an “epitope presenting sequence” or “presenting sequence.” See, e.g., FIG. 12. MAPPs typically contain one or two presenting sequence, and accordingly, duplex MAPPS typically comprise one, two, three, four presenting sequences. The presenting sequences may be integrated into a MAPP as part of a framework polypeptide or a dimerization polypeptide. A MAPP may have presenting sequences as part of either or both of framework or dimerization polypeptide. Compare, for example, FIG. 6 structures A-D and FIG. 7 structures A-D.

As an alternative to utilizing a single polypeptide to present an epitope, the MHC components (MHC-H polypeptide and β2M polypeptide sequences) and the epitope may be divided (split) into two separate polypeptide sequences, which together are denoted herein as an “epitope presenting complex” or “presenting complex.” A presenting complex is integrated into a MAPP by having a presenting complex first amino acid sequence (“presenting complex 1^{st} sequence”) as part of a framework or dimerization polypeptide. The remaining MHC sequence(s) are part of a polypeptide termed the presenting complex second amino acid sequence (“presenting complex 2^{nd} sequence”). The peptide epitope and any independently selected MODs that are present may be part of the polypeptide comprising either the presenting complex 1^{st} sequence or the presenting complex 2^{nd} sequence. The presenting complex 1^{st} sequence and presenting complex 2^{nd} sequence generally associate through non-covalent interactions between the MHC-H chain and the β2M polypeptide sequence, and may be stabilized by disulfide bonds between either the MHC sequences or peptide/polypeptide linkers attached to the N- or C-terminus of the MHC sequences. The presenting complex 1^{st} sequence and presenting complex 2^{nd} sequence may also associate through dimerization or interspecific dimerization sequences if present in those polypeptides.

Although an individual MAPP may not comprise a presenting sequence or presenting complex, for the purpose of this disclosure the MAPPs are, unless stated otherwise, understood to comprise at least one presenting sequence or presenting complex.

MAPPs that comprise a presenting sequence typically contain one or two presenting sequences. Duplex MAPPS thus typically comprise two, three or four presenting sequences, but also may comprise one presenting sequence (e.g., if one of the MAPPS does not comprise a presenting sequence). MAPPs and duplex MAPPs may comprise more presenting sequences depending on, for example, the number of dimerization sequences in the framework polypeptide. The presenting sequences may be integrated into a MAPP as part of a framework polypeptide, a dimerization polypeptide, or both. Compare, for example, FIG. 6 structures A-D and FIG. 7 structures A-D.

Likewise, MAPPs with presenting complexes typically contain one or two presenting complexes, and accordingly, duplex MAPPs with presenting complexes typically comprise two, three or four presenting complexes, but also may comprise one presenting complex (e.g., if one of the MAPPs does not comprise a presenting complex). As discussed above, MAPPs and duplex MAPPs may comprise more presenting complexes depending on, for example, the number of dimerization sequences in the framework polypeptide.

MAPPs and accordingly their higher order complexes (duplexes, triplexes etc.) comprising MHC Class I polypeptide sequences and a peptide epitope for presentation to a TCR, may present peptides to T cells (e.g., CD8+ T cells) that have a TCR specific for the epitope. Once engaged with the TCR of a T cell, the effect of a MAPP on the T cell depends on which MODs, if any, are present as part of the MAPP.

MOD-containing MAPPs can function as a means of selectively delivering the MODs to T cells specific for the MAPP-associated epitope, thereby resulting in MOD-driven responses to those MAPPs (e.g., proliferation, reduction in number and/or suppression of T cells specific for the MAPP-associated epitope). Depending on the chosen MOD, the incorporation of one or more MODs with increased affinity for their cognate receptor on T cells (“co-MOD”) may reduce the specificity of MAPPs and duplex MAPPs for epitope specific T cells where MOD—co-MOD binding interactions significantly compete with MHC/epitope binding to target cell TCR. Conversely, and again depending on the chosen MOD, the inclusion of MODs with reduced affinity for their co-MOD(s), and the affinity of the epitope for a TCR, may provide for enhanced selectivity of MAPPs and duplex MAPPs, while retaining the desired activity of the MODs. Where a MOD already possesses a relatively low affinity for its cognate receptor, mutations that reduce the affinity may be unnecessary and/or undesirable.

The ability of MAPPs (e.g., duplex MAPPs) to modulate T cells provides methods of modulating T cell activity in vitro and in vivo, and accordingly the use of MAPPs as therapeutics useful in methods of treating a variety of diseases and conditions including cancers, autoimmune diseases, and infectious diseases.

The present disclosure provides nucleic acids comprising nucleotide sequences and vectors encoding individual MAPP polypeptides and MAPPs (e.g., all polypeptides of a MAPP), as well as cells genetically modified with the nucleic acids and vectors for producing individual MAPP polypeptides and/or MAPP proteins (e.g., duplex MAPPs). The present disclosure also provides methods of producing MAPPs, duplex MAPPs, and higher order MAPPs utilizing such cells.

### III. BRIEF DESCRIPTION OF THE DRAWINGS

FIG. 1A is provided to illustrate of the terminology used to describe MAPPs and duplex MAPPs with presenting sequences. The peptides are oriented from N-terminus (left) to C-terminus (right). The figure shows first and second framework polypeptides, which in this case are different and, in this instance, have specific multimerization sequences comprising a knob and counterpart hole. Such “knob-in-hole” configurations may include knob-in-hole configurations without a stabilizing disulfide bond (herein “KiH”) or with a stabilizing disulfide bond (herein “KiHs-s”). Also shown are first and second dimerization polypeptides having an N-terminal epitope and counterpart dimerization sequences. The dashed circles indicate five potential locations for the addition of polypeptide sequences, including MODs (discussed below). The figure depicts the formation of a first and second heterodimer MAPPs, each comprising a framework polypeptide and dimerization polypeptide. The heterodimers may interact through the multimerization sequence to form a multimer (a duplex MAPP as shown). The use of knob-in-hole sequences permit the assembly of an asymmetric interspecific duplex MAPP where, for example, different MOD sequences are provided at positions 1 and 1′ and/or positions 3 and 3′. While interactions between polypeptide chains through peptide interaction sequences may initially be non-covalent in nature, interchain disulfide bond formation reactions may occur thereby providing covalently linked polypeptides at, for example, either dimerization sequences or multimerization sequences. Throughout the figures, lines connecting various elements of MAPP polypeptides are optional amino acids serving as linkers (e.g., peptide linkers).

FIG. 1B parallels FIG. 1A and is provided to illustrate of the terminology used to describe MAPPs and duplex MAPPs with presenting complexes. The word “sequence” may be abbreviated by “seq.”.

FIG. 2 provides a multiple aa sequence alignment of β2M precursors (i.e., including the leader sequence) from Homo sapiens (NP_004039.1; SEQ ID NO:1), Pan troglodytes (NP_001009066.1; SEQ ID NO:2), Macaca mulatta (NP_001040602.1; SEQ ID NO:3), Bos taurus (NP_776318.1; SEQ ID NO:4) and Mus musculus (NP_033865.2; SEQ ID NO:5). Underlined aas 1-20 are the signal peptide (sometime referred to as a leader sequence) which is absent in the mature protein, and unless stated otherwise is not present in MAPP polypeptides.

FIGS. 3A, 3B and 3C provide aa sequences of HLA Class I heavy chain polypeptides. Signal sequences, aas 1-24, are bolded and underlined. FIG. 3A entry: 3A.1 is the HLA-A heavy chain (HLA-A*01:01:01:01 or A*0101) (NCBI accession NP_001229687.1), SEQ ID NO:6; entry 3A.2 is ‘HLA-A*1101, SEQ ID NO:7; entry 3A.3 is HLA-A*2402, SEQ ID NO:8, and entry 3A.4 is HLA-A*3303, SEQ ID NO:9. FIG. 3B provides the sequence for HLA-B*07:02:01 (HLA-B*0702) (NCBI GenBank Accession NP_005505.2 (see, also GenBank Accession AUV50118.1), SEQ ID NO:10). FIG. 3C provides the sequence for HLA-C*0701 (GenBank Accession NP_001229971.1) (HLA-C*07:01:01:01 or HLA-Cw*070101), SEQ ID NO:11, (HLA-Cw*07) (see GenBank Accession CA078194.1).

FIG. 3D provides an alignment of eleven mature MHC Class I heavy chain peptide sequences without all, or substantially all, of their leader, transmembrane and intracellular domain regions. The aligned sequences include human HLA-A*0101, SEQ ID NO:12 (see also SEQ ID NO:6); HLA-B*0702, SEQ ID NO:13 (see SEQ ID NO:10); HLA-C, SEQ ID NO:14 (see SEQ ID NO:11); HLA-A*0201, SEQ ID NO:15; a mouse H2K protein sequence, SEQ ID NO:16; three variants of HLA-A (var.2, var.2C, and var.2CP, SEQ ID NOs:17, 18, and 19); and 3 human HLA-A polypeptides (HLA-A*1101 (HLA-A11), SEQ ID NO:20; HLA-A*2402 (HLA-A24), SEQ ID NO:21; and HLA-A*3303 (HLA-A33), SEQ ID NO:22)). HLA-A*0201 is a variant of the sequence marked as HLA-A. Marked as HLA-A(var. 2) is the Y84A and A236C variant of HLA-A. The seventh HLA-A sequence, marked as HLA-A (var. 2C), shows the HLA-A sequence substituted with C residues at positions 84, 139 and 236, and the 8th sequence adds one additional proline to the C-terminus of the preceding sequence. The 9th through the 11th sequences are from HLA-A11 (HLA-A*1101), HLA-A24 (HLA-A*2402); and HLA-A33 (HLA-A*3303), respectively, which are prevalent in certain Asian populations. Indicated in the alignment are aa locations (aas 84 and 139 of the mature proteins) where cysteine residues may be inserted in place of the wt. aas to form an intrachain stabilizing disulfide bond that stabilizes the MHC-H peptide epitope binding pocket, particularly when the peptide epitope is not present or does not bind the MHC pocket with high affinity. Also shown in the alignment is position 236 (of the mature polypeptide), which may be replaced by a cysteine residue that can form an interchain disulfide bond with β2M (e.g., at aa 12 of the mature polypeptide). An arrow appears above each of those locations and the residues are bolded. The boxes flanking residues 84, 139 and 236 show the groups of five aas on either side of those six sets of five residues, denoted aa cluster 1, aa cluster 2, aa cluster 3, aa cluster 4, aa cluster 5, and aa cluster 6 (shown in the figure as aac1 through aac6, respectively). Any one or more of those groups of aas may be replaced by 1 to 5 amino acids selected independently from (i) any naturally occurring amino acid or (ii) any naturally occurring amino acid except proline or glycine.

FIGS. 3E-3G provide alignments of the amino acid sequences of mature HLA-A, -B, and, -C Class I heavy chains, respectively. The sequences are provided for a portion of the mature proteins (without all or substantially all of their leader sequences, transmembrane domains or intracellular domains). As described in FIG. 3D, the positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine are also shown. A consensus sequence is also provided for each group of HLA alleles provided in the figures showing the variable aa positions as “X” residues sequentially numbered and the locations of aas 84, 139 and 236 double underlined.

FIG. 3H provides a consensus sequence for each of HLA-E, -F, and -G with the variable aa positions indicated as “X” residues sequentially numbered and the locations of aas 84, 139 and 236 double underlined.

FIG. 3I provides an alignment of the consensus aa sequences for HLA-A, -B, -C, -E, -F, and -G, which are given in FIGS. 3E to 3H. The alignment shows the correspondence of amino acids between the different sequences. Variable residues in each sequence are listed as “X” with the sequential numbering removed. The permissible amino acids at each variable residue can be determined by reference to FIGS. 3E-3H As indicated in FIG. 3D, the locations of aas 84, 139 and 236 with their flanking five-amino acid clusters that may be replaced by 1 to 5 amino acids selected independently from (i) any naturally occurring amino acid or (ii) any naturally occurring amino acid except proline or glycine are also shown.

FIGS. 4A-4H provides partial amino acid sequences of immunoglobulin polypeptides including their heavy chain constant regions (“Ig Fc” or “Fc”, e.g., the CH2-CH3 domain of IgG1) SEQ ID NOs:54 to 66).

FIG. 4I provides the sequence of an immunoglobulin heavy chain region 1 (CH1) or Ig CH1 domain (SEQ ID NO:67).

FIG. 4J provides the sequence of a human immunoglobulin J chain precursor, NCBI accession No. NP_653247.1 (SEQ ID NO:68).

FIG. 5A provides the sequence of an immunoglobulin (“Ig”) light chain constant region (“C_{L}”) or “Ig C_{L}” from a human kappa light chain (Ig C_{L} κ chain) (SEQ ID NO:69).

FIG. 5B provides the sequence of an Ig lambda light chain constant region Ig C_{L} λ chain (SEQ ID NO:70).

FIG. 6 provides a series of duplex MAPP structures based on framework polypeptides having both a multimerization sequence and first and second dimerization sequences that may be the same or different. The structure is shown generically in A with locations 1-5 and 1′-5′ indicating locations for additional peptide sequences (e.g., MOD polypeptide sequences). The MHC/epitope moiety is illustrated generically, and can be either a presenting sequence (see, e.g., FIG. 12), or a presenting complex (see FIGS. 13-15). Locations 4 and 4′ are shown at the N-terminus of a presenting sequence or the N-terminus of a presenting complex polypeptide, and locations 5 and 5′ are shown at the C-termini of those polypeptides. Locations 1 and 1′ are shown at the N-terminus of the framework peptide and locations 3 and 3′ at the C-terminus of the framework polypeptide. In A and C, the framework polypeptides are multimerized to form a duplex of heterodimers via non-covalent binding between the multimerization sequences. In B and D, the framework polypeptides are multimerized to form a duplex of heterodimers using an immunoglobulin Fc region knob-in-hole motif, although other methods of non-covalently or covalently bonding the multimerization sequences may be used. In C the duplexes contain heterodimers in which two different asymmetric interspecific dimerization sequences bind together the framework peptides and their associated dimerization peptides. In D the framework peptides are joined together by a knob-in-hole Fc motif and the dimerization peptide and framework peptide are joined together by different dimerization sequences to form a duplex of heterodimers.

FIG. 7 provides in A to D a series of MAPP structures as in FIG. 6, with the addition of presenting sequences or presenting complexes at the N-terminus of the framework peptides. Positions 4 and 4′ may still serve as locations for peptide addition (e.g., MOD polypeptide addition).

FIG. 8 provides in A to D a series of MAPP structures as in FIG. 6, where the dimerization sequences are Ig CH1 sequences (CH1) that pair with Ig light chain κ or λ sequences (CL). The framework peptides are multimerized (dimers in this instance) through the interaction of Ig Fc (e.g., CH2 and CH3) regions, with the structures in B and D having knob-in-hole motifs to permit heteroduplexes to be formed. The peptides are also joined by disulfide bonds (e.g., those that form between Ig Fc region peptides).

FIG. 9 provides a series of MAPP structures as in FIG. 8, with the addition of presenting sequences or presenting complexes at the N-terminus of the framework peptides. Positions 4 and 4′ may still serve as locations for peptide addition (e.g., MOD polypeptide addition).

FIG. 10 provides in A to J a series of MAPP structures as in FIG. 8. In each instance a presentation sequence lacking a MOD sequence is present on the dimerization peptide (marked as a single chain MHC and epitope). Locations 2, 2′, 4, 4′, 5 and 5′are unfiled and not shown. Locations 1 and 1′ are substituted with one or more MODs selected from IL-2b, PD L1, and CD80. Positions 3 and 3′ are shown for orientation in A to G. In H to J the 3 and 3′ locations are either unfiled or, for example, a TGF-β or 4-1BBL MOD may be located there.

FIG. 11 provides examples of duplex MAPPs epitope presenting sequences detailed (the “Single Chain MHC” with the epitope as part of the structure. Although shown with the specific epitope ELAGIGILTV, that peptide epitope may be replaced by any other translatable peptide epitope.

FIG. 12 shows in A and B two different MHC Class I presenting sequences (from the epitope at the N-terminus to C-terminus. In the figures (e.g., FIGS. 12 to 15) the symbol “-//-” indicates a point of connection with a dimerization peptide or a framework peptide to form a single peptide sequence. The sequences optionally comprise one or more independently selected MODs (including MODs in tandem) at the locations indicated (e.g., at the N-terminus or between the recited elements, such as in a linker polypeptide sequences).

FIGS. 13 to 15 show a series of MHC Class I presenting complexes from N- to C-terminus. The sequence bearing the symbol “-//-” is the presenting complex 1^{st} polypeptide sequence (presenting complex 1^{st} sequence). The other sequence the presenting complex 2^{nd} sequence that is associated with the presenting complex 1^{st} sequence. The symbol “-//-” also indicates a point of connection with a dimerization peptide or a framework peptide. In FIG. 15 the presenting complex 1^{st} sequence and its associated presenting complex 2^{nd} sequence are covalently attached by the formation of a disulfide bond.

FIGS. 16A to 16D provide the polypeptide sequences of the MAPP constructs in Example 1.

FIG. 17 provides the polypeptide sequences of the MAPP constructs in Example 2.

### IV. DETAILED DESCRIPTION

A. Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids having a three-dimensional configuration, which unless stated otherwise are the naturally occurring proteinogenic L-amino acids that are incorporated biosynthetically into proteins during translation in a mammalian cell. Furthermore, as used herein, a “polypeptide” and “protein” include modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References to a specific residue or residue number in a known polypeptide, e.g., position 16 or 42 of human IL-2 polypeptide, are understood to refer to the amino acid at that position in the wild-type polypeptide (i.e. H16 or F42). To the extent that the sequence of the wild-type polypeptide is altered, either by addition or deletion of one or more amino acids, the specific residue or residue number will refer to the same specific amino acid in the altered polypeptide (e.g., in the addition of one amino acid at the N-terminus of a peptide reference as position H16, will be understood to indicate the amino acid, His, that is now position 16). Substitution of an amino acid at a specific position is denoted by an abbreviation comprising, in order, the original amino acid, the position number, and the substituted amino acid, e.g., substituting the His at position 16 with a Ala is denoted as H16A.

A nucleic acid or polypeptide has a certain percent “sequence identity” to another polynucleotide acid or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including blast.ncbi.nlm.nih.gov/Blast.cgi for BLAST+2.10.0, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10. Unless otherwise indicated, the percent sequence identities described herein are those determined using the BLAST program. In the event of a conflict between the results produced by different release versions of BLAST, BLAST+2.10.0 released Dec. 23, 2019, is employed as the basis for determining sequence identity.

As used herein amino acid (“aa” singular or “aas” plural) means the naturally occurring proteogenic amino acids incorporated into polypeptides and proteins in mammalian cell translation. Unless stated otherwise: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan) Amino acid also includes the amino acids hydroxyproline and selenocysteine, which appear in some proteins found in mammalian cells, however, unless their presence is expressly indicated they are not understood to be included.

As used herein the term “in vivo” refers to any process or procedure occurring inside of the body, e.g., of a patient.

As used herein, “in vitro” refers to any process or procedure occurring outside of the body.

The term “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

The term “binding” refers to a direct association between molecules and/or atoms, due to, for example, covalent, electrostatic, hydrophobic, and ionic and/or hydrogen-bond interactions, including interactions such as salt bridges and water bridges.

Non-covalent interactions/binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Covalent bonding,” or “covalent binding” as used herein, refers to the formation of one or more covalent chemical bonds between two different molecules. The term “binding,” as used with reference to the interaction between a MAPP and a T cell receptor (TCR) on a T cell, refers to a non-covalent interaction between the MAPP and TCR.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen. Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 40-fold greater, at least 60-fold greater, at least 80-fold greater, at least 100-fold greater, or at least 1,000-fold greater, or more, than the affinity of an antibody or receptor for an unrelated aa sequence (e.g., ligand). Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

“T cell” includes all types of immune cells expressing CD3, including T-helper cells (CD4⁺ cells), cytotoxic T cells (CD8⁺ cells), T-regulatory cells (Treg), and NK-T cells.

The term “immunomodulatory polypeptide” (also referred to as a “costimulatory polypeptide” or, as noted above, “MOD”), as used herein includes a wild-type or variant of a polypeptide or portion thereof that can specifically bind a cognate co-immunomodulatory polypeptide (“co-MOD”) present on a T cell, and provide a modulatory signal to the T cell when the TCR of the T cell is engaged with an MHC-epitope moiety that is specific for the TCR. Unless stated otherwise the term “MOD” includes wild-type and/or variant MODs, and statements including reference to both wild-type and variant MODs are made to emphasize that one, the other, or both are being referenced. The signal provided by the MOD engaging its co-MOD mediates (e.g., directs) a T cell response. Such responses include, but are not limited to, proliferation, activation, differentiation, suppression/inhibition of proliferation, activation and/or differentiation, and the like. A MOD can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, lymphotoxin beta receptor, 3/TR6, ILT3, ILT4, HVEM, an agonist or antibody that binds Toll ligand receptor and a ligand that specifically binds with B7-H3. A MOD also encompasses, inter alia, an antibody or antibody fragment that specifically binds with and activates a cognate co-stimulatory molecule (co-MOD) present on a T cell, such as, but not limited to antibodies against the receptors for any of IL-2, CD27, CD28, 4-1BB, OX40, CD30, CD40, PD-1, ICOS, lymphocyte function-associated antigen-1 (LFA-1), CD2, LIGHT, NKG2C, B7-DC, B7-H2, B7-H3, and CD83.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “purifying”, “isolating”, and the like, refer to the removal of a desired substance, e.g., a MAPP, from a solution containing undesired substances, e.g., contaminates, or the removal of undesired substances from a solution containing a desired substance, leaving behind essentially only the desired substance. In some instances, a purified substance may be essentially free of other substances, e.g., contaminates. Purifying, as used herein, may refer to a range of different resultant purities, e.g., wherein the purified substance makes up more than 80% of all the substance in the solution, including more than 85%, more than 90%, more than 91%, more than 92%, more than 93%, more than 94%, more than 95%, more than 96%, more than 97%, more than 98%, more than 99%, more than 99.5%, more than 99.9%, and the like. As will be understood by those of skill in the art, generally, components of the solution itself, e.g., water or buffer, or salts are not considered when determining the purity of a substance.

Where a range of values is provided, it is understood that each intervening value between the upper and lower limit of that range to a tenth of the lower limit of the range is encompassed within the disclosure along with any other stated or intervening value in the range. The upper and lower limits of these smaller ranges may independently be included in smaller ranges, that are also encompassed within the disclosure subject to any specifically excluded limit in the stated range. Where the stated range a value (e.g., an upper or lower limit), ranges excluding those values are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Treg” includes a plurality of such Tregs and reference to “the MHC Class I heavy chain” includes reference to one or more MHC Class I heavy chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

B. Description

1. MAPP Structure and the Role of Framework and Dimerization Peptides

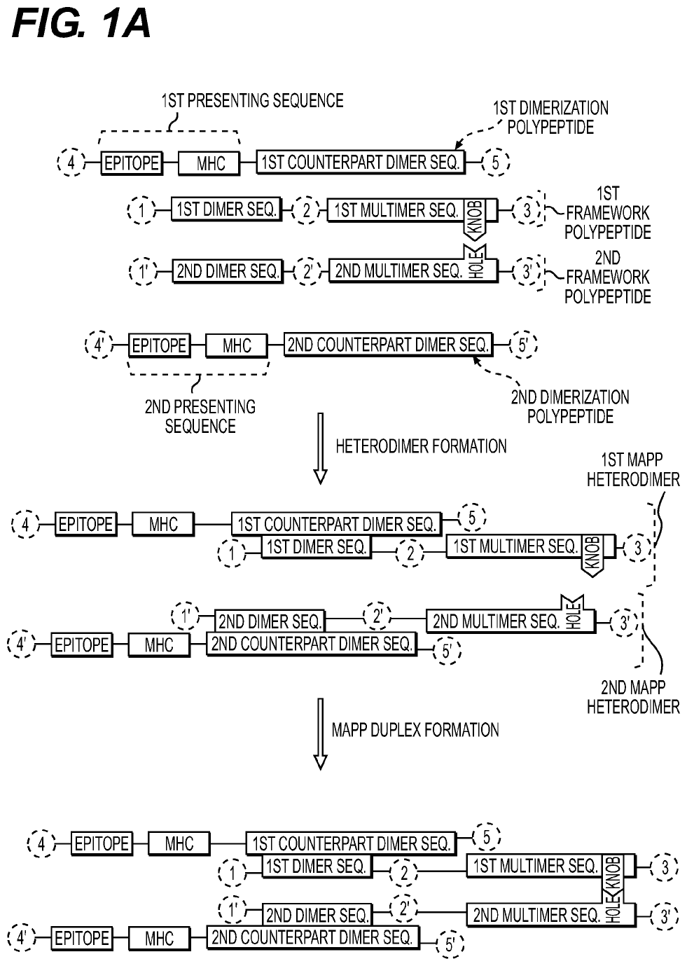

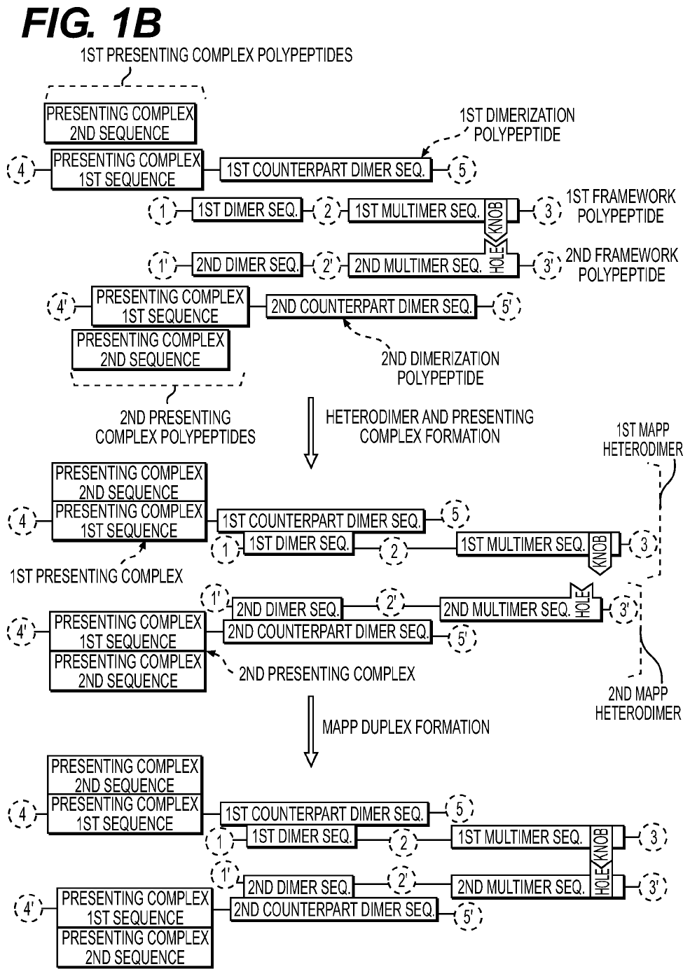

The present disclosure provides MAPPs for, among other things, use in the treatment of disease and disorders including cancers, autoimmune diseases, and infectious diseases. As discussed above, the MAPPs include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (see FIGS. 1A and 1B). Framework polypeptides also comprise a multimerization sequence(s) that permits two or more framework polypeptides to associate, thereby forming a higher order structure (e.g., a duplex of the two or more heterodimers, a “duplex MAPP” see, e.g., FIGS. 1A and 1B). Neither the dimerization sequence nor the multimerization sequence of the framework polypeptide (or the counterpart dimerization sequence) comprises an MHC-H polypeptide sequence or a β2M polypeptide sequence; and as such, interaction brought about by those sequences are not consider dimerization or multimerization of framework and/or dimerization peptides. Accordingly, the framework polypeptides provide a structure upon which other polypeptides can be organized by interactions at the dimerization sequences, and which can interact with other framework polypeptides by way of multimerization sequences. The terms “MAPP” and “MAPPs” as used herein will be understood to refer in different contexts to the heterodimer comprising a framework and dimerization peptide structure as well as higher order complexes of those MAPP heterodimers, such as duplexes (duplex MAPPs). It will be clear to the skilled artisan when specific reference to only higher order structures are intended (e.g., by reference to duplex MAPPs, etc.).

As discussed above, the framework and dimerization peptide containing MAPPs, duplex MAPPs, and MAPPs of higher order (e.g., triplex MAPPs) described herein provide a means by which peptide epitopes may be delivered in the context of MHC (e.g., HLA) polypeptides to a target T cell displaying a TCR specific for the epitope, while at the same time permitting for the flexible presentation of one or more MODs. The MAPPs, duplex MAPPs, and higher order MAPPs thereby permit deliver of one or more MODs in an epitope selective (e.g., dependent/specific) manner that permits formation of an active immune synapse with a target T cell selective for the epitope, and control/regulation of the target T cell's response to the epitope. Accordingly, where MAPPs comprise stimulatory or activating MODs (e.g., IL-2, CD80, CD86, and/or 4-1BBL) that increase T cell proliferation and/or effector functions in an epitope selective manner In contrast, where MAPPs comprise suppressive/inhibitory MODs (e.g., FasL and/or PD-L1) they decrease T cell activation, proliferation, differentiation, and/or effector functions in an epitope selective manner

The framework/dimerization polypeptide architecture of MAPPs and their higher order structures may also be understood to provide flexibility in locating MODs and epitope presenting complexes or epitope presenting sequences. Duplex MAPP and higher order MAPP architecture can be particularly useful when both the MOD and the epitope presenting complexes (or epitope presenting sequences) are positioned so as to provide the desired biological activity as well as other desired properties of the MAPP, e.g., thermal stability and manufacturability. In some cases, acceptable combinations of properties may be obtained when the MOD and presenting complex or presenting sequence are positioned at the N-terminus of a polypeptide, e.g., each may be located at the N-terminus of different framework and/or dimerization polypeptide sequences. In some cases, acceptable combinations of properties may be obtained when the MOD and presenting complex or presenting sequence are positioned at the C-terminus of a polypeptide, e.g., each may be located at the C-terminus of different framework and/or dimerization polypeptide sequences. In some cases, acceptable combinations of properties may be obtained when the MOD and presenting complex or presenting sequence are positioned at the N-terminus and C-terminus of a polypeptide, respectively, e.g., the MOD may be located at the N-terminus and the presenting complex or presenting sequence may be located at the C-terminus of different framework and/or dimerization polypeptide sequences. In some cases, acceptable combinations of properties may be obtained when the MOD and presenting complex or presenting sequence are positioned at the C-terminus and N-terminus of a polypeptide, respectively, e.g., the MOD may be located at the C-terminus and the presenting complex or presenting sequence may be located at the N-terminus of different framework and/or dimerization polypeptide sequences.

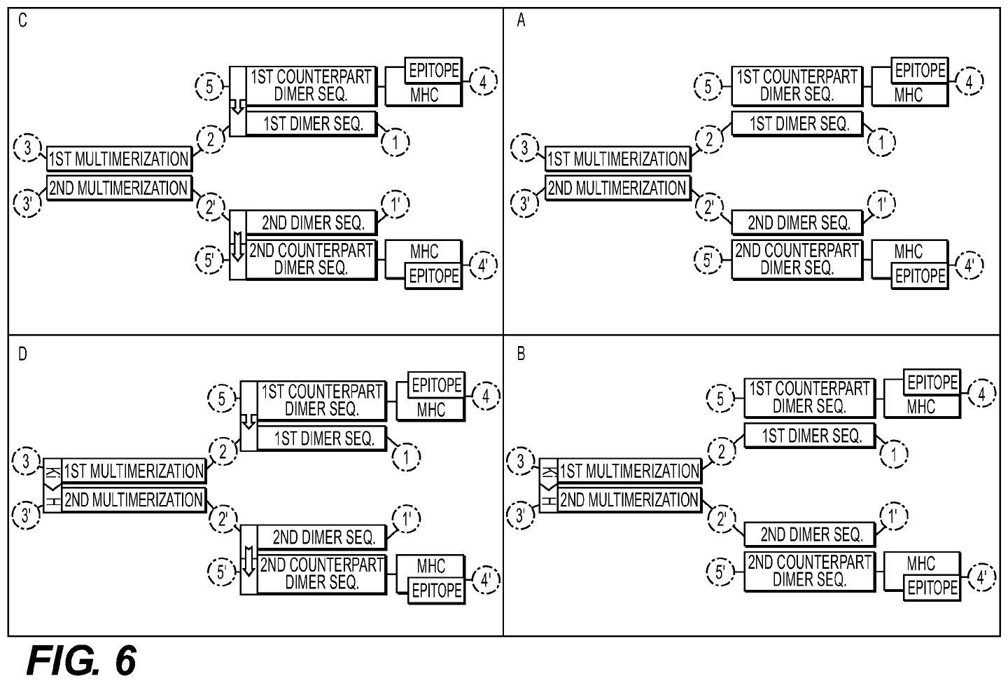

The structure of MAPPs, and particularly higher order MAPPs such as duplexes, may be specified by the use of pairs of polypeptides having different sequences that specifically pair with each other. Multimerization of framework polypeptides results from interactions between multimerization sequences, and dimerization (the interaction of a framework and dimerization polypeptide) results from the interaction of a dimerization sequence on the framework polypeptide and a counterpart dimerization on a dimerization polypeptide. For example, in a duplex MAPP the multimerization sequences may be Ig Fc heavy chain (e.g., CH2-CH3) sequences, and the dimerization sequence and counterpart dimerization sequences may be the same (e.g., all leucine zipper sequences). An additional degree of control may be obtained by utilizing non-identical peptide sequences that specifically/selectively pair with each other that are referred to herein generally as “interspecific sequences,” in the case of dimerization sequences “interspecific dimerization sequences,” or in the case of multimerization sequences “interspecific multimerization sequences,” and which give rise to asymmetric interspecific pairs of sequences. The structure of MAPPs thus permits diverse and effective placement of each polypeptide into the MAPP architecture MAPP (see e.g., FIGS. 6-10). Interspecific sequences include Ig heavy chain Fc (e.g., CH2-CH3) region modified with, for example, knob-in-hole variations; and Fos peptide sequences paired with Jun peptide sequences. Accordingly, MAPP architectures include, but are not limited to, MAPPs where each, or some, of the dimerization sequences are different (permit different peptide pairings). For example, in duplex MAPPs where each of the multimerization and dimerization sequence are different and provides separate peptide pairings.

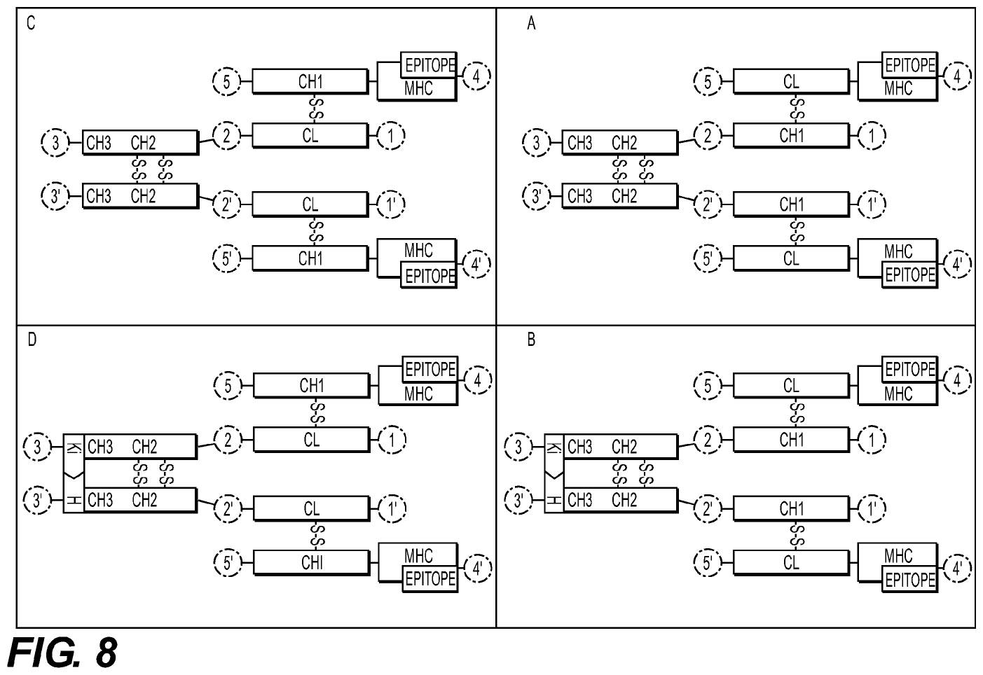

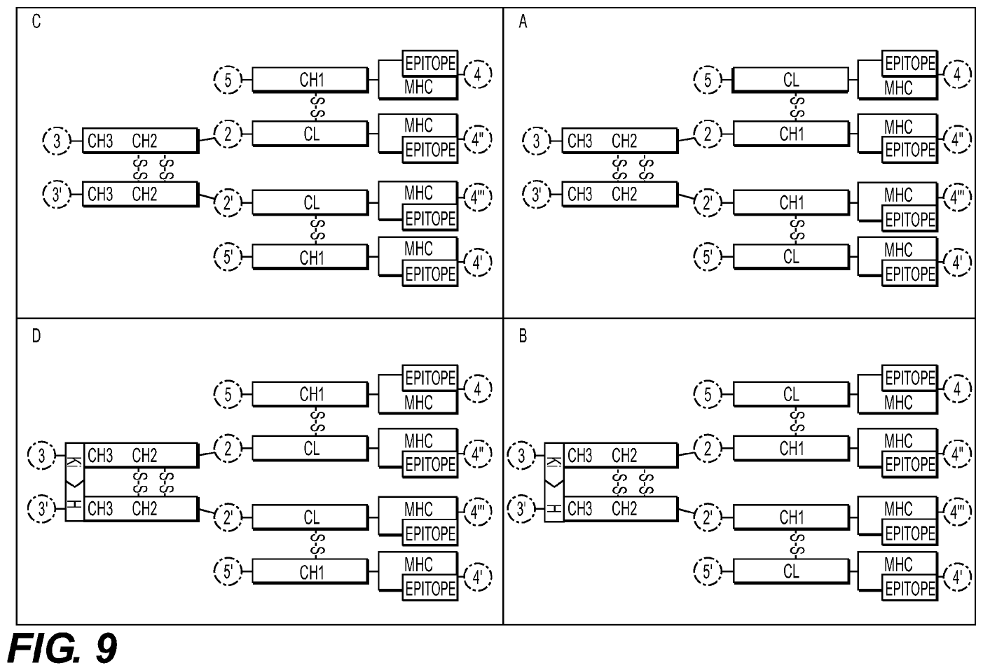

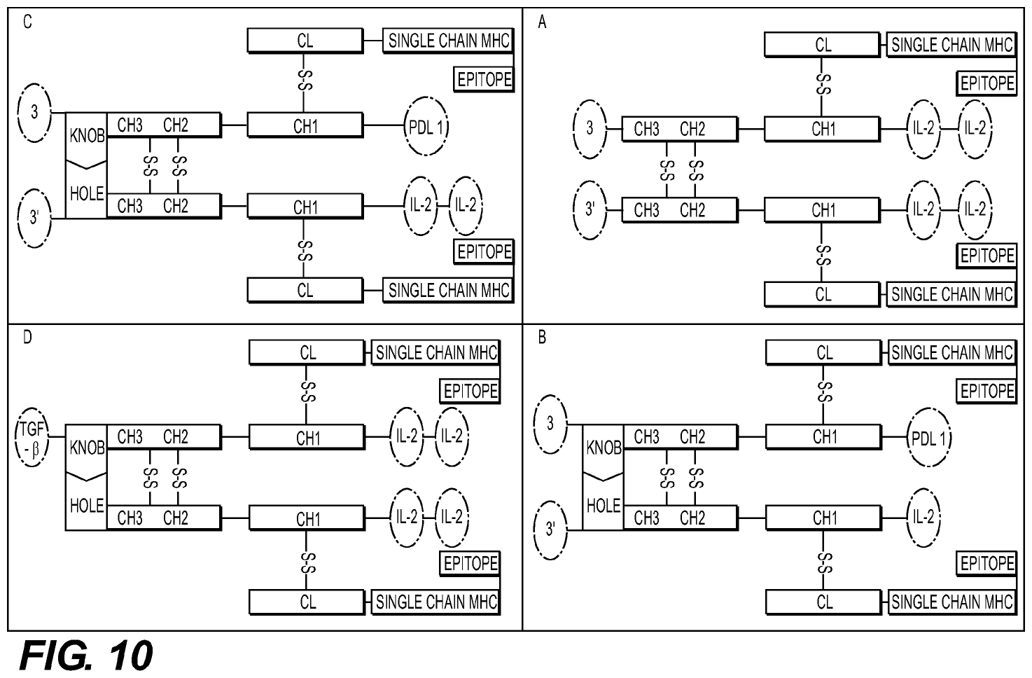

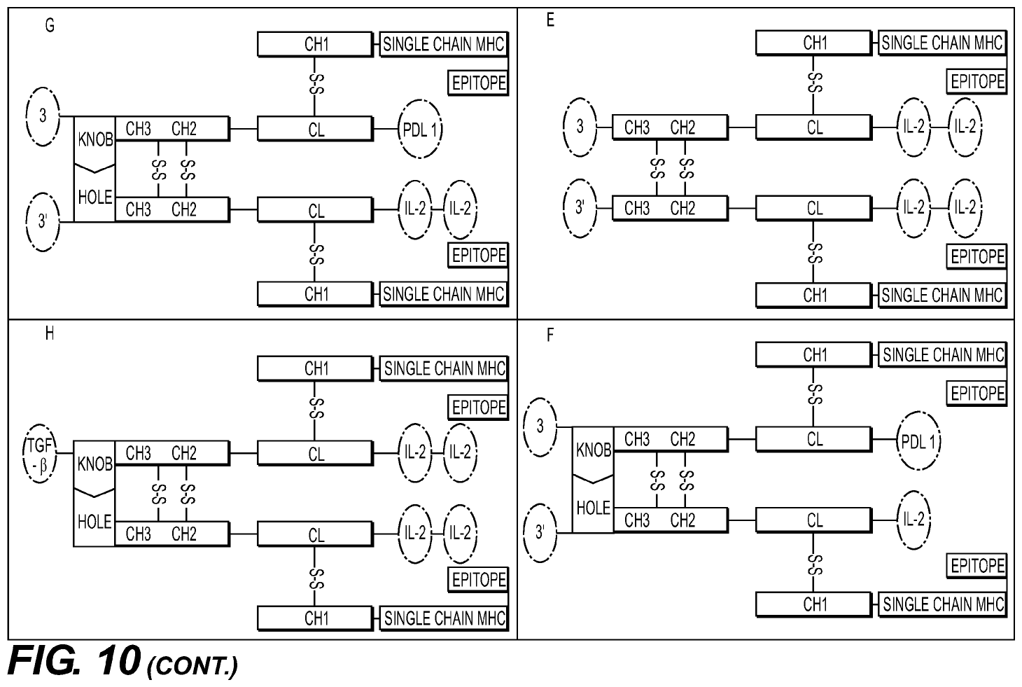

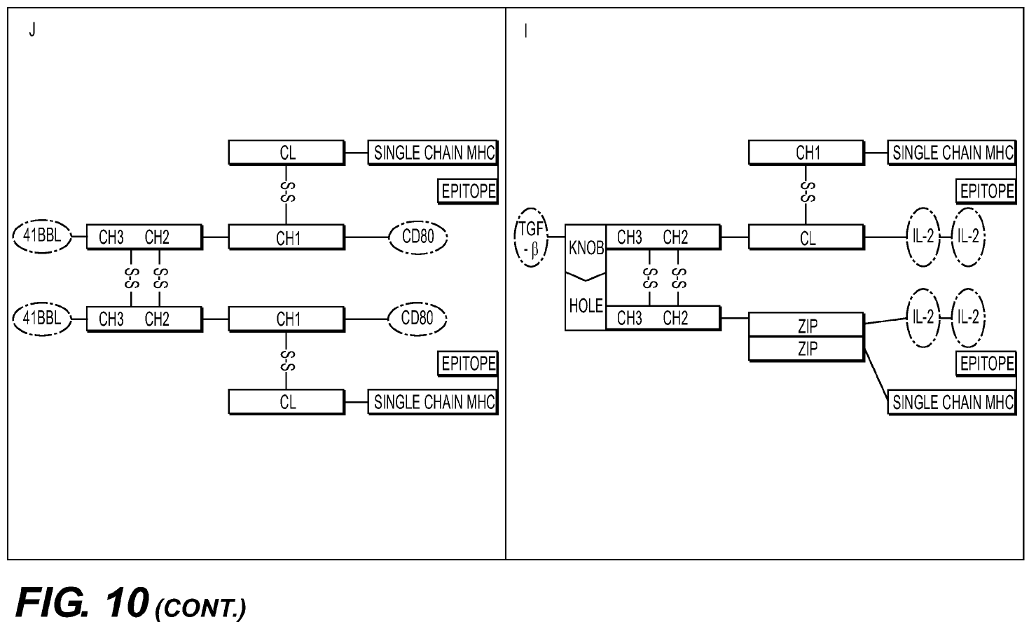

In an embodiment, the framework peptide multimerization sequence is an Fc heavy chain region (optionally a knob-in hole Fc sequence pair) and the dimerization sequences are the same (e.g., Ig CH1 sequences paired with light chain λ or κ constant region sequences) (see, for example, FIGS. 8 and 9, structures A to D). In another embodiment, the framework peptide multimerization site is an Fc heavy chain region (optionally a knob-in hole Fc sequence pair) and the dimerization sequences are selected to be different (e.g., a dimerization sequence pair comprising an Ig CH1 paired with light chain λ or κ sequence and a dimerization sequence comprising a leucine zipper pair, see for example, FIG. 10, structures E to H). For example, in a duplex MAPP the multimerization sequences may be a knob-in-hole Ig sequence, one dimerization sequence and its counterpart dimerization sequence may be leucine zipper sequences, and second dimerization sequence and its counterpart dimerization sequence may be an Ig CH1 and Ig CL λ domain pair.

MAPPs and accordingly their higher order complexes (duplexes, triplexes etc.) comprise MHC Class I polypeptide sequences that bind an epitope for presentation to a TCR, and accordingly may present peptides to T cells (e.g., CD8+ T cells). The effect of MAPPs on T cells with TCRs specific to the epitope depends on which, if any, MODs are present in the MAPP. As noted above, MAPPs, duplex MAPPS and higher order MAPPs comprising MOD(s) permit MOD delivery to T cells in an epitope selective manner and the MODs principally dictate the effect of MAPP-T cell engagement in light of the specific cell type stimulated and the environment. While not wishing to be bound by any particular theory, the effect of MAPP (e.g., duplex MAPP) presentation of MOD(s) and epitope to a T cells in some cases may be enhanced relative to the situation encountered in antigen presenting cells (APC) where epitope can diffuse away from the MHC-H/β2M complex and any MODs the APC is presenting. This situation may not occur where the epitope and MOD(s) are part of the MAPP polypeptide(s) and cannot diffuse away even if the epitope's affinity for the MHC-H/β2M complex would normally permit it to leave the comparable cell complex. The inability of epitope to diffuse away from MHC-H/β2M and MOD components of a MAPP, duplex MAPP, or higher order MAPP may be further limited where the polypeptide(s) of the MAPP (e.g., framework, dimerization sequence, and if present, the presenting complex 2^{nd} sequence) are covalently attached to each other (e.g., by disulfide bonds). Consequently, MAPPs and their higher order structures can prolong delivery of MOD(s) to T cells in an epitope selective manner relative to systems where epitope can diffuse away from the presenting MHC. In the absence of any MOD or any stimulatory MOD, prolonged exposure to the MAPP may result in T cell anergy or suppression of T cell stimulation.

Incorporation of one or more MODs with affinity for their cognate receptor on T cells (“co-MOD”) can reduce the specificity of MAPPs (e.g., duplex MAPPs) for epitope selective/specific T cells. The reduction in epitope selectivity/specificity of the MAPPs becomes more pronounced where MOD/co-MOD binding interactions increase in strength (binding energy) and significantly compete with MHC/epitope binding to target cell TCR. The inclusion of variant MODs with reduced affinity for their co-MOD(s) thus may provide a lower contribution of MOD binding energy, thereby permitting MHC-epitope interactions in which the TCR dominates the binding and provides epitope selective interactions with T cells while retaining the activity of the MODs. Variant MODs with one or more substitutions (or deletions or insertions) that reduced the affinity of the MOD for their co-MOD may be incorporated into MAPPs and their higher order complexes alone or in combination with wild-type MODs polypeptide sequences. Wild-type and variant MODs are described further below. The ability of MAPPs to modulate T cells in an epitope selective/specific manner thus provides methods of modulating activity of a T cell in vitro and in vivo, and accordingly, methods of treating disease such as cancers, infections, and disorders related to immune dysregulation/disfunction, including allergies and autoimmune diseases.

The present disclosure provides nucleic acids comprising nucleotide sequences encoding MAPP polypeptides, cells genetically modified with the nucleic acids and capable of producing the MAPP, and methods of producing MAPPs and their higher order complexes utilizing such cells.

Each presenting sequence or presenting complex present in a MAPP comprises MHC Class I heavy chain and β2M polypeptide sequences (e.g., human MHC Class I sequences) sufficient to bind a peptide epitope and present it to a TCR. MHC Class I peptides, may include sequence variations that are designed to stabilize the MHC, stabilize the MHC peptide epitope complex, and/or stabilize the MAPP. Sequence variations may also serve to enhance cellular expression of MAPPs prepared in cell-based systems as well as the stability (e.g., thermal stability) of MAPPs and their higher order complexes such as duplex MAPPs. Some MHC Class I sequences suitable for use in MAPPs are described below.

As indicated in the description of the drawings, MAPPs may comprise one or more independently selected peptide sequences or (one or more “linker” or “linkers”) between any two or more components of the MAPP, which in the figures may be shown as a line between peptide and/or polypeptide elements of the MAPPs. The same sequences used as linkers may also be located at the N- and/or C-termini of the MAPP peptides to prevent, for example, proteolytic degradation. Linker sequences include but are not limited to polypeptides comprising: glycine; glycine and serine; glycine and alanine; alanine and serine; and glycine, alanine and serine; any one which may comprise a cysteine for formation of an intra or interpeptide disulfide bond. Various linkers are described in more detail below.
2. Exemplary MAPP Architectures

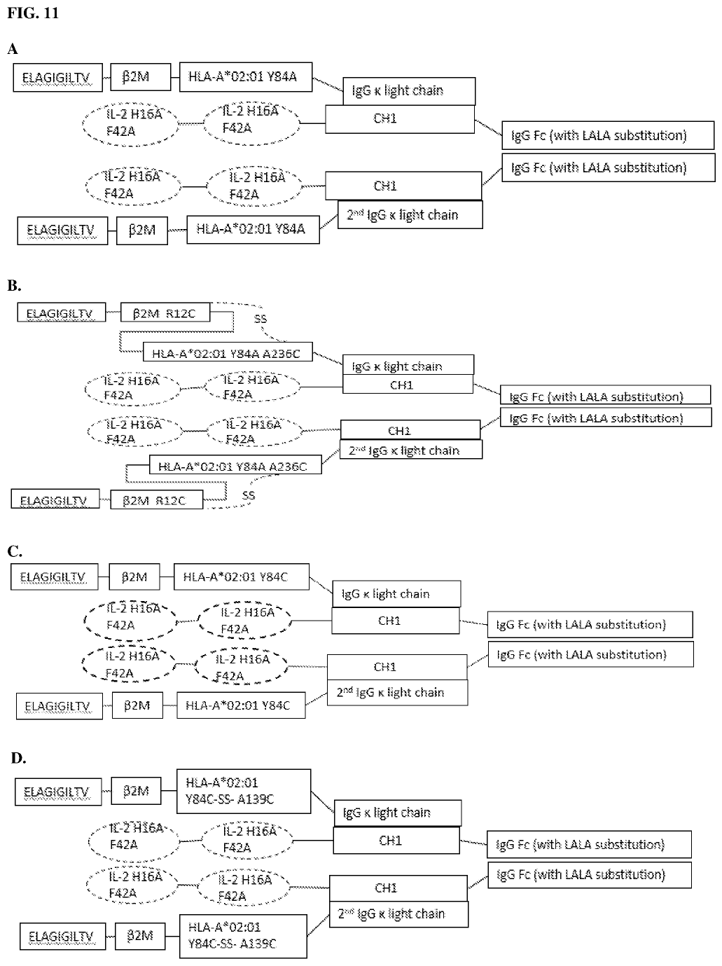

MAPPs of the present disclosure comprise (i)framework polypeptides with a multimerization sequence and at least one dimerization sequence, and (ii) dimerization polypeptides with a counterpart dimerization sequence that binds with the framework polypeptide's the dimerization sequence. As discussed above, MAPPs typically will further comprise either one or more epitope presenting sequences or one or more epitope presenting complexes. Exemplary structures for such MAPPs appear in FIG. 1A, 1B, and FIGS. 6-11. The structure depicted in FIGS. 10 and 11 represents MAPPs with multimerizing framework polypeptides and epitope presenting sequences (the “Single Chain MHC” with the “Epitope”). In FIGS. 6-9, the structures represent MAPPs with multimerizing framework polypeptides where the epitope MHC combination represents either epitope presenting sequences or epitope presenting complexes.

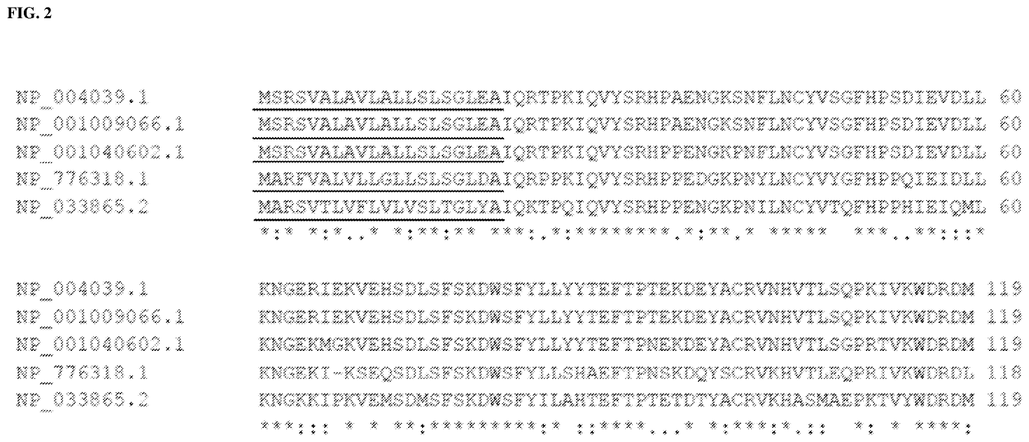

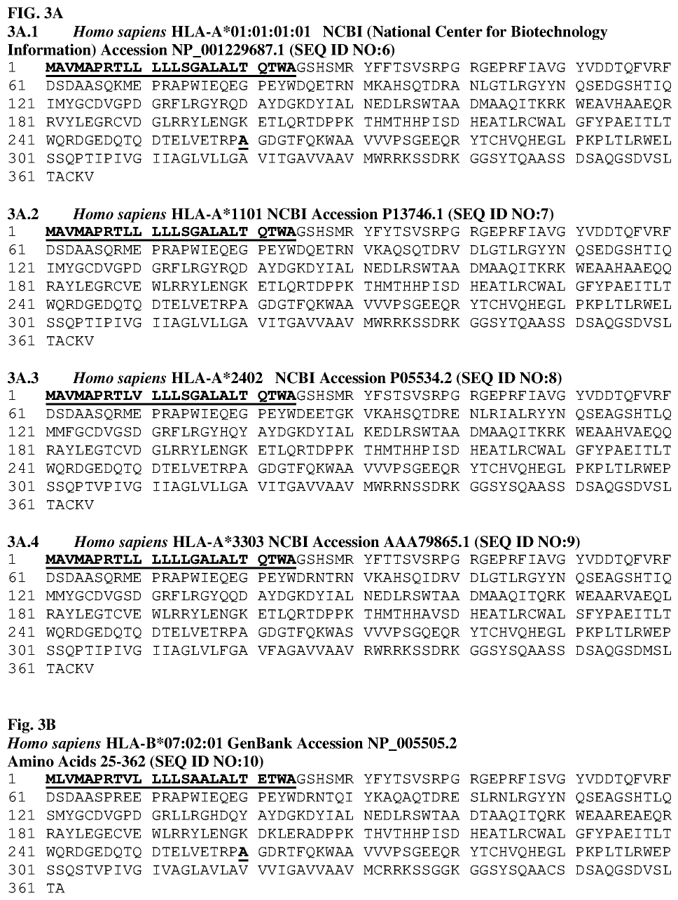

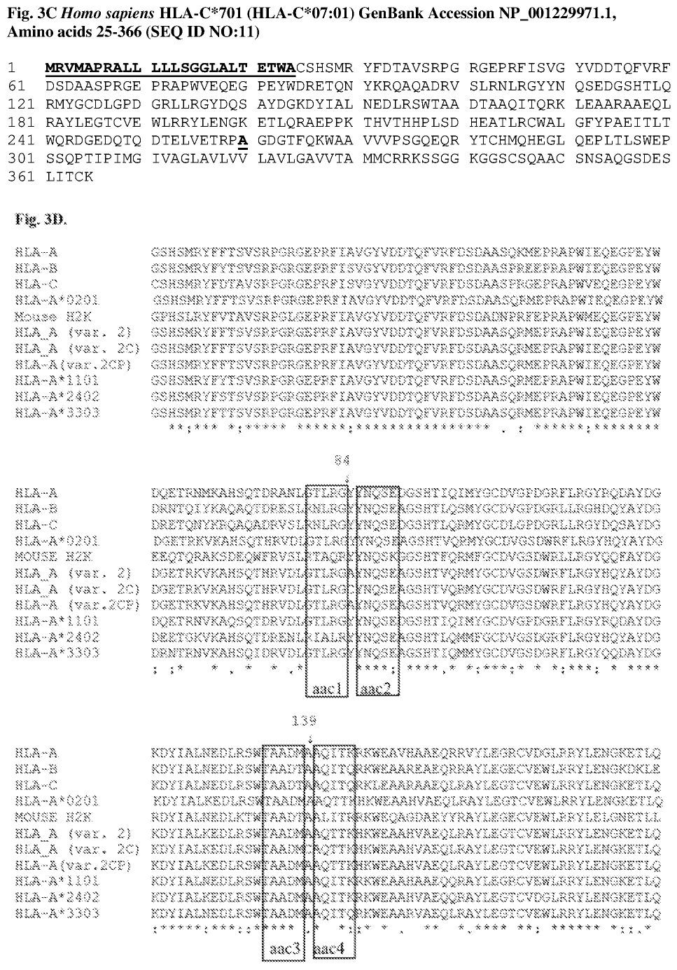

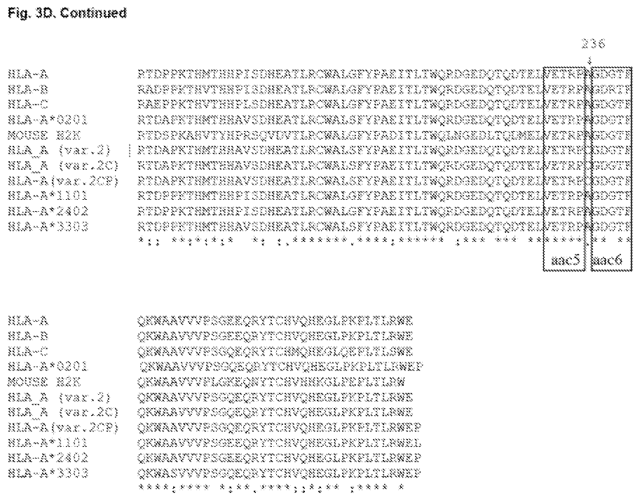

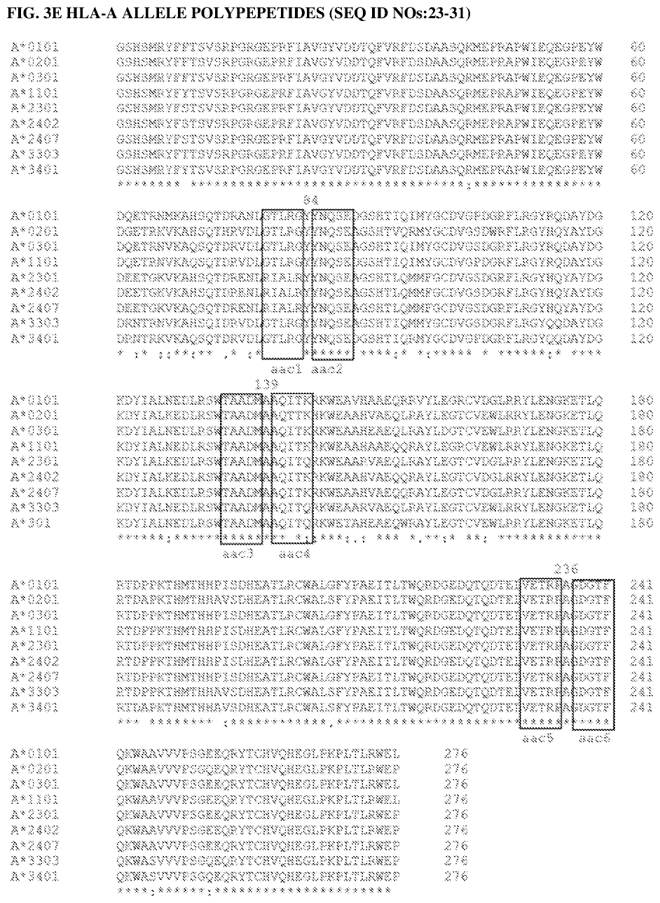

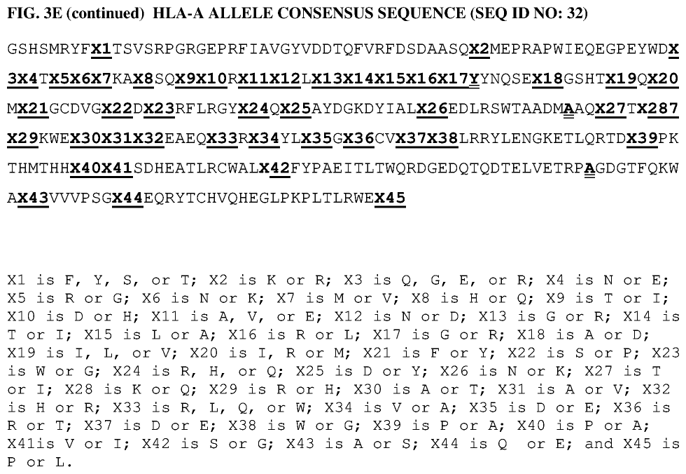

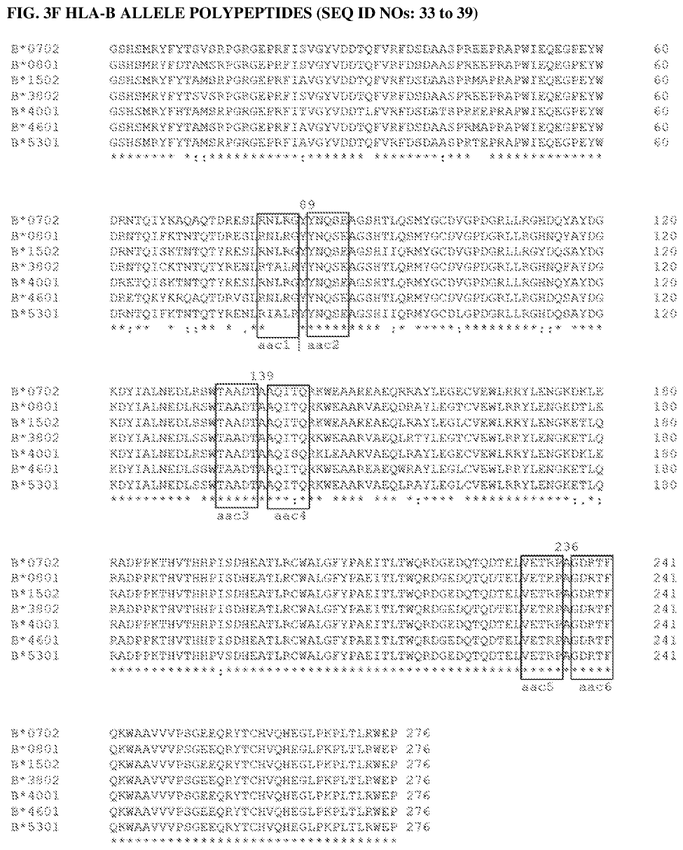

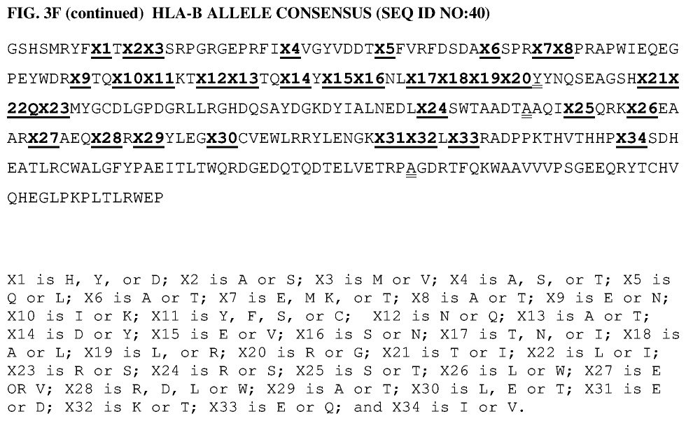

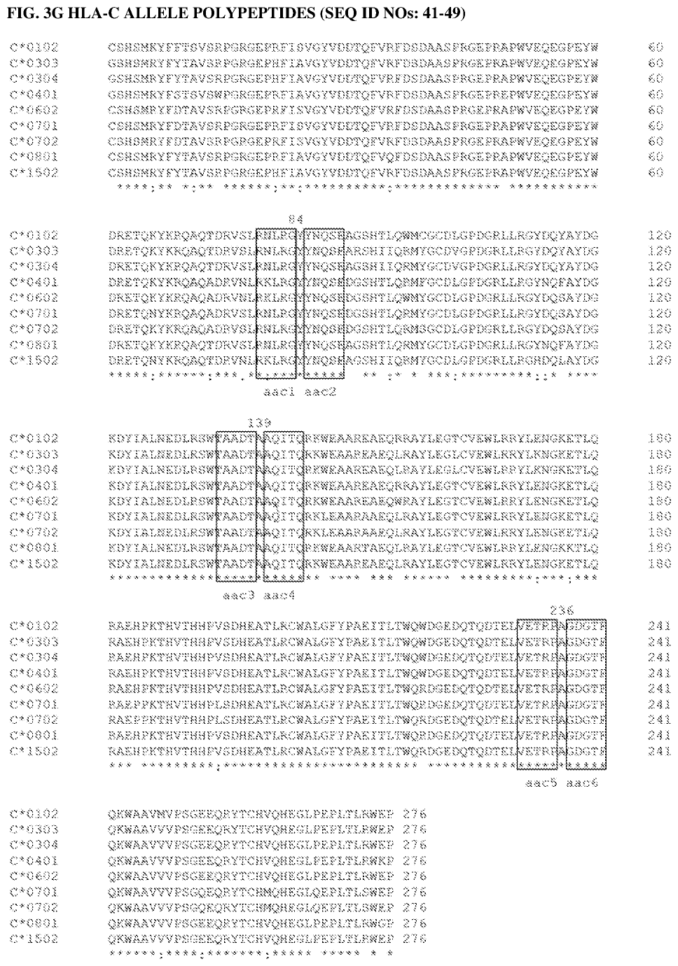

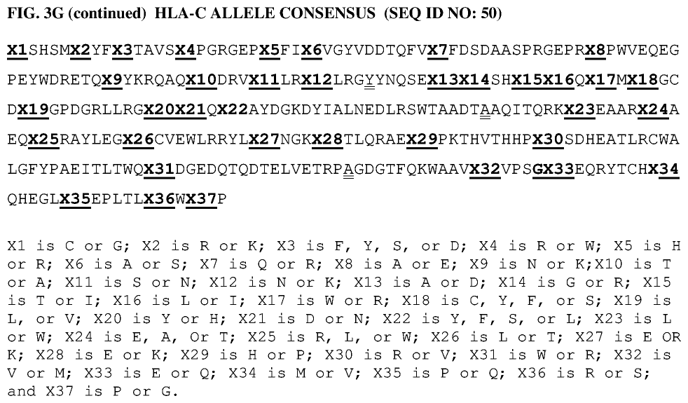

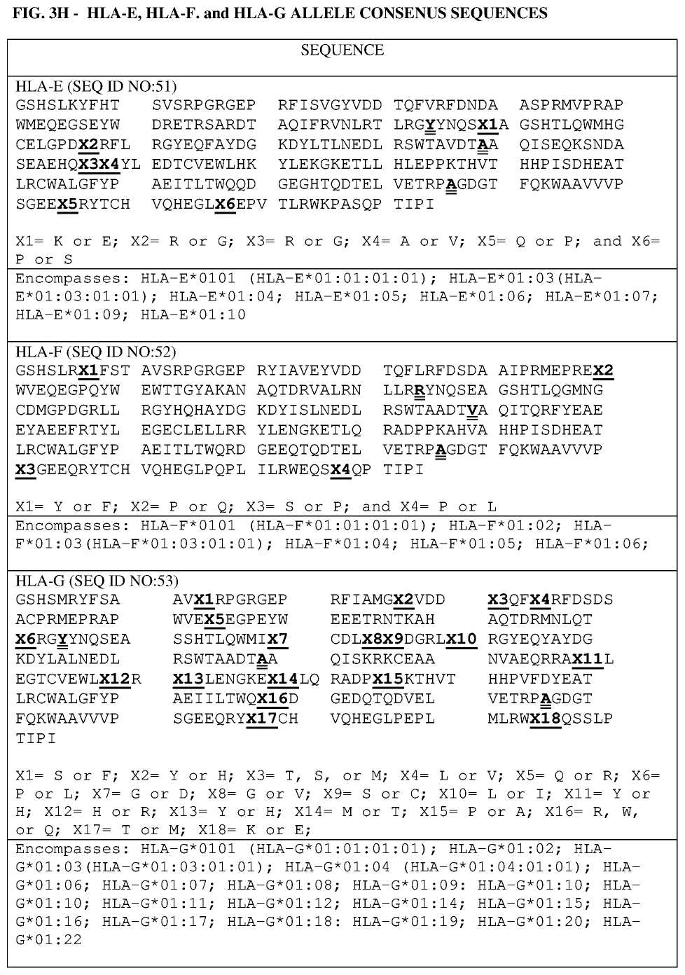

Interactions of MHC-H and β2M are not considered to result in multimerization and/or dimerization. In and embodiment, neither the dimerization sequence nor the multimerization sequence of the framework polypeptide, nor the counterpart dimerization sequence of the dimerization polypeptide comprises a Class I MHC-H or β2M polypeptide sequence having at least 90% (e.g., 95% or 98%) sequence identity to at least 15 (e.g., at least 20, 30, 40, 50, 60 or 70) contiguous aas of a MHC Class I polypeptide(e.g., a polypeptide in any of FIG. 2 or 3A to 3I). In embodiments, MAPPs comprise at least one, or at least two, dimerization peptides that comprise an epitope presenting sequence. See, e.g., FIG. 1A.

One group of MAPPs, those having epitope presenting sequences, comprise a multimerizing framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptide and the framework polypeptide comprise a presenting sequence located on the N-terminal side of their dimerization or counterpart dimerization sequences. In such a MAPP the presenting sequence may comprise a peptide epitope and one or more MHC polypeptide sequences, with the peptide epitope sequence located: (i) at or within 10 aa, 15 aa, 20 aa, or 25 aa of the N-terminus of the presenting sequence, or (ii) in a polypeptide located at the N-terminus of the presenting sequence comprising, from N-terminus to C-terminus, a MOD, one or more optional linkers, and the peptide epitope; optionally at least one (e.g., one, two or each) of the framework polypeptide, dimerization peptide, and presenting sequence comprises one or more independently selected MODs located at their N-terminus and/or C-terminus (or on the N-terminal or C-terminal side of the dimerization or counterpart dimerization sequences); wherein the MHC polypeptide sequences are MHC Class I polypeptide sequences and they comprise an MHC Class I heavy chain (e.g., MHC-H α1, α2, and α3 domains) polypeptide and β2M polypeptide sequences (e.g., human MHC-H and a β2M sequences). In an embodiment, neither the dimerization sequence nor the multimerization sequence of the framework polypeptide comprises a MHC-H peptide or β2M sequence having at least 90% (e.g., 95% or 98%) sequence identity to at least 15 (e.g., at least 20, 30, 40, 50, 60 or 70) contiguous aas of a MHC Class I polypeptide in any of FIGS. 2 to 3I.

Another group of MAPPs, those having epitope presenting complexes, comprise a framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptides and/or at least one (e.g., one or both) of the framework polypeptide comprise a presenting complex 1^{st} sequence located on the N-terminal side of their dimerization sequence. A presenting complex 2^{nd} sequence is associated with the presenting complex 1^{st} sequence (e.g., non-covalently or covalently such as by one or two interchain disulfide bonds) to form a presenting complex. In such a MAPP, each of the presenting complex 1^{st} sequence and its associated presenting complex 2^{nd} sequence are comprised of one or more the MHC-H and β2M polypeptide sequences, with one of the sequences further comprising the peptide epitope. The peptide epitope may be located (i) at or within 10aa, 15 aa, 20 aa, or 25 aa of the N-terminus of the presenting complex 1^{st} sequence or presenting complex 2^{nd} sequence, or (ii) in a polypeptide located at the N-terminus of the presenting complex 1^{st} sequence or presenting complex 2^{nd} sequence, with the polypeptide comprising, from N-terminus to C-terminus, a MOD, one or more optional linkers, and the peptide epitope. Optionally, at least one (e.g., one, two or each) of the framework polypeptide, dimerization peptide, or the peptides of a presenting complex comprise one or more independently selected MODs located at their N-terminus or C-terminus (or on the N-terminal or C-terminal side of the dimerization sequences).

MAPPs of the present disclosure may be constructed such that neither the dimerization sequence nor the multimerization sequence of the framework polypeptide comprises a Class I MHC peptide sequence having at least 90% (e.g., 95% or 98%) sequence identity to at least 15 (e.g., at least 20, 30, 40, 50, 60 or 70) contiguous aas of a MHC Class I polypeptide in any of FIGS. 2 to 3I.

As discussed above, a dimerization sequence of a framework polypeptide may interact with dimerization peptides to form heterodimers. The multimerization sequence of the framework polypeptide may associate with another framework polypeptide multimerization sequence forming a duplex (or higher order structure, such as a triplex, quadraplex or pentaplex) of the heterodimers. Where the multimerization sequences are interspecific (e.g., a knob-in-hole Fc peptide pair), and at least one heterodimer comprises an interspecific dimerization and counterpart dimerization pair, two different heterodimers may be formed. When the different heterodimers are combined to form a duplex MAPP, any one or more component (e.g., MODs) may differ (e.g., in type or location) between the two heterodimers.

C. MAPP Components 1. Framework Polypeptides and Dimerization Polypeptides

As may be understood from the preceding sections, framework polypeptides serve as the structural basis or skeleton of MAPPs, permitting the organization of other elements in the MAPP complex. Framework peptides interact with other peptides through binding interactions, principally at dimerization and multimerization sequences. Interactions at dimerization sequences permit association of non-framework peptides (e.g., dimerization peptides) with framework peptides. In contrast, multimerization sequences are involved in the interaction of two or more framework peptides.

The framework polypeptide(s) of MAPPs comprise at least one multimerization sequence, and at least one independently selected dimerization sequence that is not identical to or of the same type (e.g., not both leucine zipper variants) as the multimerization sequence. By utilizing different types of sequences for the interactions at multimerization and dimerization sequences, it becomes possible to control the interactions of the framework polypeptide with other framework polypeptides and with dimerization polypeptides. In an embodiment, framework polypeptides comprise one multimerization sequence and one dimerization sequence. In an embodiment, framework polypeptides comprise at least one multimerization sequence and at least two independently selected dimerization sequences. Framework peptides may contain peptide sequences (e.g., linker sequences and/or MOD sequences) between any of the elements of the framework polypeptide or at the ends of the framework polypeptide including the multimerization sequences and dimerization sequences.

In addition to providing for the structural organization of MAPPS through their multimerization and dimerization sequences, framework peptides, and particularly their N- and C-termini, may also serve as locations for placement of elements such as MOD sequences, an epitope, presenting sequences, and/or a presenting complex 1^{st} sequence (one polypeptide of an epitope presenting complexes), see FIG. 1B. When placed at the N- and/or C-termini of a framework polypeptide, such polypeptide elements are part of the framework polypeptide (e.g., a single translation product formed in a cell).

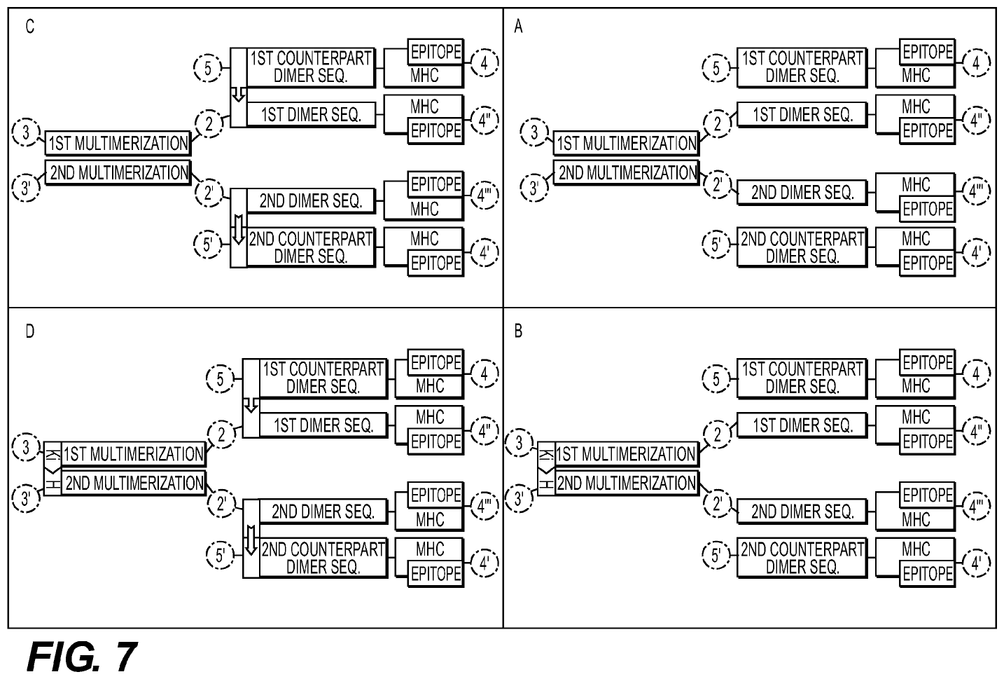

Within a MAPP, all of the dimerization sequence may be non-interspecific (such as leucine zipper pairs) while the multimerization sequences is either interspecific or non-interspecific (see e.g., structures A & B of FIGS. 6 and 7). For example, in a duplex MAPP with first and second framework polypeptides, the multimerization sequences may be a non-interspecific (e.g., IgFc (CH2, CH3) or leucine zippers) or the multimerization sequences may be an interspecific knob-in-hole sequence pair; with the dimerization sequences of the first and second framework polypeptide as a non-interspecific leucine zipper polypeptides. Where an Fc polypeptide is employed it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region.

Within a MAPP, all of the dimerization sequences may be interspecific, while the multimerization sequences are not interspecific (see e.g., FIG. 10A). For example, in a duplex MAPP with first and second framework polypeptides, the multimerization sequences may be an IgFc sequence, with the a ZW1 sequence or its counterpart employed as the dimerization sequence of the first framework polypeptide and an Ig CH1 domain or its counterpart Ig C_{L} κ sequence as the dimerization sequence of the second framework polypeptide.

All of the dimerization sequences or all of the dimerization and multimerization sequences, in a MAPP may differ in that they bind only specific binding partners present in the MAPP (e.g., each are part of a different interspecific sequence pair). For example, in a duplex MAPP with first and second framework polypeptides, the multimerization sequences may be a pair of knob-in-hole IgFc sequences, with the a ZW1 sequence or its counterpart employed as the dimerization sequence of the first framework polypeptide, and an Ig CH1 or its counterpart Ig C_{L} sequence as the dimerization sequence of the second framework polypeptide.
2. Multimerization and Dimerization Polypeptide Sequences

Amino acid sequences that permit polypeptides to interact may be utilized as dimerization sequences or counterpart dimerization sequences when they are involved in the formation of dimers between a framework polypeptide and a dimerization polypeptide. The same type of aa sequences may be utilized as multimerization sequences when they are used to form duplex or higher order structures (trimers, tetramers, pentamer, etc.) between framework polypeptides. In any given MAPP, sequences that can interact with each other are not utilized as both dimerization and multimerization sequences. Stated another way, the same aa sequence pair may serve as either dimerization or multimerization sequences depending on whether they: bring together two or more framework peptides, in which case they are multimerization sequences; or they bring together a dimerization and multimerization sequence, in which case they are designated as dimerization sequences.

Where dimerization or multimerization sequences employ identical sequences that pair or multimerize (e.g., some leucine zipper sequences), they can form symmetrical pairs or multimers (e.g., homodimers) as shown in FIG. 6 structure A. In contrast, where dimerization or multimerization sequences that pair are not identical and require a specific complementary counterpart sequence to form a dimer, they are interspecific binding sequences and can form asymmetric pairs. Both IgFc) and non-immunoglobulin polypeptides can be interspecific or non-interspecific in nature. For example, both Fos/Jun binding pairs and Ig CH1 polypeptide sequences and light chain constant region C_{L} sequences form interspecific binding pairs. Natural Ig Fc regions tend to be non-interspecific, but, as discussed below, can be made to form interspecific pairs (e.g., KiH pairs). Coiled-coil sequences, including leucine zipper sequences, can be either interspecific leucine zipper or non-interspecific leucine zipper sequences. See e.g., Zeng et al., (1997) PNAS (USA) 94:3673-3678; and Li et al., (2012), Nature Comms 3:662.

Interspecific binding sequences may in some instances form some amount of homodimers, but preferentially dimerize by binding more strongly with their counterpart interspecific binding sequence. Accordingly, specific heterodimers tend to be formed when an interspecific dimerization sequence and its counterpart interspecific binding sequence are incorporated into a pair of polypeptides. By way of example, where an interspecific dimerization sequence and its counterpart are incorporated into a pair of polypeptides, they may selectively form greater than 70%, 80%, 90%, 95%, 98% or 99% heterodimers when an equimolar mixture of the polypeptides are combined (for example in PBS buffer at 20° C.). The remainder of the polypeptides may be present as monomers or homodimers, which may be separated from the heterodimer. See, for example, FIG. 6, structure B, with an interspecific multimerization sequence and structure C with two different interspecific dimerization sequences. Moreover, because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences.

Sequence are considered orthogonal to other sequences when they do not form complexes (bind) with each other's counterpart sequences. See FIG. 6 structure D where the MAPP comprises an interspecific multimerization sequence and two independently selected interspecific dimerization sequences, all of which are orthogonal to each other. Any of the MAPPS described herein may have two or more (e.g., three, four or more) orthogonal dimerization sequences. In an embodiment, MAPPs with multimerizing framework peptides may have orthogonal multimerization and dimerization domains (where the dimerization domains may or may not be orthogonal to each other).

Some sequences permitting polypeptides to interact with sufficient affinity to be used as dimerization and/or multimerization sequences are provided for example in U.S. Patent Publication No. 2003/0138440. The sequences may be of relatively compact size (e.g., such as less than about 300, 250, 225, 200, 175, 150, 125, 100, 75, 60, 50, 40, or 30 aa). In an embodiment, at least one (e.g., at least two or all) dimerization and/or multimerization sequence is less than 300 aa. In an embodiment, at least one (e.g., at least two or all) of the dimerization and/or multimerization sequences are less than 200 aa. In an embodiment, at least one (e.g., at least two or all) of the dimerization and/or multimerization sequences are less than 100 aa. In an embodiment, at least one (e.g., at least two or all) or all dimerization and/or multimerization sequences are less than 75 aa. In another embodiment, at least one (e.g., at least two or all) of the dimerization and/or multimerization sequences are less than are less than 50 aa. In an embodiment, at least one (e.g., at least two or all) of the dimerization and/or multimerization sequences are less than 30 aa.

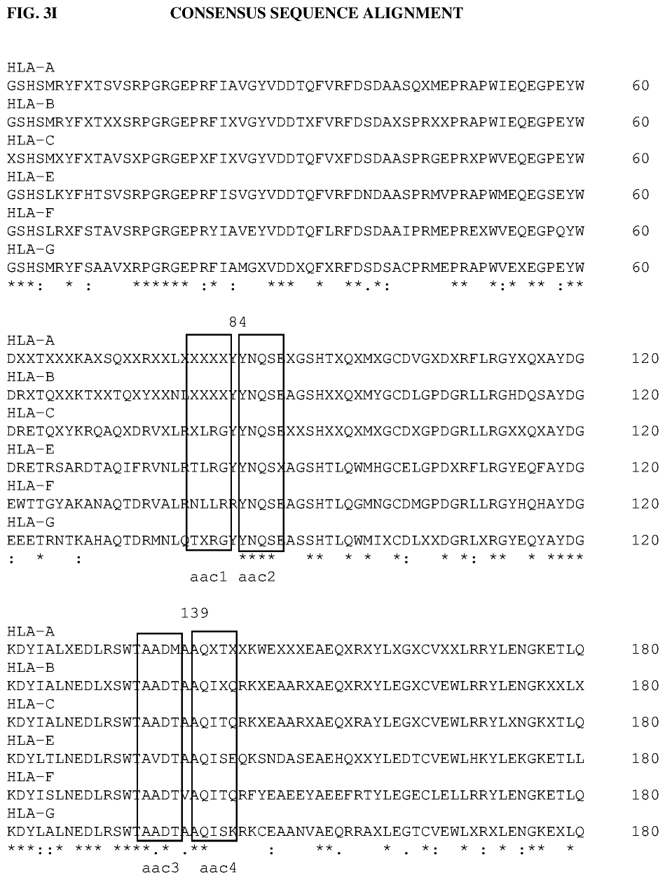

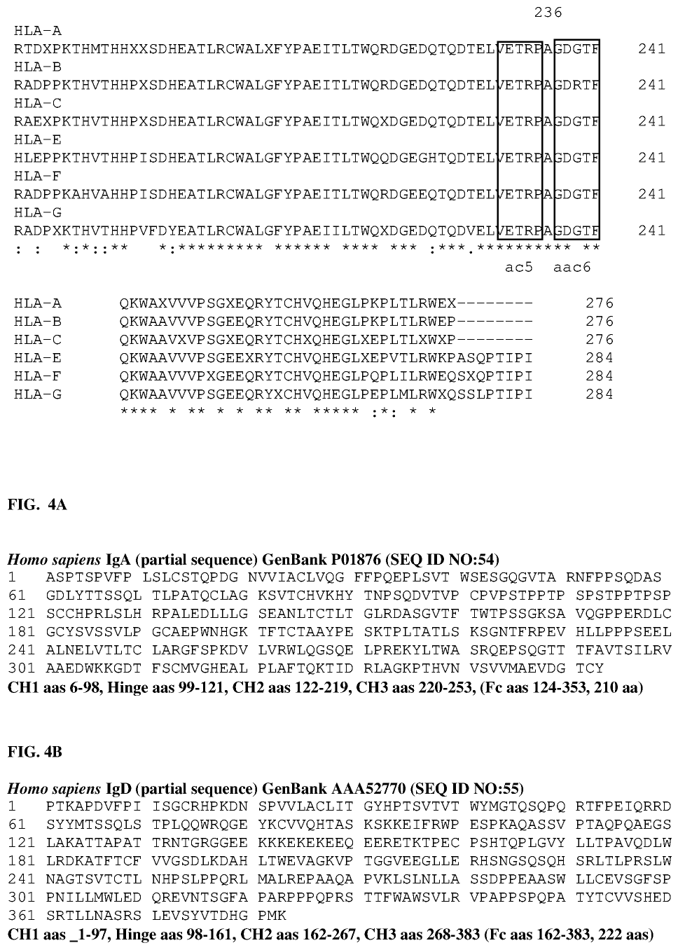

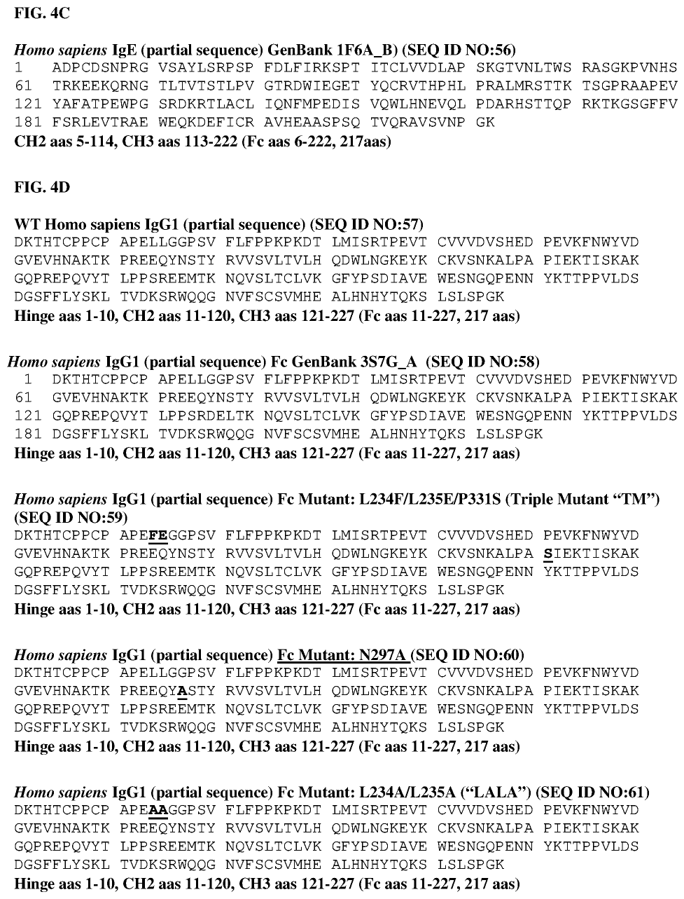

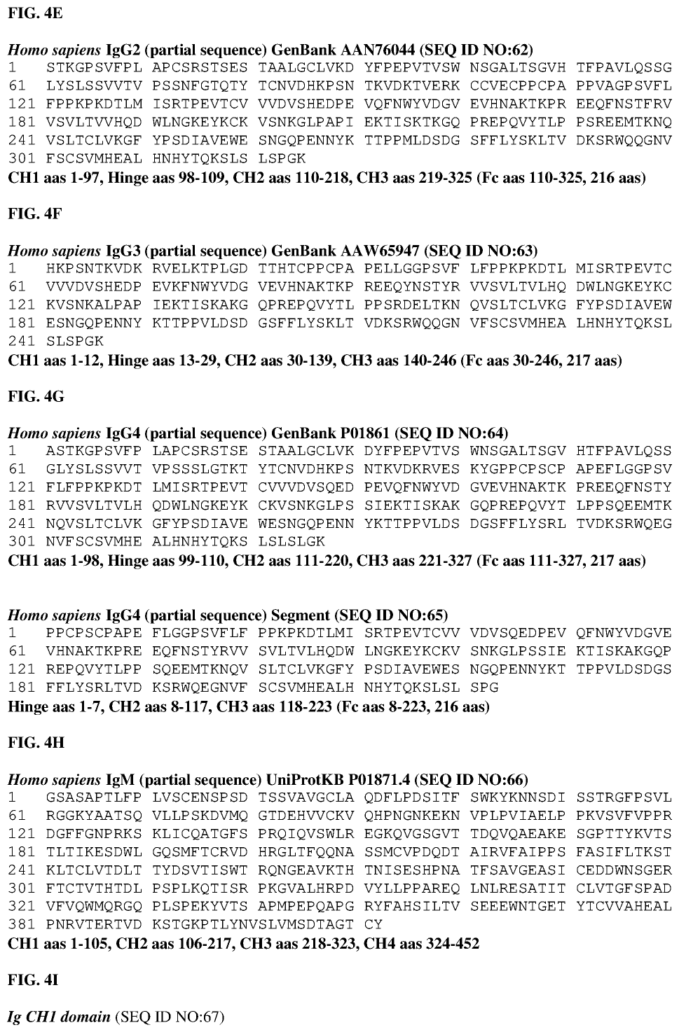

Dimerization/multimerization sequences include but are not limited to immunoglobulin heavy chain constant region (Ig Fc) polypeptide sequences (e.g., sequences comprising CH2-CH3 regions of immunoglobulins such as those provided in FIGS. 4A-4H and SEQ ID NOs: 54 to 66); polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; interspecific Ig Fc heavy chain constant regions (such as knob-in-hole sequences described in more detail below); Fos/Jun binding pairs; immunoglobulin heavy chain constant region (CH2-CH3) sequences, and; Ig CH1 and light chain constant region CL sequences (Ig CH1/CL pairs such as a Ig CH1 sequence paired with a Ig C_{L} κ or λ light chain constant region sequence).

Framework and/or dimerization polypeptides of a MAPP may comprise an immunoglobulin heavy chain constant region (e.g., CH2-CH3 domains) polypeptide sequence that functions as a dimerization or multimerization sequence. Where the framework polypeptide comprises a IgFc multimerization sequence, and a CH1 dimerization sequence it may comprise all or part a native or variant immunoglobin sequence set forth in any of FIGS. 4A to 4H that comprise the CH1, CH2 and CH3 domains and any hinge sequences that may be present. An Ig Fc sequence or any one or more of the CH1, CH2, and CH3 domains, may have at least about 70%, at least about 80%, at least about 90%, at least about 95%, at least about 98%, at least about 99%, or 100% aa sequence identity to an aa sequence of an Fc region depicted in FIGS. 4A-4H. In particular, the C-terminal lysine provided in some of the sequences provided in FIGS. 4A-4H (e.g., the IgG sequences in FIGS. 4D, 4E, 4F, and 4G) may be removed during cellular processing of the MAPPs and may not be present on some or all of the MAPP molecules as expressed. See, e.g., van den Bremer et al. (2015) mAbs 7:4; and Sissolak et al. (2019) J. Industrial Microbiol. & Biotechnol. 46:1167. Alternatively, in preparing MAPPs, the nucleotides encoding a C-terminal lysine may simply be omitted from any of the sequences provided in FIGS. 4A-4H in which a C-terminal lysine occurs.

Such immunoglobulin sequences can covalently link the polypeptides of MAPP complex together by forming one or two interchain disulfide bonds, thereby stabilizing MAPPs, particularly where a pair of interspecific Ig sequence such as knob-in-hole polypeptide pairs are employed. Where an Fc polypeptide sequence, alone or in combination with a CH1 polypeptide sequence, is employed as a multimerization or dimerization sequence it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region. As discussed below, the Ig Fc region can further contain substitutions that can substantially reduce the ability of the Ig Fc to effect complement-dependent cytotoxicity (CDC) or antibody-dependent cell cytotoxicity (ADCC). Accordingly, framework and/or dimerization polypeptides, and in particular Ig Fc sequences used as multimerization or dimerization sequences, may comprise substitutions that reduce or substantially eliminate ADCC and/or CDC responses.

Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgA Fc sequence depicted in FIG. 4A (SEQ ID NO:54). Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgD Fc sequence depicted in FIG. 4B (SEQ ID NO:55). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (at least 150, at least 175, or at least 200 contiguous aas), or all aas, of the IgE Fc sequence depicted in FIG. 4C (SEQ ID NO:56).

A MAPP may comprise one or more IgG Fc sequences as dimerization and/or multimerization sequences. The Fc polypeptide of a MAPP of the present disclosure can be a human IgG1 Fc, a human IgG2 Fc, a human IgG3 Fc, a human IgG4 Fc, etc. In some cases, the Fc sequence has at least about 70%, at least about 80%, at least about 90%, at least about 95%, at least about 98%, at least about 99%, or 100% aa sequence identity to an aa sequence of an Fc region depicted in FIG. 4D-4G. Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (e.g., at least 150, at least 175, at least 200, or at least 220 contiguous aas), or all aas, of the wt. IgG1 Fc polypeptide sequence depicted in FIG. 4D (SEQ ID NO: 57). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 225) contiguous aas, or all aas, of the IgG2 Fc polypeptide sequence depicted in FIG. 4E (SEQ ID NO:62). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, at least 225, or at least 240) contiguous aas, or all aas, of the IgG3 Fc sequence depicted in FIG. 4F (SEQ ID NO:63). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 220) contiguous aas, or all aas, of the IgG4 Fc sequence depicted in FIG. 4G (SEQ ID NO:64 or 65).

Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (at least 150, at least 175, at least 200, at least 225, or at least 250) contiguous aas, or all aas, of the IgM Fc polypeptide sequence depicted in FIG. 4H (SEQ ID NO:66).

Framework and/or dimerization polypeptides of a MAPP comprising immunoglobulin sequences (e.g., depicted in FIGS. 4A-4H) can be covalently linked together by formation of at least one or at least two interchain disulfide bonds between cysteines that are adjacent to the immunoglobulin hinge regions. Such disulfide bonds can stabilize the interaction of framework and dimerization polypeptide heterodimers, or, for example, duplexes of such heterodimers when the disulfide bonds are between framework multimerization sequences.

A framework or dimerization polypeptide may comprise an aa sequence having 100% aa sequence identity to the wt. human IgG1 Fc polypeptide depicted in FIG. 4D. A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of N297 (N77 as numbered in FIG. 4D, SEQ ID NO:60) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in CDC. K322 (e.g., K322A) substitutions shows a substantial reduction in reduction in FcγR binding affinity and ADCC, with the C1q binding and CDC functions substantially or completely eliminated. Hezareh et al., (2001) J. Virol. 75:12161-168.

Amino acid L234 and other aas in the lower hinge region (e.g., aas 234 to 239, such as L235, G236, G237, P238, S239), which correspond to aas 14-19 of SEQ ID NO:57) of IgG are involved in binding to the Fc gamma receptor (FcγR), and accordingly, mutations at that location reduce binding to the receptor (relative to the wt. protein) and result in a reduction in antibody-dependent cellular cytotoxicity (or alternatively antibody-dependent cell-mediated cytotoxicity, “ADCC”). Hezareh et al., (2001) have demonstrated that the double mutant (L234A, L235A) does not effectively bind either FcγR or C1q, and both ADCC and CDC functions were reduced or substantially eliminated. A framework or dimerization polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 4D, that includes a substitution of L234 (L14 of the aa sequence depicted in FIG. 4D) with an aa other than leucine.

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of L235 (L15 of the aa sequence depicted in FIG. 4D) with an aa other than leucine. In some cases, the framework and/or dimerization polypeptide present in a MAPP with substitutions in the lower hinge region includes L234A and L235A (“LALA”) substitutions (the positions corresponding to positions 14 and 15 of the wt. aa sequence depicted in FIG. 4D; see, e.g., SEQ ID NO:61).

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of P331 (P111 of the aa sequence depicted in FIG. 4D) with an aa other than proline. Substitutions at P331, like those at N297, lead to reduced binding to C1q relative to the wt. protein, and thus a reduction in complement dependent cytotoxicity. In one embodiment, the substitution is a P331S substitution. In another embodiment, the substitution is a P331A substitution.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, and include substitutions of D270, K322, and/or P329 (corresponding to D50, K102, and P109 of SEQ ID NO:57 in FIG. 4D) that reduce binding to C1q protein relative to the wt protein.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, including substitutions at L234 and/or L235 (L14 and/or L15 of the aa sequence depicted in FIG. 4D) with aas other than leucine such as L234A and L235A, and a substitution of P331 (P111 of the aa sequence depicted in FIG. 4D) with an aa other than proline such as P331S. In one instance, a framework or dimerization polypeptide present in a MAPP comprises the “Triple Mutant” aa sequence (SEQ ID NO:59) depicted in FIG. 4D (human IgG1 Fc) having L234F, L235E, and P331S substitutions (corresponding to aa positions 14, 15, and 111 of the aa sequence depicted in FIG. 4D).

Where an asymmetric pairing between two polypeptides of a MAPP is desired, a framework or dimerization polypeptide present in a MAPP may comprise, consist essentially of, or consist of an interspecific binding sequence. Interspecific binding sequences favor formation of heterodimers with their cognate polypeptide sequence (i.e., the interspecific sequence and its counterpart interspecific sequence), particularly those based on immunoglobulin Fc (Ig Fc) sequence variants. Such interspecific polypeptide sequences include knob-in-hole without (KiH) or with (KiHs-s) a stabilizing disulfide bond, HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Ig Fc polypeptide with Y349C, T366S, L368A, and Y407V substitutions and a second Ig Fc polypeptide with S354C, and T366W substitutions (disulfide bonds can form between the Y349C and the S354C). See e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon immunoglobulin sequences are summarized in the table that follows, with cross reference to the numbering of the aa positions as they appear in the wt. IgG1 sequence (SEQ ID NO:57) set forth in FIG. 4D shown in brackets “{ }”.

TABLE 1
Interspecific immunoglobulin sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain S—S |
| | | | bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 modified from Ha et al., Frontiers in Immunol. 7: 1-16 (2016).
*aa forms a stabilizing disulfide bond.

In addition to the interspecific pairs of sequences in Table 1, framework and/or dimerization polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol.7:1-16 (2016).

A framework or dimerization polypeptide found in a MAPP may comprise an interspecific binding sequence or its counterpart interspecific binding sequence selected from the group consisting of: KiH); (KiHs-s); HA-TF; ZW-1; 7.8.60; DD-KK; EW-RVT; EW-RVTs-s; A107; or SEED sequences.

A MAPP may comprise a framework or dimerization polypeptide comprising an IgG1 KiH or KiHs-s sequence with a T146W sequence substitution, and its counterpart interspecific KiH or KiHs-s binding partner polypeptide comprises an IgG1 sequence having T146S, L148A, and Y187V sequence substitutions, where the framework and/or dimerization polypeptides comprises a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D. One or both of the framework, or both of dimerization polypeptides optionally comprising substitutions at one of more of: L234 and L235 (e.g., L234A/L235A “LALA” or L234F/L235E); N297 (e.g., N297A); P331 (e.g., P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g., P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 4D.

A MAPP may comprise a framework or dimerization polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).See e.g., the N297A and LALA sequences in FIG. 4D.

A MAPP may comprise a framework or dimerization polypeptide comprising an IgG1 sequence with a T146W and S134C KiHs-s substitutions, and its counterpart interspecific binding partner polypeptide comprises an IgG1 sequence having T146S, L148A, Y187V and Y129C KiHs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a S144H and F185A HA-TF substitutions, and its counterpart interspecific binding partner polypeptide comprises an IgG1 sequence having Y129T and T174F HA-TF substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; with none, one, or both of the sequences comprising L14 and L15 substitutions (e.g., “LALA” substitutions L234A and L235A), and/or N77 (N297 e.g., N297A or N297G).

A MAPP may comprise a framework or dimerization polypeptide comprising an IgG1 sequence with a T130V, L131Y, F185A, and Y187V ZW1 substitutions, and its counterpart interspecific binding partner polypeptide comprises an IgG1 sequence having T130V, T146L, K172L, and T174W ZW1 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140D, D179M, and Y187A 7.8.60 substitutions, and its counterpart interspecific binding partner polypeptide comprises an IgG1 sequence having T130V E125R, Q127R, T146V, and K189V 7.8.60 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

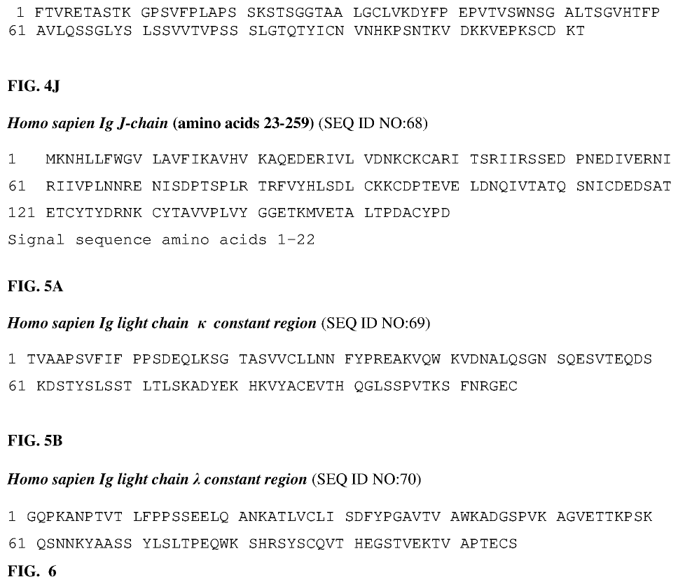

As an alternative to the use of immunoglobulin CH2 and CH3 heavy chain constant regions as dimerization or multimerization sequences, immunoglobulin light chain constant regions (See FIGS. 5A and 5B) can be paired with Ig CH1 sequences (See FIG. 4I) as multimerization or dimerization sequences and their counterpart sequences of a framework polypeptide.

In an embodiment, a MAPP framework or dimerization polypeptide comprises an Ig CH1 domain (e.g., the polypeptide of FIG. 4I), and the sequence with which it will form a complex (its counterpart binding partner) comprises an Ig κ chain constant region sequence, where the framework or dimerization polypeptide comprise a sequence having at least 80%, 85%, 90%, 95%, 98%, 99%, or 100% sequence identity to at least 70, at least 80, at least 90, at least 100, or at least 110 contiguous aas of SEQ ID NOs: 67 and/or 69 respectively See FIGS. 4I and 5A. The Ig CH1 and Ig κ sequences may be modified to increase their affinity for each other, and accordingly the stability of any heterodimer formed utilizing them as a dimerization or multimerization sequences. Among the substitutions that increase the stability of CH1-Ig κ heterodimers are those identified as the MD13 combination in Chen et al., MAbs, 8(4):761-774 (2016). In the MD13 combination two substitutions are introduced into to each of the IgCH1 and Ig κ sequences. The Ig CH1 sequence is modified to contain S64E and S66V substitutions (S70E and S72V of the sequence shown in FIG. 4I). The Ig κ sequence is modified to contain S69L and T71S substitutions (S68L and T70S of the sequence shown in FIG. 5A).

A framework or dimerization polypeptide of a MAPP comprises an Ig CH1 domain (e.g., the polypeptide of FIG. 4I SEQ ID NO:67), and its counterpart sequence comprises an Ig λ chain constant region sequence such as is shown in FIG. 5B (SEQ ID NO:70), where the framework or dimerization polypeptide comprises a sequence having at least 80%, 85%, 90%, 95%, 98%, 99%, or 100% sequence identity to at least 70 (e.g., at least 80, at least 90, or at least 100) contiguous aas of the sequences shown in FIG. 5B.

Framework and/or dimerization polypeptides of a MAPP each comprise a leucine zipper polypeptide as a dimerization or multimerization sequence. The leucine zipper polypeptides bind to one another to form dimer (e.g., homodimer). Non-limiting examples of leucine-zipper polypeptides include a peptide comprising any one of the following aa sequences: RMKQIEDKIEEILS-KIYHIENEIARIKKLIGER (SEQ ID NO:71); LSSIEKKQEEQTSWLIWISNELTLIRNELAQS (SEQ ID NO:72); LSSIEKKLEEITSQLIQISNELTLIRNELAQ (SEQ ID NO:73; LSSIEKKLE-EITSQLIQIRNELTLIRNELAQ (SEQ ID NO:74); LSSIEKKLEEITSQLQQIRNELTLIRNELAQ (SEQ ID NO:75); LSSLEKKLEELTSQLIQLRNELTLLRNELAQ (SEQ ID NO:76); ISSLEKKIEELTSQI-QQLRNEITLLRNEIAQ (SEQ ID NO:77). In some cases, a leucine zipper polypeptide comprises the following aa sequence: LEIEAAFLERENTALETRVAELRQRVQRLRNRVSQYRTRYGPLGGGK (SEQ ID NO:78). Additional leucine-zipper polypeptides are known in the art, a number of which are suitable for use as multimerization or dimerization sequences.

The framework and/or dimerization polypeptides of a MAPP may comprise a coiled-coil polypeptide sequence that forms a dimer. Non-limiting examples of coiled-coil polypeptides include, for example, a peptide of any one of the following aa sequences: LKSVENRLAVVENQLKTVIEELK-TVKDLLSN (SEQ ID NO:79); LARIEEKLKTIKAQLSEIASTLNMIREQLAQ (SEQ ID NO:410); VSRLEEKVKTLKSQVTELASTVSLLREQVAQ (SEQ ID NO:80); IQSEKKIEDISSLIG-QIQSEITLIRNEIAQ (SEQ ID NO:81); and LMSLEKKLEELTQTLMQLQNELSMLKNELAQ (SEQ ID NO:82).

A MAPP may comprise a pair of two framework polypeptides and/or a framework and dimerization polypeptide that each have an aa sequence comprising at least one cysteine residue that can form a disulfide bond permitting homodimerization or heterodimerization of those polypeptides stabilized by disulfide bond between the cysteine residues. Examples of such aa sequences include:

(SEQ ID NO: 83)
VDLEGSTSNGRQCAGIRL;
(SEQ ID NO: 84)
EDDVTTTEELAPALVPPPKGTCAGWMA;
and
(SEQ ID NO: 85)
GHDQETTTQGPGVLLPLPKGACTGQMA.

Some aa sequences suitable as multimerization (oligomerization) sequences permit formation of MAPPs capable of forming structures greater than duplexes of a heterodimers comprising a framework and dimerization polypeptide. In some instances, triplexes, tetraplexes, pentaplexes may be formed. Such aa sequences include, but are not limited to, IgM constant regions (see e.g., FIG. 4H) which forms hexamer, or pentamers (particularly when combined with a mature j-chain peptide lacking a signal sequence such as that provided in FIG. 4J. Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times such as 10-20, 20-30, or 30-40 times). In such sequences, Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times, such as 10-20, 20-30, or 30-40 times. A collagen oligomerization peptide can comprise the following aa sequence:

(SEQ ID NO: 86)
VTAFSNMDDMLQKAHLVIEGTFIYLRDSTEFFIRVRDGWKKLQLGELIP
IPADSPPPPALSSNP.

Suitable framework polypeptides (e.g., those with an Ig Fc multimerization sequence) will, in some cases, be half-life extending polypeptides. Thus, in some cases, a suitable framework polypeptide increases the in vivo half-life (e.g., the serum half-life) of the MAPPs, compared to a control MAPP having a framework polypeptide with a different aa sequence. For example, in some cases, a framework polypeptide increases the in vivo half-life (e.g., the serum half-life in a mammal such as a human) of the MAPP, compared to a control MAPP having a framework polypeptide with a different aa sequence. The half-life may be extended by at least about 10%, at least about 15%, at least about 25%, at least about 50%, at least about 100%, at least about 2-fold, at least about 5-fold, at least about 10-fold, at least about 25-fold, at least about 50-fold, at least about 100-fold, or more than 100-fold. As an example, in some cases, an Ig Fc polypeptide sequence (e.g., utilized as a multimerization sequence to form a duplex of MAPP heterodimers comprising a framework and dimerization polypeptide) increases the stability and/or in vivo half-life (e.g., the serum half-life) of a MAPP duplex compared to a control MAPP duplex lacking the Ig Fc polypeptide sequence by at least about 10%, at least about 15%, at least about 25%, at least about 50%, at least about 100%, at least about 2-fold, at least about 2.5-fold, at least about 5-fold, at least about 10-fold, at least about 25-fold, at least about 50-fold, at least about 100-fold, or more than 100-fold.
3. Presenting Sequence and Presenting Complexes

Presenting sequences (epitope presenting sequences) and presenting complexes (epitope presenting complexes) comprise MHC Class I polypeptides (MHC-H and β2M) sufficient to bind and present an epitope to a TCR. Presenting sequences and complexes may also comprise additional protein (peptide) elements including one or more independently selected MODs and/or one or more independently selected linkers (e.g., linkers placed between various domains). As discussed herein, unless stated otherwise, neither presenting sequences nor presenting complexes comprise an MHC transmembrane domain (or intracellular domain such as a cytoplasmic tail) sufficient to anchor MAPP molecules (e.g., more than 50% of the MAPP molecules) in a mammalian cell membrane (e.g., a CHO cell membrane) when expressed therein.

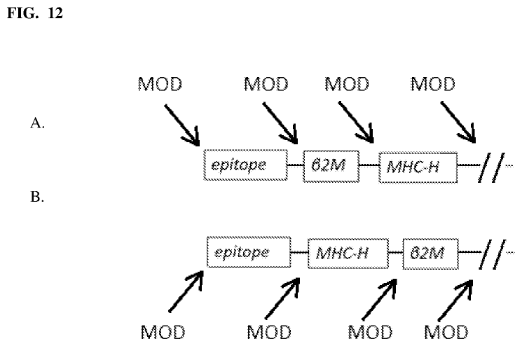

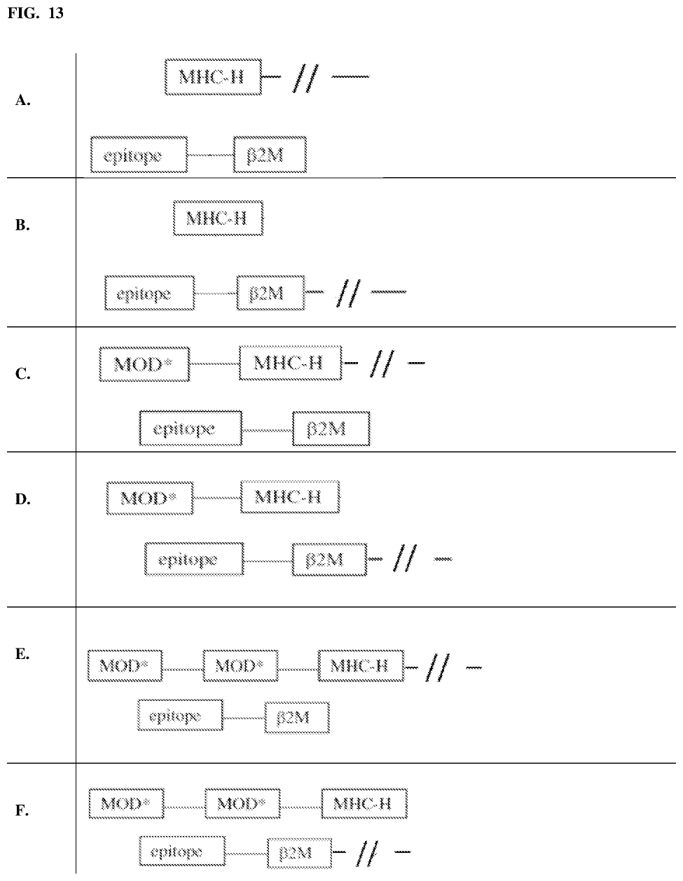

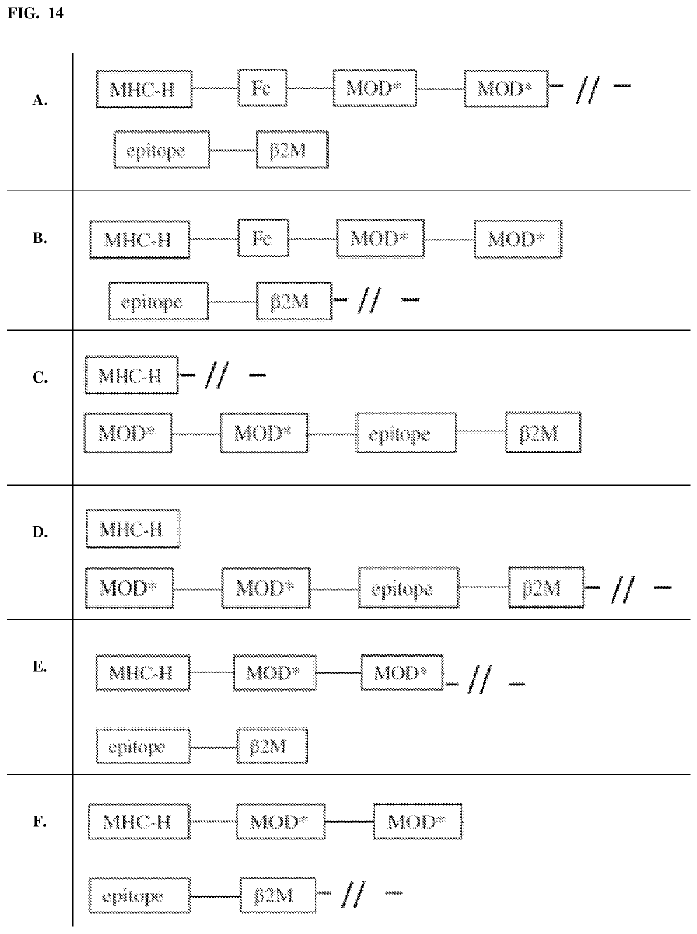

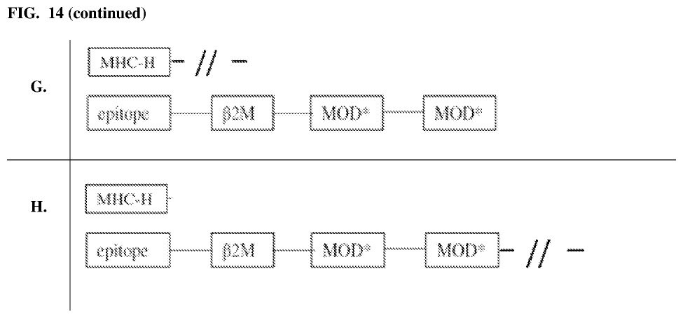

Conceptually, each of the presenting sequences and presenting complexes may be considered a “soluble MHC” that is fully capable of binding and presenting a peptide epitope (e.g., and peptide epitope that is part of a polypeptide comprising an MHC sequence) to a TCR. In presenting sequences all of the MHC sequences and optionally the peptide epitope sequence are present in a single polypeptide chain (single linear sequence of aas produced by translation). See e.g., FIG. 12 structures A and B.

Where the MHC-H and β2M polypeptide sequences are divided among two or more polypeptide chains the “soluble MHC” is termed a presenting complex. The presenting complex has one chain that is part of a framework peptide or dimerization peptide, referred to as a “presenting complex 1^{st} sequence.” The second chain of the presenting complex is termed the “presenting complex 2^{nd} sequence.” The presenting complex 2^{nd} sequence may be associated non-covalently with the MHC components present in the presenting complex 1^{st} sequence (through binding interactions between MHC-H and β2M components as in FIGS. 13 and 14), in addition, one or more disulfide bonds between the presenting complex 1^{st} sequence and the presenting complex 2^{nd} sequence (see, e.g., FIGS. 13-15). Alternatively, the presenting complex 2nd sequence may be associated non-covalently with the MHC components present in the presenting complex 1st sequence through binding interactions between MHC-H and β2M polypeptide sequences and/or through binding sequences (e.g., such as interspecific binding sequences) in the presence or absence of one or more disulfide bonds between the presenting complex 1st sequence and the presenting complex 2nd sequence.

In some cases, one or more presenting sequence of a MAPP comprises all of the Class I components required for binding and presenting the epitope of interest to a TCR; e.g., the MHC-H and β2M and epitope in a single polypeptide sequence. In some cases, a MAPP comprises one or more presenting complexes with all of the Class I components required for binding and presenting the epitope of interest to a TCR, where the epitope is part of the presenting complex 1^{st} sequence or the presenting complex 2^{nd} sequence.

Peptide presenting sequences and peptide presenting complexes in MAPPs may contain one or more substitutions in, for example, the MOD sequences and/or MHC sequences. By way of example, the presenting proteins or complexes may contain one or more reduced affinity variant MODs. The presenting sequences or complexes may also comprise substitutions in their MHC sequences, such as a Class I MHC containing a Y84A substitution (as discussed above, that opens the end of the binding pocket). A Class I MHC containing presenting sequence or presenting complex may also contain substitutions leading to stabilizing disulfide bonds, such as a cysteine substituted into the carboxyl end portion of the al helix and a cysteine in the amino end portion of the α2-1 helix (e.g., a disulfide bond formed between positions 84 and 139 in a Y84C and A139C substituted MHC-H as previously discussed). A Class I MHC containing presenting sequence or presenting complex bearing cysteine substitutions at MHC-H aa 236 (A236C) and β2M aa 12 (e.g., R12C) may form a disulfide bond between those aas. In presenting sequences or presenting complexes having peptide epitopes bound by a linker to the N-terminal side of the β2M sequence(s), a stabilizing disulfide bond joining a cysteine residue present in the linker (e.g., at position 2 of a the linker such as a G2C substitutions in the linkers of SEQ ID NOs:131-133 and 571 provided below) and a cysteine residue in the MHC-H polypeptide (e.g., a Cys introduced at position 84 such as a Y84C substitution) may also be introduced. A Class I MHC containing presenting sequence or presenting complex may also contain paired substitutions forming two disulfide bonds such as between the R12C/Y84C and the Y84C/linker (e.g., G2C) substitution pairs.
4. MHC Class I Polypeptides

For the purpose of this disclosure, the term “MHC polypeptide” is meant to include Class I MHC polypeptides, including MHC-H polypeptide sequences (e.g., α1, α2, and α3 MHC-H polypeptide domains) and the β-2 microglobulin (β2M) polypeptide sequences, which represent all or most of the extracellular Class I protein required for presentation of an epitope to a TCR. Accordingly, the MHC Class I heavy chain present in a MAPP of this disclosure may include only MHC Class I heavy chain α1, α2, and α3 domains. In some cases, the MHC Class I heavy chain sequence present in a MAPP of the present disclosure has a length from about 270 aa to about 290 aa. In some cases, the MHC Class I heavy chain present in a MAPP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

The epitope-containing MAPPs include MHC polypeptides of various species, including human MHC polypeptides (HLA polypeptides), rodents (e.g., mouse, rat, etc.), and the MHC polypeptides of other mammalian species (e.g., lagomorphs, non-human primates, canines, felines, ungulates (e.g., equines, bovines, ovines, caprines, etc.), and the like. Both the β2M and MHC-H chain sequences in a MAPP may be of human origin.

MAPPs and their higher order complexes (e.g., duplex MAPPs) are intended to be soluble in aqueous media under physiological conditions (e.g., soluble in human blood plasma at therapeutic levels). Unless expressly stated otherwise, as noted above, the MAPPs described herein are not intended to include membrane anchoring domains (such as transmembrane regions of MHC Class I polypeptides, or a part thereof sufficient to anchor MAPP molecules (e.g., more than 50% of the MAPP molecules), or a peptide thereof, in the membrane of a cell (e.g., a eukaryotic cell such as a mammalian cell such as a Chinese Hamster Ovary or “CHO” cell) in which the MAPP is expressed. Similarly, unless expressly stated otherwise, the MAPPs described herein do not include the leader and/or intracellular portions (e.g., cytoplasmic tails) that may be present in some naturally-occurring MHC Class I proteins

In some cases, a MHC polypeptide of a MAPP is a human MHC polypeptide, and may be referred to as “human leukocyte antigen” (“HLA”) polypeptides, more specifically, a Class I HLA polypeptide, e.g., a β2M polypeptide, or a Class I HLA heavy chain polypeptide. Class I HLA heavy chain polypeptides that can be included in MAPPs include, for example, HLA-A, -B, -C, -E, -F, and/or -G heavy chain polypeptide extracellular domains (e.g., fully process and lacking the signal sequence). In an embodiment, the Class I HLA heavy chain polypeptides of MAPPs comprise polypeptides having a sequence having at least 75%, at least 80%, at least 85%, at least 90%, at least 95%, at least 98%, at least 99%, or 100% aa sequence identity to all or part (e.g., 50, 75, 100, 150, 200, or 250 contiguous aa) of the aa sequence of any of the human HLA heavy chain polypeptides depicted in FIGS. 3A-3I. For example, they may comprise 1-30, 1-5, 5-10, 10-15, 15-20, 20-25 or 25-30 aa insertions, deletions, and/or substitutions (in addition to those locations indicated as being variable in the heavy chain consensus sequences of FIGS. 3E-3I).

As an example, a MHC Class I heavy chain polypeptide of a MAPP can comprise an aa sequence having at least 75%, at least 80%, at least 85%, at least 90%, at least 95%, at least 98%, at least 99%, or 100% aa sequence identity to aas 25-300 (lacking all, or substantially all, of the leader, transmembrane and cytoplasmic sequences), or aas 25-365 (lacking the leader), of the human HLA-A heavy chain polypeptides depicted in FIGS. 3A, 3B and/or 3C.

An HLA heavy chain polypeptides (human MHC-H) of a MAPP may comprise a polypeptide sequence having at least 95%, sequence identity (e.g., 95-100%) sequence identity to all or part (e.g., 50, 75, 100, 150, 200, or 250 contiguous aa) of any one of the HLA heavy chain polypeptides in FIGS. 3A-3I. An HLA heavy chain polypeptides of a MAPP may comprise a polypeptide sequence having at least 98%, sequence identity (e.g., 98-100%) sequence identity to all or part (e.g., 50, 75, 100, 150, 200, or 250 contiguous aa) of any one of the HLA heavy chain polypeptides in FIGS. 3A-3I.

An HLA heavy chain polypeptides of a MAPP may comprise a polypeptide sequence having at least 95%, sequence identity (e.g., 95-100%) sequence identity to at least 125 contiguous aa of any one of the HLA heavy chain polypeptides in FIGS. 3A-3I. An HLA heavy chain polypeptides of a MAPP may comprise a polypeptide sequence having at least 98%, sequence identity (e.g., 98-100%) sequence identity to at least 125 contiguous aa of any one of the HLA heavy chain polypeptides in FIGS. 3A-3I.

HLA heavy chain polypeptides of a MAPP may comprise a polypeptide sequence having at least 95%, sequence identity (e.g., 95-100%) sequence identity to at least 175 contiguous aa of any one of the HLA heavy chain polypeptides in FIGS. 3A-3I. HLA heavy chain polypeptides of a MAPP may comprise a polypeptide sequence having at least 98%, sequence identity (e.g., 98-100%) sequence identity to at least 175 contiguous aa of any one of the HLA heavy chain polypeptides in FIGS. 3A-3I.

HLA heavy chain polypeptides of a MAPP may comprise a polypeptide sequence having at least 95%, sequence identity (e.g., 95-100%) sequence identity to at least 225 contiguous aa of any one of the HLA heavy chain polypeptides in FIGS. 3A-3I. HLA heavy chain polypeptides of a MAPP may comprise a polypeptide sequence having at least 98%, sequence identity (e.g., 98-100%) sequence identity to at least 225 contiguous aa of any one of the HLA heavy chain polypeptides in FIGS. 3A-3I.

HLA heavy chain polypeptides of a MAPP may comprise a polypeptide sequence having at least 95%, sequence identity (e.g., 95-100%) sequence identity to at least 250 contiguous aa of any one of the HLA heavy chain polypeptides in FIGS. 3A-3I. HLA heavy chain polypeptides of a MAPP may comprise a polypeptide sequence having at least 98%, sequence identity (e.g., 98-100%) sequence identity to at least 250 contiguous aa of any one of the HLA heavy chain polypeptides in FIGS. 3A-3I.

(i) MHC Class I Heavy Chains

Class I human MHC polypeptides may be drawn from the classical HLA alleles (HLA-A, B, and C), or the non-classical HLA alleles (e.g., HLA-E, F and G). The following are non-limiting examples of MHC-H alleles and variants of those alleles that may be incorporated into MAPPs.

(a) HLA-A Heavy Chains

The HLA-A heavy chain peptide sequences, or portions thereof, that may be incorporated into a MAPP include, but are not limited to, the alleles: A*0101 (FIG. 3D SEQ ID NO: 12, FIG. 3E SEQ ID NO: 23), A*0201(FIG. 3D SEQ ID NO: 15, FIG. 3E SEQ ID NO:24), A*0301 (FIG. 3E SEQ ID NO:25), A*1101 (FIG. 3D SEQ ID NO: 20, FIG. 3E SEQ ID NO: 26), A*2301 (FIG. 3E SEQ ID NO:27), A*2402 (FIG. 3D SEQ ID NO:21, FIG. 3E SEQ ID NO:28), A*2407(FIG. 3E SEQ ID NO:29), A*3303(FIG. 3D SEQ ID NO:22, FIG. 3E SEQ ID NO:30), and A*3401(FIG. 3E SEQ ID NO:31), which are aligned without all, or substantially all, of the leader, transmembrane and cytoplasmic sequences in FIG. 3E. Any of those alleles may comprise a substitution at one or more of positions 84, 139 and/or 236 (as shown in FIG. 3E) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, a HLA-A sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of any of those HLA-A alleles may also be employed (e.g., it may comprise 1-30, 1-5, 5-10, 10-15, 15-20, 20-25, or 25-30 aa insertions, deletions, and/or substitutions). The HLA-A heavy chain polypeptide sequence of a MAPP may comprise the Y84C and A139C substitutions.

HLA-A*0101 (HLA-A*01:01:01:01)

In an embodiment, a MHC Class I heavy chain polypeptide of a MAPP comprises an aa sequence of HLA-A*01:01:01:01 (SEQ ID NOs: 12 or 23), or a sequence having at least 75% (at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A heavy chain polypeptide of a MAPP has less than 100% identity to the sequence labeled HLA-A in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*0101 heavy chain polypeptide sequence of a MAPP may comprise the Y84C and A139C substitutions.

HLA-A*0201 (HLA-A*02:01)

In an embodiment, a MHC Class I heavy chain polypeptide of a MAPP comprises an aa sequence of HLA-A*0201 (SEQ ID NOs: 15 or 24) provided in FIG. 3D or FIG. 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A*0201 heavy chain polypeptide of a MAPP has less than 100% identity to the sequence labeled HLA-A*0201 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*0201 heavy chain polypeptide of a MAPP may comprise the Y84A and A236C substitutions. In an embodiment, the HLA-A*0201 heavy chain polypeptide of a MAPP comprises the Y84C and A139C substitutions. In an embodiment, the HLA-A*0201 heavy chain polypeptide of a MAPP comprises the Y84C, A139C and A236C substitutions.

HLA-A*1101 (HLA-A*11:01)

In an embodiment, a MHC Class I heavy chain polypeptide of a MAPP comprises an aa sequence of HLA-A*1101(SEQ ID NOs: 20 or 26) provided in FIG. 3D or in FIG. 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). The HLA-A*1101 heavy chain allele may be prominent in Asian populations, including populations of individuals of Asian descent.

In an embodiment, where the HLA-A*1101 heavy chain polypeptide of a MAPP has less than 100% identity to the sequence labeled HLA-A*1101 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*1101 heavy chain polypeptide of a MAPP may comprise the Y84A and A236C substitutions. In an embodiment, the HLA-A*1101 heavy chain polypeptide of a MAPP comprises the Y84C and A139C substitutions. In an embodiment, the HLA-A*1101 heavy chain polypeptide of a MAPP comprises the Y84C, A139C and A236C substitutions.

HLA-A*2402 (HLA-A*24:02)

In an embodiment, a MHC Class I heavy chain polypeptide of a MAPP comprises an aa sequence of HLA-A*2402 (SEQ ID NOs: 21 or 28) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). The HLA-A*2402 heavy chain allele may be prominent in Asian populations, including populations of individuals of Asian descent.

In an embodiment, where the HLA-A*2402 heavy chain polypeptide of a MAPP has less than 100% identity to the sequence labeled HLA-A*2402 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*2402 heavy chain polypeptide of a MAPP may comprise the Y84A and A236C substitutions. The HLA-A*2402 heavy chain polypeptide of a MAPP may comprise the Y84C and A139C substitutions. In an embodiment, the HLA-A*2402 heavy chain polypeptide of a MAPP comprises the Y84C, A139C and A236C substitutions.

HLA-A*3303 (HLA-A*33:03)

In an embodiment, a MHC Class I heavy chain polypeptide of a MAPP comprises an aa sequence of HLA-A*3303 (SEQ ID NOs: 22 or 30) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-25, 1-5, 5-10, 10-15, 15-20, 20-25, or 25-30 aa insertions, deletions, and/or substitutions). The HLA-A*3303 heavy chain allele may be prominent in Asian populations, including populations of individuals of Asian descent.

In an embodiment, where the HLA-A*3303 heavy chain polypeptide of a MAPP has less than 100% identity to the sequence labeled HLA-A*3303 in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). The HLA-A*3303 heavy chain polypeptide of a MAPP may comprise the Y84A and A236C substitutions. The HLA-A*3303 heavy chain polypeptide of a MAPP may comprise the Y84C and A139C substitutions. The HLA-A*3303 heavy chain polypeptide of a MAPP may comprise the Y84C, A139C and A236C substitutions.

(b) HLA-B Heavy Chains

The HLA-B heavy chain peptide sequences, or portions thereof, that may be incorporated into a MAPP include, but are not limited to, the alleles B*0702 shown in FIGS. 3D and 3F (SEQ ID NOs: 13 in FIG. 3D or 33 in FIG. 3F), or in FIG. 3F as B*0801 (SED ID NO:34), B*1502(SEQ ID NO:35), B27 (subtypes B*2701-2759), B*3802 (SEQ ID NO:36), B*4001 (SEQ ID NO:37), B*4601 (SEQ ID NO:38), and B*5301 (SEQ ID NO:39), which are aligned without all, or substantially all, of the leader, transmembrane and cytoplasmic sequences in FIG. 3F. Any of those alleles may comprise a substitution at one or more of positions 84, 139 and/or 236 (as shown in FIG. 3F) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-B sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of any of those HLA-B alleles may also be employed (e.g., it may comprise 1-30, 1-5, 5-10, 10-15, 15-20, 20-25, or 25-30 aa insertions, deletions, and/or substitutions). The HLA-B heavy chain polypeptide sequence of a MAPP may comprise the Y84C and A139C substitutions.

HLA-B*0702 (HLA-B*07:02)

In an embodiment, a MHC Class I heavy chain polypeptide of a MAPP comprises an aa sequence of HLA-B*0702 (SEQ ID NO:33 and SEQ ID NO:13 in FIG. 3E labeled HLA-B in FIG. 3D), or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-B heavy chain polypeptide of a MAPP has less than 100% identity to the sequence labeled HLA-B in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B*0702 heavy chain polypeptide of a MAPP may comprise the Y84A and A236C substitutions. The HLA-B*0702 heavy chain polypeptide of a MAPP may comprise the Y84C and A139C substitutions. The HLA-B*0702 heavy chain polypeptide of a MAPP may comprise the Y84C, A139C and A236C substitutions.

(c) HLA-C Heavy Chains

The HLA-C heavy chain peptide sequences, or portions thereof, that may be incorporated into a MAPP include, but are not limited to, the alleles: C*0102 (SEQ ID NO:41), C*0303 (SEQ ID NO: 42), C*0304 (SEQ ID NO:43), C*0401 (SEQ ID NO:44), C*0602 (SEQ ID NO:45), C*0701(SEQ ID NO:46), C*0702 (SEQ ID NO:47), C*0801 (SEQ ID NO:48), and C*1502 (SEQ ID NO:49), which are aligned without all, or substantially all, of the leader, transmembrane and cytoplasmic sequences in FIG. 3G. Any of those alleles may comprise a substitution at one or more of positions 84, 139 and/or 236 (as shown in FIG. 3G) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-C sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of any of those HLA-C alleles may also be employed (e.g., it may comprise 1-30, 1-5, 5-10, 10-15, 15-20, 20-25, or 25-30 aa insertions, deletions, and/or substitutions). The HLA-C heavy chain polypeptide sequence of a MAPP may comprise the Y84C and A139C substitutions.

HLA-C*701 (HLA-C*07:01) and HLA-C*702 (HLA-C*07:02)

The MHC Class I heavy chain polypeptide of a MAPP may comprise an aa sequence of HLA-C*701 (FIG. 3C SEQ ID NO:11) or HLA-C*702, FIG. 3G (SEQ ID NO:46) in FIG. 3D (labeled HLA-C), or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of one of those sequences (e.g., it may comprise 1-30, 1-5, 5-10, 10-15, 15-20, 20-25, or 25-30 aa insertions, deletions, and/or substitutions). In an embodiment, where the HLA-C heavy chain polypeptide of a MAPP has less than 100% identity to the sequence labeled HLA-C in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-C*701 heavy chain polypeptide of a MAPP may comprise the Y84A and A236C substitutions. The HLA-C*701 heavy chain polypeptide of a MAPP may comprise the Y84C and A139C substitutions. The HLA-C*701 heavy chain polypeptide of a MAPP may comprise the Y84C, A139C and A236C substitutions.

(d) Non-Classical HLA-E, F and G Heavy Chains

The non-classical HLA heavy chain peptide sequences, or portions thereof, that may be incorporated into a MAPP include, but are not limited to, those of the HLA-E, F, and/or G alleles. Sequences for those alleles (and the HLA-A, B and C alleles) may be found on the world wide web at, for example, hla.alleles.org/nomenclature/index.html, the European Bioinformatics Institute (www.ebi.ac.uk), which is part of the European Molecular Biology Laboratory (EMBL), and at the National Center for Bioecology Information (www.ncbi.nlm.nih.gov).

Some suitable HLA-E alleles include, but are not limited to, HLA-E*0101 (HLA-E*01:01:01:01), HLA-E*01:03(HLA-E*01:03:01:01), HLA-E*01:04, HLA-E*01:05, HLA-E*01:06, HLA-E*01:07, HLA-E*01:09, and HLA-E*01:10. Some suitable HLA-F alleles include, but are not limited to, HLA-F*0101 (HLA-F*01:01:01:01), HLA-F*01:02, HLA-F*01:03(HLA-F*01:03:01:01), HLA-F*01:04, HLA-F*01:05, and HLA-F*01:06. Some suitable HLA-G alleles include, but are not limited to, HLA-G*0101 (HLA-G*01:01:01:01), HLA-G*01:02, HLA-G*01:03(HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA-E, -F, and -G alleles without all, or substantially all, of the leader, transmembrane and cytoplasmic sequences are provided in FIG. 3H as SEQ ID NOs: 51, 52 and 53 respectively. Those consensus sequences are aligned in FIG. 3I with consensus sequences of the HLA-A, -B, and -C alleles (from FIGS. 3E-G).

Any of the above-mentioned HLA-E, F and/or G alleles may comprise a substitution at one or more of positions 84, 139 and/or 236 as shown in FIG. 3I for the consensus sequences. In an embodiment, the substitutions may be selected from: a position 84 tyrosine to alanine (Y84A) or cysteine (Y84C) or, in the case of HLA-F, a R84A or R84C substitution; a position 139 alanine to cysteine (A139C) or, in the case of HLA-F, a V139C; and an alanine to cysteine substitution at position 236 (A236C). In addition, an HLA-E, -F, and/or -G sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of any of the consensus sequences set forth in FIG. 3I may also be employed (e.g., the sequences may comprise 1-30, 1-5, 5-10, 10-15, 15-20, 20-25, or 25-30 aa insertions, deletions, and/or substitutions in addition to changes at variable residues listed therein). The HLA-E, F, or G heavy chain polypeptide sequence of a MAPP may comprise a cysteine at both position 84 and 139.

(e) Mouse H2K

In an embodiment, a MHC Class I heavy chain polypeptide of a MAPP comprises an aa sequence of MOUSE H2K (SEQ ID NO:16) (MOUSE H2K in FIG. 3D), or a sequence having at least 75%, at least 80%, at least 85%, at least 90%, at least 95%, at least 98%, at least 99%, or 100% aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the MOUSE H2K heavy chain polypeptide of a MAPP has less than 100% identity to the sequence labeled MOUSE H2K in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the MOUSE H2K heavy chain polypeptide of a MAPP comprises the Y84A and A236C substitutions. In an embodiment, the MOUSE H2K heavy chain polypeptide of a MAPP comprises the Y84C and A139C substitutions. In an embodiment, the MOUSE H2K heavy chain polypeptide of a MAPP comprises the Y84C, A139C and A236C substitutions.

(f) The Effect of Substitutions and Combinations of Substitutions in Class I Polypeptides on MAPPS

Substitution of position 84, particularly when it is a tyrosine residue, with a small aa such as alanine (Y84A) or glycine tends to open one end of the MHC binding pocket, allowing a linker (e.g., attached to an peptide epitope) to “thread” through the end of the MHC Class I binding pocket. A Y84A substitution permits greater variation in epitope sizes and allows longer peptides bearing epitope sequences to fit into the binding pocket and be presented by the MAPP. As with aa position 84 substitutions that open one end of the MHC-H binding pocket, substitution of an alanine or glycine at position 167 or its equivalent (e.g., a W167A or W167G substitutions or their equivalent) open the other end of the MHC binding pocket, creating a groove that permits greater variation (e.g., longer length) in the peptide epitopes that may be present. In an embodiment, the HLA-A heavy chain polypeptide of a MAPP comprises the Y84A and A236C substitutions. In an embodiment, the HLA-A heavy chain polypeptide of a MAPP comprises the Y84C and A139C substitutions. When aas 84 and 139 are both cysteines, they may form an intrachain disulfide bond which can stabilize the MHC Class I protein, thereby permitting translation and excretion of the MAPP by eukaryotic cells (e.g., CHO cells), even when not loaded with an peptide epitope. When position 84 is a C residue, it can also form an interchain disulfide bond with a linker attached to the N-terminus of a β2M polypeptide (e.g., epitope-GCGGS(G₄S) (SEQ ID NO:87 (where the G₄5 may be repeated 1-10 times)-mature β2M polypeptide (e.g., lacking its signal sequence), see FIG. 2 and SEQ ID NOs: 1 to 5). When aa 236 is a cysteine, it can form an interchain disulfide bond with cysteine at aa 12 of a variant β2M polypeptide that comprises a R12C substitution at that position. Some possible combinations of MHC Class 1 heavy chain sequence modifications that may be incorporated into a MAPP are shown in the Table that follows.

The Table below lists some MHC heavy chain sequence modifications that may be incorporated into a MAPP.

Table of MHC Class I heavy chain mutations

| | HLA Heavy Chain | | |
|---|---|---|---|
| | Sequence | Sequence | Specific |
| | From | Identity | Substitutions at aa positions 84, 139 |
| Entry | FIGs. 3D-H | Range‡ | and/or 236 |
| 1 | HLA-A | 75%-99.8%, 80%-99.8%, 85%-99.8%, | None; Y84C; Y84A; A139C; |
| | Consensus | 90%-99.8%, 95%-99.8%, 98%-99.8%, | A236C; (Y84A & A236C); Y84C & |
| | FIG. 3E | or 99%-99.8%; or 1-25, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | |
| | | counting variable residues) | |
| 2 | A*0101, A*0201, | 75%-99.8%, 80%-99.8%, 85%-99.8%, | None; Y84C; Y84A; A139C; |
| | A*0301, A*1101, | 90%-99.8%, 95%-99.8%, 98%-99.8%, | A236C; (Y84A & A236C); Y84C & |
| | A*2402, A*2301, | or 99%-99.8%; or 1-30, 1-5, 5-10, 10- | A236C; (Y84C & A139C); or |
| | A*2402, A*2407, | 15, 15-20, 20-25 or 25-30 aa insertions, | (Y84C, A139C & A236C) |
| | A*3303, or A*3401 | deletions, and/or substitutions | |
| 3 | HLA-B | 75%-99.8%, 80%-99.8%, 85%-99.8%, | None; Y84C; Y84A; A139C; |
| | Consensus | 90%-99.8%, 95%-99.8%, 98%-99.8%, | A236C; (Y84A & A236C); Y84C & |
| | FIG. 3F | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | |
| | | counting variable residues) | |
| 4 | B*0702, B*0801, | 75%-99.8%, 80%-99.8%, 85%-99.8%, | None; Y84C; Y84A; A139C; |
| | B*1502, B*3802, | 90%-99.8%, 95%-99.8%, 98%-99.8%, | A236C; (Y84A & A236C); Y84C & |
| | B*4001, B*4601, or | or 99%-99.8%; or 1-30, 1-5, 5-10, 10- | A236C; (Y84C & A139C); or |
| | B*5301 | 15, 15-20, 20-25 or 25-30 aa insertions, | (Y84C, A139C & A236C) |
| | | deletions, and/or substitutions | |
| 5 | HLA-C | 75%-99.8%, 80%-99.8%, 85%-99.8%, | None; Y84C; Y84A; A139C; |
| | Consensus | 90%-99.8%, 95%-99.8%, 98%-99.8%, | A236C; (Y84A & A236C); Y84C & |
| | FIG. 3G | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | |
| | | counting variable residues) | |
| 6 | C*0102, C*0303, | 75%-99.8%, 80%-99.8%, 85%-99.8%, | None; Y84C; Y84A; A139C; |
| | C*0304, C*0401, | 90%-99.8%, 95%-99.8%, 98%-99.8%, | A236C; (Y84A & A236C); Y84C & |
| | C*0602, C*0701, | or 99%-99.8%; or 1-30, 1-5, 5-10, 10- | A236C; (Y84C & A139C); or |
| | C*702, C*0801, or | 15, 15-20, 20-25 or 25-30 aa insertions, | (Y84C, A139C & A236C) |
| | C*1502 | deletions, and/or substitutions | |
| 7 | HLA-E, F, or G | 75%-99.8%, 80%-99.8%, 85%-99.8%, | None; Y84C; Y84A; A139C; |
| | Consensus FIG. 3H | 90%-99.8%, 95%-99.8%, 98%-99.8%, | A236C; (Y84A & A236C); Y84C & |
| | | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | Note that in HLA F contains an R84 |
| | | counting variable residues) | and V139, but can be substituted |
| | | | with C or A giving the |
| | | | corresponding R84C, R84A and/or |
| | | | V139C substitutions |
| 8 | MOUSE H2K | 75%-99.8%, 80%-99.8%, 85%-99.8%, | None; Y84C; Y84A; A139C; |
| | | 90%-99.8%, 95%-99.8%, 98%-99.8%, | A236C; (Y84A & A236C); Y84C & |
| | | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions | |

‡The Sequence Identity Range is the permissible range in sequence identity of a MHC-H polypeptide sequence incorporated into a MAPP relative to the corresponding portion of sequences listed in FIGS. 3D-3H not counting the variable residues in the consensus sequences.

(g) β2-Microglobin Polypeptides and Their Combination with MHC-H Polypeptides

A β2M polypeptide of a MAPP can be a human β2M polypeptide, a non-human primate β2M polypeptide, a murine β2M polypeptide, and the like. In some instances, a β2M polypeptide comprises an aa sequence having at least 75%, at least 80%, at least 85%, at least 90%, at least 95%, at least 98%, at least 99%, or 100% aa sequence identity to a β2M aa sequence depicted in FIG. 2. In some instances, a β2M polypeptide comprises an aa sequence having at least 75%, at least 80%, at least 85%, at least 90%, at least 95%, at least 98%, at least 99%, or 100% aa sequence identity to aas 21 to 119 of a β2M aa sequence depicted in FIG. 2.

In some cases, a MHC Class I polypeptide comprises an aa substitution replacing another aa with a cysteine (Cys) residue. Such cysteine residues can form a disulfide bond with a cysteine residue present in a second polypeptide chain.

In some cases, a β2M polypeptide sequence and an MHC-H polypeptide sequence comprise naturally occurring cysteines substituted for another aa. Such cysteines permit formation of a disulfide bond between those sequences. For example, in some cases MAPPs may contain a disulfide bond between one of following pairs of residues in a HLA β2M (see FIG. 2) and a HLA Class I heavy chains (see FIGS. 3D-3I) (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The aa numbering of the MHC/HLA Class I heavy chain is in reference to the mature MHC/HLA Class I heavy chain, without a signal peptide. For example, in some cases, residue 236 of the mature HLA-A, -B, or -C aa sequence (i.e., residue 260 of the aa sequence depicted in FIGS. 3A-3C respectively) is substituted with a Cys. In some cases, residue 32 (corresponding to Arg-12 of mature β2M) of an aa sequence depicted in FIG. 2 is substituted with a Cys.

Separately, or in addition to, the pairs of cysteine residues in a β2M and HLA Class I heavy chain polypeptide that may be used to form interchain disulfide bonds between the first and second polypeptides of a MAPP (discussed above), the HLA-heavy chain of a MAPP or its epitope conjugate may be substituted with cysteines to form an intrachain disulfide bond between a cysteine substituted into the carboxyl end portion of the al helix and a cysteine in the amino end portion of the α2-1 helix. Such disulfide bonds stabilize the MAPP and permit its cellular processing and excretion from eukaryotic cells in the absence of a bound peptide epitope (or null peptide). In one embodiment, a disulfide bond is formed between the carboxyl end portion of the al helix is from about aa position 79 to about aa position 89 and the amino end portion of the α2-1 helix is from about aa position 134 to about aa position 144 of the MHC Class I heavy chain (the aa positions are determined based on the sequence of the heavy chains without their leader sequence (see, e.g., FIGS. 3D-3H). In one such embodiment the disulfide bond is between a cysteine located at positions 83, 84, or 85 and a cysteine located at any of positions 138, 139 or 140 of the MHC Class I heavy chain. For example, a disulfide bond may be formed from cysteines incorporated into the MHC Class I heavy chain at aa 83 and a cysteine at an aa located at any of positions 138, 139 or 140. Alternatively, a disulfide bond may be formed between a cysteine inserted at position 84 and a cysteine inserted at any of positions 138, 139 or 140, or between a cysteine inserted at position 85 and a cysteine at any one of positions 138, 139 or 140. In an embodiment, the MHC Class I heavy chain intrachain disulfide bond is between cysteines substituted into a heavy chain sequence at positions 84 and 139 (e.g., the heavy chain sequence may be one of the heavy chain sequences set forth in FIGS. 3D-3H). As noted above, any of the MHC Class I intrachain disulfide bonds, including a disulfide bond between cysteines at 84 and 139, may be combined with other interchain disulfide bonds including a bond between MHC Class 1 heavy position 236 and position 12 of a mature β2M polypeptide sequence (lacking its leader) as shown, for example, in FIG. 2 .

In another embodiment, an intrachain disulfide bond may be formed in a MHC-H sequence of a MAPP between a cysteine substituted into the region between aa positions 79 and 89 and a cysteine substituted into the region between aa positions 134 and 144 of the sequences given in FIGS. 3D-3H. In such an embodiment, the MHC Class I heavy chain sequence may have insertions, deletions and/or substitutions of 1 to 5 aas preceding or following the cysteines forming the disulfide bond between the carboxyl end portion of the al helix and the amino end portion of the α2-1 helix. Any inserted aas may be selected from the naturally occurring aas or the naturally occurring aas except proline and alanine.

In an embodiment, the β2M polypeptide sequence of a MAPP comprises a mature β2M polypeptide sequence (aas 21-119) of any one of NP_004039.1, NP_001009066.1, NP_001040602.1, NP_776318.1, or NP_033865.2 (SEQ ID NOs: 1 to 5).

In some cases, a HLA Class I heavy chain polypeptide of a MAPP or its epitope conjugate comprises any one of the HLA-A, -B, -C, -E, -F, or -G sequences in FIGS. 3D-3H. Any of the heavy chain sequences may further comprise cysteine substitutions at positions 84 and 139, which may form an intrachain disulfide bond.

In an embodiment, the β2M polypeptide of a MAPP comprises a mature β2M polypeptide sequence (aas 21-119) of any one of the sequences in FIG. 2, which further comprises a R12C substitution.

In an embodiment, a MAPP comprises a mature β2M polypeptide sequence (e.g., aas 21-119 of any one of the sequences in FIG. 2) having a R12C substitution, and any one of the HLA-A, -B, -C, -E, -F, or -G sequences in FIGS. 3D-3H having or substituted with a cysteine at position 236. In such embodiments a disulfide bond may form between the cysteines at positions 12 and 236. In addition, any of the heavy chain sequences may further comprise cysteine substitutions at positions 84 and 139, which may also form a disulfide bond.

In an embodiment, a MAPP has a β2M peptide sequence comprising aas 21-119 of a β2M sequence in FIG. 2 (e.g., NP_004039.1) with a R12C substitution, and an HLA Class I heavy chain polypeptide comprises the aa sequence:
GSHSMRYFFTSVSRPGRGEPRFIAVGYVDDTQFVRFDSDAASQRMEPRAPWIEQEGPEYWDGE TRKVKAHSQTHRVDL(aa cluster 1){C}(aa cluster 2)AGSHTVQRMYGCDVGSDWRFLRGYHQYA YDGKDYIALKEDLRSW(aa cluster 3){C}(aa cluster 4)HKWEAAHVAEQLRAYLEGTCVEWLRRY LENGKETLQRTDAPKTHMTHHAVSDHEATLRCWALSFYPAEITLTWQRDGEDQTQDTELVET RPAGDGTFQKWAAVVVPSGQEQRYTCHVQHEGLPKPLTLRWEP (SEQ ID NO:88); or, the first polypeptide comprises the sequence,
IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:89), and the second polypeptide comprises the aa sequence,
GSHSMRYFFTSVSRPGRGEPRFIAVGYVDDTQFVRFDSDAASQRMEPRAPWIEQEGPEYWDGE TRKVKAHSQTHRVDL(aa cluster 1){C}(aa cluster 2)AGSHTVQRMYGCDVGSDWRFLRGYHQYA YDGKDYIALKEDLRSW(aa cluster 3){C}(aa cluster 4))HKWEAAHVAEQLRAYLEGTCVEWLRR YLENGKETLQRTDAPKTHMTHHAVSDHEATLRCWALSFYPAEITLTWQRDGEDQTQDTEL(aa cluster 5)(C)(aa cluster 6)QKWAAVVVPSGQEQRYTCHVQHEGLPKPLTLRWEP (SEQ ID NO:90); where the cysteine residues indicated as {C} form a disulfide bond between the al and α2-1 helices and the (C) residue forms a disulfide bond with the mature β2M polypeptide cysteine at position 12.

Each occurrence of aa cluster 1, aa cluster 2, aa cluster 3, aa cluster 4, aa cluster 5, and aa cluster 6 is independently selected to be 1-5 aa residues, wherein the aa residues are each selected independently from i) any naturally occurring (proteogenic) aa or ii) any naturally occurring aa except proline or glycine.

In an embodiment where the MHC Class I heavy chain is an HLA-A chain:
aa cluster 1 may be the aa sequence GTLRG or that sequence with one or two aas deleted or substituted with other naturally occurring aas (e.g., L replaced by I, V, A or F);
aa cluster 2 may be the aa sequence YNQSE or that sequence with one or two aas deleted or substituted with other naturally occurring aas (e.g., N replaced by Q, Q replaced by N, and/or E replaced by D);
aa cluster 3 may be the aa sequence TAADM or that sequence with one or two aas deleted or substituted with other naturally occurring aas (e.g., T replaced by S, A replaced by G, D replaced by E, and/or M replaced by L, V, or I);
aa cluster 4 may be the aa sequence AQTTK or that sequence with one or two aas deleted or substituted with other naturally occurring aas (e.g., A replaced by G, Q replaced by N, or T replaced by S, and or K replaced by R or Q);
aa cluster 5 may be the aa sequence VETRP or that sequence with one or two aas deleted or substituted with other naturally occurring aas (e.g., V replaced by I or L, E replaced by D, T replaced by S, and/or R replaced by K); and/or
aa cluster 6 may be the aa sequence GDGTF or that sequence with one or two aas deleted or substituted with other naturally occurring aas (e.g., D replaced by E, T replaced by S, or F replaced by L, W, or Y).

In some cases, the β2M polypeptide sequence of a MAPP comprises the aa sequence:

(SEQ ID NO: 91)
IQRTPKIQVYSCHPAENGKSNFLNCYVSGFHPSDIEVDLLKNGERIEKV
EHSDLSFSKDWSFYLLYYTEFTPTEKDEYACRVNHVTLSQPKIVKWDRD
M.

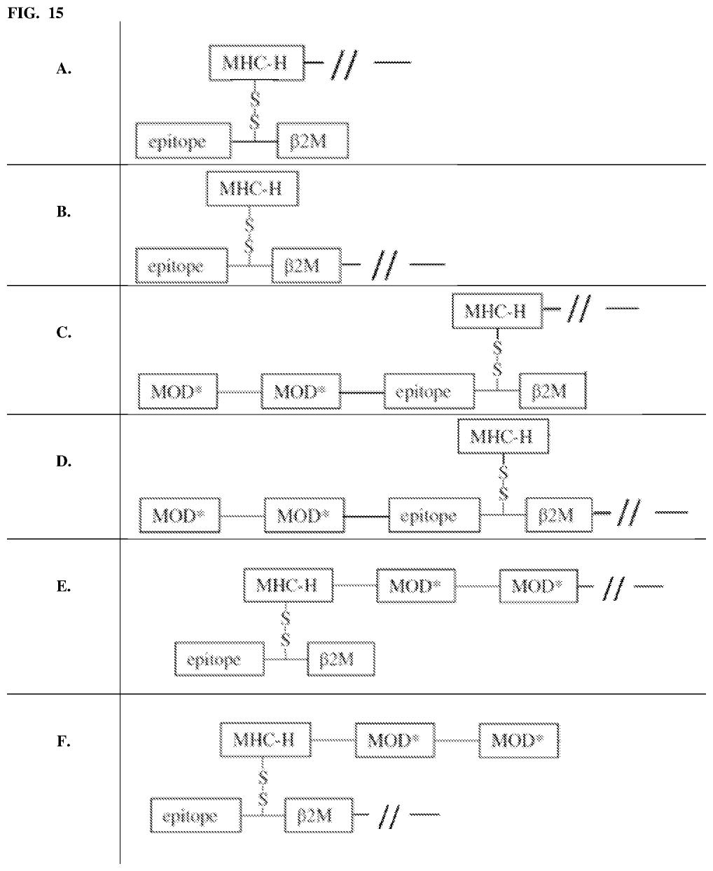

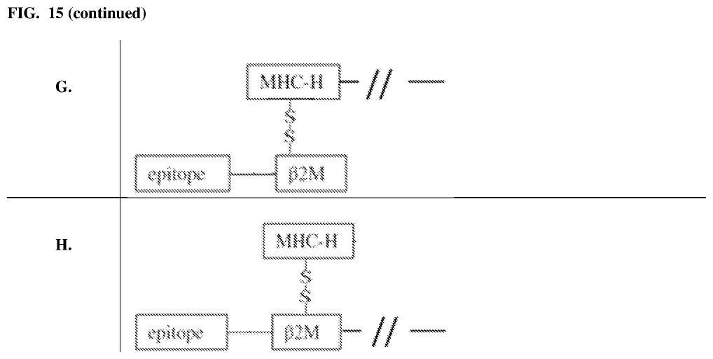

In some cases, such as where a MAPP comprises a presenting complex with an epitope and β2M sequence on one of the presenting complex 1^{st} sequence and the presenting complex 2^{nd} sequence, with the MHC-H sequences on the other of presenting complex 1^{st} sequence and the presenting complex 2^{nd} sequence, one or more disulfide bond may be used to link the presenting complex 1^{st} sequence and the presenting complex 2^{nd} sequence (see, e.g., FIG. 15). In one instance the disulfide bond joining the presenting complex 1^{st} sequence and the presenting complex 2^{nd} sequence is between a Cys residue present in a linker connecting the epitope and a β2M polypeptide sequence and a Cys residue in the MHC-H polypeptide (see, e.g., FIG. 15, structures A to F). In such an instance the Cys residue present in the MHC Class I heavy chain may be a Cys introduced as a Y84C substitution and the linker connecting the peptide epitope and the β2M polypeptide in the first polypeptide chain is GCGGS(G₄S) where the G₄S may be repeated from 1 to 9 times (SEQ ID NO:92) (e.g., epitope-GCGGS(G₄S)-mature β2M polypeptide). For example, in some cases, the linker comprises the aa sequence GCGGSGGGGSGGGGSGGGGS (SEQ ID NO:93). As another example, the linker comprises the aa sequence GCGGSGGGGSGGGGS (SEQ ID NO:94). In another instance, the disulfide bond joining the presenting complex 1^{st} sequence and the presenting complex 2^{nd} sequence is between a Cys residue in a β2M polypeptide sequence and a Cys residue present in a MHC Class I heavy sequence (see, e.g., FIG. 15, structures G and H). In such an instance the Cys residue present in the MHC Class I heavy chain may be a Cys introduced as a A-236C substitution and the Cys residue present in the β2M polypeptide sequence is a Cys introduced as a R12C substitution.
5. Immunomodulatory Polypeptides (“MODs”)

A MAPP may comprise one or more immunomodulatory polypeptides or “MODs”. MODs that are suitable for inclusion in a MAPP of the present disclosure include, but are not limited to, IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL, and fragments of any thereof, such as ectodomain fragments, capable of engaging and signaling through their cognate receptor). Some MOD polypeptides suitable for inclusion in a MAPP of the present disclosure, and their “co-MODs (“co-immunomodulatory polypeptides” or cognate costimulatory receptors) include polypeptide sequences with T cell modulatory activity from the protein pairs recited in the following table:

Exemplary Pairs of MODs and Co-MODs

| | |
|---|---|
| a) | 4-1BBL (MOD) and 4-1BB (Co-MOD); |
| b) | PD-L1 (MOD) and PD1 (Co-MOD); |
| c) | IL-2 (MOD) and IL-2 receptor (Co-MOD); |
| d) | CD80 (MOD) and CD28 (Co-MOD); |
| e) | CD86 (MOD) and CD28 (Co-MOD); |
| f) | OX40L (CD252) (MOD) and OX40 (CD134) |
| | (Co-MOD); |
| g) | Fas ligand (MOD) and Fas (Co-MOD); |
| h) | ICOS-L (MOD) and ICOS (Co-MOD); |
| i) | ICAM (MOD) and LFA-1 (Co-MOD); |
| j) | CD30L (MOD) and CD30 (Co-MOD); |
| k) | CD40 (MOD) and CD40L (Co-MOD); |
| l) | CD83 (MOD) and CD83L (Co-MOD); |
| m) | HVEM (CD270) (MOD) and CD160 (Co- |
| | MOD); |
| n) | JAG1 (CD339) (MOD) and Notch (Co- |
| | MOD); |
| o) | JAG1 (CD339) (MOD) and CD46 (Co- |
| | MOD); |
| p) | CD70 (MOD) and CD27 (Co-MOD); |
| q) | CD80 (MOD) and CTLA4 (Co-MOD); |
| r) | CD86 (MOD) and CTLA4 (Co-MOD); |
| s) | PD-L1(MOD) and CD-80 (Co-MOD); and |
| t) | TGF-β1, TGF-β2, and/or TGF-β3 (MODs) |
| | and TGF-β Receptor (e.g., TGFBR1 |
| | and/or TGFBR2) (Co-MOD) |

In some cases, the MOD is selected from an IL-2 polypeptide, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a TGFI3 polypeptide, and a PD-L2 polypeptide. In some cases, the MAPP or duplex MAPP comprises two different MODs, such as an IL-2 MOD or IL-2 variant MOD polypeptide and either a CD80 or CD86 MOD polypeptide. In another instance, the MAPP or duplex MAPP comprises an IL-2 MOD or IL-2 variant MOD polypeptide and a PD-L1 MOD polypeptide. In some case MODs, which may be the same or different, are present in a MAPP or duplex MAPP in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD polypeptide may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a MAPP or duplex MAPP does not comprise the signal peptide, intracellular domain, or a sufficient portion of the transmembrane domain to anchor a substantial amount (e.g., more than 5% or 10%) of a MAPP or duplex MAPP into a mammalian cell membrane.

In some cases, a MOD suitable for inclusion in a MAPP comprises all or a portion of (e.g., an extracellular portion of) the aa sequence of a naturally-occurring MOD. In other instances, a MOD suitable for inclusion in a MAPP is a variant MOD that comprises at least one aa substitution compared to the aa sequence of a naturally-occurring MOD. In some instances, a variant MOD exhibits a binding affinity for a co-MOD that is lower than the affinity of a corresponding naturally-occurring MOD (e.g., a MOD not comprising the aa substitution(s) present in the variant) for the co-MOD. Suitable variations in MOD polypeptide sequence that alter affinity may be identified by scanning (making aa substitution e.g., alanine substitutions or “alanine scanning” or charged residue changes) along the length of a peptide and testing its affinity. Once key aa positions altering affinity are identified those positions can be subject to a vertical scan in which the effect of one or more aa substitutions other than alanine are tested.

a. MODs and Variant MODs with Reduced Affinity

A MOD can comprise a wild-type amino acid sequence, or can comprise one or more amino acid substitutions, insertions, and/or deletions relative to a wild-type amino acid sequence. The immunomodulatory polypeptide can comprise only the extracellular portion of a full-length immunomodulatory polypeptide. Alternatively, a MOD can comprise all or a portion of (e.g., an extracellular portion of) the amino acid sequence of a naturally occurring MOD polypeptide.

Variant MODs comprise at least one amino acid substitution, addition and/or deletion as compared to the amino acid sequence of a naturally occurring immunomodulatory polypeptide. As noted above, in some instances a variant MOD exhibits a binding affinity for a co-MOD that is lower than the affinity of a corresponding naturally occurring MOD (e.g., an immunomodulatory polypeptide not comprising the amino acid substitution(s) present in the variant) for the co-MOD.

MOD polypeptides and variants, including reduced affinity variants, of proteins such as PD-L1, CD80, CD86, 4-1BBL and IL-2 are described in the published literature, e.g., published PCT application WO2020132138A1, the disclosure of which as it pertains to immunomodulatory polypeptides and specific variant immunomodulatory polypeptides of PD-L1, CD80, CD86, 4-1BBL, IL-2 are expressly incorporated herein by reference, including specifically paragraphs [00260]-[00455] of WO2020132138A1.

Suitable immunomodulatory domains that exhibit reduced affinity for a co-immunomodulatory domain can have from 1 aa to 20 aa differences from a wild-type immunomodulatory domain. For example, in some cases, a variant MOD present in a MAPP may include a single aa substitution compared to a corresponding reference (e.g., wild-type) MOD. A variant MOD present in a MAPP may include 2 aa substitutions compared to a corresponding reference (e.g., wild-type) MOD. A variant MOD present in a MAPP may include 3 or 4 aa substitutions compared to a corresponding reference (e.g., wild-type) MOD. A variant MOD present in a MAPP may include 5 or 6 aa substitutions compared to a corresponding reference (e.g., wild-type) MOD. A variant MOD present in a MAPP may include 7, 8, 9 or 10 aa substitutions compared to a corresponding reference (e.g., wild-type) MOD. A variant MOD present in a MAPP may include 11-15 or 15-20 aa substitutions compared to a corresponding reference (e.g., wild-type) MOD.

As discussed above, a variant MOD suitable for inclusion in a MAPP may exhibit reduced affinity for a cognate co-MOD, compared to the affinity of a corresponding wild-type MOD for the cognate co-MOD. In some cases, a variant MOD present in a MAPP has a binding affinity for a cognate co-MOD that is from 100 nM to 100 μM. For example, in some cases, a variant MOD present in a MAPP has a binding affinity for a cognate co-MOD that is from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 20 μM, from about 20 μM to about 30 μM, from about 30 μM to about 50 μM, from about 50 μM to about 75 μM, or from about 75 μM to about 100 μM.

Binding affinity between a MOD polypeptide sequence and its cognate co-MOD polypeptide can be determined by bio-layer interferometry (BLI) using the purified MOD polypeptide sequence and purified cognate co-MOD polypeptide, following the procedure set forth in published PCT Application WO 2020/132138 A1.

b. IL-2 and Its Variants

As one non-limiting example, a MOD or variant MOD present in a MAPP is an IL-2 or variant IL-2 polypeptide. In some cases, a variant MOD present in a MAPP is a variant IL-2 polypeptide. Wild-type IL-2 binds to an IL-2 receptor (IL-2R). A wild-type IL-2 aa sequence can be as follows:

APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT (aa 21-153 of UniProt P60568,
SEQ ID NO: 95).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ are provided in the accompanying sequence listing as SEQ ID NO:96, SEQ ID NO:97 and SEQ ID NO:98, respectively, and are also provided in, for example, U.S. Patent Pub. No. 20200407416.

In some cases, a variant IL-2 polypeptide exhibits reduced binding affinity to one or more of the IL-2Rα, IL2Rβ, and/or IL-2Rγ chains of human IL-2R, compared to the binding affinity of an IL-2 polypeptide comprising the aa sequence set forth in SEQ ID NO:95. For example, in some cases, a variant IL-2 polypeptide binds to one or more of the IL-2Rα, IL2Rβ, and/or IL-2Rγ chains of human IL-2R with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an IL-2 polypeptide comprising the aa sequence set forth in SEQ ID NO:95 for the α,β, and/or γ chains of IL-2R (e.g., an IL-2R comprising polypeptides comprising the aa sequence set forth in SEQ ID NOs:96-98), when assayed under the same conditions.

For example, IL-2 variants with a substitution of phenylalanine at position 42 (e.g., with an alanine), exhibit substantially reduced binding to the IL-2Rα chain, in which case the variant may reduce the activation of Tregs. IL-2 variants with a substitution of histidine at position 16 (e.g., with an alanine) exhibit reduced binding to the IL2Rβ chain, thereby reducing the likelihood of a MAPP binding to non-target T cells by virtue of off-target binding of the IL-2 MOD. Some IL-2 variants, e.g., those with substitutions of the F42 and H16 amino acids, exhibit substantially reduced binding to the IL-2Rα chain and also reduced binding to the IL2Rβ chain. See, e.g., Quayle, et al., Clin Cancer Res; 26(8) Apr. 15, 2020.

In some cases, a variant IL-2 polypeptide has a single aa substitution compared to the IL-2 aa sequence set forth in SEQ ID NO:95. In some cases, a variant IL-2 polypeptide has from 2 to 10 aa substitutions compared to the IL-2 aa sequence set forth in SEQ ID NO:95. In some cases, a variant IL-2 polypeptide has 2, 3, 4, 5, 6, 7, 8, 9 or 10 aa substitutions compared to the IL-2 aa sequence set forth in SEQ ID NO:95. In some cases, a variant IL-2 polypeptide has 2 or 3 aa substitutions compared to the IL-2 aa sequence set forth in SEQ ID NO:95.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:95. Potential amino acids where substitutions may be introduced include one or more of the following positions:

(i) position 15, where the aa is other than E (e.g., A);

(ii) position 16, where the aa is other than H (e.g., A, T, N, C, Q, M, V or W);

(iii) position 20 is an aa other than D (e.g., A);

(iv) position 42, where the aa is other than F (e.g., A, M, P, S, T, Y, V or H);

(v) position 45, where the aa is other than Y (e.g., A);

(vi) position 88, where the aa is other than N (e.g., A or R);

(vii) position 126, where the aa is other than Q (e.g., A);

Combinations of the above substitutions include (H16X, F42X), (D20X, F42X), (E15X, D20X, F42X), (an H16X, D20X, F42X), (H16X, F42X, R88X), (H16X, F42X, Q126X), (D20X, F42X, Q126X), (D20X, F42X, and Y4X), (H16X, D20X, F42X, and Y45X), (D20X, F42X, Y45X, Q126X), (H16X, D20X, F42X, Y45X, Q126X), where X is the substituted aa, optionally chosen from the following: positions 15, 20, 45, 126—A; position 16—A or T, or also N, C, Q, M, V or W; position 42—A, or also M, P, S, T, Y, V or H; position 88—A or R.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 16 is an aa other than H. In one case, the position of H16 is substituted by Asn, Cys, Gln, Met, Val, or Trp. In one case, the position of H16 is substituted by Ala. In another case, the position of H16 is substituted by Thr. Additionally, or alternatively, IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 42 is an aa other than F. In one case, the position of F42 is substituted by Met, Pro, Ser, Thr, Trp, Tyr, Val, or His. In one case, the position of F42 is substituted by Ala.

IL-2 variants include polypeptides comprising an aa sequence comprising all or part of human IL-2 polypeptide having a substitution at position H16 and/or F42 (e.g., H16A and/or F42A substitutions).

IL-2 variants include polypeptides having at least 90% or at least 95% (e.g., at least 95%, 98%, or 99%) aa sequence identity to at least 80 (e.g., at least 100, 110, 120, or 130) contiguous aas of SEQ ID NO:95, wherein the aa at position 16 is an aa other than H and the aa at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In a second case, the position of H16 is substituted by Thr and the position of F42 is substituted by Ala (an H16T and F42A variant). In a third case, the position of H16 is substituted by Ala and the position of F42 is substituted by Thr (an H16A and F42T variant). In a fourth case, the position of H16 is substituted by Thr and the position of F42 is substituted Thr Ala (an H16T and F42T variant). As noted above, such variants will exhibit reduced binding to both the human IL-2Rα chain and IL2Rβ chain.

In any of the wild-type or variant IL-2 sequences provided herein, the cysteine at position 125 may be substituted with an aa other than cystine, such as alanine (a C125A substitution). In addition to any stability provided by the substitution, it may be employed where, for example, an epitope containing peptide or additional peptide is to be conjugated to a cysteine residue elsewhere in a MAPP, thereby avoiding competition from the C125 of the IL-2 MOD sequence.

c. Fas Ligand (FasL) and its Variants

In some cases, a wild-type and/or a variant Fas Ligand (FasL) polypeptide sequence is present as a MOD in a MAPP. FasL is a homomeric type-II transmembrane protein in the tumor necrosis factor (TNF) family. FasL signals by trimerization of the Fas receptor in a target cell, which forms a death-inducing complex leading to apoptosis of the target cell. Soluble FasL results from matrix metalloproteinase-7 (MMP-7) cleavage of membrane-bound FasL at a conserved site.

In an embodiment, a wt. Homo sapiens FasL protein has the sequence MQQPFNYPYP QIYWVDSSAS SPWAPPGTVL PCPTSVPRRP GQRRPPPPPP PPPLPPPPPP PPLPPLPLPP LKKRGNHSTG LCLLVMFFMV LVALVGLGLG MFQLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L, (SEQ ID NO:99), NCBI Ref. Seq. NP_000630.1, UniProtKB-P48023 where residues 1-80 are cytoplasmic, 81-102 are the transmembrane domain and aas 103-281 are extracellular (ectodomain). In some cases, a FasL polypeptide suitable for inclusion in a MAPP comprises an aa sequence having at least 60%, at least 70%, at least 80%, at least 90%, at least 95%, at least 98%, at least 99%, or 100%, aa sequence identity to a contiguous stretch of at least 150 aas, at least 170, at least 180 aas, at least 200 aas, at least 225 aas, at least 250 aas, at least 270 aas, at least 280, or all aas of the aa sequence of SEQ ID NO:99).

A Fas receptor can have the sequence MLGIWTLLPL VLTSVARLSS KSVNAQVTDI NSKGLELRKT VTTVETQNLE GLHHDGQFCH KPCPPGERKA RDCTVNGDEP DCVPCQEGKE YTDKAHFSSK CRRCRLCDEG HGLEVEINCT RTQNTKCRCK PNFFCNSTVC EHCDPCTKCE HGIIKECTLT SNTKCKEEGS RSNLGWLCLL LLPIPLIVWV KRKEVQKTCR KHRKENQGSH ESPTLNPETV AINLSDVDLS KYITTIAGVM TLSQVKGFVR KNGVNEAKID EIKNDNVQDT AEQKVQLLRN WHQLHGKKEA YDTLIKDLKK ANLCTLAEKI QTIILKDITS DSENSNFRNE IQSLV, (SEQ ID NO:100) NCBI Reference Sequence: NP_000034.1, UniProtKB-P25445, where aas 26-173 form the ectodomain (extracellular domain), aas 174-190 form the transmembrane domain, and 191-335 the cytoplasmic domain The ectodomain may be used to determine binding affinity with FasL.

A FasL polypeptide suitable for inclusion in a MAPP may comprise an aa sequence having at least 60%, at least 70%, at least 80%, at least 90%, at least 95%, at least 98%, at least 99%, or 100%, aa sequence identity to the following aa sequence: IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMMSYCTTGQMWA RSSYLGAVFN LTSADHLYVN VSELSLVNFE ESQTFFGLYK (SEQ ID NO:101); and has a length of about 150 aas, including 148, 149, 150, 151, or 152 aas.

In some cases, a FasL polypeptide suitable for inclusion in a MAPP comprises an aa sequence having at least 60%, at least 70%, at least 80%, at least 90%, at least 95%, at least 98%, at least 99%, or 100%, aa sequence identity to a contiguous stretch of at least 50 aas, at least 160 aas, at least 170, at least 175, or all of the aas the following aa sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:102). Suitable variant FasL polypeptide sequences include polypeptide sequences with at least 80%, at least 85%, at least 90%, at least 95%, at least 98%, or at least 99% aa sequence identity to at least 140 contiguous aa (e.g., at least 150, at least 160, at least 170, or at least 175 contiguous aa) of SEQ ID NO:102) (e.g., which have at least one aa substitution, deletion or insertion).

In some cases, a variant FasL polypeptide (e.g., comprising a variant of SEQ ID NO:101 or SEQ ID NO:102) exhibits reduced binding affinity to a mature Fas receptor sequence (e.g., a FasL receptor comprising all or part of the polypeptide set forth in SEQ ID NO:100, such as its ectodomain), compared to the binding affinity of an FasL polypeptide comprising the aa sequence set forth in SEQ ID NO:101 or SEQ ID NO:102. For example, in some cases, a variant FasL polypeptide (e.g., comprising a variant of SEQ ID NO:102) binds an Fas receptor (e.g., comprising all or part of the polypeptides set forth in SEQ ID NO:100, such as its ectodomains), with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an FasL polypeptide comprising the aa sequence set forth in SEQ ID NO:99 or 102.

d. PD-L1 and its Variants

As one non-limiting example, a MOD or variant MOD present in a MAPP is a PD-L1 or variant PD-L1 polypeptide. Wild-type PD-L1 binds to PD1.

A wild-type human PD-L1 polypeptide can comprise the following aa sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO:103); where aas 1-18 form the signal sequence, aas 19-127 form the Ig-like V-type or IgV domain, and 133-225 for the Ig-like C2 type domain

A wild-type human PD-L1 ectodomain aa sequence can comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:104); where aas 1-109 form the Ig-like V-type or “IgV” domain, and aas 115-207 for the Ig-like C2 type domain.

A wild-type human PD-L1 ectodomain aa sequence can also comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELP LAHPPNER LNVSIKI(SEQID NO:105); where aas 1-109 form the Ig-like V-type or “IgV” domain, and aas 115-207 for the Ig-like C2 type domain. See e.g., NCBI Accession and version 3BIK_A, which includes an N-terminal alanine as its first aa.

A wild-type PD-L1 IgV domain, suitable for use as a MOD may comprise aa 18 and aas IgV aas 19-127 of SEQ ID NO:103, and a carboxyl terminal stabilization sequences, such as for instance the last seven aas (bolded and italicized) of the sequence: A FTVTVPKDLY VVEYGSNMT I ECKFPVEKQL DLAALIVYWE MEDKNIIQFV HGEEDLKTQH SSYRQRARLL KDQLSLGNAA LQITDVKLQD AGVYRCMISY GGADYKRITV KVNAPYAAAL HEH (SEQ ID NO:106). Where the carboxyl stabilizing sequence comprises a histidine (e.g., a histidine approximately 5 residues to the C-terminal side of the Tyr (Y) appearing as aa 117 of SEQ ID NO:106) to about aa 122, the histidine may form a stabilizing electrostatic bond with the backbone amide at aas 82 and 83 (bolded and italicized in SEQ ID NO:103 (Q107 and L106 of SEQ ID NO:103). As an alternative, a stabilizing disulfide bond may be formed by substituting one of aas 82 or 83) (Q107 and L106 of SEQ ID NO:103) and one of aa residues 121, 122, or 123 (equivalent to aa positions 139-141 of SEQ ID NO:103).

A wild-type PD-1 polypeptide can comprise the following aa sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:107).

In some cases, a variant PD-L1 polypeptide (e.g., a variant of SEQ ID NO:104 or PD-L1's IgV domain) exhibits reduced binding affinity to PD-1 (e.g., a PD-1 polypeptide comprising the aa sequence set forth in SEQ ID NO:107), compared to the binding affinity of a PD-L1 polypeptide comprising the aa sequence set forth in SEQ ID NO:103 or SEQ ID NO:104. For example, in some cases, a variant PD-L1 polypeptide binds PD-1 (e.g., a PD-1 polypeptide comprising the aa sequence set forth in SEQ ID NO:107) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:103 or SEQ ID NO:104.

e. CD80 and its Variants

In some cases, a wild-type and/or a variant CD80 MOD polypeptide sequence is present as a MOD in a MAPP of the present disclosure. Wild-type CD80 and variant CD80 MOD polypeptides bind to CD28 which acts as their receptor.

A wild-type aa sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:108). See NCBI Reference Sequence: NP_005182.1. The aa sequence of the IgV domain of a wild-type human CD80 can be as follows:

(SEQ ID NO: 109)
VIHVTK EVKEVATLSC GHNVSVEELA QTRIYWQKEK
KMVLTMMSGD MNIWPEYKNR TIFDITNNLS IVILALRPSD
EGTYECVVLK YEKDAFKREH LAEVTLSV,,

which is aas 1-104 of SEQ ID NO:108.

A wild-type CD28 aa sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:110).

A wild-type CD28 aa sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:111)

A wild-type CD28 aa sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:112).

Variant CD80 polypeptides suitable as a MOD in a MAPP of the present disclosure may exhibit reduced binding affinity to CD28, compared to the binding affinity of a CD80 polypeptide comprising the aa sequence set forth in SEQ ID NO:108, or the IgV domain sequence SEQ ID NO:109, for CD28. A variant CD80 MOD polypeptide may bind CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a CD80 polypeptide comprising the aa sequence set forth in SEQ ID NO:108 for CD28 (e.g., a CD28 polypeptide comprising the aa sequence set forth in one of SEQ ID NO:110, SEQ ID NO:111, or SEQ ID NO:112).

CD80 ectodomain variants suitable for use as a MOD in a MAPP include those polypeptides with at least one aa substitution having at least 90%, at least 95%, at least 98%, or at least 99% aa sequence identity to SEQ ID NO:108, or the IgV domain sequence SEQ ID NO:109.

CD80 ectodomain variants suitable for use as a MOD in a MAPP include those polypeptides having at least 90%, at least 95%, at least 98%, or at least 99% aa sequence identity to SEQ ID NO:108, or the IgV domain sequence SEQ ID NO:109, and which have at least one (e.g., at least two, or at least three) aa substitutions.

CD80 ectodomain variants suitable for use as a MOD in a MAPP include those polypeptides having at least 90% (e.g., at least 95%, 98%, or 99%) aa sequence identity to at least 80 (e.g., at least 90, 100, 104, 120, 150, 180, 200, or 208) contiguous aas of SEQ ID NO:108, or least 80 (e.g., at least 90, 100, or 104) contiguous aas of the IgV domain sequence of SEQ ID NO:109.

f. CD86 and its Variants

In some cases, a wild-type and/or a variant CD86 MOD polypeptide sequence is present as a MOD in a MAPP of the present disclosure. Wild-type CD86 and variant CD86 MOD polypeptides bind to CD28 which acts as their receptor as discussed for CD80 MOD polypeptides.

A wild-type aa sequence of the ectodomain of human CD86 can be as follows:

(SEQ ID NO: 113)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVLANFSQPEIVPISNITENVYINLTCSSIHGYPEPKKMS
VLLRTKNSTIEYDGIMQKSQDNVTELYDVSISLSVSFPDVTSNMTIFCI
LETDKTRLLSSPFSIELEDPQPPPDHIP.

The aa sequence of the IgV domain of a wild-type human CD86 can be as follows:

(SEQ ID NO: 114)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVL.

Variant CD86 polypeptides suitable as a MOD in a MAPP may exhibit reduced binding affinity to CD28, compared to the binding affinity of a CD86 polypeptide comprising the aa sequence set forth in SEQ ID NO:113 or SEQ ID NO:114 for CD28. A variant CD86 MOD polypeptide may bind CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a CD86 polypeptide comprising the aa sequence set forth in SEQ ID NO:113 or SEQ ID NO:114 for CD28 (e.g., a CD28 polypeptide comprising the aa sequence set forth in one of SEQ ID NO:110, SEQ ID NO:111, or SEQ ID NO:112).

CD86 ectodomain variants suitable for use as a MOD in a MAPP include those polypeptides with at least one aa substitution having at least 90%, at least 95%, at least 98%, or at least 99% aa sequence identity to SEQ ID NO:113, or the IgV domain sequence SEQ ID NO:114.

CD86 ectodomain variants suitable for use as a MOD in a MAPP include those polypeptides having at least 90%, at least 95%, at least 98%, or at least 99% aa sequence identity to SEQ ID NO:113, or the IgV domain sequence SEQ ID NO:114, and which have at least one (e.g., at least two, or at least three) aa substitutions.

CD86 ectodomain variants suitable for use as a MOD in a MAPP include those polypeptides with at least one aa substitution having at least 90%, at least 95%, at least 98%, or at least 99% aa sequence identity to at least 80 (e.g., at least 90, 100, or 109, 120, 150, 180, 200, or 224) contiguous aas of SEQ ID NO:113, or at least 80 (e.g., at least 90, 100, 104) contiguous aas of the IgV domain sequence SEQ ID NO:114.

g. 4-1BBL and its Variants

In some cases, a wild-type and/or a variant 4-1BBL MOD polypeptide sequence is present as a MOD in a MAPP of the present disclosure. Wild-type 4-1BBL binds to 4-1BB (CD137).

A wild-type 4-1BBL aa sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:115). NCBI Reference Sequence: NP_003802.1, where aas 29-49 are a transmembrane region.

In some cases, a variant 4-1BBL polypeptide is a variant of the tumor necrosis factor (TNF) homology domain (THD) of human 4-1BBL. A wild-type aa sequence of the THD of human 4-1BBL can comprise, e.g., one of SEQ ID NOs:116-118, as follows:

(SEQ ID NO: 116)
PAGLLDLRQG MFAQLVAQNV LLIDGPLSWY SDPGLAGVSL TGGLSYKEDT
KELVVAKAGV YYVFFQLELR RVVAGEGSGS VSLALHLQPL RSAAGAAALA LTVDLPPASS
EARNSAFGFQ GRLLHLSAGQ RLGVHLHTEA RARHAWQLTQ GATVLGLFRV TPEIPAGLPS
PRSE;
(SEQ ID NO: 117)
D PAGLLDLRQG MFAQLVAQNV LLIDGPLSWY SDPGLAGVSL TGGLSYKEDT
KELVVAKAGV YYVFFQLELR RVVAGEGSGS VSLALHLQPL RSAAGAAALA LTVDLPPASS
EARNSAFGFQ GRLLHLSAGQ RLGVHLHTEA RARHAWQLTQ GATVLGLFRV TPEIPAGLPS
PRSE;
and
(SEQ ID NO: 118)
D PAGLLDLRQG MFAQLVAQNV LLIDGPLSWY SDPGLAGVSL TGGLSYKEDT
KELVVAKAGV YYVFFQLELR RVVAGEGSGS VSLALHLQPL RSAAGAAALA LTVDLPPASS
EARNSAFGFQ GRLLHLSAGQ RLGVHLHTEA RARHAWQLTQ GATVLGLFRV TPEIPA.

A wild-type 4-1BB aa sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:119).

A variant 4-1BBL polypeptide exhibits reduced binding affinity to 4-1BB, compared to the binding affinity of a 4-1BBL polypeptide comprising the aa sequence set forth in one of SEQ ID NOs:116-118. For example, a variant 4-1BBL polypeptide may bind 4-1BB with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a 4-1BBL polypeptide comprising the aa sequence set forth in one of SEQ ID NOs:116-118 for a 4-1BB polypeptide (e.g., a 4-1BB polypeptide comprising the aa sequence set forth in SEQ ID NO:119), when assayed under the same conditions.
4-1BBL variants suitable for use as a MOD in a MAPP include those polypeptides with at least one aa substitution having at least 90%, at least 95%, at least 98%, or at least 99% aa sequence identity to one of SEQ ID NOs:116, 117 or 118.
4-1BBL variants suitable for inclusion in a MAPP include those with at least one aa substitution (e.g., two, three, or four substitutions) include those having at least 90%, at least 95%, at least 98%, or at least 99% aa sequence identity to at least 140 (e.g., at least 160, 175, 180, or 181) contiguous aas of SEQ ID NO:116.

h. Anti-CD28

In some cases, antibodies or antibody sequences directed against CD28 (e.g., an anti-CD28 antibody, an anti-body fragment binding CD28, or an scFv, nanobody, or diabody binding to CD28) may be employed as a MOD in a MAPP. The ability of anti-CD28 antibodies to act as a superagonist, agonist, or antagonist of CD28 activity has been described. See e.g., Poirier et al., (2012) Amer. J. of Transplantation, “CD28-Specific Immunomodulating Antibodies: What Can Be Learned From Experimental Models?” 12:1682-1690. Of particular interest are anti-CD28 antibodies that act as an agonist or superagonist.

Anti-CD28 antibodies or anti-CD28 sequences may be included in MAPPs in the absence of any other MOD sequences. Alternatively, antibodies or antibody sequences directed against CD28 by be incorporated into a MAPP along with one or more additional MODs, or variant MODs. In an embodiment, A MAPP comprises one or more (e.g., two) anti-CD28 antibody or anti-CD28 sequences along with one or more (e.g., two) 4-1BBL MODs or variant MODs, such as those described above. In an embodiment, A MAPP comprises one or more (e.g., two) anti-CD28 antibody or anti-CD28 sequences along with one or more (e.g., two) IL-2 MODs or variant IL-2 MODs, such as those described above. For example, the substitutions in the variant IL-2 MOD may include H16A or H16T along with an F42A or F42T substitution. By way of example, a MAPP may comprise one or more (e.g., two) anti-CD28 antibody or anti-CD28 sequences (e.g., an anti-CD28 scFv) along with one or more variant IL-2 MODs comprising H16A and/or F42A substitutions.

In some cases, an anti-CD28 antibody suitable for inclusion in a MAPP comprises: a) VL CDR1, VL CDR2, and VL CDR3 present in a light chain variable region (VL) comprising the following amino acid sequence:
QWYQQKPGQPPKLLIFAASNVESGVPARFSGSGSGTNFSLNIHPVDEDDVAMYFCQQSRKVPY TFGGGTKLEIKR (SEQ ID NO:475); and b) VH CDR1, CDR2, and CDR3 present in a heavy chain variable region (VH) comprising the following amino acid sequence:
QVKLQQSGPGLVTPSQSLSITCTVSGFSLSDYGVHWVRQSPGQGLEWLGVIWAGGGTNYNSAL MSRKSISKDNSKSQVFLKMNSLQADDTAVYYCARDKGYSYYYSMDYWGQGTTVTVSS (SEQ ID NO:476). In some cases, the V_{H} and V_{L} CDRs are as defined by Kabat (see, e.g., the CDR Table, above; and Kabat 1991). In some cases, the V_{H} and V_{L} CDRs are as defined by Chothia (see, e.g., the CDR Table, above; and Chothia 1987). In some cases, the VH CDRs are: DYGVH (VH CDR1) (SEQ ID NO:477); VIWAGGGTNYNSALMS (VH CDR2) (SEQ ID NO:478); and DKGYSYYYSMDY (VH CDR3) (SEQ ID NO:479).

In some cases, an anti-CD28 antibody suitable for inclusion in a MAPP comprises: a) a VL region comprising an amino acid sequence having at least 90%, at least 95%, at least 98%, at least 99%, or 100%, amino acid sequence identity to the following amino acid sequence: QWYQQKPGQPPKLLIFAASNVESGVPARFSGSGSGTNFSLNIHPVDEDDVAMYFCQQSRKVPY TFGGGTKLEIKR (SEQ ID NO:475); and b) a VH region comprising an amino acid sequence having at least 90%, at least 95%, at least 98%, at least 99%, or 100%, amino acid sequence identity to the following amino acid sequence:

(SEQ ID NO: 476)
QVKLQQSGPGLVTPSQSLSITCTVSGFSLSDYGVHWVRQSPGQGLEWLG
VIWAGGGTNYNSALMSRKSISKDNSKSQVFLKMNSLQADDTAVYYCARD
KGYSYYYSMDYWGQGTTVTVSS.

In some cases, an anti-CD28 antibody suitable for inclusion in a MAPP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence:
QWYQQKPGQPPKLLIFAASNVESGVPARFSGSGSGTNFSLNIHPVDEDDVAMYFCQQSRKVPY TFGGGTKLEIKR (SEQ ID NO:475); b) a peptide linker; and c) a VH region comprising an amino acid sequence having at least 90%, at least 95%, at least 98%, at least 99%, or 100%, amino acid sequence identity to the following amino acid sequence:
QVKLQQSGPGLVTPSQSLSITCTVSGFSLSDYGVHWVRQSPGQGLEWLGVIWAGGGTNYNSAL MSRKSISKDNSKSQVFLKMNSLQADDTAVYYCARDKGYSYYYSMDYWGQGTTVTVSS (SEQ ID NO:476). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-CD28 antibody suitable for inclusion in a MAPP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence:
QVKLQQSGPGLVTPSQSLSITCTVSGFSLSDYGVHWVRQSPGQGLEWLGVIWAGGGTNYNSAL MSRKSISKDNSKSQVFLKMNSLQADDTAVYYCARDKGYSYYYSMDYWGQGTTVTVSS (SEQ ID NO:476); b) a peptide linker; and c) a VL region comprising an amino acid sequence having at least 90%, at least 95%, at least 98%, at least 99%, or 100%, amino acid sequence identity to the following amino acid sequence:
QWYQQKPGQPPKLLIFAASNVESGVPARFSGSGSGTNFSLNIHPVDEDDVAMYFCQQSRKVPY TFGGGTKLEIKR (SEQ ID NO:475). In some cases, the peptide linker comprises the amino acid sequence (GGGGS)n, where n is an integer from 1 to 10 (e.g., where n is 1, 2, 3, 4, 5, 6, 7, 8, 9, or 10).

In some cases, the peptide linker comprises the amino acid sequence GGGGSGGGGSGGGGS (SEQ ID NO:454) and has a length of 15 amino acids.
6. Linkers

As noted above, a MAPP can include a linker sequence (aa, peptide, or polypeptide linker sequence) or “linker” interposed between any two elements of a MAPP, e.g., an epitope and an MHC polypeptide; between an MHC polypeptide and an Ig Fc polypeptide; between a first MHC polypeptide and a second MHC polypeptide; etc. Although termed “linkers,” sequences employed for linkers may also be placed at the N- and/or C-terminus of a MAPP polypeptide to, for example, stabilize the MAPP polypeptide or protect it from proteolytic degradation.

Suitable polypeptide linkers (also referred to as “spacers”) are known in the art and can be readily selected and can be of any of a number of suitable lengths, e.g., from 2 to 50 aa in length, e.g., from 2 aa to 10 aa, from l0aa to 20 aa, 20 aa to 30 aa, from 30 aa to 40aa, from 40aa to 50aa, or longer than 50aa. In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 aa in length. Linkers can be generally classified into three groups, i.e., flexible, rigid and cleavable. See, e.g., Chen et al. (2013) Adv. Drug Deliv. Rev. 65:1357; and Klein et al. (2014) Protein Engineering, Design & Selection 27:325. Unless stated otherwise, the linkers employed in the MAPPs of this disclosure are not the cleavable linkers generally known in the art.

Polypeptide linkers in the MAPP may include, for example, polypeptides that comprise, consist essentially of, or consists of: i) Gly and Ser; ii) Ala and Ser; iii) Gly, Ala, and Ser; iv) Gly, Ser, and Cys (e.g., a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers (including, for example polymers comprising the sequences GSGGS (SEQ ID NO:120) or GGGS (SEQ ID NO:121), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used as both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:122), GGSGG (SEQ ID NO:123), GSGSG (SEQ ID NO:124), GSGGG (SEQ ID NO:125), GGGSG (SEQ ID NO:126), GSSSG (SEQ ID NO:127), any of which may be repeated from 1 to 10 times (e.g., repeated 1, 2, 3, 4, 5, 6, 7, 8, 9, or 10 times), or combinations thereof, and the like. Linkers can also comprise the sequence Gly(Ser)₄ (SEQ ID NO:128) or (Gly)₄Ser (SEQ ID NO:129), either of which may be repeated from 1 to 10 times (e.g., 1, 2, 3, 4, 5, 6, 7, 8, 9, or 10 times). In one embodiment the linker comprises the aa sequence AAAGG (SEQ ID NO:130), which may be repeated from 1 to 10 times.

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the MAPP. Rigid peptide linkers include peptide linkers rich in proline, and peptide linkers having an inflexible helical structure, such as an α-helical structure. Examples of rigid peptide linkers include, e.g., (EAAAK)n (SEQ ID NO:547), A(EAAAK)nA (SEQ ID NO:548), A(EAAAK)nALEA(EAAAK)nA (SEQ ID NO:549), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:550) include EAAAK (SEQ ID NO:550), (EAAAK)₂ (SEQ ID NO:551), (EAAAK)₃ (SEQ ID NO:552), A(EAAAK)₄ALEA(EAAAK)₄A (SEQ ID NO:553), and AEAAAKEAAAKA (SEQ ID NO:554). Non-limiting examples of suitable rigid linkers comprising (AP)n include PAPAP (SEQ ID NO:555; also referred to herein as “(AP)2”); APAPAPAP (SEQ ID NO:556; also referred to herein as “(AP)4”); APAPAPAPAPAP (SEQ ID NO:557; also referred to herein as “(AP)6”); APAPAPAPAPAPAPAP (SEQ ID NO:558; also referred to herein as “(AP)8”); and APAPAPAPAPAPAPAPAPAP (SEQ ID NO:559; also referred to herein as “(AP)10”). Non-limiting examples of suitable rigid linkers comprising (KP)n include KPKP (SEQ ID NO:560; also referred to herein as “(KP)2”); KPKPKPKP (SEQ ID NO:561; also referred to herein as “(KP)4”); KPKPKPKPKPKP (SEQ ID NO:562; also referred to herein as “(KP)6”); KPKPKPKPKPKPKPKP (SEQ ID NO:563; also referred to herein as “(KP)8”); and KPKPKPKPKPKPKPKPKPKP (SEQ ID NO:564; also referred to herein as “(KP)10”). Non-limiting examples of suitable rigid linkers comprising (EP)n include EPEP (SEQ ID NO:565; also referred to herein as “(EP)2”); EPEPEPEP (SEQ ID NO:566; also referred to herein as “(EP)4”); EPEPEPEPEPEP (SEQ ID NO:567; also referred to herein as “(EP)6”); EPEPEPEPEPEPEPEP (SEQ ID NO:568; also referred to herein as “(EP)8”); and EPEPEPEPEPEPEPEPEPEP (SEQ ID NO:569; also referred to herein as “(EP)10”).

As with other linker sequences, rigid peptide linkers may be interposed between any two elements of a MAPP. Rigid peptide linkers find particular use in joining MOD polypeptide sequences to other elements of a MAPP. In particular, rigid peptide linkers may be employed to link a MOD polypeptide sequence to the carboxy terminus of frame work polypeptides (position 3 and/or 3′) or a dimerization polypeptide (positions 5 and/or 5′) of a duplex MAPP. For example, a MOD polypeptide comprising an immunoglobulin CH2CH3 multimerization sequence may comprise a rigid peptide linker and a MOD (e.g., a wild type variant IL-2 or PD-L1 MOD) at position 3 and/or 3′ (See e.g., FIGS. 6-10). Rigid peptide linkers may also be used to link a MOD polypeptide to the N-terminus of a MAPP polypeptide (e.g., the N-terminus of a dimerization and/or framework polypeptide at positions 1 and/or 1′).

In some cases, a linker polypeptide, present in a polypeptide of a MAPP includes a cysteine residue that can form a disulfide bond with a cysteine residue present in another polypeptide of the MAPP. In some cases, for example, the linker comprises an aa sequence selected from CGGGS (SEQ ID NO:570), GCGGS (SEQ ID NO:571), GGCGS (SEQ ID NO:572), GGGCS (SEQ ID NO:573), and GGGGC (SEQ ID NO:574) with the rest of the linker comprised of Gly and Ser residues (e.g., GGGGS (SEQ ID NO:339) units that may be repeated from 1 to 10 times, such as 1, 2, 3, 4, 5, 6, 7, 8, 9, or 10 times). Cysteine containing linkers may also be selected from the sequences GCGASGGGGSGGGGS (SEQ ID NO:131), the sequence GCGGSGGGGSGGGGSGGGGS (SEQ ID NO:132) or the sequence GCGGSGGGGSGGGGS (SEQ ID NO:133).

Accordingly, the linker to which an epitope is attached may be from about 5 to about 50 aas in length. The linker to which an epitope may be attached may, for example be from about 5 to about 50 aas in length and comprise more than 50% Gly and Ser residues with one cysteine residue. The linker to which an epitope may be attached may be from about 5 to about 50 aas in length and comprise more than 50% (Gly)₄S repeats with one optional cysteine residue. The linker to which an epitope may be attached may be a (Gly)₄S sequence repeated from 3 to 8 (e.g., 3 to 7) times, optionally having one aa replaced by a cysteine residue.
7. Epitopes

A variety of peptide epitopes (also referred to herein as “epitopes”) may be present in a MAPP or higher order complexes of MAPPs (such as duplex MAPPs of the present disclosure), and presentable to a TCR on the surface of a T cell.

An epitope-presenting peptide can have a length of from about 4 aas to about 25 aas (aa), e.g., the epitope can have a length of from 4 aa to 10 aa, from 10 aa to 15 aa, from 15 aa to 20 aa, or from 20 aa to 25 aa. For example, an epitope present in a MAPP of the present disclosure can have a length of 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, an epitope-presenting peptide present in a MAPP of the present disclosure has a length of from 5 aa to 10 aa, e.g., 5 aa, 6 aa, 7 aa, 8 aa, 9 aa, or 10 aa.

Epitope presenting peptides (or simply epitopes) may be derived from a variety of self and non-self antigens, depending upon the nature of the MAPP and its desired use. Non-self antigens (including neoantigens) may be incorporated into MAPPs for the treatment or prophylaxis of, for example, cancers, allergies, and viral and bacterial diseases. Self-antigens may be incorporated into MAPPs for the treatment or prophylaxis of, for example, cancers, infections and allergies.

A peptide epitope present in a MAPP (e.g., a duplex MAPP) is designed to be specifically bound by a target T cell that has a T cell receptor (“TCR”) that is specific for the epitope and which specifically binds the peptide epitope of the MAPP. An epitope-specific T cell thus binds a peptide epitope having a reference aa sequence, but substantially does not bind an epitope that differs from the reference aa sequence. For example, an epitope-specific T cell binds a peptide epitope having a reference aa sequence, and binds an epitope that differs from the reference aa sequence, if at all, with an affinity that is less than 10⁻⁶ M, less than 10⁻⁵ M, or less than 10⁻⁴ M. An epitope-specific T cell can bind a peptide epitope for which it is specific with an affinity of at least 10⁻⁷ M, at least 10⁻⁸ M, at least 10⁻⁹ M, or at least 10⁻¹⁰ M.

a. Epitope-Presenting Peptides in MAPPs with Class I MHC Presenting Sequences and Presenting Complexes

Among the epitopes that may be bound and presented to a TCR by a MAPP with Class I MHC presenting sequences or presenting complexes are cancer antigens, and antigens from infectious agents (e.g., viral or bacterial agents). Where T Cell dysregulation (e.g., CD8+ T cell dysregulation) result in over reaction to allergens, the epitope that may be presented include epitopes of allergens and autoantigens, e.g., those associated with Type 1 diabetes (T1D) or celiac disease. For example, an allergen may be selected from protein or non-proteins components of: nuts (e.g., tree and/or peanuts), glutens, pollens, eggs (e.g., chicken, Gallus domesticus eggs), shellfish, soy, fish, and insect venoms (e.g., bee and/or wasp venom antigens). Similarly, where dysregulation of CD8+ T reg cells and self-reactive CD8+ effector T cells result in autoimmune diseases the epitope presented may be from a protein associated with, for example, multiple sclerosis, Rasmussen's encephalitis, paraneoplastic syndromes. Grave's disease (GD), systemic lupus erythematosus (SLE), aplastic anemia (AA), or vitiligo.

(i) Cancer Epitopes

Suitable epitopes include in a MAPP or higher order MAPP complexes such as duplexes MAPPs, but are not limited to, epitopes present in a cancer-associated antigen. Cancer-associated antigens are known in the art; see, e.g., Cheever et al. (2009) Clin. Cancer Res. 15:5323. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2) (see, e.g., Vigneron et al. (2013) Cancer Immunity 13:15; and Vigneron (2015) BioMed Res. Int'l Article ID 948501); and epidermal growth factor receptor (EGFR) vIII polypeptide (see, e.g., Wong et al. (1992) PNAS. USA 89:2965; and Miao et al. (2014) PLoSOne 9:e94281).

In some cases, a suitable peptide epitope is a peptide fragment of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE Al) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, or a Fos-related antigen-1 (FOSL) polypeptide. In some cases, a human papilloma virus (HPV) antigen is specifically excluded. In some cases, an alpha-feto protein (AFP) antigen is specifically excluded. In some cases, a Wilms tumor-1 (WT1) antigen is specifically excluded.

Amino acid sequences of cancer-associated antigens are known in the art; see, e.g., MUC1 (GenBank CAA56734); LMP2 (GenBank CAA47024); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CA099273); MYCN (GenBank NP_001280157); RhoC (GenBank AAH52808); TRP-2 (GenBank AAC60627); mesothelin (GenBank AAH09272); PSCA (GenBank AAH65183); MAGE Al (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAXS (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFβ (GenBank NP_002600); MAD-CT-2 (GenBank NP_001138574); and FOSL (GenBank NP_005429). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168.

(a) Alpha Feto Protein (AFP)

MAPPs or higher order MAPP complexes such as duplex MAPPs described herein may comprise a peptide presenting an epitope of alpha-feto protein (AFP), which has been associated with hepatocellular carcinoma, pancreatic cancer, stomach cancer, colorectal cancer, hepatoblastoma, and an ovarian yolk sac tumor. The AFP epitope may be presented in the context of a Class I MHC presenting sequence. The AFP epitope may be presented in the context of a Class I MHC presenting complex. The Class I MHC may be a) an aa sequence having at least 95% aa sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702,HLA-C*0801, or HLA-C*1502 depicted in FIGS. 3A-3G.

AFP peptides for inclusion in a MAPP include, but are not limited to, AITRKMAAT (SEQ ID NO:134); AYTKKAPQL (SEQ ID NO:135); LLNQHACAV (SEQ ID NO:136); KLVLDVAHV (SEQ ID NO:137); FMNKFIYEI (SEQ ID NO:138); SIPLFQVPE (SEQ ID NO:139); LLNFTESRT (SEQ ID NO:140); FVQEATYKF (SEQ ID NO:141); ATYKEVSKM (SEQ ID NO:142); KEVSKMVKD (SEQ ID NO:143); RHNCFLAHK (SEQ ID NO:144); ATAATCCQL (SEQ ID NO:145); YIQESQALA (SEQ ID NO:146); QLTSSELMAI (SEQ ID NO:147); KLSQKFTKV (SEQ ID NO:148); KELRESSLL (SEQ ID NO:149); SLVVDETYV (SEQ ID NO:150); ILLWAARYD (SEQ ID NO:151); KIIPSCCKA (SEQ ID NO:152); CRGDVLDCL (SEQ ID NO:153); QQDTLSNKI (SEQ ID NO:154); TMKQEFLINL (SEQ ID NO:155); NLVKQKPQI (SEQ ID NO:156); AVIADFSGL (SEQ ID NO:157); LLACGEGAA (SEQ ID NO:158); LACGEGAAD (SEQ ID NO:159); KAPQLTSSEL (SEQ ID NO:160); YICSQQDTL (SEQ ID NO:161); TECCKLTTL (SEQ ID NO:162); CTAEISLADL (SEQ ID NO:163); VTKELRESSL (SEQ ID NO:164); IMSYICSQQD (SEQ ID NO:165); TRTFQAITV (SEQ ID NO:166); FQKLGEYYL (SEQ ID NO:167); RVAKGYQEL (SEQ ID NO:168); SYQCTAEISL (SEQ ID NO:169);KQEFLINLV (SEQ ID NO:170); MKWVESIFL (SEQ ID NO:171); PVNPGVGQC (SEQ ID NO:172); AADIIIGHL (SEQ ID NO:173); QVPEPVTSC (SEQ ID NO:174); TTLERGQCII (SEQ ID NO:175); KMAATAATC (SEQ ID NO:176); QAQGVALQTM (SEQ ID NO:177); FQAITVTKL (SEQ ID NO:178); LLEKCFQTE (SEQ ID NO:179); VAYTKKAPQ (SEQ ID NO:180); KYIQESQAL (SEQ ID NO:181); GVALQTMKQ (SEQ ID NO:182); GQEQEVCFA (SEQ ID NO:183); SEEGRHNCFL (SEQ ID NO:184); RHPFLYAPTI (SEQ ID NO:185); TEIQKLVLDV (SEQ ID NO:186); RRHPQLAVSV (SEQ ID NO:187); GEYYLQNAFL (SEQ ID NO:188); NRRPCFSSLV (SEQ ID NO:189); LQTMKQEFLI (SEQ ID NO:190); IADFSGLLEK (SEQ ID NO:191); GLLEKCCQGQ (SEQ ID NO:192); TLSNKITEC (SEQ ID NO:193); LQDGEKIMSY (SEQ ID NO:194); GLFQKLGBY (SEQ ID NO:195); NEYGIASILD (SEQ ID NO:196); KMVKDALTAI (SEQ ID NO:197); FLASFVHEY (SEQ ID NO:198); AQFVQEATY (SEQ ID NO:199); EYSRRHPQL (SEQ ID NO:411); AYEEDRETF (SEQ ID NO:412; SYANRRPCF (SEQ ID NO:413); CFAEEGQKL (SEQ ID NO:414); RSCGLFQKL (SEQ ID NO:415); IFLIFLLNF (SEQ ID NO:416); KPEGLSPNL (SEQ ID NO:417); and GLSPNLNRFL (SEQ ID NO:418).

In some cases, the AFP peptide present in a TMMP of the present disclosure presents an HLA-A*2402-restricted epitope. Non-limiting examples of AFP peptides that present an HLA-A*2402-restricted epitope are: KYIQESQAL (SEQ ID NO:181); EYYLQNAFL (SEQ ID NO:419); AYTKKAPQL (SEQ ID NO:135); EYSRRHPQL (SEQ ID NO:411); RSCGLFQKL (SEQ ID NO:415) and AYEEDRETF (SEQ ID NO:412).

In some cases, the AFP peptide present in a TMMP of the present disclosure is KYIQESQAL (SEQ ID NO:181). In some cases, the AFP peptide present in a TMMP of the present disclosure is EYYLQNAFL (SEQ ID NO:419. In some cases, the AFP peptide present in a TMMP of the present disclosure is AYTKKAPQL (SEQ ID NO:135). In some cases, the AFP peptide present in a TMMP of the present disclosure is EYSRRHPQL (SEQ ID NO:411). In some cases, the AFP peptide present in a TMMP of the present disclosure is RSCGLFQKL (SEQ ID NO:415).

In some cases, the AFP peptide present in a TMMP of the present disclosure presents an HLA-A*0201-restricted epitope. Non-limiting examples of AFP peptides that present an HLA-A*0201-restricted epitope are: FMNKFIYEI (SEQ ID NO:138); and GLSPNLNRFL (SEQ ID NO:418).

(b) Wilms Tumor Antigen (WT-1)

MAPPs or higher order MAPP complexes such as duplex MAPPs described herein may comprise a peptide presenting an epitope of Wilms Tumor-1 protein, which has been associated with myeloid leukemia, myeloma, ovarian cancer, pancreatic cancer, non-small cell lung cancer, colorectal cancer, breast cancer, Wilms tumor, mesothelioma, soft tissue sarcoma, neuroblastoma, and nephroblastoma. The WT-1 epitope may be presented in the context of a Class I MHC presenting sequence. The WT-1 epitope may be presented in the context of a Class I MHC presenting complex. The Class I MHC may be a) an aa sequence having at least 95% aa sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702,HLA-C*0801, or HLA-C*1502 depicted in FIGS. 3A-3G.

WT-1 peptides for inclusion in a MAPP include, but are not limited to, NLMNLGATL (SEQ ID NO:200), NYMNLGATL (SEQ ID NO:201), CMTWNQMNLGATLKG (SEQ ID NO:202), WNQMNLGATLKGVAA (SEQ ID NO:203), CMTWNYMNLGATLKG (SEQ ID NO:204), WNYMNLGATLKGVAA (SEQ ID NO:205), TWNQMNLGATLKGV (SEQ ID NO:206), TWNQMNLGATLKGVA (SEQ ID NO:207), CMTWNLMNLGATLKG (SEQ ID NO:208), MTWNLMNLGATLKGV (SEQ ID NO:209), TWNLMNLGATLKGVA (SEQ ID NO:210), WNLMNLGATLKGVAA (SEQ ID NO:211), MNLGATLK (SEQ ID NO:212), MTWNYMNLGATLKGV SEQ ID NO:213), TWNYMNLGATLKGVA (SEQ ID NO:214), CMTWNQMNLGATLKGVA (SEQ ID NO:215), CMTWNLMNLGATLKGVA (SEQ ID NO:216), CMTWNYMNLGATLKGVA (SEQ ID NO:217), GYLRNPTAC (SEQ ID NO:218), GALRNPTAL (SEQ ID NO:219), YALRNPTAC (SEQ ID NO:220), GLLRNPTAC (SEQ ID NO:221), RYRPHPGAL (SEQ ID NO:222), YQRPHPGAL (SEQ ID NO:223), RLRPHPGAL (SEQ ID NO:224), RIRPHPGAL (SEQ ID NO:225), QFPNHSFKHEDPMGQ (SEQ ID NO:226), HSFKHEDPY (SEQ ID NO:227), QFPNHSFKHEDPM (SEQ ID NO:228), QFPNHSFKHEDPY (SEQ ID NO:229), KRPFMCAYPGCNK (SEQ ID NO:230), KRPFMCAYPGCYK (SEQ ID NO:231), FMCAYPGCY (SEQ ID NO:232), FMCAYPGCK (SEQ ID NO:233), KRPFMCAYPGCNKRY (SEQ ID NO:234), SEKRPFMCAYPGCNK (SEQ ID NO:235), KRPFMCAYPGCYKRY (SEQ ID NO:236), NLMNLGATL (SEQ ID NO:237), VLDFAPPGA (SEQ ID NO:439); RMFPNAPYL (SEQ ID NO:440); YMFPNAPYL (SEQ ID NO:441); SLGEQQYSV (SEQ ID NO:442); CYTWNQMNL (SEQ ID NO:443); CMTWNQMNL (SEQ ID NO:444); NQMNLGATL (SEQ ID NO:445); and NYMNLGATL (SEQ ID NO:238).

In some cases, the WT-1 peptide present in a TMMP of the present disclosure presents an HLA-A*2402-restricted epitope. WT-1 peptides that present an HLA-A*2402-restricted epitope include, e.g., CMTWNQMN (SEQ ID NO:446); NYMNLGATL (SEQ ID NO:238) (WT-1 239-247; Q240Y); CYTWNQMNL (SEQ ID NO:443) (WT-1 235-243); CMTWNQMNL (SEQ ID NO:444) (WT-1 235-243); NQMNLGATL (SEQ ID NO:445) (WT-1 239-247); and NLMNLGATL (SEQ ID NO:237) (WT-1239-247; Q240L).

In some cases, the WT-1 peptide present in a TMMP of the present disclosure presents an HLA-A*0201-restricted epitope. WT-1 peptides that present an HLA-A*0201-restricted epitope include, e.g., VLDFAPPGA (SEQ ID NO:439) (WT-1 37-45); RMFPNAPYL (SEQ ID NO:440) (WT-1 126-134); YMFPNAPYL (SEQ ID NO:441) (WT-1 126-134; R126Y); SLGEQQYSV (SEQ ID NO:442) (WT-1 187-195); and NLMNLGATL (SEQ ID NO:237) (WT-1 239-247; Q240L).

(c) Human Papilloma Virus I (HPV)

MAPPs or higher order MAPP complexes such as duplex MAPPs described herein may comprise a peptide presenting an epitope of human papilloma virus (HPV), which has been associated with cervical cancer, prostate cancer, or ovarian cancer. HPV epitopes may be presented in the context of a Class I MHC presenting sequence. The HPV epitope may be presented in the context of a Class I MHC presenting complex. The Class I MHC may be a) an aa sequence having at least 95% aa sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702,HLA-C*0801, or HLA-C*1502 depicted in FIGS. 3A-3G. HPV is also an infectious agent and its epitopes may be employed to alter the immune response to HPV for prophylaxis or treatment of an infection.

HPV peptide epitopes include, but are not limited to, those from the E6 and E7 gene products:

E6 18-26
(KLPQLCTEL; SEQ ID NO: 239);
E6 26-34
(LQTTIHDII; SEQ ID NO: 240);
E6 49-57
(VYDFAFRDL; SEQ ID NO: 241);
E6 52-60
(FAFRDLCIV; SEQ ID NO:242);
E6 75-83
(KFYSKISEY; SEQ ID NO: 243);
E6 80-88
(ISEYRHYCY; SEQ ID NO: 244);
E7 7-15
(TLHEYMLDL; SEQ ID NO: 245);
E7 11-19
(YMLDLQPET; SEQ ID NO: 246);
E7 44-52
(QAEPDRAHY; SEQ ID NO: 247);
E7 49-57
(RAHYNIVTF (SEQ ID NO: 248);
E7 61-69
(CDSTLRLCV; SEQ ID NO: 249);
E7 67-76
(LCVQSTHVDI; SEQ ID NO: 250);
E7 82-90
(LLMGTLGIV; SEQ ID NO: 251);
E7 86-93
(TLGIVCPI; SEQ ID NO: 252);
or
E7 92-93
(LLMGTLGIVCPI; SEQ ID NO: 253).

In some cases, the epitope is HPV16E7/82-90 (LLMGTLGIV; SEQ ID NO:251). In some cases, the epitope is HPV16E7/86-93 (TLGIVCPI; SEQ ID NO:252). In some cases, the epitope is HPV16E7/11-20 (YMLDLQPETT; SEQ ID NO:254). In some cases, the epitope is HPV16E7/11-19 (YMLDLQPET; SEQ ID NO:255). See, e.g., Ressing et al. ((1995) J. Immunol. 154:5934) for additional suitable HPV epitopes.

In some cases, a suitable HPV peptide is an HPV E6 peptide that binds HLA-A24 (e.g., is an HLA-A2401-restricted epitope). Non-limiting examples include: VYDFAFRDL (SEQ ID NO:241); CYSLYGTTL (SEQ ID NO:266); EYRHYCYSL (SEQ ID NO:420); KLPQLCTEL (SEQ ID NO:239); DPQERPRKL (SEQ ID NO:399); HYCYSLYGT (SEQ ID NO:421); DFAFRDLCI (SEQ ID NO:422); LYGTTLEQQY (SEQ ID NO:423); HYCYSLYGTT (SEQ ID NO:424); EVYDFAFRDL (SEQ IDNO:425); EYRHYCYSLY (SEQ ID NO:426); VYDFAFRDLC (SEQ ID NO:427); YCYSIYGTTL (SEQ ID NO:428); VYCKTVLEL (SEQ ID NO:429); VYGDTLEKL (SEQ ID NO:430); and LTNTGLYNLL (SEQ ID NO:431).

In some cases, a suitable HPV peptide is selected from the group consisting of: DLQPETTDL (SEQ ID NO:432); TLHEYMLDL (SEQ ID NO:245); TPTLHEYML (SEQ ID NO:433); RAHYNIVTF (SEQ ID NO:248); GTLGIVCPI (SEQ ID NO:434); EPDRAHYNI (SEQ ID NO:435); QLFLNTLSF (SEQ ID NO:436); FQQLFLNTL (SEQ ID NO:437); and AFQQLFLNTL (SEQ IDNO:438).

In some cases, a suitable HPV peptide presents an HLA-A*2401-restricted epitope. Nonlimiting examples of HPV peptides presenting an HLA-A*2401-restricted epitope are: VYDFAFRDL (SEQ ID NO:241); RAHYNIVTF (SEQ ID NO:248); CDSTLRLCV (SEQ ID NO:249); and LCVQSTHVDI (SEQ ID NO:250). In some cases, an HPV peptide suitable for inclusion in a TMMP of the present disclosure is VYDFAFRDL (SEQ ID NO:241). In some cases, an HPV peptide suitable for inclusion in a TMMP of the present disclosure is RAHYNIVTF (SEQ ID NO:248). In some cases, an HPV peptide suitable for inclusion in a TMMP of the present disclosure is CDSTLRLCV (SEQ IDNO:249). In some cases, an HPV peptide suitable for inclusion in a TMMP of the present disclosure is LCVQSTHVDI (SEQ ID NO:250).

(d) Hepatitis B Virus (HBV)

MAPPs or higher order MAPP complexes such as duplex MAPPs described herein may comprise a peptide presenting an epitope of hepatitis B virus (HBV), which has been associated with hepatocellular carcinoma. HBV epitopes may be presented in the context of a Class I MHC presenting complex. The Class I MHC may be a) an aa sequence having at least 95% a sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702,HLA-C*0801, or HLA-C*1502 depicted in FIGS. 3A-3G. HBV is also an infectious agent and its epitopes may be employed to alter the immune response to HBV for prophylaxis or treatment of an infection.

HBV peptide epitopes include, but are not limited to, FLPSDFFPSV (SEQ ID NO:256), GLSRYVARLG (SEQ ID NO:257), KLHLYSHPI (SEQ ID NO:258), FLLSLGIHL (SEQ ID NO:259), ALMPLYACI (SEQ ID NO:260), SLYADSPSV (SEQ ID NO:261), STLPETTVV (SEQ ID NO:262), LIMPARFYPK (SEQ ID NO:263), AIMPARFYPK (SEQ ID NO:264), YVNVNMGLK (SEQ ID NO:265), MQWNSTALHQALQDP (SEQ ID NO:267), LLDPRVRGL (SEQ ID NO:268), SILSKTGDPV (SEQ ID NO:269), VLQAGFFLL (SEQ ID NO:270), FLLTRILTI (SEQ ID NO:271), FLGGTPVCL (SEQ ID NO:272), LLCLIFLLV (SEQ ID NO:273), LVLLDYQGML (SEQ ID NO:274), LLDYQGMLPV (SEQ ID NO:275), IPIPSSWAF (SEQ ID NO:276), WLSLLVPFV (SEQ ID NO:277), GLSPTVWLSV (SEQ ID NO:278), SIVSPFIPLL (SEQ ID NO:279), ILSPFLPLL (SEQ ID NO:280), ATVELLSFLPSDFFPSV (SEQ ID NO:281), LPSDFFPSV (SEQ ID NO:282), CLTFGRETV (SEQ ID NO:283), VLEYLVSFGV (SEQ ID NO:284), EYLVSFGVW (SEQ ID NO:285), ILSTLPETTV (SEQ ID NO:286), STLPETTVVRR (SEQ ID NO:287), NVSIPWTHK (SEQ ID NO:288), KVGNFTGLY (SEQ ID NO:289), GLYSSTVPV (SEQ ID NO:290), TLWKAGILYK (SEQ ID NO:291), TPARVTGGVF (SEQ ID NO:292), LVVDFSQFSR (SEQ ID NO:293), GLSRYVARL (SEQ ID NO:294), SIACSVVRR (SEQ ID NO:295), YMDDVVLGA(SEQ ID NO:296), PLGFFPDH (SEQ ID NO:297), QAFTFSPTYK (SEQ ID NO:298), KYTSFPWLL (SEQ ID NO:299), ILRGTSFVYV (SEQ ID NO:300), HLSLRGLFV (SEQ ID NO:301), VLHKRTLGL (SEQ ID NO:302), GLSAMSTTDL (SEQ ID NO:303), CLFKDWEEL (SEQ ID NO:304), and VLGGCRHKL (SEQ ID NO:305).

(ii) Infectious Agents

Suitable epitopes from infectious agents that may be included in MAPPs or higher order MAPP complexes such as duplex MAPPs include, but are not limited to, epitopes present in an infectious virus, bacterium, fungus, protozoan, or helminth disease causing agents, e.g., an epitope presented by a virus-encoded polypeptide.

Examples of viral infectious disease agents include, e.g., Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)-Group B Arboviruses, Dengue virus serotypes 1, 2, 3, and 4, Yellow fever virus vaccine strain 17D, Hepatitis A, B, C, D, and E viruses, Cytomegalovirus, Epstein Barr virus, Herpes simplex types 1 and 2, Herpes zoster, Human herpesvirus types 6 and 7, hepatitis C virus (HCV, see above), hepatitis B virus (HBV, see above), Influenza viruses types A, B, and C, Papovaviruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, Orbivirus (Colorado tick fever virus), Rabies virus, Vesicular stomatitis virus, Rubivirus (rubella), Semliki Forest virus, St. Louis encephalitis virus, Venezuelan equine encephalitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Lumpy skin disease virus, Malignant catarrhal fever virus, Menangle virus, Vesicular stomatitis virus (exotic), and Zika virus. Antigens encoded by such viruses are known in the art; a peptide epitope suitable for use in a MAPP of the present disclosure can include a peptide from any known viral antigen. In some cases, an HPV antigen is specifically excluded. In some cases, an HBV antigen is specifically excluded.

In some cases, the peptide epitope present in a MAPP of the present disclosure presents an epitope specific to an HLA-A, -B, -C, -E, -F or -G allele. In an embodiment, the peptide epitope present in a MAPP presents an epitope restricted to HLA-A*0101, A*0201, A*0301, A*1101, A*2301, A*2402, A*2407, A*3303, and/or A*3401. In an embodiment, the peptide epitope present in a MAPP presents an epitope restricted to HLA-B*0702, B*0801, B*1502, B*3802, B*4001, B*4601, and/or B*5301. In an embodiment, the peptide epitope present in a MAPP presents an epitope restricted to C*0102, C*0303, C*0304, C*0401, C*0602, C*0701, C*702, C*0801, and/or C*1502.

b. HLA/Peptide Binding Assays

Whether a given peptide (e.g., WT-1 peptide) binds a Class I HLA (comprising an HLA heavy chain and a β2M polypeptide), and, when bound to the HLA complex, can effectively present an epitope to a TCR, can be determined using any of a number of well-known methods. Assays include binding assays and T cell activation assays.

(i) Cell-Based Binding Assay

As one example, a cell-based peptide-induced stabilization assay can be used to determine peptide-HLA Class I binding. In this assay, a peptide of interest is allowed to bind to a TAP-deficient cell, i.e., a cell that has defective transporter associated with antigen processing (TAP) machinery, and consequently, few surface Class I molecules. Such cells include, e.g., the human T2 cell line (T2 (174×CEM.T2; American Type Culture Collection (ATCC) No. CRL-1992). Henderson et al. (1992) Science 255:1264. Without efficient TAP-mediated transport of cytosolic peptides into the endoplasmic reticulum, assembled MHC Class I complexes are structurally unstable, and retained only transiently at the cell surface. However, when T2 cells are incubated with an exogenous peptide capable of binding Class I, surface peptide-HLA Class I complexes are stabilized and can be detected by flow cytometry with, e.g., a pan anti-Class I monoclonal antibody. The stabilization and resultant increased life-span of peptide-HLA complexes on the cell surface by the addition of a peptide validates their identity. Analysis can be carried out using flow cytometry, e.g., where the pan-HLA Class I antibody comprises a fluorescent label. Binding of the peptide to various allelic forms of HLA H chains can be tested by genetically modifying the T2 cells to express an allelic HLA H chain of interest.

The following is a non-limiting example of use of a T2 assay to assess peptide binding to HLA A*0201. T2 cells are washed in cell culture medium, and concentrated to 10⁶ cells/ml. Peptides of interest are prepared in cell culture medium and serially diluted providing concentrations of 200 μM, 100 μM, 20 μM and 2 μM. The cells are mixed 1:1 with each peptide dilution to give a final volume of 200 μL and final peptide concentrations of 100 μM, 50 μM, 10 μM and 1 μM. A HLA A*0201 binding peptide, GILGFVFTL, and a non-HLA A*0201-restricted peptide, HPVGEADYF (HLA-B*3501), are included as positive and negative controls, respectively. The cell/peptide mixtures are kept at 37° C. 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

(ii) Biochemical Binding Assay

HLA polypeptides (HLA heavy chain polypeptide complexed with β2M polypeptide) can be tested for binding to a peptide of interest in a cell-free in vitro assay system. For example, a labeled reference peptide (e.g., fluorescently labeled) is allowed to bind to HLA polypeptides (HLA heavy chain polypeptide complexed with β2M polypeptide), to form an HLA-reference peptide complex. The ability of a test peptide of interest to displace the labeled reference peptide from the HLA-reference peptide complex is tested. The relative binding affinity is calculated as the amount of test peptide needed to displace the bound reference peptide. See, e.g., van der Burg et al. (1995) Human Immunol. 44:189.

As another example, a peptide of interest can be incubated with an HLA molecule (HLA heavy chain complexed with a β2M polypeptide), and the stabilization of the HLA/peptide complex can be measured in an immunoassay format. The ability of a peptide of interest to stabilize an HLA molecule is compared to that of a control peptide presenting a known T cell epitope. Detection of stabilization is based on the presence or absence of the native conformation of the HLA/peptide complex, detected using an anti-HLA antibody. See, e.g., Westrop et al. (2009) J. Immunol. Methods 341:76; Steinitz et al. (2012) Blood 119:4073; and U.S. Pat. No. 9,205,144.

(iii) T Cell Activation Assays

Whether a given peptide binds a Class I HLA (comprising an HLA heavy chain and a β2M polypeptide), and, when bound to the HLA complex, can effectively present an epitope to a TCR, can be determined by assessing T cell response to the peptide-HLA complex. T cell responses that can be measured include, e.g., interferon-gamma (IFNγ) production, cytotoxic activity, and the like.

(iv) ELISPOT Assay

Suitable assays include, e.g., an enzyme linked immunospot (ELISPOT) assay. In this assay, production of IFNγ by CD8⁺ T cells is measured following with an antigen-presenting cell (APC) that presents a peptide of interest complexed with HLA Class I. Antibody to IFNγ is immobilized on wells of a multi-well plate. APCs are added to the wells, and incubated for a period of time with a peptide of interest, such that the peptide binds HLA Class I on the surface of the APCs. CD8⁺ T cells specific for the peptide are added to the wells, and the plate is incubated for about 24 hours. The wells are then washed, and any IFNγ bound to the immobilized anti-IFNγ antibody is detected using a detectably labeled anti-IFNγ antibody. A colorimetric assay can be used. For example, the detectably labeled anti-IFNγ antibody can be a biotin-labeled anti-IFNγ antibody, which can be detected using, e.g., streptavidin conjugated to alkaline phosphatase. A BCIP/NBT (5-bromo-4-chloro-3-indolyl phosphate/nitro blue tetrazolium) solution is added, to develop the assay. The presence of IFNγ-secreting T cells is identified by colored spots. Negative controls include APCs not contacted with the peptide. APCs expressing various HLA H chain alleles can be used to determine whether a peptide of interest effectively binds to a HLA Class I molecule comprising a particular HLA H chain.

(v) Cytotoxicity Assays

Whether a given peptide binds to a particular HLA Class I H chain and, when bound to a HLA Class I complex comprising the H chain, can effectively present an epitope to a TCR, can also be determined using a cytotoxicity assay. A cytotoxicity assay involves incubation of a target cell with a cytotoxic CD8⁺ T cell. The target cell displays on its surface a peptide/HLA Class I complex comprising a peptide of interest and an HLA Class I molecule comprising an HLA H chain to be tested. The target cells can be radioactively labeled, e.g., with ⁵¹Cr. Whether the target cell effectively presents an epitope to a TCR on the cytotoxic CD8⁺ T cell, thereby inducing cytotoxic activity by the CD8⁺ T cell toward the target cell, is determined by measuring release of ⁵¹Cr from the lysed target cell. Specific cytotoxicity can be calculated as the amount of cytotoxic activity in the presence of the peptide minus the amount of cytotoxic activity in the absence of the peptide.

(vi) Detection of Antigen-Specific T Cells with Peptide-HLA Tetramers

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.
8. Additional Polypeptides

A polypeptide chain of a MAPP of the present disclosure (e.g., a dimerization or framework polypeptide) may include one or more polypeptides in addition to those described above. Suitable additional polypeptides include epitope tags and affinity domains. The one or more additional polypeptides can be included at the N-terminus of a polypeptide chain of a MAPP of the present disclosure, at the C-terminus of a polypeptide chain of a MAPP of the present disclosure, or within (internal to) a polypeptide chain of a MAPP of the present disclosure.

a. Affinity Tags, Epitope Tags and Affinity Domains

Suitable affinity/epitope tags include, but are not limited to, hemagglutinin (HA; e.g., YPYDVPDYA (SEQ ID NO:306); FLAG (e.g., DYKDDDDK (SEQ ID NO:307); c-myc (e.g., EQKLISEEDL; SEQ ID NO:308), and the like.

Affinity domains include peptide sequences that can interact with a binding partner, e.g., such as one immobilized on a solid support, useful for identification or purification. DNA sequences encoding multiple consecutive single aas, such as histidine, when fused to the expressed protein, may be used for one-step purification of the recombinant protein by high affinity binding to a resin column, such as nickel sepharose. Exemplary affinity domains include HisX5 (HHHHH) (SEQ ID NO:309), HisX6 (HHHHHH) (SEQ ID NO:310), C-myc (EQKLISEEDL) (SEQ ID NO:311), Flag (DYKDDDDK) (SEQ ID NO:312), StrepTag (WSHPQFEK) (SEQ ID NO:313), hemagglutinin, e.g., HA Tag (YPYDVPDYA) (SEQ ID NO:314), glutathione-S-transferase (GST), thioredoxin, cellulose binding domain, RYIRS (SEQ ID NO:315), Phe-His-His-Thr (SEQ ID NO:316), chitin binding domain, S-peptide, T7 peptide, SH2 domain, C-end RNA tag, WEAAAREACCRECCARA (SEQ ID NO:317), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, calretinin, inteins, biotin, streptavidin, MyoD, Id, leucine zipper sequences, and maltose binding protein.

b. Targeting Sequences

MAPPs may include, as part of any one or more framework and/or any one or more dimerization polypeptide, a targeting polypeptide or “targeting sequence.” Targeting sequences serve to bind or “localize” MAPPs to cells and/or tissues displaying the protein (or other molecule) to which the targeting sequence binds. Targeting sequences may be located, for example at or near the carboxyl terminal end of a framework or dimerization peptide (e.g., in place of a C-terminal MOD in FIG. 1A or 1B or at position 3, 3′, 5 and/or 5′ of the MAPP in any of FIG. 1A, 1B or 6-9). In an embodiment the targeting sequence may be located at position 3 and/or 3′. Targeting sequences serve to bind or “localize” MAPPs to cells and tissue displaying the protein (or other molecule) which the targeting sequence binds. In some cases, a targeting sequence is an antibody or antigen binding fragment thereof (e.g., an scFv or a nanobody such as a heavy chain nanobody or a light chain nanobody). In some cases, a targeting sequence is a single-chain T cell receptor (scTCR). Targeting sequences may be translated as part of the MAPP (e.g., part of the framework polypeptide) or incorporated by covalent attachment (e.g., using a crosslinker) of a targeting sequence, where the targeting sequence effectively becomes a payload-like molecule attached to the MAPP. Targeting sequences may also be non-covalently bound to a MAPP. For example, a MAPP having a biotin labeled framework polypeptide may be non-covalently attached to an avidin labeled targeting antibody or Fab directed to, for example, a cancer antigen). A bispecific antibody (e.g., a bispecific IgG or humanized antibody) having a first antigen binding site directed to a part of the MAPP (e.g., the framework polypeptide) may also be employed to non-covalently attach a MAPP to a targeting sequence (the second bispecific antibody binding site) directed to a cell or tissue target (e.g., a cancer antigen).

In some instances, a targeting sequence present in a MAPP targets an antigen of an infecting organism and/or of an infected cell. Targeting polypeptides may be directed to proteins/epitopes of infectious agents including, but not limited to, viruses, bacteria, fungi, protozoans, and helminths, including those proteins/epitopes of infectious agents that are expressed on cell surfaces. By way of example, cells infected with HPV may express E6 or E7 proteins (described herein as cancer associated epitope) or portions thereof to which the targeting sequence may be directed. A targeting sequence may also be a Cancer Targeting Polypeptide, or “CTP” that is specific for a cancer associated antigen (“CAA”), such as an antigen associated with a non-solid cancer (e.g., a leukemia) and/or solid tumor-associated antigen. In one instance, the targeting sequence is specific for a cancer-associated peptide/HLA (pHLA) complex on the surface of a cancer cell, where the peptide can be a cancer-associated peptide (e.g., a peptide fragment of a cancer-associated antigen). MAPPs can also be targeted to specific tissues or cell types by using targeting sequences directed toward molecules expressed selectively by cells of the desired tissue.

(a) Cancer Associated Antigens (CAAs)

CAAs that can be targeted with a CTP present in a MAPP or a higher order MAPP complex, such as a duplex MAPP, of the present disclosure include, e.g., NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, MUC1 (mucin 1), MUC16 (mucin 16), myelin, NKG2D (Natural Killer Group 2D) ligands, PSMA (prostate specific membrane antigen), and ROR1 (type I receptor tyrosine kinase like orphan receptor).

CAAs that can be targeted with a CTP present in a MAPP of the present disclosure also include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CD28, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Legamma, macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the CAA is an antigen that may be targeted with a CTP of a MAPP associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the CAA is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, a CAA that is to be targeted by a CTP is associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Legamma, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

(b) Peptide/HLA Complexes

In some cases, a CTP of a MAPP or a higher order MAPP complex, such as a duplex MAPP, targets a peptide/HLA (pHLA) complex on the surface of a cancer cell, where the peptide is a cancer-associated peptide (e.g., a peptide fragment of a cancer-associated antigen). Cancer-associated peptide antigens are known in the art. In some cases, a cancer-associated peptide is bound to a HLA complex comprising an HLA-A*0201 heavy chain and a β2M polypeptide.

In some cases, the CAA peptide epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the peptide epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the peptide epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, a CAA peptide is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of any one of the following cancer-associated antigens: a CD28 polypeptide, a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a claudin polypeptide (e.g., claudin-1, claudin-10, claudin-18 (e.g., claudin-18, isoform 2)), a Nectin-4 polypeptide, a melanoma antigen recognized by T cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE Al) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGSS) polypeptide, a squamous cell carcinoma antigen recognized by T cells (SART3) polypeptide, a carbonic anhydrase IX polypeptide, a paired box-5 (PAXS) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an Akinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, a Fos-related antigen-1 (FOSL) polypeptide; a human papilloma virus (HPV) antigen; an alpha-feto protein (AFP) antigen; and a Wilms tumor-1 (WT1) antigen.

For example, in some cases, a CTP present in a MAPP of the present disclosure binds to: a) a WT-1 peptide bound to an HLA complex comprising an HLA heavy chain (e.g., an HLA-A*0201 heavy chain or an HLA-A*2402 heavy chain) and a β2M polypeptide; b) an HPV peptide bound to an HLA complex comprising a Class I HLA heavy chain and a β2M polypeptide; c) a mesothelin peptide bound to an HLA complex comprising a Class I HLA heavy chain and a β2M polypeptide; d) a Her2 peptide bound to an HLA complex comprising a Class I HLA heavy chain and a β2M polypeptide; or e) a BCMA peptide bound to an HLA complex comprising a Class I HLA heavy chain and a β2M polypeptide.

In some cases, a CAA peptide is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a mesothelin polypeptide having at least 90%, at least 95%, at least 98%, at least 99%, or 100%, aa sequence identity to the following mesothelin aa sequence: LAGE TGQEAAPLDG VLANPPNISS LSPRQLLGFP CAEVSGLSTE RVRELAVALA QKNVKLSTEQ LRCLAHRLSE PPEDLDALPL DLLLFLNPDA FSGPQACTRF FSRITKANVD LLPRGAPERQ RLLPAALACW GVRGSLLSEA DVRALGGLAC DLPGRFVAES AEVLLPRLVS CPGPLDQDQQ EAARAALQGG GPPYGPPSTW SVSTMDALRG LLPVLGQPII RSIPQGIVAA WRQRSSRDPS WRQPERTILR PRFRREVEKT ACPSGKKARE IDESLIFYKK WELEACVDAA LLATQMDRVN AIPFTYEQLD VLKHKLDELY PQGYPESVIQ HLGYLFLKMS PEDIRKWNVT SLETLKALLE VNKGHEMSPQ VATLIDRFVK GRGQLDKDTL DTLTAFYPGY LCSLSPEELS SVPPSSIWAV RPQDLDTCDP RQLDVLYPKA RLAFQNMNGS EYFVKIQSFL GGAPTEDLKA LSQQNVSMDL ATFMKLRTDA VLPLTVAEVQ KLLGPHVEGL KAEERHRPVR DWILRQRQDD LDTLGLGLQG GIPNGYLVLD LSMQEALSGT PCLLGPGPVL TVLALLLAST LA (SEQ ID NO:318). For example, a mesothelin peptide present in a pHLA complex can be: i) KLLGPHVEGL (SEQ ID NO:319); ii) AFYPGYLCSL (SEQ ID NO:320), which can bind HLA-A*2402/β2M; iii) VLPLTVAEV (SEQ ID NO:321); iv) ELAVALAQK (SEQ ID NO:322); v) ALQGGGPPY (SEQ ID NO:323); vi) FYPGYLCSL (SEQ ID NO:324); vii) LYPKARLAF (SEQ ID NO:325); viii) LLFLLFSLGWVGPSR (SEQ ID NO:326); ix) VNKGHEMSPQAPRRP (SEQ ID NO:327); x) FMKLRTDAVLPLTVA (SEQ ID NO:328); or xi) DAALLATQMD (SEQ ID NO:329).

In some cases, a CAA peptide is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a mesothelin polypeptide having at least 90%, at least 95%, at least 98%, at least 99%, or 100%, aa sequence identity to the following Her2 (receptor tyrosine-protein kinase erbB2) aa sequence:

(SEQ ID NO: 330)
MELAALCRWG LLLALLPPGA ASTQVCTGTD
MKLRLPASPE THLDMLRHLY QGCQVVQGNL
ELTYLPTNAS LSFLQDIQEV QGYVLIAHNQ
VRQVPLQRLR IVRGTQLFED NYALAVLDNG
DPLNNTTPVT GASPGGLREL QLRSLTEILK
GGVLIQRNPQ LCYQDTILWK DIFHKNNQLA
LTLIDTNRSR ACHPCSPMCK GSRCWGESSE
DCQSLTRTVC AGGCARCKGP LPTDCCHEQC
AAGCTGPKHS DCLACLHFNH SGICELHCPA
LVTYNTDTFE SMPNPEGRYT FGASCVTACP
YNYLSTDVGS CTLVCPLHNQ EVTAEDGTQR
CEKCSKPCAR VCYGLGMEHL REVRAVTSAN
IQEFAGCKKI FGSLAFLPES FDGDPASNTA
PLQPEQLQVF ETLEEITGYL YISAWPDSLP
DLSVFQNLQV IRGRILHNGA YSLTLQGLGI
SWLGLRSLRE LGSGLALIHH NTHLCFVHTV
PWDQLFRNPH QALLHTANRP EDECVGEGLA
CHQLCARGHC WGPGPTQCVN CSQFLRGQEC
VEECRVLQGL PREYVNARHC LPCHPECQPQ
NGSVTCFGPE ADQCVACAHY KDPPFCVARC
PSGVKPDLSY MPIWKFPDEE GACQPCPINC
THSCVDLDDK GCPAEQRASP LTSIISAVVG
ILLVVVLGVV FGILIKRRQQ KIRKYTMRRL
LQETELVEPL TPSGAMPNQA QMRILKETEL
RKVKVLGSGA FGTVYKGIWI PDGENVKIPV
AIKVLRENTS PKANKEILDE AYVMAGVGSP
YVSRLLGICL TSTVQLVTQL MPYGCLLDHV
RENRGRLGSQ DLLNWCMQIA KGMSYLEDVR
LVHRDLAARN VLVKSPNHVK ITDFGLARLL
DIDETEYHAD GGKVPIKWMA LESILRRRFT
HQSDVWSYGV TVWELMTFGA KPYDGIPARE
IPDLLEKGER LPQPPICTID VYMIMVKCWM
IDSECRPRFR ELVSEFSRMA RDPQRFVVIQ
NEDLGPASPL DSTFYRSLLE DDDMGDLVDA
EEYLVPQQGF FCPDPAPGAG GMVHHRHRSS STRNM.

In some cases, a CAA peptide is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a B-cell maturation protein (BCMP) polypeptide having at least 90%, at least 95%, at least 98%, at least 99%, or 100%, aa sequence identity to the following BCMA aa sequence:

(SEQ ID NO: 331)
MLQMAGQCSQ NEYFDSLLHA CIPCQLRCSS
NTPPLTCQRY CNASVTNSVK GTNAILWTCL
GLSLIISLAV FVLMFLLRKI SSEPLKDEFK
NTGSGLLGMA NIDLEKSRTG DEIILPRGLE
YTVEECTCED CIKSKPKVDS DHCFPLPAME
EGATILVTTK TNDYCKSLPA ALSATEIEKS
ISAR.

In some cases, a CAA peptide is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a mesothelin polypeptide having at least 90%, at least 95%, at least 98%, at least 99%, or 100%, aa sequence identity to the following WT-1 aa sequence: MDFLLLQDPA STCVPEPASQ HTLRSGPGCL QQPEQQGVRD PGGIWAKLGA AEASAERLQG RRSRGASGSE PQQMGSDVRD LNALLPAVPS LGGGGGCALP VSGAAQWAPV LDFAPPGASA YGSLGGPAPP PAPPPPPPPP PHSFIKQEPS WGGAEPHEEQ CLSAFTVHFS GQFTGTAGAC RYGPFGPPPP SQASSGQARM FPNAPYLPSC LESQPAIRNQ GYSTVTFDGT PSYGHTPSHH AAQFPNHSFK HEDPMGQQGS LGEQQYSVPP PVYGCHTPTD SCTGSQALLL RTPYSSDNLY QMTSQLECMT WNQMNLGATL KGHSTGYESD NHTTPILCGA QYRIHTHGVF RGIQDVRRVP GVAPTLVRSA SETSEKRPFM CAYPGCNKRY FKLSHLQMHS RKHTGEKPYQ CDFKDCERRF SRSDQLKRHQ RRHTGVKPFQ CKTCQRKFSRSDHLKTHTRT HTGEKPFSCR WPSCQKKFAR SDELVRHHNM HQRNMTKLQL AL (SEQ ID NO:332).

Non-limiting examples of WT-1 peptides include RMFPNAPYL (SEQ ID NO:397), CMTWNQMN (SEQ ID NO:403), CYTWNQMNL (SEQ ID NO:400), CMTWNQMNLGATLKG (SEQ ID NO:202), WNQMNLGATLKGVAA (SEQ ID NO:203), CMTWNYMNLGATLKG (SEQ ID NO:204), WNYMNLGATLKGVAA (SEQ ID NO:205), MTWNQMNLGATLKGV (SEQ ID NO:447), TWNQMNLGATLKGVA (SEQ ID NO:207), CMTWNLMNLGATLKG (SEQ ID NO:208), MTWNLMNLGATLKGV (SEQ ID NO:209), TWNLMNLGATLKGVA (SEQ ID NO:210), WNLMNLGATLKGVAA (SEQ ID NO:211), MNLGATLK (SEQ ID NO:212), MTWNYMNLGATLKGV (SEQ ID NO:213), TWNYMNLGATLKGVA (SEQ ID NO:214), CMTWNQMNLGATLKGVA (SEQ ID NO:215), CMTWNLMNLGATLKGVA (SEQ ID NO:216), CMTWNYMNLGATLKGVA (SEQ ID NO:217), GYLRNPTAC (SEQ ID NO:218), GALRNPTAL (SEQ ID NO:219), YALRNPTAC (SEQ ID NO:220), GLLRNPTAC (SEQ ID NO:221), RYRPHPGAL (SEQ ID NO:222), YQRPHPGAL (SEQ ID NO:223), RLRPHPGAL (SEQ ID NO:224), RIRPHPGAL (SEQ ID NO:225), QFPNHSFKHEDPMGQ (SEQ ID NO:226), HSFKHEDPY (SEQ ID NO:227), QFPNHSFKHEDPM (SEQ ID NO:228), QFPNHSFKHEDPY (SEQ ID NO:229), KRPFMCAYPGCNK (SEQ ID NO:230), KRPFMCAYPGCYK (SEQ ID NO:231), FMCAYPGCY (SEQ ID NO:232), FMCAYPGCK (SEQ ID NO:233), KRPFMCAYPGCNKRY (SEQ ID NO:234), SEKRPFMCAYPGCNK (SEQ ID NO:235), KRPFMCAYPGCYKRY (SEQ ID NO:236), NLMNLGATL (SEQ ID NO:237), NYMNLGATL (SEQ ID NO:238), and those WT-1 peptides recited in section IV.C.7.a.(i)(b).

In some cases, a CAA is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of an HPV polypeptide having at least 90%, at least 95%, at least 98%, at least 99%, or 100%, aa sequence identity to a human papilloma virus (HPV) peptide. An HPV peptide can be a peptide of an HPV E6 polypeptide or an HPV E7 protein. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. Non-limiting examples of HPV peptides include: E6 18-26 (KLPQLCTEL; SEQ ID NO:239); E6 26-34 (LQTTIHDII; SEQ ID NO:240); E6 49-57 (VYDFAFRDL; SEQ ID NO:241); E6 52-60 (FAFRDLCIV; SEQ ID NO:242); E6 75-83 (KFYSKISEY; SEQ ID NO:243); E6 80-88 (ISEYRHYCY; SEQ ID NO:244); E7 7-15 (TLHEYMLDL; SEQ ID NO:245); E7 11-19 (YMLDLQPET; SEQ ID NO:246); E7 44-52 (QAEPDRAHY; SEQ ID NO:247); E7 49-57 (RAHYNIVTF (SEQ ID NO:248); E7 61-69 (CDSTLRLCV; SEQ ID NO:249); and E7 67-76 (LCVQSTHVDI; SEQ ID NO:250); E7 82-90 (LLMGTLGIV; SEQ ID NO:251); E7 86-93 (TLGIVCPI; SEQ ID NO:252); E7 92-93 (LLMGTLGIVCPI; SEQ ID NO:253); and those HPV peptides recited herein (see, e.g., section IV.C.7.a.(i).(c)).

In some cases, a CAA peptide is a peptide of a claudin polypeptide having at least 90%, at least 95%, at least 98%, at least 99%, or 100%, amino acid sequence identity to the following claudin-18 (isoform 2) (CLDN 18.2) amino acid sequence: MAVTACQGLG FVVSLIGIAG IIAATCMDQW STQDLYNNPV TAVFNYQGLW RSCVRESSGF TECRGYFTLL GLPAMLQAVR ALMIVGIVLG AIGLLVSIFA LKCIRIGSME DSAKANMTLT SGIMFIVSGL CAIAGVSVFA NMLVTNFWMS TANMYTGMGG MVQTVQTRYT FGAALFVGWV AGGLTLIGGV MMCIACRGLA PEETNYKAVS YHASGHSVAY KPGGFKASTG FGSNTKNKKI YDGGARTEDE VQSYPSKHDY V (SEQ ID NO:448). In some cases, a cancer-associated peptide is a peptide of a claudin polypeptide having the amino acid sequence TEDEVQSYPSKHDYV (SEQ ID NO:449) (and having a length of about 15 amino acids) or EVQSYPSKHDYV (SEQ ID NO:450) (and having a length of about 12 amino acids.

In some cases, a CAA peptide is a peptide of a trophoblast cell-surface antigen-2 (Trop-2) polypeptide. Trop-2 (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene. In some cases, a cancer-associated peptide is a peptide of a TROP-2 polypeptide having at least 90%, at least 95%, at least 98%, at least 99%, or 100%, amino acid sequence identity to the following TROP-2 amino acid sequence: QDNCTCPTNK MTVCSPDGPG GRCQCRALGS GMAVDCSTLT SKCLLLKARM SAPKNARTLV RPSEHALVDN DGLYDPDCDP EGRFKARQCN QTSVCWCVNS VGVRRTDKGD LSLRCDELVR THHILIDLRH RPTAGAFNHS DLDAELRRLF RERYRLHPKF VAAVHYEQPT IQIELRQNTS QKAAGDVDIG DAAYYFERDI KGESLFQGRG GLDLRVRGEP LQVERTLIYY LDEIPPKFSM KRLTAGLIAV IVVVVVALVA GMAVLVITNR RKSGKYKKVE IKELGELRKE PSL (SEQ ID NO:451).

(ii) Antibodies

As noted above, in some cases, a CTP present in a MAPP or a higher order MAPP complex, such as a duplex MAPP, of the present disclosure is an antibody or an antigen binding fragment thereof. In some cases, the CTP is an antibody that is specific for a CAA. In some cases, the CTP is an antibody specific for a peptide on the surface of an infected cell (e.g., viral, bacterial, or mycoplasma). In some cases, the CTP is an antibody specific for a peptide/HLA complex on the surface of a cancer cell, where the peptide can be a cancer-associated peptide (e.g., a peptide fragment of a cancer-associated antigen).

There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

The term “humanized immunoglobulin” as used herein refers to an immunoglobulin comprising portions of immunoglobulins of different origin, wherein at least one portion comprises amino acid sequences of human origin. Chimeric or CDR-grafted single chain antibodies are also encompassed by the term humanized immunoglobulin.

The terms “antibodies” and “immunoglobulin” include antibodies or immunoglobulins of any isotype, fragments of antibodies that retain specific binding to antigen, including, but not limited to, Fab, F(ab′)₂, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1).

“Fv” is the minimum antibody fragment that contains a complete antigen-recognition and -binding site. This region consists of a dimer of one heavy- and one light-chain variable domain in tight, non-covalent association.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth in the CDR-table below as a comparison.

CDR Table

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. The terms “CDR-L1”, “CDR-L2”, and “CDR-L3” may be used interchangeably with “VL CDR1,” “VL CDR2,” and “VL CDR3,” respectively. As used herein, the terms “CDR-H1”, “CDR-H2”, and “CDR-H3” refer, respectively, to the first, second, and third CDRs in a heavy chain variable region. The terms “CDR-H1”, “CDR-H2”, and “CDR-H3” may be used interchangeably with “VH CDR1,” “VH CDR2,” and “VH CDR3,” respectively. As used herein, the terms “CDR-1”, “CDR-2”, and “CDR-3” refer, respectively, to the first, second and third CDRs of either chain's variable region.

Non-limiting examples of CAA-targeted antibodies (or antigen binding fragments thereof) that can be included in a MAPP of the present disclosure include, but are not limited to, abituzumab (anti-CD51), LL1 (anti-CD74), LL2 or RFB4 (anti-CD22), veltuzumab (hA20, anti-CD20), rituxumab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), R1 (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

In some cases, a CAA-targeted antibody (or antigen binding fragments thereof) that can be included in a MAPP is a single-chain antibody. In some cases, a CAA-targeted antibody (or antigen binding fragments thereof) that can be included in a MAPP is a scFv. In some cases, the tumor-targeting polypeptide is a nanobody (also referred to as a single domain antibody (sdAb)). In some cases, the tumor-targeting polypeptide is a heavy chain nanobody. In some cases, the tumor-targeting polypeptide is a light chain nanobody.

VH and VL aa sequences of various tumor antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of tumor antigen-binding antibodies.

(a) Anti-Her2

In some cases, an anti-Her2 antibody (or antigen binding fragments thereof) useful as a CTP comprises: a) a light chain comprising an aa sequence having at least 90%, at least 95%, at least 98%, at least 99%, or 100%, aa sequence identity to the following aa sequence:
DIQMTQSPSSLSASVGDRVTITCRASQDVNTAVAWYQQKPGKAPKLLIYSASFLYSGVPSRFSG SRSGTDFTLTIS SLQPEDFATYYCQQHYTTPPTFGQGT KVEIKRTVAAPSVFIFPPSDEQLKSGTA SVVCLLNNFYPREAKVQWKVDNALQSGNSQESVTEQDSKDSTYSLSSTLTLSKADYEKHKVY ACEVTHQGLSSPVTKSFNRGEC (SEQ ID NO:333); and b) a heavy chain comprising an aa sequence having at least 90%, at least 95%, at least 98%, at least 99%, or 100%, aa sequence identity to the following aa sequence: EVQLVESGGGLVQPGGSLRLSCAASGFNIKDTYIH WVRQAPGKGLEWVARIYPTNGYTRYADSVKGRFTISADTSKNTAYLQMNSLRAEDTAVYYCS RWGGDGFYAMDYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKVDKKVEPKSCD KTHTCPPCPAPELLGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALPAPIEKTISKAKGQPREPQV YTLPPSREEMTKNQV SLTCLVKGFYPSDIAVEWESNGQPENNYKTTPPVLDSDGSFFLYSKLTV DKSRWQQGNVFSCSVMHEALHNHYTQKSLSLSPGK (SEQ ID NO:334).

In some cases, an anti-Her2 antibody (or antigen binding fragments thereof) comprises a light chain variable region (VL) present in the light chain aa sequence provided above; and a heavy chain variable region (VH) present in the heavy chain aa sequence provided above. For example, an anti-Her2 antibody can comprise: a) a VL comprising an aa sequence having at least 90%, at least 95%, at least 98%, at least 99%, or 100%, aa sequence identity to the aa sequence:

(SEQ ID NO: 335)
DIQMTQSPSSLSASVGDRVTITCRASQDVNTAVAWYQQKPGKAPKLLI
YSASFLYSGVPSRFSGSRSGTDFTLTISSLQPEDFATYYCQQHYTTPP
TFGQGTKVEIK;

and b) a VH comprising an aa sequence having at least 90%, at least 95%, at least 98%, at least 99%, or 100%, aa sequence identity to the aa sequence: EVQLVESGGGLVQPGGSLRLSCA ASGFNIKDTYIHWVRQAPGKGLEWVARIYPTNGYTRYADSVKGRFTISADTSKNTAYLQMNSL RAEDTAVYYCSRWGGDGFYAMDYWGQGTLVTVSS (SEQ ID NO:336). In some cases, an anti-Her2 antibody comprises, in order from N-terminus to C-terminus: a) a VH comprising an aa sequence having at least 90%, at least 95%, at least 98%, at least 99%, or 100%, aa sequence identity to the aa sequence: EVQLVESGGGLVQPGGSLRLSCAASGFNIKDTYIHWVRQAPGKGL EWVARIYPTNGYTRYADSVKGRFTISADTSKNTAYLQMNSLRAEDTAVYYCSRWGGDGFYAM DYWGQGTLVTVSS (SEQ ID NO:337); b) a linker; and c) a VL comprising an aa sequence having at least 90%, at least 95%, at least 98%, at least 99%, or 100%, aa sequence identity to the aa sequence: DIQMTQSPSSLSASVGDRVTITCRASQDVNTAVAWYQQKPGKAPKLLIYSA SFLYSGVPSRFSGSRSGTDFTLTISSLQPEDFATYYCQQHYTTPPTFGQGTKVEIK (SEQ ID NO:338). Suitable linker sequences described elsewhere herein and include, e.g., (GGGGS) (SEQ ID NO:339), which may be repeated from 1 to 10 times (e.g., 1, 2, 3, 4, 5, 6, 7, 8, 9, or 10 times).

In some cases, an anti-Her2 antibody (or antigen binding fragments thereof) comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991. In some cases, the VH and VL CDRs are as defined by Chothia et al., J. Mol. Biol. 196:901-917 (1987) (also referred to herein as Chothia 1987). For example, an anti-Her2 antibody (or antigen binding fragments thereof) can comprise a VL CDR1 having the aa sequence RASQDVNTAVA (SEQ ID NO:340); a VL CDR2 having the aa sequence SASFLY (SEQ ID NO:341); a VL CDR3 having the aa sequence QQHYTTPP (SEQ ID NO:342); a VH CDR1 having the aa sequence GFNIKDTY (SEQ ID NO343); a VH CDR2 having the aa sequence IYPTNGYT (SEQ ID NO:344); and a VH CDR3 having the aa sequence SRWGGDGFYAMDY (SEQ ID NO:345).

In some cases, an anti-Her2 antibody (or antigen binding fragments thereof) is a scFv antibody. For example, an anti-Her2 scFv can comprise an aa sequence having at least 90%, at least 95%, at least 98%, at least 99%, or 100%, aa sequence identity to the following aa sequence: EVQLVESGGGLVQPG GSLRLSCAASGFNIKDTYIHWVRQAPGKGLEWVARIYPTNGYTRYADSVKGRFTISADTSKNTA YLQMNSLRAEDTAVYYCSRWGGDGFYAMDYWGQGTLVTVSSGGGGSGGGGSGGGGSDIQM TQSPSSLSASVGDRVTITCRASQDVNTAVAWYQQKPGKAPKLLIYSASFLYSGVPSRFSGSRSGT DFTLTISSLQPEDFATYYCQQHYTTPPTFGQGTKVEIK (SEQ ID NO:346).

As another example, in some cases, an anti-Her2 antibody (or antigen binding fragments thereof) comprises: a) a light chain variable region (VL) comprising an aa sequence having at least 90%, at least 95%, at least 98%, at least 99%, or 100%, aa sequence identity to the following aa sequence: DIQMTQSPSSLSASVGDRVTITCKASQDVSIGVAWYQQKPGKAPKL LIYSASYRYTGVPSRFSGSGSGTDFTLTISSLQPEDFATYYCQQYYIYPYTFGQGTKVEIKRTVAA PSVFIFPPSDEQLKSGTASVVCLLNNFYPREAKVQWKVDNALQSGNSQESVTEQDSKDSTYSLS STLTLSKADYEKHKVYACEVTHQGLSSPVTKSFNRGEC (SEQ ID NO:347); and b) a heavy chain variable region (VH) comprising an aa sequence having at least 90%, at least 95%, at least 98%, at least 99%, or 100%, aa sequence identity to the following aa sequence: EVQLVESGGG LVQPGGSLRLSCAASGFTFTDYTMDWVRQAPGKGLEWVADVNPNSGGSIYNQRFKGRFTLSV DRSKNTLYLQMNSLRAEDTAVYYCARNLGPSFYFDYWGQGTLVTVSSASTKGPSVFPLAPSSK STSGGTAALGCLVKDYFPEPVTVSWNSGALTSGVHTFPAVLQSSGLYSLSSVVTVPSSSLGTQT YICNVNHKPSNTKVDKKVEPKSCDKTHTCPPCPAPELLGGPSVFLFPPKPKDTLMISRTPEVTCV VVDVSHEDPEVKFNWYVDGVEVHNAKTKPREEQYNSTYRVVSVLTVLHQDWLNGKEYKCKV SNKALPAPIEKTISKAKGQPREPQVYTLPPSREEMTKNQVSLTCLVKGFYPSDIAVEWESNGQPE NNYKTTPPVLDSDGSFFLYSKLTVDKSRWQQGNVFSCSVMHEALHNHYTQKSLSLSPG (SEQ ID NO:348).

In some cases, an anti-Her2 antibody (or antigen binding fragments thereof) useful as a CTP comprises a VL present in the light chain aa sequence provided above; and a VH present in the heavy chain aa sequence provided above. For example, an anti-Her2 antibody can comprise: a) a VL comprising an aa sequence having at least 90%, at least 95%, at least 98%, at least 99%, or 100%, aa sequence identity to the aa sequence: DIQMTQSPSSLSASVGDRVTITCKASQDVSIGVAWYQQKP GKAPKLLIYSASYRYTGVPSRFSGSGSGTDFTLTISSLQPEDFATYYCQQYYIYPYTFGQGTKVEI K (SEQ ID NO:349); and b) a VH comprising an aa sequence having at least 90%, at least 95%, at least 98%, at least 99%, or 100%, aa sequence identity to the aa sequence: EVQLVESGGGLVQPGGSLRLS CAASGFTFTDYTMDWVRQAPGKGLEWVADVNPNSGGSIYNQRFKGRFTLSVDRSKNTLYLQM NSLRAEDTAVYYCARNLGPSFYFDYWGQGTLVTVSS (SEQ ID NO:350).

In some cases, an anti-Her2 antibody (or antigen binding fragments thereof) used as a CTP comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat (see, e.g., Kabat 1991). In some cases, the VH and VL CDRs are as defined by Chothia (see, e.g., Chothia 1987). For example, an anti-HER2 antibody can comprise a VL CDR1 having the aa sequence KASQDVSIGVA (SEQ ID NO:351); a VL CDR2 having the aa sequence SASYRY (SEQ ID NO:352); a VL CDR3 having the aa sequence QQYYIYPY (SEQ ID NO:353); a VH CDR1 having the aa sequence GFTFTDYTMD (SEQ ID NO:354); a VH CDR2 having the aa sequence ADVNPNSGGSIYNQRFKG (SEQ ID NO:355); and a VH CDR3 having the aa sequence ARNLGPSFYFDY (SEQ ID NO:356).

In some cases, an anti-Her2 antibody (or antigen binding fragments thereof) is a scFv. For example, in some cases, an anti-Her2 scFv comprises an aa sequence having at least 90%, at least 95%, at least 98%, at least 99%, or 100%, aa sequence identity to the following aa sequence:

(SEQ ID NO: 357)
EVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYC
SRWGGDGFYAMDYWGQGTLVTVSSGGGGSGGGGSGGGGSDIQMTQSPS
SLSASVGDRVTITCRASQDVNTAVAWYQQKPGKAPKLLIYSASFLYSG
VPSRFSGSRSGTDFTLTISSLQPEDFATYYCQQHYTTPPTFGQGTKVE
IK.

(b) Anti-CD19

Anti-CD19 antibodies (and antigen binding fragments thereof) useful as a CTP are known in the art; and the VH and VL, or the VH and VL CDRs, of any anti-CD19 antibody can be used in a MAPP of the present disclosure. See e.g., WO 2005/012493.

In some cases, an anti-CD19 antibody (or antigen binding fragments thereof) includes a VL CDR1 comprising the aa sequence KASQSVDYDGDSYLN (SEQ ID NO:358); a VL CDR2 comprising the aa sequence DASNLVS (SEQ ID NO:359); and a VL CDR3 comprising the aa sequence QQSTEDPWT (SEQ ID NO:360). In some cases, an anti-CD19 antibody (or antigen binding fragments thereof) includes a VH CDR1 comprising the aa sequence SYWMN (SEQ ID NO:361); a VH CDR2 comprising the aa sequence QIWPGDGDTNYNGKFKG (SEQ ID NO:362); and a VH CDR3 comprising the aa sequence RETTTVGRYYYAMDY (SEQ ID NO:363). In some cases, an anti-CD19 antibody includes a VL CDR1 comprising the aa sequence KASQSVDYDGDSYLN (SEQ ID NO:364); a VL CDR2 comprising the aa sequence DASNLVS (SEQ ID NO:365); a VL CDR3 comprising the aa sequence QQSTEDPWT (SEQ ID NO:366); a VH CDR1 comprising the aa sequence SYWMN (SEQ ID NO:367); a VH CDR2 comprising the aa sequence QIWPGDGDTNYNGKFKG (SEQ ID NO:368); and a VH CDR3 comprising the aa sequence RETTTVGRYYYAMDY (SEQ ID NO:369).

In some cases, an anti-CD19 antibody (or antigen binding fragments thereof) is a scFv. For example, in some cases, an anti-CD19 scFv comprises an aa sequence having at least 90%, at least 95%, at least 98%, at least 99%, or 100%, aa sequence identity to the following aa sequence: DIQLTQSPAS LAVSLGQRATISCKASQSVDYDGDSYLNWYQQIPGQPPKLLIYDASNLVSGIPPRFSGSGSGTDF TLNIHPVEKVDAATYHCQQSTEDPWTFGGGTKLEIKGGGGSGGGGSGGGGSQVQLQQSGAEL VRPGSSVKISCKASGYAFSSYWMNWVKQRPGQGLEWIGQIWPGDGDTNYNGKFKGKATLTAD ESSSTAYMQLSSLASEDSAVYFCARRETTTVGRYYYAMDYWGQGTTVTVS (SEQ ID NO:370).

(c) Anti-Mesothelin

Anti-mesothelin antibodies (or antigen binding fragments thereof) useful as a CTP are known in the art; and the VH and VL, or the VH and VL CDRs, of any anti-mesothelin antibody can be used in a MAPP of the present disclosure. See, e.g., U.S. 2019/0000944; WO 2009/045957; WO 2014/031476; U.S. Pat. No. 8,460,660; US 2013/0066055; and WO 2009/068204.

In some cases, an anti-mesothelin antibody (or antigen binding fragments thereof) comprises: a) a light chain comprising an aa sequence having at least 90%, at least 95%, at least 98%, at least 99%, or 100%, aa sequence identity to the following aa sequence: DIALTQPASVSGSPGQSITISCTGTSSDIGGYNSVSWYQQHPGKAPKLMIYGVNN RPSGVSNRFSGSKSGNTASLTISGLQAEDEADYYCSSYDIESATPVFGGGTKLTVLGQPKAAPSV TLFPPSSEELQANKATLVCLISDFYPGAVTVAWKGDSSPVKAGVETTTPSKQSNNKYAASSYLS LTPEQWKSHRSYSCQVTHEGSTVEKTVAPTESS (SEQ ID NO:371); and b) a heavy chain comprising an aa sequence having at least 90%, at least 95%, at least 98%, at least 99%, or 100%, aa sequence identity to the following aa sequence: QVELVQSGAEVKKPGESLKISCKGSGYS FTSYWIGWVRQAPGKGLEWMGIIDPGDSRTRYSPSFQGQVTISADKSISTAYLQWSSLKASDTA MYYCARGQLYGGTYMDGWGQGTLVTVSSASTKGPSVFPLAPSSKSTSGGTAALGCLVKDYFP EPVTVSWNSGALTSGVHTFPAVLQSSGLYSLSSVVTVPSSSLGTQTYICNVNHKPSNTKVDKKV EPKSCDKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTISKAKG QPREPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPGK (SEQ ID NO:372).

In some cases, an anti-mesothelin antibody (or antigen binding fragments thereof) comprises a VL present in the light chain aa sequence provided above; and a VH present in the heavy chain aa sequence provided above. For example, an anti-mesothelin antibody can comprise: a) a VL comprising an aa sequence having at least 90%, at least 95%, at least 98%, at least 99%, or 100%, aa sequence identity to the aa sequence: DIALTQPASVSGSPGQSITISCTGTSSDIGGYNSVSWYQQHPGKAPK LMIYGVNNR PSGVSNRFSGSKSGNTASLTISGLQAEDEADYYCSSYDIESATPVFGGGTK (SEQ ID NO:373); and b) a VH comprising an aa sequence having at least 90%, at least 95%, at least 98%, at least 99%, or 100%, aa sequence identity to the aa sequence: QVELVQSGAEVKKPGESLKISCKGS GYSFTSYWIGWVRQAPGKGLEWMGIIDPGDSRTRYSPSFQGQVTISADKSISTAYLQWSSLKAS DTAMYYCARGQLYGGTYMDGWGQGTLVTVSS (SEQ ID NO:374).

In some cases, an anti-mesothelin antibody (or antigen binding fragments thereof) comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat (see, e.g., Kabat 1991). In some cases, the VH and VL CDRs are as defined by Chothia (see, Chothia 1987). For example, an anti-mesothelin antibody (or antigen binding fragments thereof) can comprise a VL CDR1 having the aa sequence TGTSSDIGGYNSVS (SEQ ID NO:375); a VL CDR2 having the aa sequence LMIYGVNNRPS (SEQ ID NO:376); a VL CDR3 having the aa sequence SSYDIESATP (SEQ ID NO:377); a VH CDR1 having the aa sequence GYSFTSYWIG (SEQ ID NO:378); a VH CDR2 having the aa sequence WMGIIDPGDSRTRYSP (SEQ ID NO:379); and a VH CDR3 having the aa sequence GQLYGGTYMDG (SEQ ID NO:380).

An anti-mesothelin antibody can be a scFv. As one non-limiting example, an antimesothelin scFv can comprise the following aa sequence: QVQLQQSGAEVKKPGASV KVSCKASGYTFTGYYMHWVRQAPGQGLEWMGRINPNSGGTNYAQKFQGRVTMTRDTSISTA YMELSRLRSEDTAVYYCARGRYYGMDVWGQGTMVTVSSGGGGSGGGGSGGGGSGGGGSEIV LTQSPATLSLSPGERATISCRASQSVSSNFAWYQQRPGQAPRLLIYDASNRATGIPPRFSGSGSG TDFTLTISSLEPEDFAAYYCHQRSNWLYTFGQGTKVDIK (SEQ ID NO:381), where VH CDR1, CDR2, and CDR3 are underlined; and VL CDR1, CDR2, and CDR3 are bolded and underlined.

As one non-limiting example, an anti-mesothelin scFv can comprise the following aa sequence: QVQLVQSGAEVKKPGASVKVSCKASGYTFTGYYMHWVRQAPGQGLEWMGWINPN SGGTNYAQKFQGRVTMTRDTSISTAYMELSRLRSDDTAVYYCARDLRRTVVTPRAYYGMDV WGQGTTVTVSSGGGGSGGGGSGGGGSGGGGSDIQLTQSPSTLSASVGDRVTITCQASODISNSL NWYQQKAGKAPKWYDASTLETGVPSRFSGSGSGTDFSFTISSLQPEDIATYYCQQHDNLPLT FGQGTKVEIK (SEQ ID NO:382), where VH CDR1, CDR2, and CDR3 are underlined; and VL CDR1, CDR2, and CDR3 are bolded and underlined.

In some cases, an anti-mesothelin antibody suitable for inclusion in a MAPP as a targeting sequence comprises: a) VL CDR1, VL CDR2, and VL CDR3 present in a light chain variable region (VL) comprising the following amino acid sequence: EIVLTQSPGTLSLSPGERATLSCRASQSVSSSY LAWYQQKPGQAPRLLIYGASSRATGIPDRFSGSGSGTDFTLTISRLEPEDFAVYYCQQYGSSPIFT FGPGTKVDIK (SEQ ID NO:452); and b) VH CDR1, CDR2, and CDR3 present in a heavy chain variable region (VH) comprising the following amino acid sequence: QMQLVESGGGVVQPGRSLRLS CTASGFTFSNNGMHWVRQAPGKGLEWVAVIWFDGMNKFYVDSVKGRFTISRDNSKNTLYLE MNSLRAEDTAIYYCAREGDGSGIYYYYGMDVWGQGTTVTVSS (SEQ ID NO:453). In some cases, the VH and VL CDRs are as defined by Kabat (see, e.g., the CDR Table, above; and Kabat 1991). In some cases, the VH and VL CDRs are as defined by Chothia (see, e.g., the CDR Table, above; and Chothia 1987).

In some cases, an anti-mesothelin antibody suitable for inclusion in a MAPP as a targeting sequence comprises: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRATG IPDRFSGSGSGTDFTLTISRLEPEDFAVYYCQQYGSSPIFTFGPGTKVDIK (SEQ ID NO:452); and b) a VH region comprising an amino acid sequence having at least 90%, at least 95%, at least 98%, at least 99%, or 100%, amino acid sequence identity to the following amino acid sequence: QMQLVESG GGVVQPGRSLRLSCTASGFTFSNNGMHWVRQAPGKGLEWVAVIWFDGMNKFYVDSVKGRFTI SRDNSKNTLYLEMNSLRAEDTAIYYCAREGDGSGIYYYYGMDVWGQGTTVTVSS (SEQ ID NO:453).

In some cases, an anti-mesothelin antibody suitable for inclusion in a MAPP as a targeting sequence is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFT FSNNGMHWVRQAPGKGLEWVAVIWFDGMNKFYVDSVKGRFTISRDNSKNTLYLEMNSLRAE DTAIYYCAREGDGSGIYYYYGMDVWGQGTTVTVSS (SEQ ID NO:453); b) a peptide linker; and c) a VL region comprising an amino acid sequence having at least 90%, at least 95%, at least 98%, at least 99%, or 100%, amino acid sequence identity to the following amino acid sequence: EIVLTQSPGT LSLSPGERATLSCRASQSVSSSYLAWYQQKPGQAPRLLIYGASSRATGIPDRFSGSGSGTDFTLTI SRLEPEDFAVYYCQQYGSSPIFTFGPGTKVDIK (SEQ ID NO:452). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP as a targeting sequence is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSS SYLAWYQQKPGQAPRLLIYGASSRATGIPDRFSGSGSGTDFTLTISRLEPEDFAVYYCQQYGSSPI FTFGPGTKVDIK (SEQ ID NO:452); b) a peptide linker; and c) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTFSNNGM HWVRQAPGKGLEWVAVIWFDGMNKFYVDSVKGRFTISRDNSKNTLYLEMNSLRAEDTAIYYC AREGDGSGIYYYYGMDVWGQGTTVTVSS (SEQ ID NO:453). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP as a targeting sequence comprises: a) VL CDR1, VL CDR2, and VL CDR3 present in a light chain variable region (VL) comprising the following amino acid sequence: DIELTQSPAIMSASPGEKVTMTCSASSSVSYM HWYQQKSGTSPKRWIYDTSKLASGVPGRFSGSGSGNSYSLTISSVEAEDDATYYCQQWSKHPL TFGSGTKVEIK (SEQ ID NO:455); and b) VH CDR1, CDR2, and CDR3 present in a heavy chain variable region (VH) comprising the following amino acid sequence: QVQLQQSGPELEKPGASVKISC KASGYSFTGYTMNWVKQSHGKSLEWIGLITPYNGASSYNQKFRGKATLTVDKSSSTAYMDLLS LTSEDSAVYFCARGGYDGRGFDYWGSGTPVTVSS (SEQ ID NO:456). In some cases, the VH and VL CDRs are as defined by Kabat (see, e.g., the CDR Table, above; and Kabat 1991). In some cases, the VH and VL CDRs are as defined by Chothia (see, e.g., the CDR Table, above; and Chothia 1987).

In some cases, an anti-mesothelin antibody suitable for inclusion in a MAPP as a targeting sequence comprises: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGV PGRFSGSGSGNSYSLTISSVEAEDDATYYCQQWSKHPLTFGSGTKVEIK (SEQ ID NO:455); and b) a VH region comprising an amino acid sequence having at least 90%, at least 95%, at least 98%, at least 99%, or 100%, amino acid sequence identity to the following amino acid sequence: QVQLQQSGP ELEKPGASVKISCKASGYSFTGYTMNWVKQSHGKSLEWIGLITPYNGASSYNQKFRGKATLTV DKSSSTAYMDLLSLTSEDSAVYFCARGGYDGRGFDYWGSGTPVTVSS (SEQ ID NO:456).

In some cases, an anti-mesothelin antibody suitable for inclusion in a MAPP as a targeting sequence is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVS YMHWYQQKSGTSPKRWIYDTSKLASGVPGRFSGSGSGNSYSLTISSVEAEDDATYYCQQWSKH PLTFGSGTKVEIK (SEQ ID NO:455); b) a peptide linker; and c) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSF TGYTMNWVKQSHGKSLEWIGLITPYNGASSYNQKFRGKATLTVDKSSSTAYMDLLSLTSEDSA VYFCARGGYDGRGFDYWGSGTPVTVSS (SEQ ID NO:456). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP as a targeting sequence is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSF TGYTMNWVKQSHGKSLEWIGLITPYNGASSYNQKFRGKATLTVDKSSSTAYMDLLSLTSEDSA VYFCARGGYDGRGFDYWGSGTPVTVSS (SEQ ID NO:456); b) a peptide linker; and c) a VL region comprising an amino acid sequence having at least 90%, at least 95%, at least 98%, at least 99%, or 100%, amino acid sequence identity to the following amino acid sequence: DIELTQSPAIMSASPGEK VTMTCSASSSVSYMHWYQQKSGTSPKRWIYDTSKLASGVPGRFSGSGSGNSYSLTISSVEAEDD ATYYCQQWSKHPLTFGSGTKVEIK (SEQ ID NO:455). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP as a targeting sequence comprises: a) VL CDR1, VL CDR2, and VL CDR3 present in a light chain variable region (VL) comprising the following amino acid sequence: DIALTQPASVSGSPGQSITISCTGTSSDIGGYN SVSWYQQHPGKAPKLMIYGVNNRPSGVSNRFSGSKSGNTASLTISGLQAEDEADYYCSSYDIES ATPVFGGGTKLTVLG (SEQ ID NO:457); and b) VH CDR1, CDR2, and CDR3 present in a heavy chain variable region (VH) comprising the following amino acid sequence: QVELVQSGAEVKKPGES LKISCKGSGYSFTSYWIGWVRQAPGKGLEWMGIIDPGDSRTRYSPSFQGQVTISADKSISTAYLQ WSSLKASDTAMYYCARGQLYGGTYMDGWGQGTLVTVSS (SEQ ID NO:458). In some cases, the VH and VL CDRs are as defined by Kabat (see, e.g., the CDR Table, above; and Kabat 1991). In some cases, the VH and VL CDRs are as defined by Chothia (see, e.g., the CDR Table, above; and Chothia 1987).

In some cases, an anti-mesothelin antibody suitable for inclusion in a MAPP as a targeting sequence comprises: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNRP SGVSNRFSGSKSGNTASLTISGLQAEDEADYYCSSYDIESATPVFGGGTKLTVLG (SEQ ID NO:457); and b) a VH region comprising an amino acid sequence having at least 90%, at least 95%, at least 98%, at least 99%, or 100%, amino acid sequence identity to the following amino acid sequence:

(SEQ ID NO: 458)
QVELVQSGAEVKKPGESLKISCKGSGYSFTSYWIGWVRQAPGKGLEWM
GIIDPGDSRTRYSPSFQGQVTISADKSISTAYLQWSSLKASDTAMYYC
ARGQLYGGTYMDGWGQGTLVTVSS.

In some cases, an anti-mesothelin antibody suitable for inclusion in a MAPP as a targeting sequence is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIG GYNSVSWYQQHPGKAPKLMIYGVNNRPSGVSNRFSGSKSGNTASLTISGLQAEDEADYYCSSY DIESATPVFGGGTKLTVLG (SEQ ID NO:457); b) a peptide linker; and c) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYS FTSYWIGWVRQAPGKGLEWMGIIDPGDSRTRYSPSFQGQVTISADKSISTAYLQWSSLKASDTA MYYCARGQLYGGTYMDGWGQGTLVTVSS (SEQ ID NO:458). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP as a targeting sequence is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYSF TSYWIGWVRQAPGKGLEWMGIIDPGDSRTRYSPSFQGQVTISADKSISTAYLQWSSLKASDTAM YYCARGQLYGGTYMDGWGQGTLVTVSS (SEQ ID NO:458); b) a peptide linker; and c) a VL region comprising an amino acid sequence having at least 90%, at least 95%, at least 98%, at least 99%, or 100%, amino acid sequence identity to the following amino acid sequence: DIALTQPASVSGSPGQ SITISCTGTSSDIGGYNSVSWYQQHPGKAPKLMIYGVNNRPSGVSNRFSGSKSGNTASLTISGLQ AEDEADYYCSSYDIESATPVFGGGTKLTVLG (SEQ ID NO:457). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP comprises: a) VL CDR1, VL CDR2, and VL CDR3 present in a light chain variable region (VL) comprising the following amino acid sequence: DIQMTQSPSSLSASVGDRVTITCSASSSVSYMHWYQQKSGKAPKLLIYDT SKLASGVPSRFSGSGSGTDFTLTISSLQPEDFATYYCQQWSKHPLTFGQGTKLEIK (SEQ ID NO:459); and b) VH CDR1, CDR2, and CDR3 present in a heavy chain variable region (VH) comprising the following amino acid sequence: QVQLVQSGAEVKKPGASVKVSCKASGYSFTGYT MNWVRQAPGQGLEWMGLITPYNGASSYNQKFRGKATMTVDTSTSTVYMELSSLRSEDTAVY YCARGGYDGRGFDYWGQGTLVTVSS (SEQ ID NO:460). In some cases, the VH and VL CDRs are as defined by Kabat (see, e.g., the CDR Table, above; and Kabat 1991). In some cases, the VH and VL CDRs are as defined by Chothia (see, e.g., the CDR Table, above; and Chothia 1987).

In some cases, an anti-mesothelin antibody suitable for inclusion in a MAPP comprises: a) a VL region comprising an amino acid sequence having at least 90%, at least 95%, at least 98%, at least 99%, or 100%, amino acid sequence identity to the following amino acid sequence: DIQMTQSPSSLSASV GDRVTITCSASSSVSYMHWYQQKSGKAPKLLIYDTSKLASGVPSRFSGSGSGTDFTLTIS SLQPEDFATYYCQQWSKHPLTFGQGTKLEIK (SEQ ID NO:459); and b) a VH region comprising an amino acid sequence having at least 90%, at least 95%, at least 98%, at least 99%, or 100%, amino acid sequence identity to the following amino acid sequence: QVQLVQSGAEVKKPG ASVKVSCKASGYSFTGYTMNWVRQAPGQGLEWMGLITPYNGASSYNQKFRGKATMT VDTSTSTVYMELSSLRSEDTAVYYCARGGYDGRGFDYWGQGTLVTVSS (SEQ ID NO:460).

In some cases, an anti-mesothelin antibody suitable for inclusion in a MAPP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQK SGKAPKLLIYDTSKLASGVPSRFSGSGSGTDFTLTISSLQPEDFATYYCQQWSKHPLTFG QGTKLEIK (SEQ ID NO:459); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFT GYTMNWVRQAPGQGLEWMGLITPYNGASSYNQKFRGKATMTVDTSTSTVYMELSSL RSEDTAVYYCARGGYDGRGFDYWGQGTLVTVSS (SEQ ID NO:460). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-mesothelin antibody suitable for inclusion in a MAPP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFTGYTMNWV RQAPGQGLEWMGLITPYNGASSYNQKFRGKATMTVDTSTSTVYMELSSLRSEDTAVYYCARG GYDGRGFDYWGQGTLVTVSS (SEQ ID NO:460); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCSASS SVSYMHWYQQKSGKAPKLLIYDTSKLASGVPSRFSGSGSGTDFTLTISSLQPEDFATYYCQQWS KHPLTFGQGTKLEIK (SEQ ID NO:459). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

(d) Anti-TROP-2

Trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene.

In some cases, the CTP of a MAPP is an anti-TROP-2 scFv or an anti-TROP-2 nanobody comprising VH and VL CDRs present in any one of the amino acid sequences set forth in FIG. 23A-23D. In some cases, the CTP is an anti-TROP-2 scFv comprising an amino acid sequence as set forth in any one of FIG. 23A-23D.

Anti-TROP-2 antibodies are known in the art; and the VH and VL, or the VH and VL CDRs, of any anti-TROP-2 antibody can be used in a MAPP of the present disclosure as a targeting sequence. See, e.g., U.S. Pat. No. 7,238,785). In some cases, an anti-TROP-2 antibody comprises: i) light chain CDR sequences CDR1 (KASQDVSIAVA; SEQ ID NO:461); CDR2 (SASYRYT; SEQ ID NO:462); and CDR3 (QQHYITPLT; SEQ ID NO:463); and ii) heavy chain CDR sequences CDR1 (NYGMN; SEQ ID NO:464); CDR2 (WINTYTGEPTYTDDFKG; SEQ ID NO:465); and CDR3 (GGFGSSYWYFDV; SEQ ID NO:466).

In some cases, an anti-TROP-2 antibody comprises: i) heavy chain CDR sequences CDR1 (TAGMQ; SEQ ID NO:467); CDR2 (WINTHSGVPKYAEDFKG (SEQ ID NO:468); and CDR3 (SGFGSSYWYFDV; SEQ ID NO:469); and ii) light chain CDR sequences CDR1 (KASQDVSTAVA; SEQ ID NO:470); CDR2 (SASYRYT; SEQ ID NO:462); and CDR3 (QQHYITPLT; SEQ ID NO:463).

In some cases, an anti-TROP2 antibody suitable for inclusion in a MAPP comprises: a) VL CDR1, VL CDR2, and VL CDR3 present in a light chain variable region (VL) comprising the following amino acid sequence: DIQLTQSPSSLSASVGDRVSITCKASQDVSIAVAWYQQKPGKAPKLLIYSA SYRYTGVPDRFSGSGSGTDFTLTISSLQPEDFAVYYCQQHYITPLTFGAGTKVEIK (SEQ ID NO:471); and b) VH CDR1, CDR2, and CDR3 present in a heavy chain variable region (VH) comprising the following amino acid sequence: QVQLQQSGSELKKPGASVKVSCKASGYTFTNYG MNWVKQAPGQGLKWMGWINTYTGEPTYTDDFKGRFAFSLDTSVSTAYLQISSLKADDTAVYF CARGGFGSSYWYFDVWGQGSLVTVSS (SEQ ID NO:472). In some cases, the V_{H} and V_{L} CDRs are as defined by Kabat (see, e.g., the CDR Table, above; and Kabat 1991). In some cases, the V_{H} and V_{L} CDRs are as defined by Chothia (see, e.g., the CDR Table, above; and Chothia 1987).

In some cases, an anti-TROP-2 antibody suitable for inclusion in a MAPP comprises: a) a VL region comprising an amino acid sequence having at least 90%, at least 95%, at least 98%, at least 99%, or 100%, amino acid sequence identity to the following amino acid sequence: DIQLTQSPSSLSASVGD RVSITCKASQDVSIAVAWYQQKPGKAPKLLIYSASYRYTGVPDRFSGSGSGTDFTLTISSLQPED FAVYYCQQHYITPLTFGAGTKVEIK (SEQ ID NO:471); and b) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYT FTNYGMNWVKQAPGQGLKWMGWINTYTGEPTYTDDFKGRFAFSLDTSVSTAYLQISSLKADD TAVYFCARGGFGSSYWYFDVWGQGSLVTVSS (SEQ ID NO:472).

In some cases, an anti-TROP-2 antibody suitable for inclusion in a MAPP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAP KLLIYSASYRYTGVPDRFSGSGSGTDFTLTISSLQPEDFAVYYCQQHYITPLTFGAGTKVEIK (SEQ ID NO:471); b) a peptide linker; and c) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQAP GQGLKWMGWINTYTGEPTYTDDFKGRFAFSLDTSVSTAYLQISSLKADDTAVYFCARGGFGSS YWYFDVWGQGSLVTVSS (SEQ ID NO:472). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-TROP-2 antibody suitable for inclusion in a MAPP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQ APGQGLKWMGWINTYTGEPTYTDDFKGRFAFSLDTSVSTAYLQISSLKADDTAVYFCARGGFG SSYWYFDVWGQGSLVTVSS (SEQ ID NO:472); b) a peptide linker; and c) a VL region comprising an amino acid sequence having at least 90%, at least 95%, at least 98%, at least 99%, or 100%, amino acid sequence identity to the following amino acid sequence: DIQLTQSPSSLSASVGDRVSITCKAS QDVSIAVAWYQQKPGKAPKLLIYSASYRYTGVPDRFSGSGSGTDFTLTISSLQPEDFAVYYCQQ HYITPLTFGAGTKVEIK (SEQ ID NO:471). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-TROP2 antibody suitable for inclusion in MAPP comprises: a) VL CDR1, VL CDR2, and VL CDR3 present in a light chain variable region (VL) comprising the following amino acid sequence: DIQMTQSPSSLSASVGDRVTITCKASQDVSTAVAWYQQKPGKAPKLL IYSASYRYTGVPSRFSGSGSGTDFTLTISSLQPEDFAVYYCQQHYITPLTFGQGTKLEIK (SEQ ID NO:473); and b) VH CDR1, CDR2, and CDR3 present in a heavy chain variable region (VH) comprising the following amino acid sequence: QVQLVQSGAEVKKPGASVKVSCKASGYTFTTAG MQWVRQAPGQGLEWMGWINTHSGVPKYAEDFKGRVTISADTSTSTAYLQLSSLKSEDTAVYY CARSGFGSSYWYFDVWGQGTLVTVSS (SEQ ID NO:474). In some cases, the V_{H} and V_{L} CDRs are as defined by Kabat (see, e.g., the CDR Table, above; and Kabat 1991). In some cases, the V_{H} and V_{L} CDRs are as defined by Chothia (see, e.g., the CDR Table, above; and Chothia 1987).

In some cases, an anti-TROP-2 antibody suitable for inclusion in a MAPP comprises: a) a VL region comprising an amino acid sequence having at least 90%, at least 95%, at least 98%, at least 99%, or 100%, amino acid sequence identity to the following amino acid sequence: DIQMTQSPSSLSAS VGDRVTITCKASQDVSTAVAWYQQKPGKAPKLLIYSASYRYTGVPSRFSGSGSGTDFTLTISSL QPEDFAVYYCQQHYITPLTFGQGTKLEIK (SEQ ID NO:473); and b) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGY TFTTAGMQWVRQAPGQGLEWMGWINTHSGVPKYAEDFKGRVTISADTSTSTAYLQLSSLKSE DTAVYYCARSGFGSSYWYFDVWGQGTLVTVSS (SEQ ID NO:474).

In some cases, an anti-TROP-2 antibody suitable for inclusion in a MAPP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGK APKLLIYSASYRYTGVPSRFSGSGSGTDFTLTISSLQPEDFAVYYCQQHYITPLTFGQGTKLEIK (SEQ ID NO:473); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVRQA PGQGLEWMGWINTHSGVPKYAEDFKGRVTISADTSTSTAYLQLSSLKSEDTAVYYCARSGFGS SYWYFDVWGQGTLVTVSS (SEQ ID NO:474). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

In some cases, an anti-TROP-2 antibody suitable for inclusion in a MAPP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVRQ APGQGLEWMGWINTHSGVPKYAEDFKGRVTISADTSTSTAYLQLSSLKSEDTAVYYCARSGFG SSYWYFDVWGQGTLVTVSS (SEQ ID NO:474); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQ DVSTAVAWYQQKPGKAPKLLIYSASYRYTGVPSRFSGSGSGTDFTLTISSLQPEDFAVYYCQQH YITPLTFGQGTKLEIK (SEQ ID NO:473). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:454) and has a length of 15 amino acids.

(e) Anti-BCMA

Anti-BCMA (B-cell maturation antigen) antibodies (or antigen binding fragments thereof) are known in the art; and the VH and VL, or the VH and VL CDRs, of any anti-BCMA antibody can be used in a MAPP of the present disclosure. See, e.g., WO 2014/089335; and US 2019/0153061.

In some cases, an anti-BCMA antibody (or antigen binding fragments thereof) comprises: a) a light chain comprising an aa sequence having at least 90%, at least 95%, at least 98%, at least 99%, or 100%, aa sequence identity to the following aa sequence: QSVLTQPPSASGTPGQRVTISCSGSSSNIGSNTVNWYQQLPGTAPKLLIFNYHQRPSGVPDRFSGS KSGSSASLAISGLQSEDEADYYCAAWDDSLNGWVFGGGTKLTVLGQPKAAPSVTLFPPSSEELQ ANKATLVCLISDFYPGAVTVAWKADSSPVKAGVETTTPDSKQSNNKYAASSYLSLTPEQWKSH RSYSCQVTHEGSTVEKTVAPTECS (SEQ ID NO:383); and b) a heavy chain comprising an aa sequence having at least 90%, at least 95%, at least 98%, at least 99%, or 100%, aa sequence identity to the following aa sequence: EVQLVESGGGLVKPGGSLRLSCAASGFTFGDYALSWFRQAPGKGLE WVGVSRSKAYGGTTDYAASVKGRFTISRDDSKSTAYLQMNSLKTEDTAVYYCASSGYSSGWT PFDYWGQGTLVTVSSASTKGPSVFPLAPSSKSTSGGTAALGCLVKDYFPEPVTVSWNSGALTSG VHTFPAVLQSSGLYSLSSVVTVPSSSLGTQTYICNVNHKPSNTKVDKKVEPKSCDKTHTCPPCPA PELLGGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLPPSREE MTKNQVSLTCLVKGFYPSDIAVEWESNGQPENNYKTTPPVLDSDGSFFLYSKLTVDKSRWQQG NVFSCSVMHEALHNHYTQKSLSLSPGK (SEQ ID NO:384).

In some cases, an anti-BCMA (or antigen binding fragments thereof) comprises a VL present in the light chain aa sequence provided above; and a VH present in the heavy chain aa sequence provided above. For example, an anti-BCMA antibody can comprise: a) a VL comprising an aa sequence having at least 90%, at least 95%, at least 98%, at least 99%, or 100%, aa sequence identity to the aa sequence:
QSVLTQPPSASGTPGQRVTISCSGSSSNIGSNTVNWYQQLPGTAPKLLIFNYHQRPSGVPDRFSGS KSGSSASLAISGLQSEDEADYYCAAWDDSLNGWVFGGGTKLTVL G (SEQ ID NO:385); and b) a VH comprising an aa sequence having at least 90%, at least 95%, at least 98%, at least 99%, or 100%, aa sequence identity to the aa sequence: EVQLVESGGGLVKPGGSLRLSCAASGFTFGDYALSWFRQA PGKGLEWVGVSRSKAYGGTTDYAASVKGRFTISRDDSKSTAYLQMNSLKTEDTAVYYCASSG YSSGWTPFDYWGQGTLVTVSSASTKGPSV (SEQ ID NO:386).

In some cases, an anti-BCMA antibody (or antigen binding fragments thereof) comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat (see, e.g., Kabat 1991). In some cases, the VH and VL CDRs are as defined by Chothia (see, e.g., Chothia 1987).

For example, an anti-BCMA antibody (or antigen binding fragments thereof) can comprise a VL CDR1 having the aa sequence SSNIGSNT (SEQ ID NO:387), a VL CDR2 having the aa sequence NYH, a VL CDR3 having the aa sequence AAWDDSLNGWV (SEQ ID NO:388)), a VH CDR1 having the aa sequence GFTFGDYA (SEQ ID NO:389), a VH CDR2 having the aa sequence SRSKAYGGTT (SEQ ID NO:390), and a VH CDR3 having the aa sequence ASSGYSSGWTPFDY (SEQ ID NO:391).

An anti-BCMA antibody can be a scFv. As one non-limiting example, an anti-BCMA scFv can comprise the following aa sequence: QVQLVQSGAEVKKPGSSVKVSCKASGGTFSNYWMHW VRQAPGQGLEWMGATYRGHSDTYYNQKFKGRVTITADKSTSTAYMELSSLRSEDTAVYYCAR GAIYNGYDVLDNWGQGTLVTVSSGGGGSGGGGSGGGGSGGGGSDIQMTQSPSSLSASVGDRV TITCSASQDISNYLNWYQQKPGKAPKLLIYYTSNLHSGVPSRFSGSGSGTDFTLTISSLQPEDFAT YYCQQYRKLPWTFGQGTKLEIKR (SEQ ID NO:392), or the sequence: QVQLVQSGAEVKKPGSS VKVSCKASGGTFSNYWMHWVRQAPGQGLEWMGATYRGHSDTYYNQKFKGRVTITADKSTST AYMELSSLRSEDTAVYYCARGAIYNGYDVLDNWGQGTLVTVSSGGGGSGGGGSGGGGSGGG GSDIQMTQSPSSLSASVGDRVTITCSASQDISNYLNWYQQKPGKAPKLLIYYTSNLHSGVPSRFS GSGSGTDFTLTISSLQPEDFATYYCQQYRKLPWTFGQGTKLEIKR (SEQ ID NO:480).

As another example, an anti-BCMA scFv can comprise the following aa sequence:

(SEQ ID NO: 393)
DIQMTQSPSSLSASVGDRVTITCSASQDISNYLNWYQQKPGKAPKLLI
YYTSNLHSGVPSRFSGSGSGTDFTLTISSLQPEDFATYYCQQYRKLPW
TFGQGTKLEIKRGGGGSGGGGSGGGGSGGGGSQVQLVQSGAEVKKPGS
SVKVSCKASGGTFSNYWMHWVRQAPGQGLEWMGATYRGHSDTYYNQKF
KGRVTITADKSTSTAYMELSSLRSEDTAVYYCARGAIYNGYDVLDNWG
QGTLVTVSS.

In some cases, an anti-BCMA antibody can comprise a VL CDR1 having the amino acid sequence SASQDISNYLN (SEQ ID NO:481); a VL CDR2 having the amino acid sequence YTSNLHS (SEQ ID NO:482); a VL CDR3 having the amino acid sequence QQYRKLPWT (SEQ ID NO:483); a VH CDR1 having the amino acid sequence NYWMH (SEQ ID NO:484); a VH CDR2 having the amino acid sequence ATYRGHSDTYYNQKFKG (SEQ ID NO:485); and a VH CDR3 having the amino acid sequence GAIYNGYDVLDN (SEQ ID NO:486).

In some cases, an anti-BCMA antibody comprises: a) a light chain comprising an amino acid sequence having at least 90%, at least 95%, at least 98%, at least 99%, or 100%, amino acid sequence identity to the following amino acid sequence: DIQMTQSPSSLSASVGDRVTITCSASQDISNYLNWY QQKPGKAPKLLIYYTSNLHSGVPSRFSGSGSGTDFTLTISSLQPEDFATYYCQQYRKLPWTFGQG TKLEIKR (SEQ ID NO:487).

In some cases, an anti-BCMA antibody comprises: a) a heavy chain comprising an amino acid sequence having at least 90%, at least 95%, at least 98%, at least 99%, or 100%, amino acid sequence identity to the following amino acid sequence: QVQLVQSGAEVKKPGSSVKVSCKASGGT FSNYWMHWVRQAPGQGLEWMGATYRGHSDTYYNQKFKGRVTITADKSTSTAYMELSSLRSE DTAVYYCARGAIYDGYDVLDNWGQGTLVTVSS (SEQ ID NO:488).

In some cases, an anti-BCMA antibody (e.g., an antibody referred to in the literature as belantamab) comprises a light chain comprising the amino acid sequence: DIQMTQSPSSLSASVGDR VTITCSASQDISNYLNWYQQKPGKAPKLLIYYTSNLHSGVPSRFSGSGSGTDFTLTISSLQPEDFA TYYCQQYRKLPWTFGQGTKLEIKR (SEQ ID NO:487); and a heavy chain comprising the amino acid sequence: QVQLVQSGAEVKKPGSSVKVSCKASGGTFSNYWMHWVRQAPGQGLEWMG ATYRGHSDTYYNQKFKGRVTITADKSTSTAYMELSSLRSEDTAVYYCARGAIYDGYDVLDNW GQGTLVTVSS (SEQ ID NO:488).

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

(f)Anti-MUC1

In some cases, a targeting sequence present in a MAPP of the present disclosure is an antibody specific for MUC1. For example, a targeting sequence can be specific for a MUC1 polypeptide present on a cancer cell. In some cases, the targeting sequence is specific for the cleaved form of MUC1; see, e.g., Fessler et al. (2009) Breast Cancer Res. Treat. 118:113. In some cases, the targeting sequence is an antibody specific for a glycosylated MUC1 peptide; see, e.g., Naito et al. (2017) ACS Omega 2:7493; and U.S. Pat. No. 10,017,580.

As one non-limiting example, a targeting sequence can be a single-chain Fv specific for MUC1. See, e.g., Singh et al. (2007) Mol. Cancer Ther. 6:562; Thie et al. (2011) PLoSOne 6:e15921; Imai et al. (2004) Leukemia 18:676; Posey et al. (2016) Immunity 44:1444; EP3130607; EP3164418; WO 2002/044217; and US 2018/0112007. In some cases, a targeting sequence is a scFv specific for the MUC1 peptide VTSAPDTRPAPGSTAPPAHG (SEQ ID NO:489). In some cases, a targeting sequence is a scFv specific for the MUC1 peptide:
SNIKFRPGSVVVQLTLAFREGTINVHDVETQFNQYKTEAASRY (SEQ ID NO:490). In some cases, a targeting sequence is a scFv specific for the MUC1 peptide SVVVQLTLAFREGTINVHDVETQFNQ YKTEAASRY (SEQ ID NO:491). In some cases, a targeting sequence is a scFv specific for the MUC1 peptide LAFREGTINVHDVETQFNQY (SEQ ID NO:492). In some cases, a targeting sequence is a scFv specific for the MUC1 peptide SNIKFRPGSVVVQLTLAAFREGTIN (SEQ ID NO:493).

As an example, an anti-MUC1 antibody can comprise: a VH CDR1 having the amino acid sequence RYGMS (SEQ ID NO:494); a VH CDR2 having the amino acid sequence TISGGGTYIYYPDSVKG (SEQ ID NO:495); a VH CDR3 having the amino acid sequence DNYGRNYDYGMDY (SEQ ID NO:496); a VL CDR1 having the amino acid sequence SATSSVSYIH (SEQ ID NO:497); a VL CDR2 having the amino acid sequence STSNLAS (SEQ ID NO:498); and a VL CDR3 having the amino acid sequence QQRSSSPFT (SEQ ID NO:499). See, e.g., US PAT PUB. NO. 2018/0112007.

As another example, an anti-MUC1 antibody can comprise a VH CDR1 having the amino acid sequence GYAMS (SEQ ID NO:500); a VH CDR2 having the amino acid sequence TISSGGTYIYYPD SVKG (SEQ ID NO:501); a VH CDR3 having the amino acid sequence LGGDNYYEYFDV (SEQ ID NO:502); a VL CDR1 having the amino acid sequence RASKSVSTSGYSYMH (SEQ ID NO:503); a VL CDR2 having the amino acid sequence LASNLES (SEQ ID NO:504); and a VL CDR3 having the amino acid sequence QHSRELPFT (SEQ ID NO:505). See, e.g., US 2018/0112007.

As another example, an anti-MUC1 antibody can comprise a VH CDR1 having the amino acid sequence DYAMN (SEQ ID NO:506); a VH CDR2 having the amino acid sequence VISTFSGNINFN QKFKG (SEQ ID NO:507); a VH CDR3 having the amino acid sequence SDYYGPYFDY (SEQ ID NO:508); a VL CDR1 having the amino acid sequence RSSQTIVHSNGNTYLE (SEQ ID NO:509); a VL CDR2 having the amino acid sequence KVSNRFS (SEQ ID NO:510); and a VL CDR3 having the amino acid sequence FQGSHVPFT (SEQ ID NO:511). See, e.g., US 2018/0112007.

As another example, an anti-MUC1 antibody can comprise a VH CDR1 having the amino acid sequence GYAMS (SEQ ID NO:500); a VH CDR2 having the amino acid sequence TISSGGTYIYYPDSVKG (SEQ ID NO:512); a VH CDR3 having the amino acid sequence LGGDNYYEY (SEQ ID NO:513); a VL CDR1 having the amino acid sequence TASKSVSTSGYSYMH (SEQ ID NO:514); a VL CDR2 having the amino acid sequence LVSNLES (SEQ ID NO:515); and a VL CDR3 having the amino acid sequence QHIRELTRSE (SEQ ID NO:516). See, e.g., US 2018/0112007.

(g)Anti-MUC16

In some cases, a targeting sequence present in a MAPP of the present disclosure is an antibody specific for MUC16 (also known as CA125). See, e.g., Yin et al. (2002) Int. J. Cancer 98:737. For example, a targeting sequence can be specific for a MUC16 polypeptide present on a cancer cell. See, e.g., US 2018/0118848; and US 2018/0112008. In some cases, a MUC16-specific targeting sequence is a scFv. In some cases, a MUC16-specific targeting sequence is a nanobody.

As one example, an anti-MUC16 antibody can comprise a VH CDR1 having the amino acid sequence GFTFSNYY (SEQ ID NO:517); a VH CDR2 having the amino acid sequence ISGRGSTI (SEQ ID NO:518); a VH CDR3 having the amino acid sequence VKDRGGYSPY (SEQ ID NO:519); a VL CDR1 having the amino acid sequence QSISTY (SEQ ID NO:520); a VL CDR2 having the amino acid sequence TAS; and a VL CDR3 having the amino acid sequence QQSYSTPPIT (SEQ ID NO:521). See, e.g., US 2018/0118848.

(h)Anti-Claudin-18.2

In some cases, a targeting sequence present in a MAPP of the present disclosure is an antibody specific for claudin-18 isoform 2 (“claudin-18.2”). See, e.g., WO 2013/167259. In some cases, a claudin-18.2-specific targeting sequence is a scFv. In some cases, a claudin-18.2-specific targeting sequence is a nanobody. In some cases, a CTP present in a MAPP of the present disclosure is an antibody specific for TEDEVQSYPSKHDYV (SEQ ID NO:449) or EVQSYPSKHDYV (SEQ ID NO:450).

As one example, an anti-claudin-18.2 antibody can comprise a VH CDR1 having the amino acid sequence GYTFTDYS (SEQ ID NO:522); a VH CDR2 having the amino acid sequence INTETGVP (SEQ ID NO:523); a VH CDR3 having the amino acid sequence ARRTGFDY (SEQ ID NO:524); a VL CDR1 having the amino acid sequence KNLLHSDGITY (SEQ ID NO:525); a VL CDR2 having the amino acid sequence RVS; and a VL CDR3 having the amino acid sequence VQVLELPFT (SEQ ID NO:526).

As another example, an anti-claudin-s antibody can comprise a VH CDR1 having the amino acid sequence GFTFSSYA (SEQ ID NO:527); a VH CDR2 having the amino acid sequence ISDGGSYS (SEQ ID NO:528); a VH CDR3 having the amino acid sequence ARDSYYDNSYVRDY (SEQ ID NO:529); a VL CDR1 having the amino acid sequence QDINTF (SEQ ID NO:530); a VL CDR2 having the amino acid sequence RTN; and a VL CDR3 having the amino acid sequence LQYDEFPLT (SEQ ID NO:531).

(iii) Single-Chain T Cell Receptors

In some cases, a CTP present in a MAPP of the present disclosure is a scTCR. A CTP can be a scTCR specific for a peptide/HLA complex on the surface of a cancer cell, where the peptide can be a cancer-associated peptide (e.g., a peptide fragment of a cancer-associated antigen) Amino acid sequences of scTCRs specific for cancer-associated peptides bound to an HLA complex are known in the art. See, e.g., US 2019/0135914; US 2019/0062398; and US 2018/0371049.

A scTCR includes an alpha chain variable region (Vα) and a beta chain variable region (Vβ) covalently linked through a suitable peptide linker sequence. For example, the Vα can be covalently linked to the Vβ through a suitable peptide linker (L) sequence fused to the C-terminus of the Vα and the N-terminus of the Vβ. A scTCR can have the structure Vα-Vβ. A scTCR can have the structure Vβ-L-Vα. A scTCR can also comprise a constant domain (also referred to as constant region). In some cases, a scTCR comprises, in order from N-terminus to C-terminus: i) a TCR α chain variable domain polypeptide; ii) a peptide linker; iii) a TCR β chain variable domain polypeptide; and iv) a TCR β chain constant region extracellular domain polypeptide. In some cases, a scTCR comprises, in order from N-terminus to C-terminus: i) a TCR β chain variable domain polypeptide; ii) a peptide linker; iii) a TCR α chain variable domain polypeptide; and iv) a TCR α chain constant region extracellular domain polypeptide.

Amino acid sequences of scTCRs specific for peptide/HLA complexes, where the peptide is a cancer-associated peptide, are known in the art. See, e.g., US 2019/0135914; US 2019/0062398; US 2018/0371049; US 2019/0144563; and US 2019/0119350.

For example, a scTCR can be specific for an NY-ESO epitope such as an SLLMWITQC (SEQ ID NO:545) peptide bound to an HLA complex comprising an HLA-A*0201 heavy chain and a β2M polypeptide. As an example, such a scTCR can comprise: i) a TCR α chain variable region comprising an aa sequence having at least 90%, at least 95%, at least 98%, at least 99%, or 100%, aa sequence identity to the aa sequence: MQEVTQIPAALSVPEGENLVLNCSFTDSAIYNL QWFRQDPGKGLTSLLLIQSSQREQTSGRLNASLDKSSGRSTLYIAASQPGDSATYLCAVRPTSGG SYIPTFGRGTSLIVHPY (SEQ ID NO:394), where aa 20 can be V or A; aa 51 can be Q, P, S, T, or M; aa 52 can be S, P, F, or G, aa 53 can be S, W, H, or T; aa 94 can be P, H, or A; aa 95 can be T, L, M, A, Q, Y, E, I, F, V, N, G, S, D, or R; aa 96 can be S, L, T, Y, I, Q, V, E, A, W, R, G, H, D, or K; aa 97 can be G, D, N, V, S, T, or A; aa 98 can be G, P, H, S, T, W, or A; aa 99 can be S, T, Y, D, H, V, N, E, G, Q, K, A, I, or R; aa 100 can be Y, F, M, or D; aa 101 can be I, P, T, or M; and aa 103 can be T or A; and ii) a TCR β chain variable region comprising an aa sequence having at least 90%, at least 95%, at least 98%, at least 99%, or 100%, aa sequence identity to the aa sequence: MGVTQTPKFQVLKT GQSMTLQCAQDMNHEYMSWYRQDPGMGLRLIHYSVGAGITDQGEVPNGYNVSRSTTEDFPLR LLSAAPSQTSVYFCASSYVGNTGELFFGEGSR LTVL (SEQ ID NO:395), where aa 18 can be M or V; aa 50 can be G, V, or I; aa 52 can be G or Q; aa 53 can be I, T, or M; aa 55 can be D or R; aa 56 can be Q or R; aa 70 can be T or I; aa 94 can be Y, N, or F; aa 95 can be V or L; and aa 97 can be N, G, or D. For example, in some cases, a scTCR can comprise: i) a TCR α chain variable region comprising the aa sequence: MQEVTQIPAALSVPEGENLVLNCSFTDSAIYNLQWFRQDPGKGLTSLLLIMSHQR EQTSGRLNASLDKSSGRSTLYIAASQPGDSATYLCAVRPTSGGSYIPTFGRGTSLIV HPY (SEQ ID NO:396); and a TCR β chain variable region comprising the aa sequence: MGVTQTPKFQVLKT GQSMTLQCAQDMNHEYMSWYRQDPGMGLRLIHYSVSAGITDQGEVPNGYNVSRSTTEDFPLR LLSAAPSQTSVYFCASSYVGNTGELFFGEGSR LTVL (SEQ ID NO:397).

As another example, a scTCR can be specific for an HPV peptide epitope (e.g., an HPV peptide of the aa sequence YIIFVYIPL (HPV 16 E563-71; SEQ ID NO:398), KLPQLCTEL (HPV 16 E611-19; SEQ ID NO:399), TIHEIILECV (HPV 16 E6; SEQ ID NO:400), YMLDLQPET (HPV 16 E711-19; SEQ ID NO:401), TLGIVCPI (HPV 16 E786-93; SEQ ID NO:252), KCIDFYSRI (HPV 18 E667-75; SEQ ID NO:402), or FQQLFLNTL (HPV 18 E786-94; SEQ ID NO:403)) bound to an HLA complex comprising an HL heavy chain and a β2M polypeptide. As an example, such a scTCR can comprise: i) a TCR α chain variable region comprising an aa sequence having at least 90%, at least 95%, at least 98%, at least 99%, or 100%, aa sequence identity to the aa sequence: METLLGLLILQLQLQWVSSKQEVTQIPAALSVPEGENLVLNCSFTDSAIYNLQWFRQDPGKGLT SLLLIQSSQREQTSGRLNASLDKSSGRSTLYIAASQPGDSATYLCAVRETSGSRLTFGEGTQLTV NPD (SEQID NO:404); and ii) a TCR β chain variable region comprising an aa sequence having at least 90%, at least 95%, at least 98%, at least 99%, or 100%, aa sequence identity to the aa sequence:

(SEQ ID NO: 405)
MGIRLLCRVAFCFLAVGLVDVKVTQSSRYLVKRTGEKVFLECVQDMDH
ENMFWYRQDPGLGLRLIYFSYDVKMKEKGDIPEGYSVSREKKERFSLI
LESASTNQTSMYLCASSFWGRSTDTQYFGPGTRLTVL.

9. Payloads—Drug and Other Conjugates

A polypeptide chain of a MAPP can comprise a payload such as a therapeutic (e.g., a small molecule drug or therapeutic) a label (e.g., a fluorescent label or radio label), or other biologically active agent that is linked (e.g., covalently attached) to the polypeptide chain. For example, where a MAPP comprises an Fc polypeptide, the Fc polypeptide may comprise a covalently linked payload such as an agent that treats a cancer, infectious disease, or an autoimmune disease, potentates the action of the MAPP, or is an agent that relieves a symptom of such diseases.

A payload can be linked directly or indirectly to a polypeptide chain of a MAPP (e.g., to an Ig Fc polypeptide in the MAPP). Direct linkage can involve linkage to an aa side chain without an intervening linker. Indirect linkage can be linkage via a cross-linker, such as a bifunctional cross-linker. A payload can be linked to a MAPP by any acceptable chemical linkage including, but not limited to a thioether bond, an amide bond, a carbamate bond, a disulfide bond, or an ether bond, including those formed by reaction with a crosslinking agent.

Crosslinkers (crosslinking agents) include cleavable crosslinkers and non-cleavable cross-linkers. The cross-linkers may be homobifunctional or heterobifunctional cross-linkers. In some cases, the cross-linker is a protease-cleavable cross-linker. Suitable cross-linkers may include as moieties, for example, peptides (e.g., from 2 to 10 aas in length; e.g., 2, 3, 4, 5, 6, 7, 8, 9, or 10 aas in length), alkyl chains, poly(ethylene glycol), disulfide groups, thioether groups, acid labile groups, photolabile groups, peptidase labile groups, and esterase labile groups. Non-limiting example of suitable cross-linkers are: N-succinimidyl-[(N-maleimidopropionamido)-tetraethyleneglycol]estr (NHS-PEG4-maleimide); N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

MAPP payload conjugates may be formed by reaction of a MAPP polypeptide (e.g., an IgFc polypeptide) with a cross-linking reagent to introduce 1-10 reactive groups. The polypeptide is then reacted with the molecule to be conjugated (e.g., a thiol-containing payload drug, label or agent) to produce a MAPP-payload conjugate. For example, where a MAPP comprises an IgFc polypeptide, the conjugate can be of the form (A)-(L)-(C), where (A) is the polypeptide chain comprising the IgFc polypeptide; where (L), if present, is a cross-linker; and where (C) is a payload. (L), if present, links (A) to (C). In some cases, the MAPP includes an IgFc polypeptide that comprises one or more (e.g., 2, 3, 4, 5, or more than 5) molecules of a payload. Introducing payloads into a MAPP using an excess of cross-linking agents can result in multiple molecules of payload being incorporated into the MAPP.

Suitable payloads (e.g., drugs) include virtually any small molecule (e.g., less than 2,000 Daltons in molecular weight) approved by the U.S. Food and Drug Administration, and/or listed in the 2020 U.S. Pharmacopeia or National Formulary. In an embodiment, those drugs are less than 1,000 molecular weight. Suitable drugs include antibiotics, chemotherapeutic (antineoplastic), anti-fungal, or anti-helminth agents and the like (e.g., sulfasalazine, azathioprine, cyclophosphamide, leflunomide; methotrexate, antimalarials, D-penicillamine, cyclosporine). Suitable chemotherapeutics may be alkylating agents, cytoskeletal disruptors (taxanes), epothilone, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids. Suitable drugs also include non-steroidal anti-inflammatory drugs and glucocorticoids, and the like.

D. Nucleic Acids

The present disclosure provides a nucleic acid comprising a nucleotide sequence encoding one or more polypeptides of a MAPP of the present disclosure. In some cases, the nucleic acid is a recombinant expression vector; thus, the present disclosure provides a recombinant expression vector comprising a nucleotide sequence encoding a MAPP of the present disclosure.
1. Nucleic Acids Encoding a MAPP or MAPP Forming a Higher Order Complex, such as a Duplex MAPP, that Comprises at Least One Dimerization Sequence and a Multimerization Sequence

The present disclosure provides nucleic acids comprising a nucleotide sequence encoding a MAPP having a framework polypeptide that comprises at least one dimerization sequence and at least one multimerization sequence that permits two molecules of the framework polypeptide to form dimers or higher order complexes. The nucleic acids may additionally comprise a nucleotide sequence encoding a dimerization peptide. Where the MAPP comprises a presenting sequence the nucleic acids encoding either or both of the framework polypeptide and/or dimerization peptide may include a sequence encoding a presenting sequence. Where the MAPP comprises a presenting complex, the nucleic acids encoding either or both of the framework polypeptide and/or dimerization peptide may further comprise sequences encoding a presenting complex 1^{st} sequence and/or a presenting complex 2^{nd} sequence. Where desired, the nucleic acid sequences encoding a MAPP may also exclude a sequence encoding a peptide epitope. The nucleotide sequence(s) comprising any of the MAPP polypeptides can be operably linked to a transcription control element(s), e.g., a promoter. It will be apparent that individual polypeptides of a MAPP (e.g., a framework polypeptide and dimerization polypeptide) may be encoded on a single nucleic acid (e.g., under the control of separate promoters), or alternatively, may be located on two or more separate nucleic acids (e.g., plasmids).
2. Recombinant Expression Vectors

The present disclosure provides recombinant expression vectors comprising nucleic acids encoding one or more polypeptides of a MAPP or its higher order complexes. In some cases, the recombinant expression vector is a non-viral vector. In some cases, the recombinant expression vector is a viral construct, such as a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Virol. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In some cases, a nucleotide sequence encoding one or more polypeptides of a MAPP is operably linked to a control element, e.g., a transcriptional control element, such as a promoter. The transcriptional control element may be functional in either a eukaryotic cell, e.g., a mammalian cell such as a human, hamster, or mouse cell; or a prokaryotic cell (e.g., bacterial). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include the cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

E. Genetically Modified Host Cells

The present disclosure provides a genetically modified host cell, where the host cell is genetically modified with a nucleic acid(s) of the present disclosure that encode, or encode and express, MAPP proteins or higher order complexes of MAPPs (e.g., duplex MAPPs).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2™), CHO cells (e.g., ATCC Nos. CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL-10™), PC12 cells (ATCC No. CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. In some cases, the host cell is a mammalian cell that has been genetically modified such that it does not synthesize endogenous MHC β2M and/or such that it does not synthesize endogenous MHC Class I heavy chains (MHC-H).

Genetically modified host cells can be used to produce a MAPP and higher order complexes of MAPPs. For example, a genetically modified host cell can be used to produce a duplex MAPP. For example, an expression vector(s) comprising nucleotide sequences encoding the MAPP polypeptide(s) is/are introduced into a host cell, generating a genetically modified host cell, which genetically modified host cell produces the polypeptide(s) (e.g., as an excreted soluble protein).

F. Methods of Producing MAPPs

The present disclosure provides methods of producing the MAPPs (e.g., duplex MAPPs) described herein. The methods generally involve culturing, in a culture medium, a host cell that is genetically modified with a recombinant expression vector(s) comprising a nucleotide sequence(s) encoding the MAPP (e.g., a genetically modified host cell of the present disclosure); and isolating the MAPP from the genetically modified host cell and/or the culture medium. As noted above, in some cases, the individual polypeptide chains of a MAPP are encoded in separate nucleic acids (e.g., recombinant expression vectors). In some cases, all polypeptide chains of a MAPP are encoded in a single recombinant expression vector.

Isolation of the MAPP from the host cell employed for expression (e.g., from a lysate of the expression host cell) and/or the culture medium in which the host cell is cultured, can be carried out using standard methods of protein purification. For example, a lysate of the host cell may be prepared, and the MAPP purified from the lysate using high performance liquid chromatography (HPLC), exclusion chromatography (e.g., size exclusion chromatography), gel electrophoresis, affinity chromatography, or other purification technique. Alternatively, where the MAPP is secreted from the expression host cell into the culture medium, the MAPP can be purified from the culture medium using HPLC, exclusion chromatography, gel electrophoresis, affinity chromatography, or other purification technique. In some cases, the MAPP is purified, e.g., a composition is generated that comprises at least 80% by weight, at least about 85% by weight, at least about 95% by weight, or at least about 99.5% by weight, of the MAPP in relation to contaminants related to the method of preparation of the product and its purification. The percentages can be based upon total protein.

In some cases, e.g., where the expressed MAPP comprises an affinity tag or affinity domain, the MAPP can be purified using an immobilized binding partner of the affinity tag. For example, where a MAPP comprises an Ig Fc polypeptide, the MAPP can be isolated from genetically modified mammalian host cell and/or from culture medium comprising the MAPP by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

The polypeptides of the MAPP will self-assemble into heterodimers, and where applicable, spontaneously form disulfide bonds between, for example, framework polypeptides, or framework and dimerization polypeptides. As also noted above, when both framework polypeptides include Ig Fc polypeptides, disulfide bonds will spontaneously form between the respective Ig Fc polypeptides to covalently link the two heterodimers of framework and dimerization polypeptides to one another to form a covalently linked duplex MAPP.

G. Compositions

1. Compositions Comprising a MAPP

The present disclosure provides compositions, including pharmaceutical compositions, comprising a MAPP and/or higher order complexes of MAPPs (e.g., duplex MAPPs). Pharmaceutical composition can comprise, in addition to a MAPP , one or more known carriers, excipients, diluents, buffers, salts, surfactants (e.g., non-ionic surfactants), amino acids (e.g., arginine), etc., a variety of which are known in the art and need not be discussed in detail herein. For example, see “Remington: The Science and Practice of Pharmacy”, 19^{th} Ed. (1995), or latest edition, Mack Publishing Co.

In some cases, a subject pharmaceutical composition will be suitable for administration to a subject, e.g., will be sterile and/or substantially free of pyrogens. For example, in some embodiments, a subject pharmaceutical composition will be suitable for administration to a human subject, e.g., where the composition is sterile and is substantially free of detectable pyrogens and/or other toxins, or such detectable pyrogens and/or other toxins are below a permissible limit.

The compositions may, for example, be in the form of aqueous or other solutions, powders, granules, tablets, pills, suppositories, capsules, suspensions, sprays, and the like. The composition may be formulated according to the various routes of administration described below.

Where a MAPP or higher order MAPP complex (e.g., duplex MAPP) is administered as an injectable (e.g., subcutaneously, intraperitoneally, intramuscularly, intralymphatically, and/or intravenously) directly into a tissue, a formulation can be provided as a ready-to-use dosage form, or as non-aqueous form (e.g., a reconstitutable storage-stable powder) or an aqueous form, such as liquid composed of pharmaceutically acceptable carriers and excipients. MAPPs may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a MAPP composition comprises: a) a MAPP higher order MAPP complex (e.g., a duplex MAPP) of the present disclosure; and b) saline (e.g., 0.9% NaCl). In some cases, the composition is sterile and/or substantially pyrogen free. In some cases, the composition is suitable for administration to a human subject, e.g., where the composition is sterile and is free of detectable pyrogens and/or other toxins. Thus, the present disclosure provides a composition comprising: a) a MAPP or higher order MAPP complex (e.g., duplex MAPP) of the present disclosure; and b) saline (e.g., 0.9% NaCl), where the composition is sterile and is substantially free of detectable pyrogens and/or other toxins, or such detectable pyrogens and/or other toxins are below a permissible limit.

Other examples of components suitable for inclusion in formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. A pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a MAPP in a formulation can vary widely. For example, a MAPP or higher order MAPP complex (e.g., duplex MAPP) may be present from less than about 0.1% (usually at least about 2%) to as much as 20% to 50% or more by weight (e.g., from 1% to 10%, 5% to 15%, 10% to 20% by weight, or 20-50% by weight) by weight. The concentration will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.
2. Compositions Comprising a Nucleic Acid or a Recombinant Expression Vector

The present disclosure provides compositions (e.g., pharmaceutical compositions) comprising a nucleic acid or a recombinant expression vector that comprise one or more nucleic acid sequences encoding any one or more MAPP polypeptides (or each of the polypeptides of a MAPP). As discussed above, a wide variety of pharmaceutically acceptable excipients is known in the art and need not be discussed in detail herein.

A nucleic acid or a recombinant expression vector composition can include one or more nucleic acids or one or more recombinant expression vectors comprising a nucleic acid (e.g., DNA or RNA) sequences encoding a MAPP polypeptide or all polypeptides of a MAPP. Such compositions may further include one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative.

A pharmaceutically acceptable formulation may comprise a nucleic acid or recombinant expression vector encoding one or more polypeptides of a MAPP (e.g., in an amount of from about 0.001% to about 90% (w/w)). In some cases, such pharmaceutical compositions will be suitable for administration to a subject, e.g., will be sterile and/or substantially free of pyrogens. For example, in some embodiments, a the pharmaceutical composition will be suitable for administration to a human subject, e.g., where the composition is sterile and is substantially free of detectable pyrogens and/or other toxins, or such detectable pyrogens and/or other toxins are below a permissible limit.

A composition comprising a nucleic acid or a recombinant expression vector encoding one or more polypeptides of a MAPP, including pharmaceutically acceptable formulations, may be:
(i) admixed, encapsulated, conjugated or otherwise associated with other compounds or mixtures of compounds (e.g., liposomes or receptor-targeted molecules), or combined in a formulation with one or more components that assist in uptake, distribution and/or absorption of the nucleic acids or vectors;
(ii) formulated into dosage forms including, but not limited to, tablets, capsules, gel capsules, liquid syrups, soft gels, or suspensions in aqueous, non-aqueous or mixed media; and
(iii) formulated as a liposomal formulation. As used herein, the term “liposome” means a vesicle composed of amphiphilic lipids.

The compositions comprising a nucleic acid or a recombinant expression vector described herein may include penetration enhancers to effect the efficient delivery of nucleic acids or expression vectors. Penetration enhancers may be classified as belonging to one of five broad categories, i.e., surfactants, fatty acids, bile salts, chelating agents, and non-chelating non-surfactants. Penetration enhancers and their uses are further described in, for example, U.S. Pat. No. 6,287,860, which is incorporated for its discussion penetration enhancers.

H. Methods

MAPPs and higher order MAPP complexes (e.g., duplex MAPP) of the present disclosure are useful for modulating an activity of a T cell. Thus, the present disclosure provides methods of modulating an activity of a T cell, the methods generally involving contacting a target T cell with a MAPP or a higher order MAPP complex (e.g., duplex MAPP) of the present disclosure.
1. Methods of Modulating T Cell Activity

The present disclosure provides a method of selectively modulating the activity of a T cell that is specific for an epitope presented by a MAPP, the method comprising contacting, in vitro or in vivo, the T cell with a MAPP, where contacting the T cell with a MAPP or higher order MAPP, such as a duplex MAPP, presenting the epitope selectively modulates the activity of the epitope-specific T cell. The contacting may occur in vivo, typically in a human, but potentially in another animal such as a rat, mouse, dog, cat, pig, horse, or primate. In some cases, the contacting occurs in vitro.

In some cases, contacting a MAPP with a T cell that is specific for the epitope presented by the MAPP result in one or more of: i) activating or suppression of a cytotoxic (e.g., CD8+) T cell; ii) inducing or suppression of a cytotoxic activity of a cytotoxic (e.g., CD8⁺) T cell; iii) inducing or suppressing the production and release of one or more cytotoxic molecules (e.g., a perforin; a granzyme; a granulysin) by a cytotoxic (e.g., CD8+) T cell; iv) inhibiting activity of an autoreactive T cell; v) induction of CD8+ T regulatory cells; and the like.

In some cases, a MAPP or higher order MAPP complex (e.g., duplex MAPP) of the present disclosure interacts with an epitope specific CD 8+ T cell and increases granule-dependent and/or granule-independent CD8⁺ effector T cell responses. Granule-independent responses include, but are not limited to, changes in the number or percentage of active epitope-specific CD 8+ T cell (e.g., in a population of cells such as in blood, lymphatics, and/or in a target tissue), changes in the expression of Fas ligand (Fas-L, which can result in activation of caspases and target cell death through apoptosis), and cytokine/chemokine production (e.g., production and release of interferon gamma (IFN-γ). Granule-dependent effector actions include the release of granzymes, perforin, and/or granulysin. Activation of epitope specific CD8⁺ cytotoxic T cells (e.g., CD8⁺ cytotoxic effector T cells) can result in the targeted killing of, for example, cancer cells and/or infected cell by epitope specific T cells that recognize the epitope presented by the MAPP or MAPP complex through granule-dependent and/or independent responses.

In some cases a MAPP or higher order MAPP complex of the present disclosure comprises a cancer epitope, and the MAPP or MAPP complex activates a CD8⁺ T cell response (e.g., a CD8⁺ T cell response to a cancer cell). In some cases, a MAPP or higher order MAPP complex of the present disclosure comprises an epitope of an infectious agent, and it activates a CD8⁺ T cell response (e.g., a CD8⁺ T cell response to a cell expressing an antigen of an infectious agent).

The present disclosure provides a method of increasing proliferation and/or number of CD 8+ effector T cells specific to the epitope presented by a MAPP or MAPP complex, the method comprising contacting (e.g., in vitro or in vivo) T cells with a MAPP or higher order MAPP complex (such as by administering to a subject one or more doses of a epitope-presenting MAPP or MAPP complex with a MOD (e.g., IL-2)). The contacting or administering may increase the number of CD8+ effector T cells having a TCR capable of binding the epitope present in the MAPP or MAPP complex relative to the number (e.g., total number or percentage) of T cells present (e.g., in a population of cells such as in blood, lymphatics, and/or in a target tissue). For example, the number of CD 8+ effector T cells specific to the epitope presented by the MAPP or MAPP complex (e.g., duplex MAPP) can be increased by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold following one or more contacts with doses or administrations of the MAPP or higher order MAPP complex. The increase may be calculated relative the CD+ T cell numbers present prior to the contacting or administrations, or relative to the population of T cells present in a sample (e.g., a sample of blood or tissue) that has not been contacted with the MAPP or MAPP complex.

The present disclosure provides a method of increasing granule-dependent and/or granule-independent responses of epitope-specific CD 8+ T cell comprising contacting (e.g., in vitro or in vivo) T cells with a epitope presenting MAPP or higher order MAPP complex (such as by administering to a subject one or more does of a MAPP or MAPP complex with, for example a CD80 or CD86 MOD). The contacting or administering results in increasing the expression of Fas ligand expression, cytokines/chemokines (e.g., IL-2, IL-4, and/or IL-5), release of interferons (e.g., IFN-γ), release of granzymes, release of perforin, and/or release of granulysin. For example, contacting CD 8+ effector cell with a MAPP or MAPP complex (e.g., duplex MAPP) presenting epitope specific to the effector cell can increase one or more of Fas ligand expression, interferon gamma (IFN-γ) release, granzyme release, perforin release, and/or granulysin release by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold. The increase may be calculated relative the level of expression or release prior to the contacting or administrations, or relative to the population of T cells present in a sample (e.g., a sample of blood or tissue) that has not been contacted with the MAPP or MAPP complex.

Dysregulation of CD8⁺ T reg cells and self-reactive CD8⁺ effector T cells have both been associated with the pathogenesis of autoimmune diseases including, but not limited to, multiple sclerosis, Rasmussen's encephalitis, paraneoplastic syndromes, systemic sclerosis (SSc), Grave's disease (GD), systemic lupus erythematosus (SLE), aplastic anemia (AA), and vitiligo (see e.g., Pilli et al, Frontiers in Immunology, Article 652, vol. 8, June 2017; Coppieters et al, J. Exp. Med. Vol. 209 No. 1 51-60 (2012); Han et al., PNAS (USA), 110(32):13074-13078 (2013) and Pellegrino et al. PLOS ONE, https://doi.org/10.1371/journal.pone.0210839 January 16, (2019). Deng et al, has reviewed the epigenetic role of CD8+ T cell in autoimmune diseases (see Deng et al, Frontiers in Immunology, Article 856, vol. 10, April 2019).

CD8+ effectors may promote autoimmune diseases via dysregulated secretion of inflammatory cytokines, skewed differentiation profiles and inappropriate apoptosis or induction of effector T cells directed against target cells. In some cases, a MAPP or a higher order MAPP complex (e.g., duplex MAPP) of the present disclosure presenting an auto-antigen (self-antigen or self-epitope) may reduce the activity of an autoreactive CD8+ effector T cells by direct interaction with the cell. Contacting a MAPP or higher order MAPP complex (e.g., duplex MAPP) of the present disclosure presenting an autoantigen and one or more independently selected inhibitory MODs (e.g., PDL1, or FasL) with an autoreactive CD8+ effector T cell may be employed to block autoimmune disease by regulating (e.g., reducing) the release of proinflammatory molecules by such T cells and/or by eliminating self-reactive cells.

Where it is desirable to reduce the activity of epitope specific T cells (e.g., where they are directed against an autoantigen) they may be contacting MAPPs or MAPP complexes (e.g., duplex MAPP) presenting the epitope and bearing MODs that modulate their epitope specific response. Modulation of the cytotoxic CD8+ T cells by MAPP and MAPP complexes may result in, but is not limited to, one or more of: i) suppression of FasL expression by the T cell; ii) suppression of chemokine and/or cytokine release (e.g., IFN-γ); and/or iii) suppression of cytotoxin (e.g., a perforin; a granzyme; a granulysin) synthesis or release. The disclosure includes and provides for a method of reducing (e.g., in vivo, or in vitro) effector T cell activity in an epitope specific manner, such as where the T cell is directed to an autoantigen. The method comprises contacting (such as by administering to a subject) an epitope-specific T cell with one or more doses of a MAPP or MAPP complex presenting the epitope and bearing a MOD (e.g., TGF-β); with the contacting or administering resulting in a reduction in one or more of: i) FasL expression by the T cell; ii) suppression of chemokine and/or cytokine release (e.g., IFN-γ); and iii) suppression of cytotoxin (e.g., a perforin; a granzyme; a granulysin) synthesis or release by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold. The change may be calculated relative the level of expression or release prior to the contacting or administrations, or relative to the population of T cells present in a sample (e.g., a sample of blood or tissue) that has not been contacted with the MAPP or MAPP complex.

In other instances, a MAPP or a higher order MAPP complex presenting and antigen (e.g., an autoantigen) may interact with and increase the number or activity of CD8+ regulatory T cells (CD8+ T regs, characterized e.g., as CD8+ FOXP3+ or CD8+ FOXP3+ CD25+) specific to the antigen. Various CD8⁺ Treg subsets function by, for example, secreting cytokines and chemokines, including IL-10, TGF-β, IL-16, IFN-γ and chemokine (C-C motif) ligand 4 (CCL4), and thereby suppressing the activity of effector T cells and potentially the activity of CD4+ T cells subject to those cytokines. CD8⁺ Tregs may also inhibit T cell function through cell-to-cell contact in which surface proteins such as TGF-β and cytotoxic T-lymphocyte associated protein 4 (CTLA-4) act on the T effector cell. See e.g., Yu et al. Oncol. Lett 15(6):8187-8194 (2018).

The present disclosure provides methods of increasing the number (proliferation) of epitope specific CD8+ Tregs directed to a self-antigen and/or the release of one or more of IL-10, TGF-β, IL-16, IFN-γ and CCL4 and thereby suppressing immune/autoimmune responses. One method of increasing the number of self-antigen (self-epitope) specific CD8+ Tregs (e.g., in a subject) comprises contacting (in vitro or in vivo such as by administering to a subject) an epitope-specific T cell with one or more doses of a MAPP or MAPP complex presenting the self-antigen (self-epitope) and bearing a MOD stimulatory to CD8+ T reg proliferation, where the contacting increases proliferation of CD8+ Tregs by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold relative to the number of CD8+ T regs present in a sample (e.g., a sample of blood or tissue) that has not been contacted with the MAPP or MAPP complex.

The present disclosure also provides methods of increasing one or more of IL-10, TGF-β, IL-16, IFN-γ and CCL4 and thereby suppressing immune/autoimmune responses. The method comprising contacting (in vitro or in vivo such as by administering to a subject) an epitope-specific T cell with one or more doses of a MAPP or MAPP complex presenting the antigen and bearing a MOD stimulatory to CD8+ T reg proliferation. The contacting increases the release at least one of IL-10, TGF-β, IL-16, IFN-γ and CCL4 by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold relative to the amounts prior to the contacting or relative to the population of T cells present in a sample (e.g., a sample of blood or tissue) that has not been contacted with the MAPP or MAPP complex.

Where it is desirable to reduce or substantially eliminate the number of epitope specific CD8+ effectors (e.g., where they are directed against an autoantigen) they may be contacted with a MAPP or MAPP complexes (e.g., duplex MAPP) presenting the epitope and comprising one or more MODs that lead to suppression of the CD8+ T cells (e.g., PDL-1) or apoptosis and/or comprise an Ig Fc region facilitating antibody-dependent cell-mediated cytotoxicity (ADCC) and/or complement-dependent cytotoxicity (CDC). Apoptosis may occur, for example, when the MAPP or MAPP complex comprises both an epitope (e.g., an autoantigen) and a MOD such as FasL that induces FAS mediated apoptosis. Elimination of epitope specific T cells may also occur as a result of antibody-dependent cell-mediated cytotoxicity (ADCC) and/or complement-dependent cytotoxicity (CDC) where the MAPP or MAPP complex presents an epitope and comprises an immunoglobulin Fc polypeptide with wt. or enhanced ADCC and/or CDC functionality. Accordingly, the disclosure includes and provides for a method of reducing or substantially eliminating (e.g., in vivo or in vitro) the number of effector T cells in an epitope specific manner, such as where the T cell is directed to an autoantigen. The method comprises contacting (such as by administering to a subject in vivo, or to a cell in vitro) an epitope-specific T cell with one or more doses of a MAPP or MAPP complex presenting the epitope and bearing a MOD resulting in T cell suppression and/or apoptosis. The contacting or administering resulting elimination of at least 5%, at least 10%, at least 20%, at least 30%, at least 40%, at least 50%, at least 75%, at least 80%, at least 85%, at least 90%, at least 95%, or 100% of the CD8+ cells specific to the epitope presented by the MAPP or MAPP complex. The change may be calculated relative the number of T cells present in a sample (e.g., a sample of blood or tissue) prior to the contacting (administration) of the MAPP or MAPP complex, or a sample that has not been contacted with the MAPP or MAPP complex.
2.Methods of Detecting an Antigen-Specific T Cell

The present disclosure provides a method of detecting an antigen-specific T-cell. The methods comprise contacting a T cell with a MAPP either lacking a MOD or bearing a MOD with minimal affinity for its co-MOD; and detecting binding of the MAPP to the T cell. Binding of such a MAPP to the T cell indicates that the T cell is specific for the epitope present in the MAPP. In some cases, the MAPP comprises a detectable label. Suitable detectable labels include, but are not limited to, a radioisotope, a fluorescent polypeptide, or an enzyme that generates a fluorescent product, and an enzyme that generates a colored product. Where the MAPP comprises a detectable label, binding of the MAPP to the T cell is detected by detecting the detectable label.

In some cases, an MAPP comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacyclododecane)), iron chelates, magnesium chelates, manganese chelates, copper chelates, chromium chelates, iodine-based materials, and radionuclides.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), and the like. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes that may be employed as labels include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of the MAPP to the T cell is detected using a detectably labeled antibody specific for the MAPP. An antibody specific for the MAPP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.
3. Treatment Methods

The present disclosure provides treatment methods, the methods comprising administering to the individual a composition comprising an amount of a MAPP or higher order MAPP complex (e.g., duplex MAPP) of the present disclosure, or one or more nucleic acids or expression vectors encoding a MAPP that may assemble into a higher order complex (e.g., duplex MAPP), effective to selectively modulate the activity of an epitope-specific T cell in an individual and to treat the individual. In some cases, a treatment method comprises administering to an individual in need thereof a composition comprising one or more recombinant expression vectors comprising nucleotide sequences encoding a MAPP (e.g., a MAPP that may assemble into a higher order MAPP complex) of the present disclosure. In some cases, a treatment method comprises administering to an individual in need thereof one or more mRNA molecules comprising nucleotide sequences encoding a MAPP of the present disclosure. In some cases, a treatment method comprises administering to an individual in need thereof a MAPP or higher order MAPP complex (e.g., duplex MAPP) of the present disclosure. The conditions that can be treated include autoimmune disorders.

The present disclosure provides a method of selectively modulating the activity of an epitope-specific T cell in an individual, the method comprising administering to the individual an effective amount of a MAPP or higher order MAPP complex (e.g., duplex MAPP) of the present disclosure, or one or more nucleic acids (e.g., expression vectors; mRNA; etc.) comprising nucleotide sequences encoding the MAPP which may assemble into a higher order complex, where the MAPP or its complex selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a MAPP (e.g., duplex MAPP) of the present disclosure) sufficient to effect treatment of a disease or disorder.

In some cases, a MAPP comprises an inhibitory MOD polypeptide sequence, and the MAPP inhibits activity of the epitope-specific T cell (e.g., effector functions or proliferation). In some cases, the epitope is an epitope of an autoantigen (self-epitope), and a MAPP selectively inhibits the activity of a T cell specific for the epitope of the autoantigen.

The present disclosure provides a method of treating an autoimmune disorder in an individual, the method comprising administering to the individual pharmaceutical composition comprising an effective amount of a MAPP or higher order MAPP complex such as a duplex MAPP of the present disclosure, or one or more nucleic acids comprising nucleotide sequences encoding the MAPP (which may assemble into a higher order complex such as an duplex MAPP), wherein the MAPP (e.g., a MAPP comprising an epitope of an autoantigen), and wherein the MAPP or higher order MAPP complex (e.g., duplex MAPP) comprises an inhibitory MOD (e.g., FasL and/or PDL1). In some cases an “effective amount” of a MAPP or higher order MAPP complex is an amount that, when administered in one or more doses to an individual in need thereof, reduces the number self-reactive T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the MAPP, or in the absence of MAPP administration. In some cases, an “effective amount” of a MAPP is an amount that, when administered in one or more doses to an individual in need thereof, reduces production of Th2 cytokines (e.g., IL-4, IL-5, and IL-13) in the individual or a tissue of the individual. In some cases, an “effective amount” of a MAPP or higher order MAPP complex (e.g., duplex MAPP) of the present disclosure is an amount that, when administered in one or more doses to an individual in need thereof, ameliorates one or more symptoms associated with an autoimmune disease in the individual. In some instances, the MAPP or higher order MAPP complex (e.g., duplex MAPP) reduces the number of CD8⁺ self-reactive T cells. In some instances, the MAPP or higher order MAPP complex (e.g., duplex MAPP) increases the number of CD8⁺ Tregs, which in turn reduces the number of CD8⁺ self-reactive T effector cells and/or the cytokines or cytotoxic components (e.g., a perforin; a granzyme; a granulysin) released by activated CD8+ effector cells.

In some cases, the MOD is an activating polypeptide, and the MAPP with its associated epitope activates an epitope-specific T cell that recognizes a cancer or pathogen specific antigen (e.g., a viral or bacterial antigen). In some cases, the T cells are cytotoxic T cells (CD8⁺ cells). In some cases, a MAPP with its associated epitope increases the activity of a CD8+ effector T cell specific for a cancer cell expressing the epitope. Activation of CD8⁺ T cells can include increasing proliferation of CD8⁺ T cells and/or inducing or enhancing release of chemokines and/or cytokines by CD8⁺ T cells.

“In some cases, a MAPP with its associated epitope reduces proliferation and/or activity of a CD8+ regulatory T cell. In some cases, e.g., where a MAPP or MAPP complex of the present disclosure comprises an inhibitory MOD (e.g., PD-L1, FasL, and the like), the MAPP epitope conjugate reduces the proliferation and/or activity of a CD8+ Treg.

As noted above, in some cases, in carrying out a subject treatment method, a MAPP (e.g., duplex MAPP or higher order MAPP complex) is administered to an individual in need thereof, as the polypeptide per se. In other instances, in carrying out a subject treatment method, one or more nucleic acids comprising nucleotide sequences encoding a MAPP is/are administering to an individual in need thereof. Thus, in other instances, one or more nucleic acids of the present disclosure, e.g., one or more recombinant expression vectors of the present disclosure, is/are administered to an individual in need thereof.

A MAPP or higher order MAPP complex (e.g., duplex MAPP), or one or more nucleic acids encoding such molecules may be administered alone or with one or more additional therapeutic agents or drugs. The therapeutic agents may be administered before, during, or subsequent to MAPP or higher order MAPP complex (e.g., duplex MAPP) or nucleic acids encoding such molecules. When the additional therapeutic agents are administered with a composition or formulation comprising a MAPP or higher order MAPP complex (e.g., duplex MAPP) or nucleic acids encoding such molecules, the therapeutic agent may be administered concurrently with the MAPP. Alternatively, the therapeutic agents may be co-administered with the MAPP as part of a formulation or composition comprising the MAPP or higher order MAPP complex (e.g., duplex MAPP).

Suitable therapeutic agents or drugs that may be administered with a MAPP or higher order MAPP complex include virtually any therapeutic agent, including small molecule therapeutics (e.g., less than 2,000 Daltons in molecular weight) approved by the U.S. Food and Drug Administration, and/or listed in the 2020 U.S. Pharmacopeia or National Formulary. In an embodiment, those therapeutic agents or drugs are less than 1,000 molecular weight. Suitable drugs include antibiotics, chemotherapeutic (antineoplastic), anti-fungal, or anti-helminth agents and the like (e.g., sulfasalazine, azathioprine, cyclophosphamide, leflunomide; methotrexate, antimalarials, D-penicillamine, cyclosporine). Suitable chemotherapeutics may be alkylating agents, cytoskeletal disruptors (taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids. Suitable drugs also include non-steroidal anti-inflammatory drugs and glucocorticoids, and the like.

In an embodiment, a suitable therapeutic agent that may be administered with a MAPP or higher order MAPP complex comprises an anti-TGF-β antibody, such as Metelimumab (CAT192) directed against TGF-β1 and/or Fresolimub directed against TGF-β1 and TGF-β2, or a TGF-β trap (e.g., Cablivi® caplacizumab-yhdp). Such antibodies would, as a generality, not be administered in conjunction with a MAPP or higher order MAPP complex (e.g., a duplexed MAPP) that comprise a sequence to which the antibodies bind such as a TGF-β1 or TGF-β2 MOD.

In an embodiment, a suitable therapeutic agent that may be administered with a MAPP or higher order MAPP complex comprises one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab) disialoganglioside (GD2); CD38 (e.g., daratumumab); SLAMF7 (Elotuzumab); both EpCAM and CD3 (e.g., catumaxomab); or both CD19 and CD3 (blinatumomab). Such antibodies would, as a generality, not be administered in conjunction with a MAPP or higher order MAPP complex (e.g., a duplexed MAPP) that comprise a sequence to which any of the administered antibodies bind, or which may block the action of a MOD present in the administered MAPP.

In an embodiment, a suitable therapeutic agent that may be administered with a MAPP or higher order MAPP complex comprises an antibiotic, anti-fungal, and/or anti-helminth agent.

In an embodiment, a suitable therapeutic agent that may be administered with a MAPP or higher order MAPP complex comprises one or more chemotherapeutic agents. Such therapeutic agents may be selected from: alkylating agents, cytoskeletal disruptors (taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids and their derivatives. In an embodiment, the chemotherapeutic agents are selected from actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

The present disclosure provides treatment methods, the methods comprising administering to the individual an amount of a MAPP or higher order MAPP complex of the present disclosure, or one or more nucleic acids or expression vectors encoding the MAPP, effective to selectively modulate the activity of an epitope-specific T cell in an individual and to treat the individual. In some cases, a treatment method comprises administering to an individual in need thereof one or more recombinant expression vectors comprising nucleotide sequences encoding a MAPP or higher order MAPP complex of the present disclosure. In some cases, a treatment method comprises administering to an individual in need thereof one or more mRNA molecules comprising nucleotide sequences encoding a MAPP or higher order MAPP complex of the present disclosure. In some cases, a treatment method comprises administering to an individual in need thereof a MAPP or higher order MAPP complex of the present disclosure.

The present disclosure also provides treatment methods, the methods comprising administering to the individual a pharmaceutical composition comprising an effective amount of a MAPP of the present disclosure, or one or more nucleic acids or expression vectors encoding the MAPP optionally bearing an immunoglobulin sequence that can support complement-dependent cytotoxicity (CDC) or antibody-dependent cell cytotoxicity (ADCC). Such MAPPs can selectively engage with an epitope-specific T cell in an individual in order to treat the individual, (e.g., by depleting epitope-specific T cells by inducing cell lysis through activation of CDC, and/or ADCC).

The present disclosure provides a method of selectively modulating the activity of an epitope-specific T cell in an individual, the method comprising administering to the individual an effective amount of a MAPP or higher order MAPP complex of the present disclosure, or one or more nucleic acids (e.g., expression vectors; mRNA; etc.) comprising nucleotide sequences encoding the MAPP or higher order MAPP complex, which selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a MAPP or higher order MAPP complex in order to treat a disease or disorder.

In some cases, the MOD is an inhibitory polypeptide, and a MAPP or higher order MAPP complex inhibits activity of the epitope-specific T cell. In some cases, the epitope an epitope of an autoantigen, and a MAPP or higher order MAPP complex selectively inhibits the activity of a T cell specific for the epitope of the autoantigen.

The present disclosure provides a method of treating an autoimmune disorder in an individual, the method comprising administering to the individual an effective amount of a MAPP or higher order MAPP complex that comprises an epitope of an autoantigen and an inhibitory MOD (or one or more nucleic acids comprising nucleotide sequences encoding those molecules. In some cases, an “effective amount” of a MAPP or higher order MAPP complex is an amount that, when administered in one or more doses to an individual in need thereof, reduces the number of self-reactive T cells specific to the epitope presented by the MAPP or higher order MAPP complex by at least 10%, at least 15%, at least 20%, at least 25%, at least 30%, at least 40%, at least 50%, at least 60%, at least 70%, at least 80%, at least 90%, or at least 95%, compared to number of those self-reactive T cells in the individual before or in the absence of administration of the MAPP or higher order MAPP complex. In some cases, an “effective amount” of a MAPP or higher order MAPP complex is an amount that, when administered in one or more doses to an individual in need thereof, reduces production of Th2 cytokines in the individual. In some cases, an “effective amount” of a MAPP or higher order MAPP complex is an amount that, when administered in one or more doses to an individual in need thereof, ameliorates one or more symptoms associated with an autoimmune disease in the individual. In some instances, the MAPP or higher order MAPP complex reduces the number of CD8⁺ self-reactive T cells specific to the epitope presented by those molecules, which may in turn may lead to a reduction in CD4⁺ self-reactive T cells. In some instances, a MAPP or higher order MAPP complex increases the number of CD8⁺ Tregs specific to the epitope presented by those molecules, which in turn reduces the number or activity of CD8⁺ self-reactive T cells.

In some cases, the MOD is an activating polypeptide, and the MAPP with its associated epitope activates an epitope-specific T cell that recognizes a cancer or pathogen specific antigen (e.g., a viral or bacterial antigen). In some cases, the T cells are cytotoxic T cells (CD8⁺ cells). In some cases MAPP or higher order MAPP complex with its associated epitope increases the activity of a T cell specific for a cancer cell expressing the epitope (e.g., cytotoxic CD8⁺ T cells). Activation of CD8⁺ T cells can include increasing proliferation of CD8⁺ T cells and/or inducing or enhancing release cytokines by CD8⁺ T cells such as interferon γ CD8+ cells.

In some cases where a MAPP or higher order MAPP complex comprises an inhibitory MOD (e.g., PD-L1, FasL, and the like) it may reduce the proliferation and/or activity of a CD8⁺ regulatory T (Treg) cell (e.g., FoxP3⁺, CD8⁺ T cells) specific to the epitope presented by the MAPP or higher order MAPP complex.

As noted above, in some cases, in carrying out a subject treatment method, a MAPP or higher order MAPP complex is administered to an individual in need thereof, as the polypeptide per se. In other instances, in carrying out a subject treatment method, one or more nucleic acids comprising nucleotide sequences encoding a MAPP is/are administering to an individual in need thereof. Thus, in other instances, one or more nucleic acids of the present disclosure, e.g., one or more recombinant expression vectors of the present disclosure, is/are administered to an individual in need thereof.
4. Methods of Selectively Delivering a MOD

The present disclosure provides a method of delivering a MOD polypeptide such as IL-2, 4-1BBL, CD-80, CD-86, Fas-L, PD-L1, or a reduced-affinity variant of any thereof (e.g., PD-L1 and/or an IL-2 variant disclosed herein) to a selected T cell or a selected T cell population, e.g., in a manner such that a TCR specific for a given epitope is targeted. The present disclosure thus provides a method of delivering a MOD polypeptide such as a IL-2 polypeptide, or a reduced-affinity variant of a naturally occurring MOD polypeptide such as an IL-2 variant, selectively to a target T cell bearing a TCR specific for the peptide epitope sequence present in a MAPP or higher order MAPP complex (e.g., duplex MAPP). The method comprises contacting a population of T cells with a MAPP or higher order MAPP complex (e.g., duplex MAPP). The population of T cells can be a mixed population that comprises: i) the target T cell with a TCR specific to a target epitope; and ii) non-target T cells that are not specific for the target epitope presented by the peptide epitope (e.g., T cells that are specific for an epitope(s) other than the epitope to which the epitope-specific T cell binds). The epitope-specific T cell is specific for the peptide epitope present in the MAPP or higher order MAPP complex, and binds to the peptide MHC complex provided by the MAPP or higher order MAPP complex. Contacting the population of T cells with the MAPP or higher order MAPP complex delivers the MOD polypeptide (e.g., IL-2 or a reduced-affinity variant of IL-2) selectively to the T cell(s) that are specific for the epitope present in the MAPP or higher order complex.

Thus, the present disclosure provides a method of delivering a MOD polypeptide such as PD-L1, or a reduced-affinity variant of a naturally occurring MOD polypeptide such as a PD-L1 variant disclosed herein, or a combination of both, selectively to a target T cell selective for the epitope presented by the peptide epitope as presented by the MAPP. Similarly, the disclosure provides a method of delivering an IL-2, MOD polypeptide or a reduced-affinity variant of a naturally occurring IL-2 MOD polypeptide such as disclosed herein, or a combination of both, to a target T cell that is selective for the epitope presented by the peptide epitope as presented by the MAPP. In some cases, the IL-2 MOD bears a substitution at position H16 and/or F42 (e.g., H16 and F42 such as H16A and F42A) (see supra).

For example, a MAPP (such as a duplex MAPP or higher order MAPP complex) is contacted with a population of T cells comprising: i) target T cells that are specific for the epitope present in the MAPP or a higher order MAPP complex; and ii) non-target T cells, e.g., a T cells that are specific for a second epitope(s) that is not the epitope present in the MAPP or a higher order MAPP complex. Contacting the population results in substantially selective delivery of the MOD polypeptide(s) (e.g., naturally occurring or variant MOD polypeptide) to the target T cell. Less than 50%, less than 40%, less than 30%, less than 25%, less than 20%, less than 15%, less than 10%, less than 5%, or less than 4%, 3%, 2% or 1%, of the MAPP or higher order MAPP complex (e.g., duplex MAPP) may bind to non-target T cells and, as a result, the MOD polypeptide (e.g., IL-2 or IL-2 variant) is selectively delivered to target T cell (and accordingly, not effectively delivered to the non-target T cells).

The population of T cells to which a MOD and/or variant MOD is selectively delivered may be in vivo. In some cases, the population of T cells to which a MOD and/or variant MOD is selectively delivered is in vitro.

In some cases, the population of T cells to which a MOD and/or variant MOD is selectively delivered is in vitro. For example, a mixed population of T cells is obtained from an individual, and is contacted with a MAPP (such as a duplex MAPP or higher order MAPP complex) in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the MAPP (e.g., duplex MAPP or higher order MAPP complex). The presence of T cells that are specific for the epitope of the MAPP or higher order MAPP complex can be determined by assaying a sample comprising a mixed population of T cells, which population of T cells comprises T cells that are not specific for the epitope (non-target T cells) and may comprise T cells that are specific for the epitope (target T cells). Known assays can be used to detect the desired modulation of the target T cells, thereby providing an in vitro assay that can determine whether a particular MAPP or higher order MAPP complex possesses an epitope that binds to T cells present in the individual, and thus whether the MAPP or higher order complex has potential use as a therapeutic composition for that individual. Suitable known assays for detection of the desired modulation (e.g., activation/proliferation or inhibition/suppression) of target T cells include, e.g., flow cytometric characterization of T cell phenotype, numbers, and/or antigen specificity. Such an assay to detect the presence of epitope-specific T cells, e.g., a companion diagnostic, can further include additional assays (e.g., effector cytokine ELISpot assays) and/or appropriate controls (e.g., antigen-specific and antigen-nonspecific multimeric peptide-HLA staining reagents) to determine whether the MAPP or higher order MAPP complex is selectively binding, modulating (activating or inhibiting), and/or expanding the target T cells. Thus, for example, the present disclosure provides a method of detecting, in a mixed population of T cells obtained from an individual, the presence of a target T cell that binds an epitope of interest, the method comprising: a) contacting in vitro the mixed population of T cells with a MAPP or higher order MAPP complex comprising an epitope of the present disclosure; and b) detecting modulation (activation or inhibition) and/or proliferation of T cells in response to said contacting, wherein modulation of and/or proliferation of T cells indicates the presence of the target T cell. Alternatively, and/or in addition, if activation and/or expansion (proliferation) of the desired T cell population is obtained using a MAPP or higher order MAPP complex (e.g., a duplex MAPP), then all or a portion of the population of T cells comprising the activated/expanded T cells can be administered back to the individual as a therapy.

In some instances, the population of T cells is in vivo in an individual. In such instances, a method of the present disclosure for selectively delivering a MOD polypeptide (e.g., PD-L1 or a reduced-affinity PD-L1) to an epitope-specific T cell comprises administering the MAPP or higher order MAPP complex (e.g., duplex MAPP) to the individual.

In some instances, the epitope-specific T cell to which a MOD polypeptide sequence (e.g., wild type or reduced affinity IL-2 and/or PD-L1 MOD) is being selectively delivered is referred to herein is a target regulatory T cell (Treg) that may inhibit or suppresses activity of an autoreactive T cell.

I. Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A MAPP (whether as a single heterodimer or, as described above, as a higher order complex such as a duplex MAPP) may be administered in amounts between 1 ng/kg body weight and 20 mg/kg body weight per dose; for example from 0.1 μg/kg body weight to 1.0 mg/kg body weight, from 0.1 mg/kg body weight to 0.5 mg/kg body weight, from 0.5 mg/kg body weight to 1 mg/kg body weight, from 1.0 mg/kg body weight to 5 mg/kg body weight, from 5 mg/kg body weight to 10 mg/kg body weight, from 10 mg/kg body weight to 15 mg/kg body weight, and from 15 mg/kg body weight to 20 mg/kg body weight. Doses below 0.1 mg/kg body weight or above 20 mg/kg are envisioned, especially considering the aforementioned factors. Amounts thus include from about 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight.

Those of skill will readily appreciate that dose levels can vary as a function of the MAPP or higher order MAPP complex (e.g., duplex MAPP), the severity of the symptoms and the susceptibility of the subject to side effects. Preferred dosages for a given compound are readily determinable by those of skill in the art by a variety of means.

In some cases, multiple doses of a MAPP or higher order MAPP complex (e.g., duplex MAPP) are administered. The frequency of administration of a MAPP or higher order MAPP complex (e.g., duplex MAPP) can vary depending on any of a variety of factors, e.g., severity of the symptoms, patient response, etc. For example, in some cases, a MAPP or higher order MAPP complex (e.g., duplex MAPP) is administered once per month, less frequently than once per month, e.g., once every 6 weeks, once every two months, once every three months, or more frequently than once per month, e.g., twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid).

The duration of administration of a MAPP, e.g., the period of time over which a MAPP is administered, can vary, depending on any of a variety of factors, e.g., patient response, etc. For example, a MAPP can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo periodic maintenance therapy to prevent the recurrence of the disease state, wherein a MAPP is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight. The periodic maintenance therapy can be once per month, once every two months, once every three months, once every four months, once every five months, once every six months, or less frequently than once every six months.

J. Routes of Administration

A MAPP or higher order MAPP complex (e.g., a duplex MAPP), or a nucleic acid or recombinant expression vectors comprising nucleic acids encoding one or more polypeptides of a MAPP or its higher order complexes, is administered to an individual using any available method and route suitable for drug delivery, including in vivo and in vitro methods, as well as systemic and localized routes of administration. A MAPP or higher order MAPP complex can be administered to a host using any available conventional methods and routes suitable for delivery of conventional drugs, including systemic or localized routes. In general, routes of administration contemplated for use in a method include, but are not necessarily limited to, enteral, parenteral, and inhalational routes.

Conventional and pharmaceutically acceptable routes of administration include intramuscular, intratracheal, subcutaneous, intradermal, topical application, intravenous, intraarterial, intralymphatic, rectal, nasal, oral, intratumoral, peritumoral, and other enteral and parenteral routes of administration. Of these, intravenous, intramuscular and subcutaneous may be more commonly employed. MAPPS and their higher order complexes, nucleic acids and expression vectors encoding them may be administered, for example, intravenously. Routes of administration may be combined, if desired, or adjusted depending upon, for example, the MAPP or higher order MAPP complex (e.g., duplex MAPP) and/or the desired effect. A MAPP or higher order MAPP complex can be administered in a single dose or in multiple doses.

A MAPP or higher order MAPP complex (e.g., a duplex MAPP), or a nucleic acid or recombinant expression vectors comprising nucleic acids encoding one or more polypeptides of a MAPP or its higher order complexes may also be contacted with cells in vitro. The cells subject to such in vitro treatment and/or their progeny, may then be administered to a patient or subject (e.g., the subject from which the cells treated in vitro were obtained.

K. Subjects Suitable for Treatment

Subjects suitable for treatment include those with a cancer, infectious diseases (e.g., including those with viral, bacterial, and/or mycoplasma causative agents), allergic reactions, and/or autoimmune diseases.

Subjects suitable for treatment who have a cancer include, but are not limited to, individuals who have been provided other treatments for the cancer but who failed to respond to the treatment. Cancers that can be treated with a method include, but are not limited to, those displaying any of the cancer epitopes recited herein including, but not limited to peptide epitopes of AFP, WT-1, HPV and HBV.

Subjects suitable for treatment may also include individuals who have an infectious disease include, but are not limited to, individuals who have been provided other treatments for the infectious disease but who failed to respond to the treatment. Infectious diseases that can be treated with a method include, but are not limited to, those having an infectious agent recited herein including, but not limited to, EBV, HPV and HBV epitopes.

Subjects suitable for treatment also include individuals who have an allergy include, but are not limited to, individuals who have been provided other treatments for the allergy but who failed to respond to the treatment. Allergic conditions that can be treated with a method include, but are not limited to, those resulting from exposure to nuts (e.g., tree and/or peanuts), pollen, and insect venoms (e.g., bee and/or wasp venom antigens).

Subjects suitable for treatment include those with an autoimmune disease or a genetic disposition to develop an autoimmune disease including a family history of an autoimmune disease (e.g., a grandparent, parent, or sibling with the autoimmune disease. Subjects suitable for treatment who have an autoimmune disease include, but are not limited to, individuals who have been provided other treatments for the autoimmune disease but who failed to respond to the treatment. Autoimmune diseases that can be treated with a method include, but are not limited to, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Celiac disease, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), Type 1 Diabetes, vasculitis, and vitiligo.

### V. CERTAIN ASPECTS

Certain aspects, including embodiments/aspects of the present subject matter described above, may be beneficial alone or in combination, with one or more other aspects recited hereinbelow. In addition, while the present disclosure has been disclosed with reference to certain aspects recited below and in the claims, numerous modifications, alterations, and changes to the described aspects/embodiments are possible without departing from the sphere and scope of the present disclosure. Accordingly, it is intended that the present disclosure not be limited to the described embodiments, aspects and claims, but that it has the full scope defined by the language of this disclosure and equivalents thereof.
1. A multimeric antigen-presenting polypeptide complex (MAPP) comprising:
   (i) a framework polypeptide comprising (e.g., from N-terminus to C-terminus) a dimerization sequence and a multimerization sequence;
   (ii) a dimerization polypeptide comprising a counterpart dimerization sequence complementary to the dimerization sequence of the framework polypeptide, and dimerizing therewith through covalent (e.g., disulfide bonds) and/or non-covalent interactions to form a MAPP heterodimer; and
   (iii) at least one (e.g., at least two) presenting sequence and/or presenting complex;
   wherein
   (a) each presenting sequence comprises an epitope, MHC Class I heavy chain (“MHC-H”), and β2M polypeptide sequences,
   (b) each presenting complex comprises a presenting complex 1^{st} sequence and a presenting complex 2^{nd} sequence that together comprise epitope, MHC-H, and β2M polypeptide sequences (the epitope is part of the presenting complex 1^{st} sequence or presenting complex 2^{nd} sequence along with either the MHC-H or β2M polypeptide sequence),
   (c) one or both of the dimerization polypeptide and/or the framework polypeptide (e.g., either the framework polypeptide, dimerization polypeptide, or both polypeptides) comprise a presenting sequence or a presenting complex 1^{st} sequence (e.g., located on the N-terminal side of the framework polypeptide' dimerization sequence, or the N-terminal side of the dimerization polypeptide' counterpart dimerization sequence), and
   (d) wherein optionally at least one (e.g., one, two, or more) of the framework polypeptide, dimerization peptide, presenting sequence(s), presenting complex(s) 1^{st} sequence and/or presenting complex 2^{nd} sequence comprises one, two, three or more independently selected MOD and/or variant MOD polypeptide sequences (e.g., located at their N-terminus, C-terminus, or on the N-terminal or C-terminal side of the dimerization sequences); and
   wherein the framework polypeptide, dimerization polypeptide, presenting sequence, presenting complex 1^{st} sequence and/or presenting complex 2^{nd} sequence optionally comprise one or more linker sequences selected independently. (See e.g., FIGS. 1A and 1B).
      It is understood that the dimerization sequence and multimerization sequences are different polypeptide sequences and do not bind in any substantial manner to each other, e.g., the framework polypeptides do not, to any substantial extent, form hair pin structures, self-polymerize, or self-aggregate. Similarly, such aspects may be subject to the proviso that neither the dimerization sequence nor the multimerization sequence of the framework polypeptide comprises an MHC-H polypeptide sequence having at least 90% (e.g., at least 95% or at least 98%) or 100% sequence identity to at least 40 (e.g., at least 50, 60 or 70) contiguous aas of a β2M or MHC-H polypeptide in any of FIG. 2 or 3A through 3H).
2. The MAPP of aspect 1, wherein the MHC-H polypeptide sequence comprises a human Class I MHC-H chain polypeptide sequence selected from an HLA-A, HLA-B, HLA-C, HLA-E, HLA-F, and HLA-G MHC-H polypeptide sequences or a portion thereof.
3. The MAPP of aspect 1, wherein at least one presenting sequence or presenting complex (e.g., at least two or all presenting sequences and/or complexes) comprises:
   a MHC-H sequence having at least 90% (e.g., at least 95% or 98%) or 100% sequence identity to at least 125 (e.g., at least 150, 175, 200, 250, or 275) contiguous aas of a MHC-H polypeptide provided in any of FIGS. 3A to 3H, wherein the MHC-H sequences do not include the MHC-H transmembrane domain, or a portion thereof, that will anchor the MAPP in a cell membrane; and/or
   a β2M sequence having at least 90% (e.g., at least 95% or 98%) or 100% sequence identity to at least 50 (e.g., 60, 70 80 or 89) contiguous aas of a mature β2M polypeptide (lacking its signal sequence) provided in FIG. 2.
4. The MAPP of any of aspects 1 to 3, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-A allele that does not include the MHC-H transmembrane domain or a portion thereof that will anchor the MAPP in a cell membrane.
5. The MAPP of any of aspects 1 to 4, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-A*0101, HLA-A*0201, HLA-A*0301, HLA-A*1101, HLA-A*2301, HLA-A*2402, HLA-A*2407, HLA-A*3303, or HLA-A*3401 polypeptide sequence provided in FIG. 3E.
6. The MAPP of any of aspects 1 to 5, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-HLA-A*0101, HLA-A*0201, HLA-A*1101, HLA-A*2402, or HLA-A*3303 polypeptide sequence (e.g., as provided in FIG. 3E).
7. The MAPP of any of aspects 1 to 3, wherein the MHC-H polypeptide sequences have at least 90% (e.g., at least 95% or 98%) or 100% sequence identity, such as 90% to 100% or 95% to 100% sequence identity, to at least125 (e.g., at least 150, 175, 200, 250, or 275) contiguous aas of a HLA-B allele that does not include the MHC-H transmembrane domain or a portion thereof that will anchor the MAPP in a cell membrane.
8. The MAPP of any of aspects 1 to 3 or 7, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-B*0702, HLA-B*0801, HLA-B*1502, B27 (subtypes HLA-B*2701-2759), HLA-B*3802, HLA-B*4001, HLA-B*4601, or HLA-B*5301 polypeptide sequence (e.g., as provided in FIG. 3F).
9. The MAPP of any of aspects 1 to 3 or 7, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of HLA-B*0702.
10. The MAPP of any of aspects 1 to 3, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-C allele that does not include the MHC-H transmembrane domain or a portion thereof that will anchor the MAPP in a cell membrane.
11. The MAPP of any of aspects 1 to 3 or 10, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence (e.g., as provided in FIG. 3G).
12. The MAPP of any of aspects 1 to 3 or 10, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of HLA-HLA-C*0701.
13. The MAPP of any of aspects 1 to 3, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-E allele that does not include the MHC-H transmembrane domain or a portion thereof that will anchor the MAPP in a cell membrane.
14. The MAPP of any of aspects 1 to 3 or 13, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-E*0101, HLA-E*01:03, HLA-E*01:04, HLA-E*01:05, HLA-E*01:06, HLA-E*01:07, HLA-E*01:09, or HLA-E*01:10 polypeptide sequence (e.g., as provided in FIG. 3H).
15. The MAPP of any of aspects 1 to 3 or 13, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 225, or all) of the non-variable aas of the HLA-E allele consensus sequence:

GSHSLKYFHT SVSRPGRGEP RFISVGYVDD TQFVRFDNDA
ASPRMVPRAP WMEQEGSEYW DRETRSARDT AQIFRVNLRT
LRG [symbol] YNQSX1A GSHTLQWMHG CELGPDX2RFL RGYEQFAYDG
KDYLTLNEDL RSWTAVDT [symbol] A QISEQKSNDA SEAEHQX3X4YL
EDTCVEWLHK YLEKGKETLL HLEPPKTHVT HHPISDHEAT
LRCWALGFYP AEITLTWQQD GEGHTQDTEL VETRP [symbol] GDGT
FQKWAAVVVP SGEEX5RYTCH VQHEGLX6EPV TLRWKPASQP
TIPI,

wherein X1=K or E, X2=R or G, X3=R or G, X4=A or V, X5=Q or P, and X6=P or S.
16. The MAPP of any of aspects 1 to 3, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-F allele that does not include the MHC-H transmembrane domain or a portion thereof that will anchor the MAPP in a cell membrane.
17. The MAPP of any of aspects 1 to 3 or 16, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-F*0101 (HLA-F*01:01:01:01), HLA-F*01:02, HLA-F*01:03(HLA-F*01:03:01:01), HLA-F*01:04, HLA-F*01:05, and HLA-F*01:06. polypeptide sequence (e.g., as provided in FIG. 3H).
18. The MAPP of any of aspects 1 to 3 or 16, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 225, or all) of the non-variable aas of the HLA-F allele consensus sequence:

GSHSLRX1FST AVSRPGRGEP RYIAVEYVDD TQFLRFDSDA
AIPRMEPREX2 WVEQEGPQYW EWTTGYAKAN AQTDRVALRN
LLRRYNQSEA GSHTLQGMNG CDMGPDGRLL RGYHQHAYDG
KDYISLNEDL RSWTAADTVA QITQRFYEAE EYAEEFRTYL
EGECLELLRR YLENGKETLQ RADPPKAHVA HHPISDHEAT
LRCWALGFYP AEITLTWQRD GEEQTQDTEL VETRPAGDGT
FQKWAAVVVP X3GEEORYTCH VQHEGLPOPL ILRWEQSX4QP
TIPI,

wherein X1=Y or F; X2=P or Q; X3=S or P; and X4=P or L.
19. The MAPP of any of aspects 1 to 3, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-G allele that does not include the MHC-H transmembrane domain or a portion thereof that will anchor the MAPP in a cell membrane.
20. The MAPP of any of aspects 1 to 3 or 19, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 250, or 275) contiguous aas of a HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, or HLA-G*01:22 polypeptide sequence (e.g., as provided in FIG. 3H).
21. The MAPP of any of aspects 1 to 3 or 19, wherein the MHC-H polypeptide sequences have at least 90% (e.g., at least 95% or 98%) or 100% sequence identity, such as 90% to 100% or 95% to 100% sequence identity, to at least 125 (e.g., at least 150, 175, 200, 225, or all) of the non-variable aas of the HLA-G allele consensus sequence:

GSHSMRYFSA AVX1RPGRGEP RFIAMGX2VDD X3QFX4RFDSDS
ACPRMEPRAP WVEX5EGPEYW EEETRNTKAH AQTDRMNLQT
X6RG [symbol] YNQSEA SSHTLQWMIX7 CDLX8X9DGRLX10 RGYEQYAYDG
KDYLALNEDL RSWTAADT [symbol] A QISKRKCEAA NVAEQRRAX11L
EGTCVEWLX12R X13LENGKEX14LQ RADPX15KTHVT HHPVFDYEAT
LRCWALGFYP AEIILTWQX16D GEDQTQDVEL VETRP [symbol] GDGT
FQKWAAVVVP SGEEQRYX17CH VQHEGLPEPL MLRWX18QSSLP
TIPI,

wherein X1=S or F, X2=Y or H, X3=T, S, or M, X4=L or V; X5=Q or R, X6=P or L, X7=G or D, X8=G or V, X9=S or C, X10=L or I, X11=Y or H, X12=H or R, X13=Y or H, X14=M or T, X15=P or A, X16=R, W, or Q, X17=T or M, X18=K or E.
22. The MAPP of any of any preceding aspect, wherein the β2M sequence has at least 90% (e.g., at least 95% or 98%) or 100% sequence identity, such as 90% to 100% or 95% to 100% sequence identity, to at least 50 (e.g., 60, 70 80 or 89) contiguous aas of a mature human β2M polypeptide (e.g., aas 21-119 of NCBI accession number NP_004039.1 provided in FIG. 2). In such an aspect the at least 90% to 100% or 95% to 100% sequence identity may be at least 98% to 100% sequence identity.
23. The MAPP of any of aspects 1 to 22, wherein at least one of the MHC-H polypeptide sequences comprises at least one mutation (e.g., two, or three mutations) selected from the group consisting of: an alanine at position 84 (e.g., Y84A or R84A in the case of HLA-F), a cysteine at position 84 (e.g., Y84C or R84C in the case of HLA-F), a cysteine at position 139 (e.g., A139C or V139C in the case of HLA-F), and a cysteine at position 236 (e.g., A236C). See FIG. 3I for the location of those aa positions
24. The MAPP of any of aspects 1 to 23, wherein at least one of the MHC Class I polypeptide sequences comprises a combination of mutations selected from the group consisting of: Y84A & A139C; Y84A and A236C; Y84C and A139C; Y84C and A236C; and Y84C, A139C and A236C.
25. The MAPP of any of aspects 1 to 24, wherein the at least one presenting sequence/complex (e.g., two, three, four, or each) comprises:
   a β2M sequence having a cysteine at position 12 of the mature β2M peptide (e.g., an R12C substitution, see FIG. 2 for numbering) and a MHC-H sequence with a cysteine at position 236 (e.g., a A236C substitution, see e.g., FIG. 3I for numbering) with the cysteines forming a disulfide bond; and/or
   a β2M sequence having an amino terminal epitope and cysteine-containing peptide linker at its amino terminus (e.g., with the linker comprising a cysteine as the second, third fourth, or fifth aa from the epitiope's C-terminal aa) and a MHC-H sequence with a cysteine at position 84 (e.g., a Y84C substitution, see, e.g., FIG. 3I for numbering) with the cysteines forming a disulfide bond.
26. The MAPP of any preceding aspect, wherein the MAPP comprises at least one linker that comprises one or more sequences selected from (e.g., combinations of): polyG (e.g., polyglycine), GA, AG, AS, SA, GS, GSGGS, GGGS, GGSG, GGSGG, GSGSG, GSGGG, GGGSG, GSSSG, GGGGS, or AAAGG, any of which may be repeated 1, 2, 3, 4, 5, 6, 7, 8, 9, or 10 times.
27. The MAPP of any preceding aspect, wherein the MAPP comprises at least one aa sequence independently selected from GCGASGGGGSGGGGS, GCGGSGGGGSGGGGSGGGGS, GCGGSGGGGSGGGGS, and GCGGS(G4S) where the G₄5 unit may be repeated from 1 to 10 times (e.g., repeated 1, 2, 3, 4, 5, 6, 7, 8, 9, or 10 times); wherein the linker cysteine residue optionally forms a disulfide bond (e.g., with another peptide sequence of the MAPP).
28. The MAPP of any preceding aspect, wherein:
   a) the at least one presenting sequence (e.g., two, three, four, or each presenting sequence) comprises (e.g., from N-terminus to C-terminus), either
      (i) the peptide epitope, the β2M sequence, and the MHC-H polypeptide, or
      (ii) the peptide epitope, the MHC-H polypeptide, and the β2M polypeptide (see e.g., FIG. 12); and/or
   b) the at least one presenting complex (e.g., two, three, four, or each presenting complex) comprises
   (i) the presenting complex 1^{st} sequence comprising the MHC-H polypeptide, and its associated presenting complex 2^{nd} sequence comprises the peptide epitope sequence and the β2M polypeptide (e.g., the epitope is placed N-terminal to the β2M) (see e.g., FIG. 13, structures A, C, and E and FIG. 14, structures A, C, and E),
   or
   (ii) the presenting complex 1^{st} sequence comprising (e.g., from N-terminus to C-terminus) the peptide epitope sequence and a β2M sequence (e.g., the epitope is placed N-terminal to the β2M), and the associated presenting complex 2^{nd} sequence comprises the MHC-H polypeptide sequence (see e.g., FIG. 13 structures B, D, and F and FIG. 14. Structures B, D and F), wherein the presenting complex 1^{st} sequence and its associated presenting complex 2^{nd} sequence are optionally joined by at least one disulfide bond (see e.g., FIG. 15);
   wherein any one or more of the peptide epitope, the β2M sequence, and the MHC-H polypeptide are joined by optional linkers; and
   wherein the at least one presenting sequence and/or the at least one presenting complex optionally comprises one or more, or two more independently selected MODs or variant MODs.
29. The MAPP of aspect 28, wherein a linker cysteine residue forms a disulfide bond between a between a presenting sequence and another polypeptide of a MAPP, or between presenting complex 1st sequence and another polypeptide of the MAPP (e.g., with a presenting complex 2nd sequence.
30. The MAPP of any preceding aspect, wherein the dimerization and/or multimerization sequences are independently selected from non-interspecific sequences or interspecific sequences.
31. The MAPP of aspect 30, wherein the interspecific and non-interspecific sequences are selected from the group consisting of: immunoglobulin heavy chain constant regions (Ig Fc e.g., CH2-CH3); collectin polypeptides, coiled-coil domains, leucine-zipper domains; Fos polypeptides; Jun polypeptides; Ig CH1; Ig C_{L} κ; Ig C_{L} λ; knob-in-hole without disulfide (KiH); knob-in hole with a stabilizing disulfide bond (KiHs-s); HA-TF; ZW-1; 7.8.60; DD-KK; EW-RVT; EW-RVTs-s; and A107 sequences.
32. The MAPP of any of any preceding aspect, complexed to form a duplex or higher order MAPP comprising at least a first MAPP heterodimer and a second MAPP heterodimer of any of aspects 1-31, wherein:
   (i) the first heterodimer comprises a first framework polypeptide having a first multimerization sequence and a first dimerization sequence, and a first dimerization polypeptide having first counterpart dimerization sequence complementary to the first dimerization sequence; and
   (ii) the second heterodimer comprises a second framework polypeptide having a second multimerization sequence and a second dimerization sequence, and a second dimerization polypeptide having second counterpart dimerization sequence complementary to the second dimerization sequence; and
   wherein the first and second framework polypeptides are associated by binding interactions between the first and second multimerization sequences optionally including one or more interchain covalent bonds (e.g., one or two disulfide bonds), and the multimerization sequences are not the same (e.g., not the same type and/or not identical to) as, and do not substantially associate with or bind to, the dimerization or counterpart dimerization sequences. See e.g., the duplexes in FIGS. 6 to 10.
33. The duplex MAPP of aspect 32, wherein the first and second dimerization sequence are identical, and the first and second counterpart dimerization sequences are identical. See e.g., FIG. 6 structure A.
34. The duplex MAPP of aspect 33, wherein the first and second dimerization sequences do not substantially associate with or bind to each other.
35. The duplex MAPP of aspect 32, wherein the first and second multimerization sequences are interspecific multimerization sequences that form an interspecific pair, the first and second dimerization sequence are identical, and the first and second counterpart dimerization sequences are identical. See e.g., FIG. 6 structure B.
36. The duplex MAPP of aspect 35, wherein the first and second dimerization sequences do not substantially associate with or bind each other.
37. The duplex MAPP of any of aspects 35 to 36, wherein the first or second framework polypeptide comprises at least one MOD or variant MOD (e.g., two or three MODs or variant MODs) not present on the other framework polypeptide.
38. The duplex MAPP of aspect 32, wherein the first and second multimerization sequences are identical, the first dimerization sequence and the first counterpart dimerization sequence are interspecific dimerization sequences forming a first interspecific pair, and the second dimerization sequence and second counterpart dimerization sequence are interspecific dimerization sequences forming a second interspecific pair. See e.g., FIG. 6 structure C.
39. The duplex MAPP of aspect 38, wherein the first and second dimerization sequences are identical and the first and second counterpart dimerization sequences are identical. See e.g., FIG. 8 structures A.
40. The duplex MAPP of aspect 38, wherein the first and second dimerization sequences are not identical do not bind in any substantial manner to each other.
41. The duplex MAPP of aspect 38, wherein the polypeptides of the first interspecific pair are different from (not identical to), and do not bind or interact with the polypeptides of the second interspecific pair.
42. The duplex MAPP of any of aspects 40 to 41, wherein the first or second dimerization polypeptide comprises at least one MOD (e.g., two or three MODs) not present on the other dimerization polypeptide.
43. The duplex MAPP of aspect 32, wherein
   the first and second multimerization sequences are interspecific multimerization sequences that form an interspecific multimerization pair,
   the first dimerization sequence and the first counterpart dimerization sequence are interspecific dimerization sequences forming a first interspecific pair, and
   the second dimerization sequence and second counterpart dimerization sequence are interspecific dimerization sequences forming a second interspecific pair. See e.g., FIG. 6 structure D.
44. The duplex MAPP of aspect 43, wherein the first and second dimerization sequences are identical and the first and second counterpart dimerization sequences are identical. See e.g., FIG. 8 structure D.
45. The duplex MAPP of aspect 43, wherein the first and second dimerization sequences do not substantially associate with or bind with each other.
46. The duplex MAPP of any of aspects 38 to 45, wherein the polypeptides of the first interspecific pair polypeptides are different from (not identical to), and do not bind or interact with the polypeptides of the second interspecific pair.
47. The duplex MAPP of any of aspects 45 to 46, wherein the first or second dimerization polypeptide comprises at least one MOD or variant MOD (e.g., two or three MODs or variant MODs or variant MODs) not present on the other dimerization polypeptide.
48. The duplex MAPP of any of aspects 43 to 47, wherein the first or second framework polypeptide comprises at least one MOD or variant MOD (e.g., two or three MODs or variant MODs or variant MODs) not present on the other framework polypeptide.
49. The duplex MAPP of any of aspects 32 to 48, wherein, when the multimerization sequences are not an interspecific multimerization pair, the multimerization sequences are selected from the group consisting of immunoglobulin heavy chain constant regions (e.g., IgFc CH2-CH3), collectin family dimerization sequences, coiled-coil domains, and leucine-zipper domains; and wherein, when the multimerization sequences are an interspecific multimerization pair, the multimerization sequences are selected from the group consisting of a Fos and Jun polypeptide pair, Ig CH1 and Ig C_{L} κ or λ constant region polypeptide pair, a knob-in-hole without disulfide (KiH) pair, a knob-in hole with a stabilizing disulfide bond (KiHs-s) pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair.
50. The duplex MAPP of aspect 49, wherein the multimerization sequences, the first dimerization sequence and its counterpart first dimerization sequence, and second dimerization sequence and its counterpart dimerization sequence are each selected from the group consisting of: immunoglobulin heavy chain constant regions (e.g., IgFc CH2-CH3), collectin family dimerization sequences, coiled-coil domains, and leucine-zipper domains; and
   wherein the first and second dimerization sequences, which are selected independently and may be the same or different.
51. The duplex MAPP of aspect 49, wherein the multimerization sequences are selected from the group consisting of immunoglobulin heavy chain constant regions (e.g., Ig CH2-CH3), collectin family dimerization sequences, coiled-coil domains, and leucine-zipper domains;
   wherein a pair comprising the first dimerization sequence and its counterpart dimerization sequence pair, and a pair comprising the second dimerization sequence and it counterpart dimerization sequence, are independently selected from the group consisting of a Fos and Jun polypeptide pair, Ig CH1 and Ig C_{L} κ or λ constant region polypeptide pair, a knob-in-hole without disulfide (KiH) pair, a knob-in hole with a stabilizing disulfide bond (KiHs-s) pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair; and
   wherein the pairs may be the same or different.
52. The duplex MAPP of aspect 49, wherein the multimerization sequences are selected from the group consisting of a Fos and Jun polypeptide pair, Ig CH1 and Ig C_{L} κ or λ constant region polypeptide pair, a knob-in-hole without disulfide (KiH) pair, a knob-in hole with a stabilizing disulfide bond (KiHs-s) pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair;
   wherein the first and second dimerization sequences, which may be the same or different, and their counterpart dimerization sequences are independently selected from the group consisting of immunoglobulin heavy chain constant regions (e.g., Ig CH2-CH3), collectin family dimerization sequences, coiled-coil domains, and leucine-zipper domains.
53. The duplex MAPP of aspect 49, wherein the multimerization sequences, the first dimerization sequence and its counterpart first dimerization sequence, and second dimerization sequence and its counterpart dimerization sequence are each selected as a pair from the group consisting of: Fos and Jun polypeptide pairs, Ig CH1 and Ig C_{L} κ or λ constant region polypeptide pairs, knob-in-hole without disulfide (KiH) pairs, knob-in hole with a stabilizing disulfide bond (KiHs-s) pairs, HA-TF polypeptide pairs, ZW-1 polypeptide pairs, 7.8.60 polypeptide pairs, a DD-KK polypeptide pairs, EW-RVT polypeptide pairs, EW-RVTs-s polypeptide pairs, and A107 polypeptide pairs; and
   wherein the pairs comprising the first and second dimerization sequences may be the same or different.
54. The duplex MAPP of aspect 49, wherein the multimerization sequences comprise Ig Fc regions and the first and second dimerization sequences comprise independently selected Ig CH1, Ig C_{L} κ or λ, leucine zipper, Fos or Jun domains.
55. The duplex MAPP of aspect 49, wherein the multimerization sequences comprise Ig Fc regions and the first and second dimerization sequences comprise independently selected Ig CH1 or Ig C_{L} κ or λ domains.
56. The duplex MAPP of aspect 54 to 55, wherein the Ig CH2-CH3 domains are selected from the group consisting of IgA, IgD, IgE, IgG and IgM Fc regions.
57. The duplex MAPP of aspect 54 to 56, wherein the Ig Fc regions are selected from IgG1, IgG2, IgG3, and IgG4 CH2-CH3 domains.
58. The duplex MAPP of aspect 54 to 56, wherein the Ig Fc regions are IgG1CH2-CH3 domains.
59. The duplex MAPP of aspect 49, wherein the multimerization sequences are a pair of interspecific immunoglobulin sequences.
60. The duplex MAPP of aspect 59, wherein the pair of interspecific immunoglobulin sequences are selected from the group consisting of knob-in-hole without disulfide (KiH) pairs, knob-in hole with a stabilizing disulfide bond (KiHs-s) pairs, HA-TF polypeptide pairs, ZW-1 polypeptide pairs, 7.8.60 polypeptide pairs, a DD-KK polypeptide pairs, EW-RVT polypeptide pairs, EW-RVTs-s polypeptide pairs, and A107 polypeptide pairs
61. The duplex MAPP of aspect 59 or 60, wherein the multimerization sequences are pair of interspecific immunoglobulin sequences comprising a knob-in-hole without disulfide (KiH) pair.
62. The duplex MAPP of aspect 59 or 60, wherein the multimerization sequences are pair of interspecific immunoglobulin sequences comprising a knob-in hole pair that comprises at least one stabilizing disulfide bond (e.g., a KiHs-s pair).
63. The duplex MAPP of any of aspects 59 to 62, wherein the first and second dimerization sequences comprise independently selected Ig CH1, Ig C_{L} κ or λ leucine zipper, Fos or Jun domains.
64. The duplex MAPP of any of aspects 59 to 63, wherein the first and second dimerization sequences comprise independently selected Ig CH1 or Ig CL κ or λ domains.
65. The duplex MAPP of any of aspects 59 to 64, wherein the first and second dimerization sequences comprise Ig CH1 domains.
66. The duplex MAPP of any of aspects 59 to 64, wherein the first and/or second dimerization sequences do not comprise Ig CH1 domains.
67. The MAPPs of any of aspects 1 to 66, wherein the dimerization polypeptide and framework polypeptide are covalently linked by at least one (e.g., two) disulfide bond(s).
68. The duplex MAPPs of any of aspects 32 to 67, wherein the first MAPP heterodimer and/or the second MAPP heterodimer are covalently linked by at least one (e.g., two) disulfide bond(s).
69. The duplex MAPPs of any of aspects 32 to 68, wherein the multimerization sequences of the first and second framework polypeptides are covalently linked by at least one (e.g., two) disulfide bond(s).
70. The duplex MAPPs of any of aspects 32 to 66, wherein the first dimerization sequence and its counterpart dimerization sequence and/or the second dimerization sequence and its counterpart dimerization sequence are covalently linked by at least one (e.g., two) disulfide bond(s); and the multimerization sequences of the first and second framework polypeptides are covalently linked by at least one (e.g., two) disulfide bond(s).
71. The MAPP or duplex MAPP of any preceding aspect, wherein each MAPP comprises only one presenting sequence or one presenting complex. See, e.g., the 1st or 2nd heterodimer in FIG. 1.
72. The MAPP or duplex of aspect 71, wherein the one presenting sequence or the presenting complex 1^{st} sequence of the presenting complex is part of the dimerization polypeptide (e.g., located on the N-terminal side of the counterpart dimerization sequence). See, e.g., the 1st or 2nd heterodimer in FIG. 1.
73. The MAPP or duplex of aspect 71, wherein the one presenting sequence or the presenting complex 1^{st} sequence of the presenting complex is part of the framework polypeptide (e.g., located on the N-terminal side of the dimerization sequence).
74. The duplex MAPP of any of aspects 32-70, wherein the duplex MAPP comprises only one presenting sequence or one presenting complex.
75. The duplex MAPP of aspect 74, wherein the one presenting sequence or the presenting complex 1^{st} sequence of the presenting complex is part of one dimerization polypeptide (e.g., located on the N-terminal side of the counterpart dimerization sequence).
76. The duplex MAPP of aspect 74, wherein the one presenting sequence or the presenting complex 1^{st} sequence of the presenting complex is part of one framework polypeptide (e.g., located on the N-terminal side of the dimerization sequence).
77. The duplex MAPP of any of aspects 32 to 70, wherein the duplex MAPP comprises at least two presenting sequences or at least two presenting complexes. See, e.g., the duplex in FIG. 1.
78. The duplex MAPP of aspect 77, wherein one of the at least two presenting sequences or presenting complex 1^{st} sequences of the at least two presenting complexes is part of the first dimerization polypeptide, and the second of the at least two presenting sequences or presenting complex 1^{st} sequences is part of the second dimerization polypeptides (e.g., located on the N-terminal side of their counterpart dimerization sequences). See, e.g., the duplex in FIG. 1.
79. The duplex MAPP of aspect 77, wherein one of the at least two presenting sequences or each of the presenting complex 1^{st} sequences of the at least two presenting complexes is part of the first framework polypeptide, and the second of the at least two presenting sequences or presenting complex 1^{st} sequences is part of the second framework polypeptide (e.g., located on the N-terminal side of their dimerization sequence).
80. The duplex MAPP of any of aspects 32 to 70, wherein the duplex MAPP comprises at least four presenting sequences and/or presenting complexes. See e.g., FIG. 7 structures A-D.
81. The duplex MAPP of aspect 80, wherein each one of the four presenting sequences or each one of the presenting complex 1^{st} sequences of the four presenting complexes are each part of a different one of the first dimerization polypeptide, second dimerization polypeptide, first framework polypeptide and second framework polypeptide (e.g., located on the N-terminal side of their dimerization sequence or counterpart dimerization sequence). See e.g., FIG. 7 structures A-D.
82. The MAPP or duplex MAPP of any preceding aspect, wherein when a framework or dimerization polypeptides of the MAPP or duplex MAPP comprises one or more IgFc regions, at least one of the one or more IgFc regions comprises one or more substitutions that limit complement activation.
83. The MAPP or duplex MAPP of any preceding aspect, wherein when a framework or dimerization polypeptides of the MAPP or duplex MAPP comprises one or more IgFc regions, at least one of the one or more IgFc regions comprises one or more substitutions at L234, L235, G236, G237, P238, S239, D270, N297, K322, P329, and/or P331 (respectively, aas L14, L15, G16, G17, P18, S19, N77, D50, K102, P109, and P111 of the wt. IgG1 aa sequence in FIG. 4D).
84. The MAPP or the duplex MAPP of aspect 83, the framework or dimerization polypeptides comprises an IgFc region having a substitution at N297 (e.g., N297A).
85. The MAPP or the duplex MAPP of aspect 83, wherein the framework or dimerization polypeptide comprises an IgFc region having a substitution at L234, and/or L235 (e.g., L234A, and/or L235A).
86. The MAPP or the duplex MAPP of aspect 83, wherein the framework or dimerization polypeptide comprises an IgFc region having a substitution at P331 (e.g., P331A or P331S.
87. The MAPP or the duplex MAPP of aspect 83, wherein the framework or dimerization polypeptide comprises an IgFc region having a substitution at: (i) L234, L235, and/or P331 (e.g., L234F, L235E, and P331S), or (ii) D270, K322, and/or P329) (e.g., D270, K322, and/or P329).
88. The MAPP of any of aspects 1 to 32, complexed to form a triplex MAPP of three heterodimers, a quadraplex MAPP of four heterodimers, a pentaplex MAPP of five heterodimers, or hexaplex MAPP of six heterodimers.
89. The MAPP or duplex MAPP of any preceding aspect, comprising: at least one MOD, at least one variant MOD, or at least one pair of MODs and/or variant MODs in tandem, optionally located at one or more of positions 1, 1′, 2, 2′, 3, 3′, 4, 4′, 4″, 4′″, 5, and/or 5′ (see FIGS. 1A and 1B).
90. The duplex MAPP of aspect 89, comprising:
   (a) at least one MOD, at least one variant MOD, or at least one pair of MODs and/or variant MODs in tandem located:
      (i) on the N-terminal side (e.g., at the N-terminus) of at least one framework polypeptide dimerization sequence (see e.g., positions 1 and 1′ in any of FIGS. 6 and 8),
      (ii) on the N-terminal side (e.g., at the N-terminus) of at least one framework polypeptide dimerization sequence and any MHC-H or β2M polypeptide sequences that may be part of the framework polypeptide (see e.g., positions 4″ and 4′″ in FIGS. 7 and 9), and/or
      (iii) on the C-terminal side (e.g., at the C-terminus) of at least one framework polypeptide multimerization sequence (see e.g., positions 3 and 3′ in any of FIGS. 1, 6 to 10); and/or
   (b) at least one MOD, at least one variant MOD, or at least one pair of MODs and/or variant MODs in tandem located:
      (i) on the N-terminal side (e.g., at the N-terminus) of each framework polypeptide dimerization sequence (see e.g., positions 1 and 1′ in any of FIGS. 6 and 8);
      (ii) on the N-terminal side (e.g., at the N-terminus) of each framework polypeptide dimerization sequence and any MHC-H or β2M polypeptide sequences that may be part of the framework polypeptide (see e.g., positions 4″ and 4′″ in FIGS. 7 and 9); and/or
      (iii) on the C-terminal side (e.g., at the C-terminus) of each framework polypeptide multimerization sequence (see e.g., positions 3 and 3′ in any of FIGS. 1 and 6 to 10).
91. The MAPP or duplex MAPP of aspect 90 comprising:
   (i) at least one MOD or variant MOD, or pair of MODs and/or or variant MODs in tandem located on the N-terminal side (e.g., at the N-terminus) of at least one (e.g., each) framework polypeptide dimerization sequence (see e.g., position 1 and 1′ in any of FIGS. 6 and 8), or on the N-terminal side (e.g., at the N-terminus) of at least one (e.g., each) framework polypeptide dimerization sequence and any MHC-H or β2M polypeptide sequences that may be part of the framework polypeptide (see e.g., positions 4″ and 4′″ in FIGS. 7 and 9); and/or
   (ii) at least one MOD or variant MOD, or pair of MODs and/or or variant MODs in tandem located on the C-terminal side (e.g., at the C-terminus) of at least one (e.g., each) framework polypeptide multimerization sequence (see e.g., position 3 and 3′ in any of FIGS. 1, 6 to 10).
92. The MAPP or duplex MAPP of any preceding aspect, comprising: at least one MOD or variant MOD, or pair of MODs and/or or variant MODs in tandem located:
   (i) on the N-terminal side (e.g., at the N-terminus) of at least one (e.g., two or each) dimerization polypeptide counterpart dimerization sequence (see e.g., positions 4 and 4′ in FIGS. 1, and 7 to 9); and/or
   (ii) on the C-terminal side (e.g., at the C-terminus) of at least one (e.g., two or each) dimerization polypeptide counterpart dimerization sequence (see e.g., position 5 and 5′ in any of FIGS. 1 and 6 to 9).
93. The MAPP or duplex MAPP of any of aspects 89 to 92, wherein when the at least one (e.g., two or each) dimerization polypeptide comprises a presenting sequence, the at least one MOD or variant MOD, or pair of MODs and/or or variant MODs in tandem may be located:
   (i) between the counterpart dimerization sequence and the MHC-H, β2M and epitope sequences:
   (ii) between MHC-H and β2M sequences;
   (iii) between the epitope and either the MHC-H or β2M sequence; and/or
   (iv) at the N-terminus of the presenting sequence. See FIG. 12.
94. The MAPP or duplex MAPP of any of aspects 89 to 92, wherein when the dimerization polypeptide comprises a presenting complex, the at least one MOD, or variant MOD, or pair of MODs and/or or variant MODs in tandem may be located:
   (i) between the counterpart dimerization sequence and any of the MHC-H, β2M or epitope sequences present in the presenting complex 1^{st} sequence;
   (ii) at the N-terminus of the presenting complex 1^{st} sequence;
   (iii) at the N-terminus of the presenting complex 2^{st} sequence;
   (iv) between either the MHC-H and epitope sequences, or between the β2M and epitope sequences of the presenting complex 2′ sequence; and/or
   (v) at the C-terminus of the presenting complex 2′ sequence. See e.g., FIGS. 13 to 15.
95. The duplex MAPP of any of any of aspects 32 to 94, comprising: at least one MOD, or variant MOD, or pair of MODs and/or or variant MODs in tandem at position 1 and/or 1′.
96. The duplex MAPP of any of any of aspects 32 to 94, comprising: at least one MOD or variant MOD, or pair of MODs and/or or variant MODs in tandem at position 1 and/or 1′, and at least one MOD or variant MOD, or pair of MODs and/or or variant MODs in tandem at position 3 and/or 3′.
97. The duplex MAPP of any of any of aspects 32 to 94, comprising: at least one MOD or variant MOD, or pair of MODs and/or or variant MODs in tandem at position 1 and/or 1′, and at least one MOD or variant MOD, or pair of MODs and/or or variant MODs in tandem at position 5 and/or 5′.
98. The MAPP or duplex MAPP of any preceding aspect, comprising: at least one (e.g., at least two, or at least three) MOD and/or variant MOD polypeptide sequence is selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL polypeptide sequences, variant MOD polypeptide sequences of any of the foregoing, and anti-CD28.
99. The MAPP or duplex MAPP of any preceding aspect, comprising at least one (e.g., at least two, or at least three) MOD or variant MOD polypeptide sequence is selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAG1 (CD339), CD70, CD80, CD86, TGF-β1, TGF-β2,TGF-β3 polypeptide sequences, variant MOD polypeptide sequences of any of the foregoing, and anti-CD28.
100. The MAPP or duplex MAPP of any preceding aspect, comprising at least one (e.g., at least two, or at least three) MOD or variant MOD polypeptide sequence is selected independently from the group consisting of 4-1BBL, PD-L1 IL-2, CD80, CD86 and FasL MOD or variant MOD polypeptide sequences. For example, the MAPP or duplex MAPP may comprise at least one IL-2 MOD or variant MOD polypeptide sequence, and at least one anti-CD28, CD80, CD86, variant CD80 or variant CD86 polypeptide sequence.
101. The MAPP or duplex MAPP of any preceding aspect, comprising at least one IL-2 MOD or variant MOD polypeptide sequence, or at least one pair of IL-2 MOD or variant MOD polypeptide sequences in tandem (optionally located at position 1 or 1′), and/or at least one CD80 and/or CD86 MOD or variant MOD polypeptide sequence.
102. The MAPP or duplex MAPP of aspect 100, further comprising at least one CD80 and/or CD86 MOD or variant MOD polypeptide sequence, optionally located at position 1 or 1′.
103. The MAPP or duplex MAPP of aspect 100, further comprising at least one PD-L1 MOD or variant MOD polypeptide sequence, optionally located at position 1 or 1′.
104. The MAPP or duplex MAPP of aspect 100, further comprising at least one FasL MOD or variant MOD polypeptide sequence.
105. The MAPP or duplex MAPP of any preceding aspect further comprising an additional peptide, or a payload covalently attached to one or more framework polypeptides and/or dimerization polypeptides.
106. The MAPP or duplex MAPP of aspect 105, wherein the additional peptide is an epitope tag or an affinity domain.
107. The MAPP or duplex MAPP of aspect 105, wherein the additional peptide is a targeting sequence.
108. The MAPP or duplex MAPP of aspect 107, wherein the targeting sequence is an antibody or an antigen binding fragment thereof, or a single chain T cell receptor.
109. The MAPP or duplex MAPP of any of aspects 107 to 108, wherein the targeting sequence is directed to a protein or non-protein epitope of an infectious agent.
110. The MAPP or duplex MAPP of any of aspect 109, were the infectious agent is a viruses, bacteria, fungi, protozoa, and helminths
111. The MAPP or duplex MAPP of any of aspects 107 to 108, wherein the targeting sequence is directed to a self-antigen (autoantigen) or allergen.
112. The MAPP or duplex MAPP of any of aspects 107 to 108, wherein the targeting sequence is directed to a cancer-associated antigen (“CAA”).
113. The MAPP or duplex MAPP of aspect 112, wherein the cancer associated antigen is selected from those recited in Section IV.C.8.c.(i).b.(1) “Cancer Associated Antigens (CAAs).”
114. The MAPP or duplex MAPP of aspect 112, wherein the targeting sequence is selected from an anti-CD51, anti-CD74, anti-CD22, anti-CD20, anti-CD20, anti-CD20, anti-CD38, anti-PD-1 receptor, anti-CTLA-4, anti-TROP-2, anti-mucin, anti-CEA, anti-CEACAM6), anti-colon-specific antigen-p), anti-alpha-fetoprotein, anti-IGF-1R, anti-CD19, anti-PSMA, anti-PSMA dimer, anti-carbonic anhydrase IX, anti-HLA-DR, anti-CD52, anti-EpCAM, anti-VEGF, anti-EGFR, anti-CD33, anti-CD20, anti-EGFR, anti-CD20, anti-HER2, anti-CD79b, anti-BCMA, or anti-mesothelin antibody or antigen binding fragment thereof.
115. The MAPP or duplex MAPP of aspect 112, wherein the targeting sequence is selected from an anti-CD19, anti-HER2, anti-TROP-2, anti-BCMA anti-MUC1, anti-MUC16, anti-claudin-18.2, or anti-mesothelin antibody or antigen binding fragment of any of the foregoing.
116. The MAPP or duplex MAPP of aspect 112, wherein the CAA is a peptide presented by an HLA as a peptide/HLA complex.
117. The MAPP or duplex MAPP of aspect 116, wherein the targeting sequence targets a peptide/HLA recited in Section IV.C.8.c.(i).b.(2) “Peptide/HLA complexes.”
118. The MAPP or duplex MAPP of any of aspects 1 to 117, wherein the epitope is a cancer epitope, infectious agent epitope, self-epitope (autoantigen), or allergen epitope.
119. The MAPP or duplex MAPP any of aspects 1 to 118, wherein the peptide epitope is from about 4 aas (aa) to about 25 aa (e.g., the epitope can have a length of from 4 aa to 10 aa, from about 6 aa to about 12 aa, from 10 aa to 15 aa, from 15 aa to 20 aa, or from 20 aa to 25 aa).
120. The MAPP or duplex MAPP of aspect 119, wherein the peptide epitope is from about 6 aa to about 12 aa.
121. The MAPP or duplex MAPP of any of aspects 118 to 120, wherein the epitope is a cancer epitope
122. The MAPP or duplex MAPP of aspect 121, where the cancer epitope is set forth in section IV.C.7.a.(i) “Cancer Epitopes.”
123. The MAPP or duplex MAPP of aspect 121, where the cancer epitope is an Alpha Feto Protein (AFP) epitope set forth in section IV.C.7.a.(i)(a) “Alpha Feto Protein (AFP)”.
124. The MAPP or duplex MAPP of aspect 121, where the cancer epitope is an epitope of Wilms Tumor Antigen (WT-1) protein set forth in section IV.C.7.a.(i)(b) “Wilms Tumor Antigen (WT-1)”.
125. The MAPP or duplex MAPP of aspect 121, where the cancer epitope is a Human Papilloma Virus I (HPV) epitope set forth in section IV.C.7.a.(i)(c) “Human Papilloma Virus I (HPV)”.
126. The MAPP or duplex MAPP of aspect 121, where the cancer epitope is a Hepatitis B Virus (HBV) epitope set forth in section IV.C.7.a.(i)(d) “Hepatitis B Virus (HBV)”.
127. The MAPP or duplex MAPP of any of aspects 118 to 120, wherein the epitope is a self-epitope.
128. The MAPP or duplex MAPP of any of aspects 118 to 120, wherein the epitope is an epitope of an allergen (e.g., an allergenic protein).
129. The MAPP or duplex MAPP of aspect 128, where the allergen is selected from protein or non-proteins components of: nuts (e.g., tree and/or peanuts), glutens, pollens, eggs (e.g., chicken, Gallus domesticus eggs), shellfish, soy, fish, and insect venoms (e.g., bee and/or wasp venom antigens).
130. The MAPP or duplex MAPP of any of aspects 118 to 120, wherein the epitope is an epitope presented by an infectious agent.
131. The MAPP or duplex MAPP of aspect 130, where the infectious agent is a virus, bacterium, fungi, protozoan, or helminth
132. The MAPP or duplex MAPP of any of aspects 130 to 131, wherein the infectious agent is a virus.
133. The MAPP or duplex MAPP of aspect 132, wherein the epitope is an epitope presented by a viral infectious disease agent set forth in section IV.C.7.a.(ii).
134. A method of treatment or prophylaxis of a disease (e.g., a cancer or infection) or condition (e.g., an allergy), comprising:
   (i) administering to a patient/subject (e.g., a patient in need thereof) an effective amount of one or more MAPPs or duplex MAPPs of any of aspects 1 to 133;
   (ii) administering to a patient/subject (e.g., a patient in need thereof) an effective amount of one or more nucleic acids encoding a MAPP or duplex MAPP according to any of aspects 1 to 133;
   (iii) contacting a cell or tissue in vitro or in vivo with one or more MAPPs or duplex MAPPs of any of aspects 1 to 127 and administering the cell, tissue, or progeny thereof to the patient/subject; or
   (iv) contacting a cell or tissue in vitro or in vivo with one or more nucleic acids encoding a MAPP or duplex MAPP of any of aspects 1 to 133 and administering the cell, tissue, or progeny thereof to the patient/subject.
135. The method of aspect 134, wherein the MAPP or duplex MAPP further comprises at least one targeting sequence (e.g., a targeting sequence specific for an antigen associated with a cell or tissue).
136. The method of any of aspects 134 to 135, wherein the one or more MAPPs or duplex MAPPs are administered to a mammalian patient or subject.
137. The method of any of aspects 134 to 136, wherein the subject is human.
138. The method of any of aspect 134 to 136, wherein the subject non-human (e.g., rodent, lagomorph, bovine, canine, feline, rodent, murine, caprine, simian, ovine, equine, lappine, porcine, etc.).
139. The method any of aspects 134 to 138, wherein the disease or condition is a cancer, and wherein when the one or more MAPPs or duplex MAPPs comprises a targeting sequence it is a CTP.
140. The method of any of aspects 134 to 139, wherein the epitope is a cancer epitope (antigen).
141. The method of any of aspects 134 to 138, wherein the disease or condition is an infection.
142. The method of any of aspects 134 to 138, wherein the disease is a viral infection.
143. The method of any of aspects 134 to 138, wherein the disease is a bacterial, fungal or protozoan infection.
144. The method of any of aspects 134 to 143, further comprising administering one or more therapeutic agents that enhance CD 8+ T cell functions (e.g., effector function) and/or treats the disease or condition before, during (concurrent or combined administration) or after administering the one or more MAPP, duplexed MAPP or one or more nucleic acids encoding the one or more MAPPs or duplexed MAPPs.
145. The method of aspect 144, wherein the therapeutic agent that enhances CD 8+ function and/or treats the disease or condition comprises an anti-TGF-β antibody such as Metelimumab (CAT192) directed against TGF-β1 and Fresolimub directed against TGF-β1 and TGF-β2, or a TGF-β trap (subject to the proviso that the MAPP or duplexed MAPP does not comprise an aa sequence to which the antibodies or TGF-β trap bind such as a TGF-β1 or TGF-β2 MOD or variant MOD aa sequence).
146. The method of aspect 144 to 145, wherein the therapeutic agent that enhances CD 8+ function and/or treats the disease or condition comprises one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab); CD38 (e.g., daratumumab); SLAMF7 (Elotuzumab); both EpCAM and CD3 (e.g., catumaxomab); or both CD19 and CD3 (blinatumomab) (subject to the proviso that the MAPP or duplexed MAPP does not comprise a aa sequence to which the antibodies bind).
147. The method of any of aspects 134 to 146, further comprising administering one or more chemotherapeutic agents antibiotics, chemotherapeutics, antifungals, and/or anti-helminths.
148. The method of aspect 147, where the disease is a cancer and method further comprises administering one or more chemotherapeutic agents.
149. The method of aspect 148, wherein the one or more chemotherapeutic agents are selected from: alkylating agents, cytoskeletal disruptors (taxane), epothilones, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids and their derivatives.
150. The method of aspect 148, wherein the one or more chemotherapeutic agents are selected from the group consisting of actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.
151. The method of any of aspects 134 to 138, further comprising administering one or more therapeutic agents that suppress CD 8+ T cell functions (e.g., suppress effector function), suppress immune response, and/or treat the disease or condition before, during (concurrent or combined administration) or after administering the one or more MAPP, duplexed MAPP or one or more nucleic acids encoding the one or more MAPPs or duplexed MAPPs.
152. The method of aspect 151, wherein the disease or condition is an autoimmune disease and the epitope is an autoantigen (self-epitope).
153. The method of aspect 151, wherein the disease or condition is an allergy and the epitope is an allergen.
154. The method of any of aspects 151 to 153, further comprising administering an NSAID (e.g., Cox-1 and/or Cox-2 inhibitors such as celecoxib, diclofenac, diflunisal, etodolac, ibuprofen, indomethacin, ketoprofen, and naproxen).
155. The method of any of aspects 151 to 154, further comprising administering a corticosteroid (e.g., cortisone, dexamethasone, hydrocortisone, ethamethasoneb, fludrocortisone, methylprednisolone, prednisone, prednisolone and triamcinolone) before, during (concurrent or combined administration) or after administering the one or more masked TGF-β constructs or complexes.
156. The method of any of aspects 151 to 155, further comprising administering an agent that blocks one or more actions of tumor necrosis factor alpha (e.g., an anti-TNF alpha such as golimumab, infliximab, certolizumab, adalimumab or a TNF alpha decoy receptor such as etanercept) (subject to the proviso that the MAPP or duplexed MAPP does not comprise tumor necrosis factor alpha MOD or variant MOD and/or an aa sequence to which the agent binds).
157. The method of any of aspects 151 to 156, further comprising administering one or more agents that bind to the IL-1 receptor competitively with IL-1 (e.g., anakinra) (subject to the proviso that the MAPP or duplexed MAPP does not comprise an IL-1 MOD or variant MOD and/or an aa sequence to which the agent binds).
158. The method of any of aspects 151 to 157, further comprising administering one or more agents that bind to the IL-6 receptor and inhibits IL-6 from signaling through the receptor (e.g., tocilizumab) subject to the proviso that the MAPP or duplexed MAPP does not comprise an IL-6 MOD or variant MOD and/or an aa sequence to which the agent binds).
159. The method of any of aspects 151 to 158, further comprising administering one or more agents that bind to CD80 and/or CD86 receptors and inhibit T cell proliferation and/or B cell immune response (e.g., abatacept) (subject to the proviso that the MAPP or duplexed MAPP does not comprise a CD80 and/or CD86 MOD or variant MOD and/or an aa sequence to which the agent binds).
160. The method of any of aspects 151 to 159, further comprising administering one or more agents that bind to CD20 resulting in B-cell death (e.g., rituximab) (subject to the proviso that the MAPP or duplexed MAPP does not comprise a CD20 MOD or variant MOD, and/or an aa sequence to which the agent binds).
161. The method of any of aspects 134 to 160, wherein the MAPP or duplex MAPP, or the nucleic acid encoding a MAPP or duplex MAPP is administered in a composition comprising the MAPP or duplex MAPP and at least one pharmaceutical acceptable excipient.
162. A framework polypeptide of a MAPP or duplex MAPP according to any of aspects 1 to 133, optionally comprising an additional polypeptide.
163. A dimerization polypeptide of a MAPP or duplex MAPP, according to any of aspects 1 to 133, optionally comprising an additional polypeptide.
164. A nucleic acid sequence encoding the framework polypeptide of any of aspects 1 to 133, wherein the framework polypeptide optionally comprises an additional polypeptide.
165. A nucleic acid sequence encoding the dimerization polypeptide of any of aspects 1 to 133, wherein the dimerization polypeptide optionally comprises an additional polypeptide.
166. One or more nucleic acids comprising a nucleic acid sequence encoding a MAPP or duplex MAPP according to any of aspects 1 to 133.
167. The nucleic acid of any of aspects 164 to 166, wherein the nucleic acid sequence encoding the framework polypeptide and/or the dimerization polypeptide are operably linked to a promoter.
168. A method of producing cells expressing a MAPP or duplex MAPP, the method comprising introducing one or more nucleic acids according to aspect 167 into the cells in vitro or in vivo; selecting for cells that produce the MAPP or duplex MAPP; and optionally selecting for cells comprising all or part of the one or more nucleic acids either unintegrated or integrated into at least one cellular chromosome.
169. The method of aspect 168, wherein the cell is a cell of a mammalian cell line selected from the HeLa cells, CHO cells, 293 cells, Vero cells, NIH 3T3 cells, Huh-7 cells, BHK cells, PC12, COS cells, COS-7 cells, RAT1 cells, mouse L cells, human embryonic kidney (HEK) cells, and HLHepG2 cells.
170. A cell transiently or stably expressing a MAPP or duplex MAPP prepared by the method of aspect 168 or 169.
171. The cell of aspect 170, wherein cells express from about 25 to about 350 (e.g., 20-50, 50-100, 100-200, 200-300, 300-350) mg/liter or more of the MAPP or duplex MAPP without a substantial reduction (e.g., less than a 5%, 10%, or 15% reduction) in cell viability relative to otherwise identical cells not expressing the MAPP or duplex MAPP.
172. A method of selectively delivering one or more MOD polypeptides and/or variant MOD polypeptides to a cell, tissue, patient or subject, the method comprising:
   (i) administering to a patient/subject (e.g., a patient in need thereof) an effective amount of one or more MAPPs or duplex MAPPs of any of aspects 1 to 133;
   (ii) administering to a patient/subject (e.g., a patient in need thereof) an effective amount of one or more nucleic acids encoding a MAPP or duplex MAPP according to any of aspects 1 to 133;
   (iii) contacting a cell or tissue in vitro or in vivo with one or more MAPPs or duplex MAPPs of any of aspects 1 to 133 and optionally administering the cell, tissue, or progeny thereof to the patient/subject; or
   (iv) contacting a cell or tissue in vitro or in vivo with one or more nucleic acids encoding one or more MAPPs or duplex MAPPs of any of aspects 1 to 133 and optionally administering the cell, tissue, or progeny thereof to the patient/subject;
   wherein the one or more MAPPs or duplex MAPPs comprise one or more MODs and/or variant MODs.
173. The method of aspect 172, wherein the one or more MOD polypeptides and/or variant MOD polypeptides are selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAG1 (CD339), CD70, CD80, CD86, TGF-β1, TGF-β2, TGF-β3 MOD, variant MOD polypeptide sequences of any of the foregoing, and anti-CD28.
174. The method of aspect 172, wherein the one or more MOD polypeptides and/or variant MOD polypeptides are selected independently from the group consisting of: 4-1BBL, PD-L1 IL-2, CD80, CD86 and FasL MOD and variant MOD polypeptide sequences of any thereof.
175. The method of aspect 172, wherein the MAPP or duplex MAPP comprises at least one IL-2 MOD or variant MOD polypeptide sequence, and at least one CD80, CD86, variant CD80 or variant CD86 polypeptide sequence.
176. The method of aspect 172, wherein the MAPP or duplex MAPP comprises at least one IL-2 MOD or variant MOD polypeptide sequence, or at least one pair of IL-2 MOD or variant MOD polypeptide sequences in tandem.
177. The method of aspect 172, wherein the MAPP or duplex MAPP comprises at least one CD80 and/or CD86 MOD or variant MOD polypeptide sequence.
178. The method of aspect 172, wherein the MAPP or duplex MAPP comprises at least one PD-L1 MOD or variant MOD polypeptide sequence.
179. The method of aspect 172, wherein the MAPP or duplex MAPP comprises at least one FasL MOD or variant MOD polypeptide sequence.

### VI. EXAMPLES

### Example 1

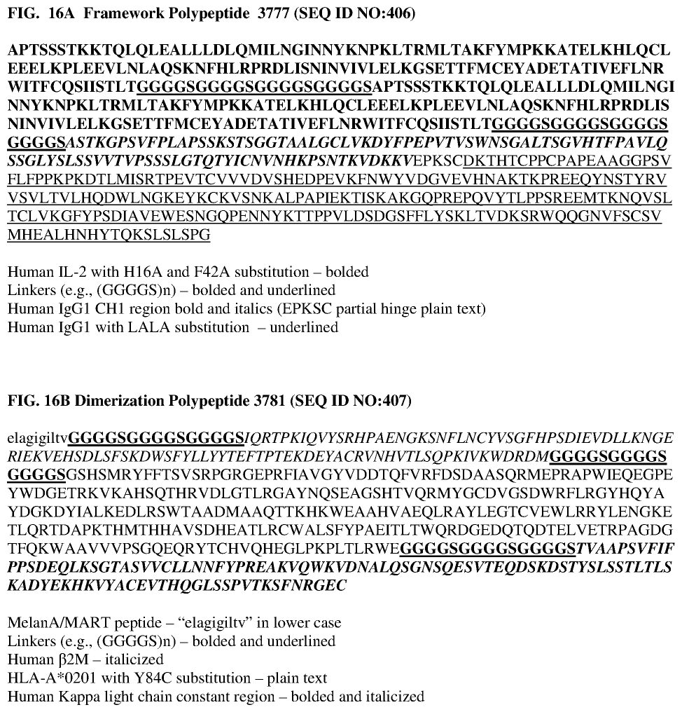

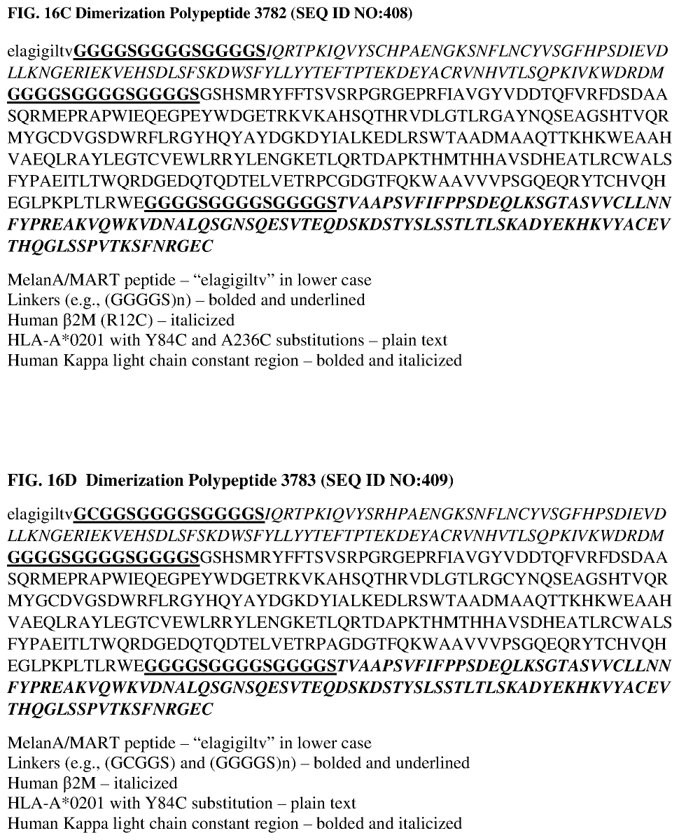

Example 1 illustrates three related MAPP heterodimer constructs that have multimerized into duplexes with the overall structures given in FIG. 11 as structures A, B, C, and D. Each of the heterodimers share a common framework polypeptide that comprises human IgG heavy chain constant region (CH2-CH3 domain) multimerization sequences with a LALA substitution and a CH1 dimerization sequence provide in FIG. 16A. Three dimerization polypeptides that can form heterodimers with the framework polypeptide are provided in FIGS. 16B, 16C, and 16D. The heterodimers each comprise a framework and dimerization polypeptide and pair to form duplex MAPP structures through interactions between IgG heavy elements in each of the framework polypeptides as shown in FIG. 11 structures A, B, C, and D.

FIG. 16A, construct 3777, provides the aa sequence and location of elements of the framework polypeptide, which comprises from N-terminus to C-terminus: IL-2 leader sequence; tandem IL-2 sequences each having H16A, F42A substitutions separated by a linker comprising four repeats of GGGGS; four repeats of the linker sequence GGGGS; a human IgG1 CH1 domain that acts as dimerization sequence, and a human IgG1 Fc region comprising “LALA” substitutions that acts as a multimerization sequence.

FIG. 16B, construct 3781, provides the aa sequence and location of elements in a dimerization polypeptide that comprises, from N-terminus to C-terminus: a peptide epitope (melanocyte-melanoma tumor-antigen or “MART” peptide ELAGIGILTV shown in lower case letters); three repeats of the linker sequence GGGGS, a human β2M polypeptide sequence; three repeats of the linker sequences GGGGS; a human HLA-A*0201 (HLA-A*02:01) polypeptide comprising a Y84A substitution; three repeats of the linker sequences GGGGS; and a human IgG κ light chain constant region dimerization sequence.

FIG. 16C, construct 3782, provides the aa sequence and location of elements in a dimerization polypeptide that comprises, from N-terminus to C-terminus: a peptide epitope (melanocyte-melanoma tumor-antigen or “MART” peptide ELAGIGILTV (SEQ ID NO:546) shown in lower case letters); three repeats of the linker sequence GGGGS, a human β2M polypeptide sequence comprising an R12C substitution; three repeats of the linker sequences GGGGS; a human HLA-A*0201 (HLA-A*02:01) polypeptide comprising Y84A and A236C substitutions; three repeats of the linker sequences GGGGS; and a human IgG κ light chain constant region dimerization sequence.

FIG. 16D, construct 3783, provides the aa sequence and location of elements in a dimerization polypeptide that comprises, from N-terminus to C-terminus: a peptide epitope (melanocyte-melanoma tumor-antigen or “MART” peptide ELAGIGILTV shown in lower case letters); a linker comprising the sequence GCGGS followed by two repeats of the sequence GGGGS, a human β2M polypeptide sequence; three repeats of the linker sequences GGGGS; a human HLA-A*0201 (HLA-A*02:01) polypeptide comprising a Y84A substitution; three repeats of the linker sequences GGGGS; and a human IgG κ light chain constant region dimerization sequence.

FIG. 11 structure A shows a duplex MAPP formed from constructs 3777 and 3781. FIG. 11 structure B shows a duplex MAPP formed from constructs 3777 and 3782 with an additional disulfide bond between the R12C cysteine of the β2M polypeptide and the Y84C cysteine of the HLA-A*02:01 sequence. FIG. 11 structure C shows a duplex MAPP formed from constructs 3777 and 3783. FIG. 11 structure D shows modification of structure C in which the MHC-H sequence has a internal Y84C A139C disulfide bond (denoted Y84C-SS-A139C), and the same substitutions may be made in structures A and B. Structures A and C of FIG. 11 may also include a disulfide bond between position 84 of the MHC-H (Y84C) and a cysteine the linker between the the epitope and β2M polypeptide.

The three duplex MAPPs in FIG. 11 are prepared by cellular expression of the framework and dimerization sequences in Expi-CHO cells by transient transfection using an expression vector containing a nucleic acid construct encoding the polypeptides. The assembled duplex MAPPs are purified over Protein A (MabSelect SuRe™; GE), followed by further purification by size exclusion chromatography.

Testing, conducted by contacting the MAPPs with a population of PBMCs obtained from one or more individuals that have not been exposed to the MART peptide epitope, demonstrates an expansion in the number of MART specific T cells as demonstrated by MART-tetramer staining In contrast, exposure of PBMC to MAPPs bearing an unrelated epitope does not increase MART specific T cell numbers.

### Example 2

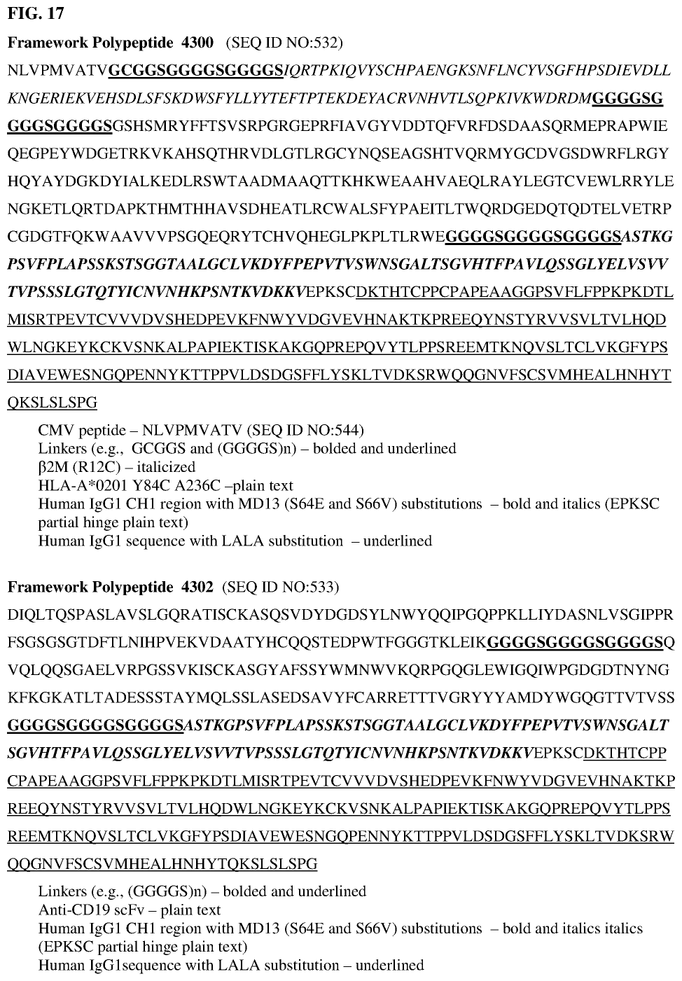

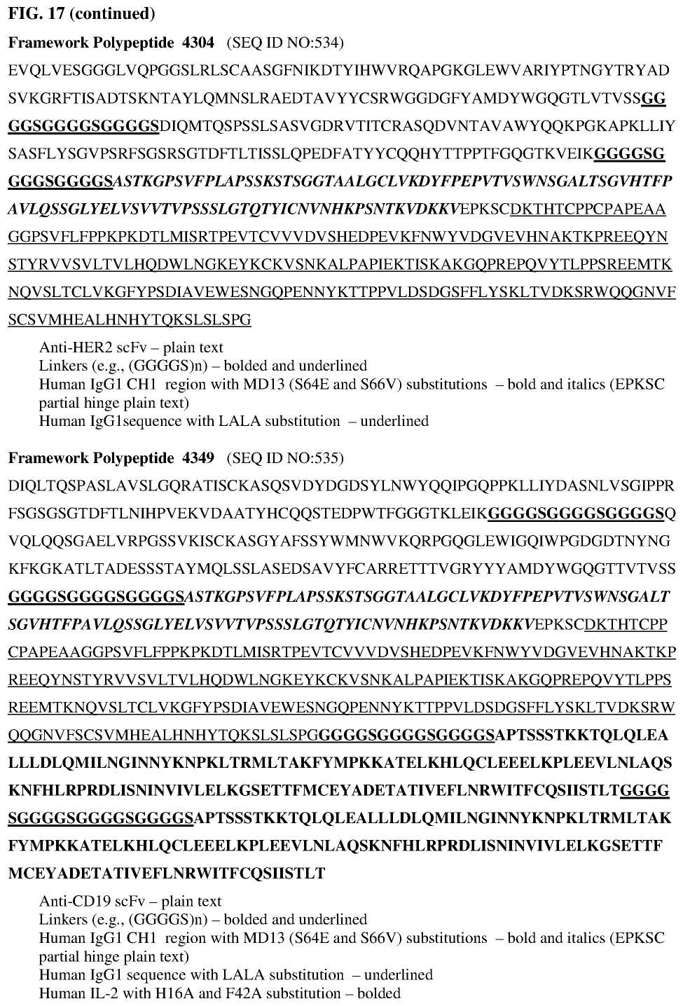

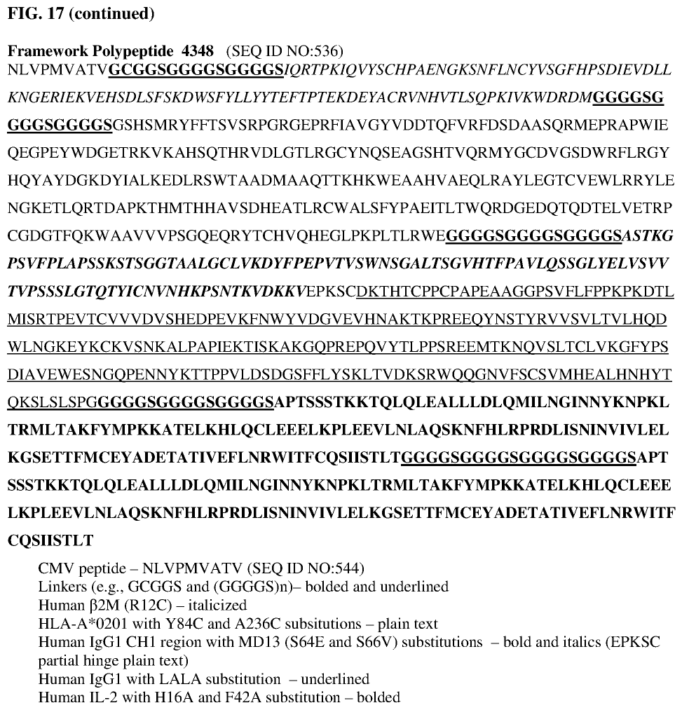

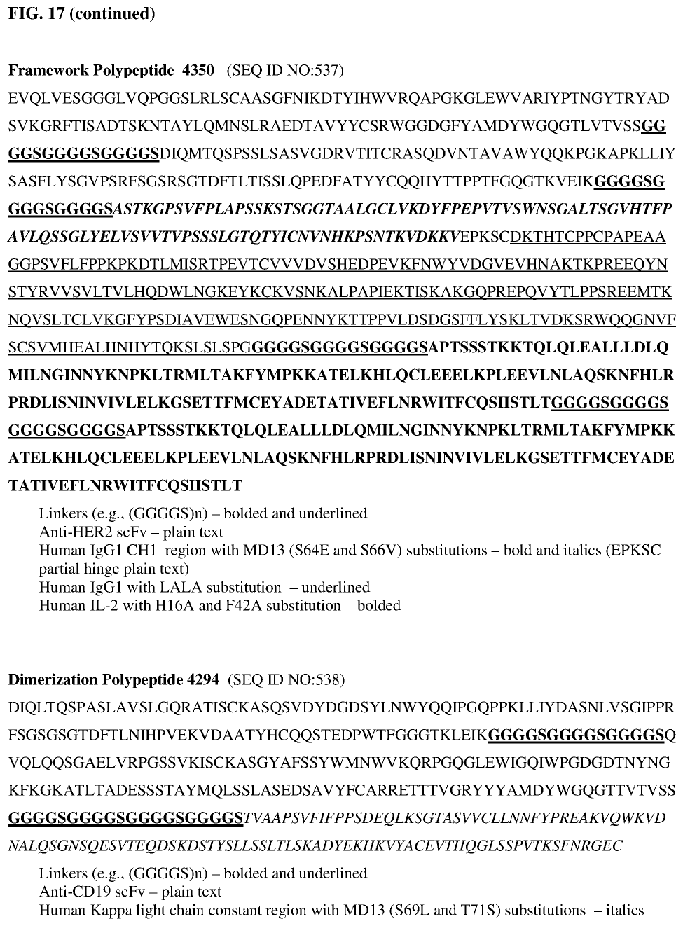

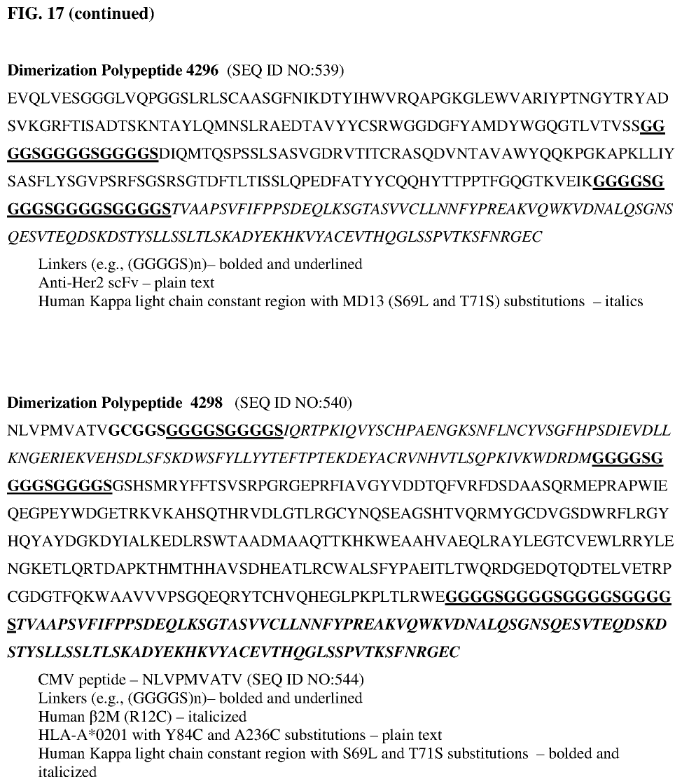

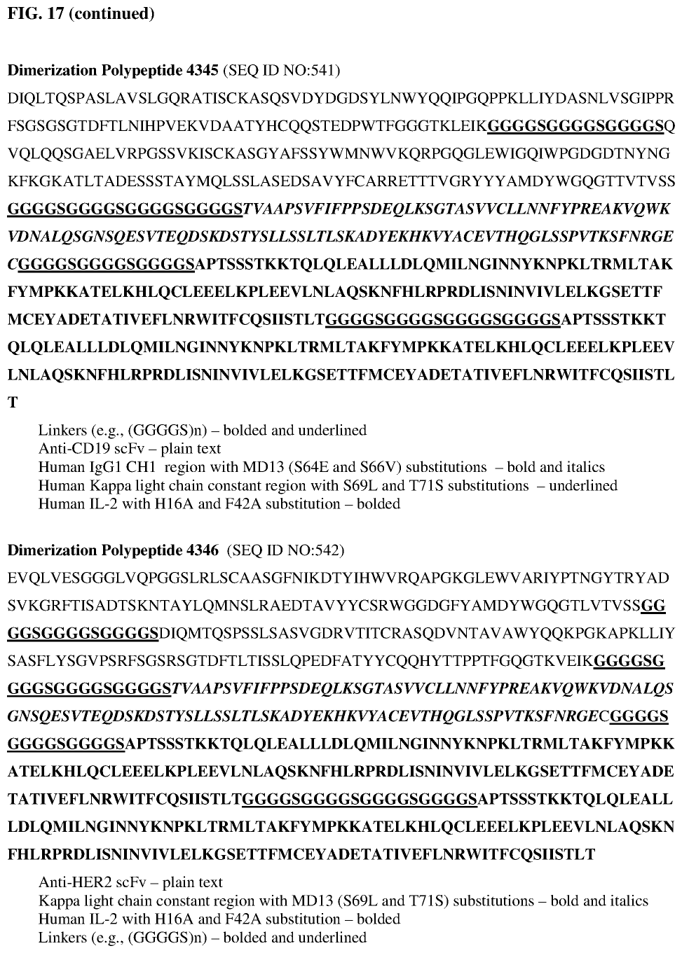

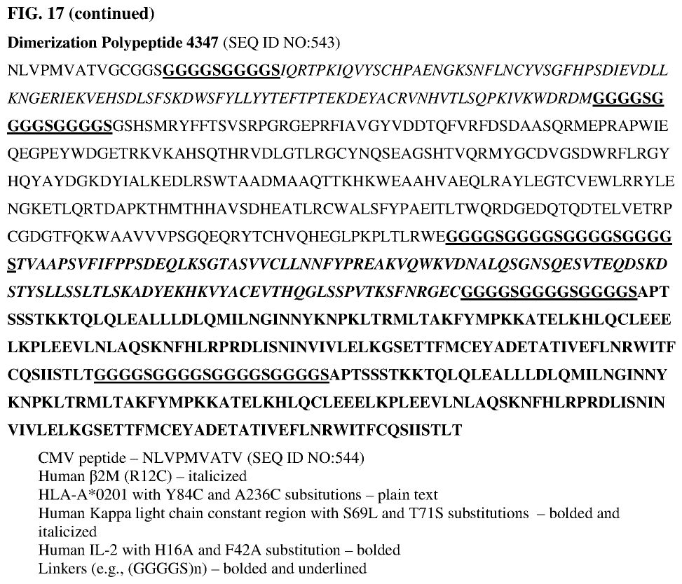

Example 2 illustrates eight MAPP constructs that multimerized into duplexes due to interactions between antibody Fc multimerization sequences in the framework polypeptides, with the dimerization sequences pairing through CH1-Cκ light chain interactions. The constructs are made from six different framework polypeptides—4300, 4348, 4302, 4304, 4349, and 4550, that pair with one of six dimerization poly peptides—4345, 4346, 4294, 4296, 4347, and 4298. The sequences of those polypeptides are provided in FIG. 17.

Construct 1 comprises two 4300 framework polypeptides and two 4345 dimerization polypeptides.

Construct 2 comprises two 4300 framework polypeptides and two 4346 dimerization polypeptides.

Construct 3 comprises two 4348 framework polypeptides and two 4294 dimerization polypeptides.

Construct 4 comprises two 4348 framework polypeptides and two 4296 dimerization polypeptides.

Construct 5 comprises two 4302 framework polypeptides and two 4347 dimerization polypeptides.

Construct 6 comprises two 4304 framework polypeptides and two 4347 dimerization polypeptides.

Construct 7 comprises two 4349 framework polypeptides and two 4298 dimerization polypeptides.

Construct 8 comprises two 4350 framework polypeptides and two 4298 dimerization polypeptides.

1. A multimeric antigen-presenting polypeptide complex (MAPP) comprising:
(i) a framework polypeptide comprising from N-terminus to C-terminus a dimerization sequence and a multimerization sequence;
(ii) a dimerization polypeptide comprising a counterpart dimerization sequence complementary to the dimerization sequence of the framework polypeptide, and dimerizing therewith through covalent and/or non-covalent interactions to form a MAPP heterodimer, and
(iii) at least one presenting sequence;
wherein
(a) each presenting sequence comprises an epitope, MHC Class I heavy chain (“MHC-H”), and β2M polypeptide sequences,
(b) one or both of the dimerization polypeptide or the framework polypeptide comprise a presenting sequence located on the N-terminal side of the framework polypeptide's dimerization sequence, or the N-terminal side of the dimerization polypeptide's counterpart dimerization sequence, and
(c) at least one of the framework polypeptide, dimerization peptide, and/or presenting sequence(s) comprises one, two, three or more independently selected MOD and/or variant MOD polypeptide sequences; and
wherein the framework polypeptide, dimerization polypeptide, and/or presenting sequence optionally comprise one or more linker sequences selected independently.

2. A multimeric antigen-presenting polypeptide complex (MAPP) comprising:
(i) a framework polypeptide comprising a dimerization sequence and a multimerization sequence,
(ii) a dimerization polypeptide comprising a counterpart dimerization sequence complementary to the dimerization sequence of the framework polypeptide, and dimerizing therewith through covalent and/or non-covalent interactions to form a MAPP heterodimer, and
(iii) at least one presenting complex;
wherein
(a) each presenting complex comprises a presenting complex 1^{st} sequence and a presenting complex 2^{nd} sequence that together comprise epitope, MHC-H, and β2M polypeptide sequences (the epitope is part of the presenting complex 1^{st} sequence or presenting complex 2^{nd} sequence along with either the MHC-H or β2M polypeptide sequence),
(b) one or both of the dimerization polypeptide and/or the framework polypeptide comprise a presenting complex 1^{st} sequence located on the N-terminal side of the framework polypeptide's dimerization sequence, or the N-terminal side of the dimerization polypeptide's counterpart dimerization sequence, and
(c) at least one of the framework polypeptide, dimerization peptide, and presenting complex(s) 1^{st} sequence and/or presenting complex 2^{nd} sequence comprises one, two, three or more independently selected MOD and/or variant MOD polypeptide sequences; and
wherein the framework polypeptide, dimerization polypeptide, presenting complex 1^{st} sequence and/or presenting complex 2^{nd} sequence optionally comprise one or more linker sequences selected independently.

3. The MAPP of any preceding claim, wherein the MHC-H polypeptide sequence comprises a human Class I MHC-H chain polypeptide sequence selected from a HLA-A, HLA-B, HLA-C, HLA-E, HLA-F, and HLA-G MHC-H polypeptide sequences or a portion thereof.

4. The MAPP of claim 3, wherein at least one presenting sequence or presenting complex comprises:
a MHC-H sequence having 90% to 100% or 95% to 100% sequence identity to at least 175 contiguous aas of a MHC-H polypeptide provided in any of SEQ ID NOs: 6-53 (provided in FIGS. 3A to 3H), wherein the MHC-H sequences do not include the MHC-H transmembrane domain, or a portion thereof, that will anchor the MAPP in a cell membrane; and/or
a β2M sequence having 90% to 100% or 95% to 100% sequence identity to at least 60 contiguous aas of a mature β2M polypeptide in any of SEQ ID NOs:1-5 (provided in FIG. 2).

5. The MAPP of claim 4, wherein the MHC-H polypeptide sequences have 90% to 100% or 95% to 100% sequence identity to at least 175 contiguous aas of a HLA-A*0101, HLA-A*0201, HLA-A*0301, HLA-A*1101, HLA-A*2301, HLA-A*2402, HLA-A*2407, HLA-A*3303, or HLA-A*3401 (SEQ ID NOs:23-31, respectively).

6. The MAPP of claim 3, wherein the MHC-H polypeptide sequences have 90% to 100% or 95% to 100% sequence identity to at least 175 contiguous aas of a HLA-B allele that does not include the MHC-H transmembrane domain or a portion thereof that will anchor the MAPP in a cell membrane.

7. The MAPP of claim 6, wherein the MHC-H polypeptide sequences have 90% to 100% or 95% to 100% sequence identity to at least 175 contiguous aas of a HLA-B*0702, HLA-B*0801, HLA-B*1502, HLA-B*3802, HLA-B*4001, HLA-B*4601, or HLA-B*5301 polypeptide sequences (SEQ ID NOs: 33 to 39, respectively).

8. The MAPP of claim 4, wherein the MHC-H polypeptide sequences have 90% to 100% or 95% to 100% sequence identity to at least 175 contiguous aas of a HLA-C allele that does not include the MHC-H transmembrane domain or a portion thereof that will anchor the MAPP in a cell membrane.

9. The MAPP of claim 8, wherein the MHC-H polypeptide sequences have 90% to 100% or 95% to 100% sequence identity to at least 175 contiguous aas of a HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence (SEQ ID NOs:41-49, respectively).

10. The MAPP of claim 4, wherein the MHC-H polypeptide sequence has 90% to 100% or 95% to 100% sequence identity to at least 175 contiguous aas of a HLA-E allele that does not include the MHC-H transmembrane domain or a portion thereof that will anchor the MAPP in a cell membrane.

11. The MAPP of claim 4, wherein the MHC-H polypeptide sequences have at least 90% to 100% sequence identity to at least 175 contiguous aas of a HLA-F or HLA G allele that does not include the MHC-H transmembrane domain or a portion thereof that will anchor the MAPP in a cell membrane.

12. The MAPP of any of claims 4-11, wherein the at least 90% to 100% or 95% to 100% sequence identity is at least 98% to 100% sequence identity.

13. The MAPP of claim 4, wherein:
a) the at least one presenting sequence comprises from N-terminus to C-terminus, either
(i) the peptide epitope, the β2M sequence, and the MHC-H polypeptide, or
(ii) the peptide epitope, the MHC-H polypeptide, and the β2M polypeptide; and/or
b) the at least one presenting complex comprises
(i) the presenting complex 1^{st} sequence comprising the MHC-H polypeptide, and its associated presenting complex 2^{nd} sequence comprises the peptide epitope sequence and the β2M polypeptide,
or
(ii) the presenting complex 1^{st} sequence comprising from N-terminus to C-terminus the peptide epitope sequence and a β2M sequence, and the associated presenting complex 2^{nd} sequence comprises the MHC-H polypeptide sequence,
wherein the presenting complex 1st sequence and its associated presenting complex 2nd sequence are optionally joined by at least one disulfide bond;
wherein any one or more of the peptide epitope, the β2M sequence, and the MHC-H polypeptide are joined by optional linkers; and
wherein the at least one presenting sequence and/or the at least one presenting complex optionally comprises one or more, or two more independently selected MODs or variant MODs.

14. The MAPP of claim 4, wherein the dimerization and/or multimerization sequences are independently selected from non-interspecific sequences or interspecific sequences.

15. The MAPP of claim 14, wherein the interspecific and non-interspecific sequences are selected from the group consisting of: immunoglobulin heavy chain constant regions (Ig Fc e.g., CH2-CH3);
collectin polypeptides, coiled-coil domains, leucine-zipper domains; Fos polypeptides; Jun polypeptides; Ig CH1; Ig C_{L} Ig C_{L} knob-in-hole without disulfide (KiH); knob-in hole with a stabilizing disulfide bond (KiHs-s); HA-TF; ZW-1; 7.8.60; DD-KK; EW-RVT; EW-RVTs-s; and A107 sequences.

16. The MAPP of claim 14, complexed to form a duplex or higher order MAPP comprising at least a first MAPP heterodimer and a second MAPP heterodimer, wherein:
(i) the first heterodimer comprises a first framework polypeptide having a first multimerization sequence and a first dimerization sequence, and a first dimerization polypeptide having a first counterpart dimerization sequence complementary to the first dimerization sequence; and
(ii) the second heterodimer comprises a second framework polypeptide having a second multimerization sequence and a second dimerization sequence, and a second dimerization polypeptide having a second counterpart dimerization sequence complementary to the second dimerization sequence; and
wherein the first and second framework polypeptides are associated by binding interactions between the first and second multimerization sequences optionally including one or more interchain disulfide bonds, and the multimerization sequences are not the same as the dimerization or counterpart dimerization sequences.

17. The duplex MAPP of claim 16, comprising: at least one MOD, at least one variant MOD, or at least one pair of MODs and/or variant MODs in tandem, optionally located at one or more of positions 1, 1′, 2, 2′, 3, 3′, 4, 4′, 4″, 4′″, 5, and/or 5′.

18. The duplex MAPP of claim 17, comprising at least one MOD, at least one variant MOD, or at least one pair of MODs and/or variant MODs in tandem, located:
(i) on the N-terminal side of each framework polypeptide dimerization sequence (position 1 or 1′);
(ii) on the N-terminal side of each framework polypeptide dimerization sequence and any MHC-H or β2M polypeptide sequences that may be part of the framework polypeptide (positions 4″ or 4′″); and/or
(iii) on the C-terminal side (e.g., at the C-terminus) of each framework polypeptide multimerization sequence (positions 3 or 3′).

19. The duplex MAPP of claim 17, comprising at least one MOD and/or variant MOD polypeptide sequence selected independently from the group consisting of 4-1BBL, PD-L1 IL-2, CD80, CD86 and FasL MOD or variant MOD polypeptide sequences.

20. The duplex MAPP of claim 19, comprising at least one IL-2 MOD and/or variant IL-2 MOD polypeptide sequence, wherein the variant IL-2 MOD optionally comprises an H16 and/or F42 substitution.

21. The duplex MAPP of claim 17, wherein the epitope peptide is from about 6 aa to about 12 aa.

22. The duplex MAPP of claim 21, wherein the epitope is a cancer epitope, infectious agent epitope, self-epitope (autoantigen), or allergen epitope.

23. The MAPP or duplex MAPP of claim 22, wherein the epitope is a cancer epitope, and wherein, when a targeting sequence is present, it is directed to a cancer associated antigen.

24. The MAPP or duplex MAPP of claim 23, wherein the cancer epitope is an Alpha Feto Protein (AFP) epitope; a Wilms Tumor Antigen (WT-1) protein epitope; a Human Papilloma Virus I (HPV) epitope; or a Hepatitis B Virus (HBV) epitope.

25. The MAPP or duplex MAPP of claim 22, wherein the epitope is a self-epitope, an epitope of an allergen, or an epitope presented by an infectious agent.

26. A method of treatment or prophylaxis of a disease or a condition comprising:
(i) administering to a patient/subject (e.g., a patient in need thereof) an effective amount of one or more duplex MAPPS according to claim 17;
(ii) administering to a patient/subject (e.g., a patient in need thereof) an effective amount of one or more nucleic acids encoding one or more duplex MAPPs according to claim 17;
(iii) contacting a cell or tissue in vitro or in vivo with one or more duplex MAPPs according to claim 17 and administering the cell, tissue, or progeny thereof to the patient/subject; or
(iv) contacting a cell or tissue in vitro or in vivo with one or more nucleic acids encoding one or more MAPPs or duplex MAPPs according to claim 17, and administering the cell, tissue, or progeny thereof to the patient/subject.

27. The method of claim 26, wherein the duplex MAPP comprises at least one targeting sequence.

28. The method of claim 26, wherein the one or more MAPPs or duplex MAPPs are administered to a mammalian patient or subject.

29. The method of claim 28, wherein the disease or condition is a cancer, the epitope is a cancer epitope, and wherein when the duplex MAPPs comprise a targeting sequence it is a CTP.

30. The method of claim 26, wherein the disease or condition is a viral, bacterial, fungal or protozoan infection.

31. A framework polypeptide or a dimerization polypeptide of a duplex MAPP according to any of claims 1 to 2, wherein the framework or dimerization polypeptide optionally comprises an additional polypeptide.

32. A nucleic acid sequence encoding the framework polypeptide or a dimerization polypeptide of any of claims 1 to 2, wherein the framework or dimerization polypeptide optionally comprises an additional polypeptide.

33. One or more nucleic acids comprising a nucleic acid sequence encoding a MAPP according to any of claims 1 to 2, wherein the MAPP optionally forms a duplex or other higher order complex.

34. A method of producing cells expressing a duplex MAPP, the method comprising introducing one or more nucleic acids according to claim 33 into the cells in vitro; selecting for cells that produce the duplex MAPP; and optionally selecting for cells comprising all or part of the one or more nucleic acids either unintegrated or integrated into at least one cellular chromosome.

35. A cell transiently or stably expressing a duplex MAPP prepared by the method of claim 34, wherein cells express from about 25 to about 350 mg/liter or more of the duplex MAPP without a substantial reduction in cell viability relative to otherwise identical cells not expressing the duplex MAPP.

36. A method of selectively delivering one or more MOD polypeptides and/or variant MOD polypeptides to a cell, tissue, patient or subject, the method comprising:
(i) administering to a patient/subject (e.g., a patient in need thereof) an effective amount of one or more duplex MAPPs according to claim 17;
(ii) administering to a patient/subject an effective amount of one or more nucleic acids encoding a duplex MAPP according to claim 17;
(iii) contacting a cell or tissue in vitro or in vivo with one or more duplex MAPPs according to claim 17, and optionally administering the cell, tissue, or progeny thereof to the patient/subject; or
(iv) contacting a cell or tissue in vitro or in vivo with one or more nucleic acids encoding one or more duplex MAPPs according to claim 17 and optionally administering the cell, tissue, or progeny thereof to the patient/subject;
wherein the one or more duplex MAPPs comprises one or more MODs and/or variant MODs; and
wherein at least one of the one or more MODs is optionally a valiant IL-2 MOD comprising an H16 and/or F42 substitution.


SEQUENCE LISTING (574 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 119 aa, Pan troglodytes; identical to SEQ ID NO: 1

SEQ ID NO: 3 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 4 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 5 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 6 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 7 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 8 - protein, 365 aa, Homo sapiens [sequence omitted]

SEQ ID NO: 9 - protein, 365 aa, Homo sapiens [sequence omitted]

SEQ ID NO: 10 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 11 - protein, 365 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCK

SEQ ID NO: 12 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 13 - protein, 275 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 14 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 15 - protein, 276 aa, Homo sapiens [sequence omitted]

SEQ ID NO: 16 - protein, 274 aa, Mouse
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 17 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 18 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 19 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 20 - protein, 276 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 21 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 22 - protein, 276 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 23 - protein, 275 aa, Homo sapiens; identical to SEQ ID NO: 12

SEQ ID NO: 24 - protein, 276 aa, Homo sapiens [sequence omitted]

SEQ ID NO: 25 - protein, 276 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 26 - protein, 276 aa, Homo sapiens; identical to SEQ ID NO: 20

SEQ ID NO: 27 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 28 - protein, 276 aa, Homo sapiens; identical to SEQ ID NO: 21

SEQ ID NO: 29 - protein, 276 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 30 - protein, 276 aa, Homo sapiens; identical to SEQ ID NO: 22

SEQ ID NO: 31 - protein, 276 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 32 - protein, 276 aa, Homo sapiens
  VARIANT (9)..(9): X1 is F, Y, S, or T
  VARIANT (44)..(44): X2 is K or R
  VARIANT (62)..(62): X3 is Q, G, E, or R
  VARIANT (63)..(63): X4 is N or E
  VARIANT (65)..(65): X5 is R or G
  VARIANT (66)..(66): X6 is N or K
  VARIANT (67)..(67): X7 is M or V
  VARIANT (70)..(70): X8 is H or Q
  VARIANT (73)..(73): X9 is T or I
  VARIANT (74)..(74): X10 is D or H
  VARIANT (76)..(76): X11 is A, V, or E
  VARIANT (77)..(77): X12 is N or D
  VARIANT (79)..(79): X13 is G or R
  VARIANT (80)..(80): X14 is T or I
  VARIANT (81)..(81): X15 is L or A
  VARIANT (82)..(82): X16 is R or L
  VARIANT (83)..(83): X17 is G or R
  VARIANT (90)..(90): X18 is A or D
  VARIANT (95)..(95): X19 is I, L, or V
  VARIANT (97)..(97): X20 is I, R or M
  VARIANT (99)..(99): X21 is F or Y
  VARIANT (105)..(105): X22 is S or P
  VARIANT (107)..(107): X23 is W or G
  VARIANT (114)..(114): X24 is R, H, or Q
  VARIANT (116)..(116): X25 is D or Y
  VARIANT (127)..(127): X26 is N or K
  VARIANT (142)..(142): X27 is T or I
  VARIANT (144)..(144): X28 is K or Q
  VARIANT (145)..(145): X29 is R or H
  VARIANT (149)..(149): X30 is A or T
  VARIANT (150)..(150): X31 is A or V
  VARIANT (151)..(151): X32 is H or R
  VARIANT (156)..(156): X33 is R, L, Q, or W
  VARIANT (158)..(158): X34 is V or A
  VARIANT (161)..(161): X35 is D or E
  VARIANT (163)..(163): X36 is R or T
  VARIANT (166)..(166): X37 is D or E
  VARIANT (167)..(167): X38 is W or G
  VARIANT (184)..(184): X39 is P or A
  VARIANT (193)..(193): X40 is P or A
  VARIANT (194)..(194): X41 is V or I
  VARIANT (207)..(207): X42 is S or G
  VARIANT (246)..(246): X43 is A or S
  VARIANT (253)..(253): X44 is Q or E
  VARIANT (276)..(276): X45 is P or L
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 33 - protein, 276 aa, Homo sapiens [sequence omitted]

SEQ ID NO: 34 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 35 - protein, 276 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 36 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 37 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 38 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 39 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 40 - protein, 276 aa, Homo sapiens
  VARIANT (9)..(9): X1 is H, Y, or D
  VARIANT (11)..(11): X2 is A or S
  VARIANT (12)..(12): X3 is M or V
  VARIANT (24)..(24): X4 is A, S, or T
  VARIANT (32)..(32): X5 is Q or L
  VARIANT (41)..(41): X6 is A or T
  VARIANT (45)..(45): X7 is E, M K, or T
  VARIANT (46)..(46): X8 is A or T
  VARIANT (63)..(63): X9 is E or N
  VARIANT (66)..(66): X10 is I or K
  VARIANT (67)..(67): X11 is Y, F, S, or C
  VARIANT (70)..(70): X12 is N or Q
  VARIANT (71)..(71): X13 is A or T
  VARIANT (74)..(74): X14 is D or Y
  VARIANT (76)..(76): X15 is E or V
  VARIANT (77)..(77): X16 is S or N
  VARIANT (80)..(80): X17 is T, N, or I
  VARIANT (81)..(81): X18 is A or L
  VARIANT (82)..(82): X19 is L, or R
  VARIANT (83)..(83): X20 is R or G
  VARIANT (94)..(94): X21 is T or I
  VARIANT (95)..(95): X22 is L or I
  VARIANT (97)..(97): X23 is R or S
  VARIANT (131)..(131): X24 is R or S
  VARIANT (143)..(143): X25 is S or T
  VARIANT (147)..(147): X26 is L or W
  VARIANT (152)..(152): X27 is E OR V
  VARIANT (156)..(156): X28 is R, D, L or W
  VARIANT (158)..(158): X29 is A or T
  VARIANT (163)..(163): X30 is L, E or T
  VARIANT (177)..(177): X31 is E or D
  VARIANT (178)..(178): X32 is K or T
  VARIANT (180)..(180): X33 is E or Q
  VARIANT (194)..(194): X34 is I or V
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 41 - protein, 276 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 42 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 43 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 44 - protein, 276 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 45 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 46 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 47 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 48 - protein, 276 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 49 - protein, 276 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 50 - protein, 276 aa, Homo sapiens
  VARIANT (1)..(1): X1 is C or G
  VARIANT (6)..(6): X2 is R or K
  VARIANT (9)..(9): X3 is F, Y, S, or D
  VARIANT (14)..(14): X4 is R or W
  VARIANT (21)..(21): X5 is H or R
  VARIANT (24)..(24): X6 is A or S
  VARIANT (35)..(35): X7 is Q or R
  VARIANT (49)..(49): X8 is A or E
  VARIANT (66)..(66): X9 is N or K
  VARIANT (73)..(73): X10 is T or A
  VARIANT (77)..(77): X11 is S or N
  VARIANT (80)..(80): X12 is N or K
  VARIANT (90)..(90): X13 is A or D
  VARIANT (91)..(91): X14 is G or R
  VARIANT (94)..(94): X15 is T or I
  VARIANT (95)..(95): X16 is L or I
  VARIANT (97)..(97): X17 is W or R
  VARIANT (99)..(99): X18 is C, Y, F, or S
  VARIANT (103)..(103): X19 is L, or V
  VARIANT (113)..(113): X20 is Y or H
  VARIANT (114)..(114): X21 is D or N
  VARIANT (116)..(116): X22 is Y, F, S, or L
  VARIANT (147)..(147): X23 is L or W
  VARIANT (152)..(152): X24 is E, A, Or T
  VARIANT (156)..(156): X25 is R, L, or W
  VARIANT (163)..(163): X26 is L or T
  VARIANT (173)..(173): X27 is E OR K
  VARIANT (173)..(173): X27 is E or K
  VARIANT (177)..(177): X28 is E or K
  VARIANT (184)..(184): X29 is H or P
  VARIANT (194)..(194): X30 is R or V
  VARIANT (219)..(219): X31 is W or R
  VARIANT (248)..(248): X32 is V or M
  VARIANT (253)..(253): X33 is E or Q
  VARIANT (261)..(261): X34 is M or V
  VARIANT (267)..(267): X35 is P or Q
  VARIANT (273)..(273): X36 is R or S
  VARIANT (275)..(275): X37 is P or G
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 51 - protein, 284 aa, Homo sapiens
  VARIANT (89)..(89): X1= K or E
  VARIANT (107)..(107): X2= R or G
  VARIANT (157)..(157): X3= R or G
  VARIANT (158)..(158): X4= A or V
  VARIANT (255)..(255): X5= Q or P
  VARIANT (267)..(267): X6= P or S
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 52 - protein, 284 aa, Homo sapiens
  VARIANT (7)..(7): X1= Y or F
  VARIANT (50)..(50): X2= P or Q
  VARIANT (251)..(251): X3= S or P
  VARIANT (278)..(278): X4= P or L
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 53 - protein, 284 aa, Homo sapiens
  VARIANT (13)..(13): X1= S or F
  VARIANT (27)..(27): X2= Y or H
  VARIANT (31)..(31): X3= T, S, or M
  VARIANT (34)..(34): X4= L or V
  VARIANT (54)..(54): X5= Q or R
  VARIANT (81)..(81): X6= P or L
  VARIANT (100)..(100): X7= G or D
  VARIANT (104)..(104): X8= G or V
  VARIANT (105)..(105): X9= S or C
  VARIANT (110)..(110): X10= L or I
  VARIANT (159)..(159): X11= Y or H
  VARIANT (169)..(169): X12= H or R
  VARIANT (171)..(171): X13= Y or H
  VARIANT (178)..(178): X14= M or T
  VARIANT (185)..(185): X15= P or A
  VARIANT (219)..(219): X16= R, W, or Q
  VARIANT (258)..(258): X17= T or M
  VARIANT (275)..(275): X18= K or E
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 54 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 55 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 56 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 57 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 63 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 66 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 67 - protein, 112 aa, Homo sapiens
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 68 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 69 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 71 - protein, 33 aa, Artificial Sequence
  Leucine-zipper polypeptide
RMKQIEDKIEEILSKIYHIENEIARIKKLIGER

SEQ ID NO: 72 - protein, 32 aa, Artificial Sequence
  Leucine-zipper polypeptide
LSSIEKKQEEQTSWLIWISNELTLIRNELAQS

SEQ ID NO: 73 - protein, 31 aa, Artificial Sequence
  Leucine-zipper polypeptide
LSSIEKKLEEITSQLIQISNELTLIRNELAQ

SEQ ID NO: 74 - protein, 31 aa, Artificial Sequence
  Leucine-zipper polypeptide
LSSIEKKLEEITSQLIQIRNELTLIRNELAQ

SEQ ID NO: 75 - protein, 31 aa, Artificial Sequence
  Leucine-zipper polypeptide
LSSIEKKLEEITSQLQQIRNELTLIRNELAQ

SEQ ID NO: 76 - protein, 31 aa, Artificial Sequence
  Leucine-zipper polypeptide
LSSLEKKLEELTSQLIQLRNELTLLRNELAQ

SEQ ID NO: 77 - protein, 31 aa, Artificial Sequence
  Leucine-zipper polypeptide
ISSLEKKIEELTSQIQQLRNEITLLRNEIAQ

SEQ ID NO: 78 - protein, 47 aa, Artificial Sequence
  Leucine-zipper polypeptide
LEIEAAFLERENTALETRVAELRQRVQRLRNRVSQYRTRYGPLGGGK

SEQ ID NO: 79 - protein, 31 aa, Artificial Sequence
  Coiled-coil polypeptide
LKSVENRLAVVENQLKTVIEELKTVKDLLSN

SEQ ID NO: 80 - protein, 31 aa, Artificial Sequence
  Coiled-coil polypeptide
VSRLEEKVKTLKSQVTELASTVSLLREQVAQ

SEQ ID NO: 81 - protein, 30 aa, Artificial Sequence
  Coiled-coil polypeptide
IQSEKKIEDISSLIGQIQSEITLIRNEIAQ

SEQ ID NO: 82 - protein, 31 aa, Artificial Sequence
  Coiled-coil polypeptide
LMSLEKKLEELTQTLMQLQNELSMLKNELAQ

SEQ ID NO: 83 - protein, 18 aa, Artificial Sequence
  Framework/dimerization polypeptide
VDLEGSTSNGRQCAGIRL

SEQ ID NO: 84 - protein, 27 aa, Artificial Sequence
  Framework/dimerization polypeptide
EDDVTTTEELAPALVPPPKGTCAGWMA

SEQ ID NO: 85 - protein, 27 aa, Artificial Sequence
  Framework/dimerization polypeptide
GHDQETTTQGPGVLLPLPKGACTGQMA

SEQ ID NO: 86 - protein, 64 aa, Homo sapiens
VTAFSNMDDMLQKAHLVIEGTFIYLRDSTEFFIRVRDGWKKLQLGELIPIPADSPPPPALSSNP

SEQ ID NO: 87 - protein, 10 aa, Artificial Sequence
  Linker
GCGGSGGGGS

SEQ ID NO: 88 - protein, 276 aa, Artificial Sequence
  T-Cell-MMP or its epitope conjugate which may have one or more substitutions as indicated in the feature field.
  VARIANT (79)..(83): Residues 79-83 each can be any amino acid or any amino acid other than glycine in an embodiment it is the sequence GTLRG
  DISULFID (84)..(84): Residue 84 can form a disulfide bond with residue 139
  VARIANT (85)..(89): Residues 85-89 each can be any amino acid or any amino acid other than glycine, in an embodiment it is the sequence YNQSE
  VARIANT (134)..(138): Residues 134-138 each can be any amino acid or any amino acid other than glycine, in an embodiment it is the sequence TAADM
  DISULFID (139)..(139): Residue 139 can form a disulfide bond with residue 84
  VARIANT (140)..(144): Residues 140-144 each can be any amino acid or any amino acid other than glycine, in an embodiment it is the sequence AQITK
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 89 - protein, 99 aa, Artificial Sequence
  T-Cell-MMP or its epitope conjugate polypeptide with substitutions.
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 90 - protein, 276 aa, Artificial Sequence
  T-Cell-MMP or its epitope conjugate which may have one or more substitutions as indicated in the feature field.
  VARIANT (79)..(83): Residues 79-83 each can be any amino acid or any amino acid other than glycine in an embodiment it is the sequence GTLRG
  DISULFID (84)..(84): Residue 84 can form a disulfide bond with residue 139
  VARIANT (85)..(89): Residues 85-89 each can be any amino acid or any amino acid other than glycine, in an embodiment it is the sequence YNQSE
  VARIANT (134)..(138): Residues 134-138 each can be any amino acid or any amino acid other than glycine, in an embodiment it is the sequence TAADM
  DISULFID (139)..(139): Residue 139 can form a disulfide bond with residue 84
  VARIANT (140)..(144): Residues 140-144 each can be any amino acid or any amino acid other than glycine, in an embodiment it is the sequence AQITK
  VARIANT (231)..(235): Residues 231-235 each can be any amino acid or any amino acid other than glycine, in an embodiment it is the sequence VETRP
  DISULFID (236)..(236): Residue 236 can form a disulfide bond with beta-2M-containing polypeptide
  VARIANT (237)..(241): Residues 237-241 each can be any amino acid or any amino acid other than glycine, in an embodiment it is the sequence GDGTF
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELXXXXXCXXXXXQKWAAVVVPSGQEQRYTCHVQHEGLPKPLTLRWEP

SEQ ID NO: 91 - protein, 99 aa, Homo sapiens; identical to SEQ ID NO: 89

SEQ ID NO: 92 - protein, 10 aa, Artificial Sequence
  Linker
GCGGSGGGGS

SEQ ID NO: 93 - protein, 20 aa, Artificial Sequence
  Linker
GCGGSGGGGSGGGGSGGGGS

SEQ ID NO: 94 - protein, 15 aa, Artificial Sequence
  Linker
GCGGSGGGGSGGGGS

SEQ ID NO: 95 - protein, 133 aa, Homo sapiens [sequence omitted]

SEQ ID NO: 96 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 97 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 98 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 99 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 100 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 101 - protein, 150 aa, Homo sapiens
IGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYK

SEQ ID NO: 102 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 103 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 104 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 105 - protein, 227 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLNVSIKI

SEQ ID NO: 106 - protein, 124 aa, Homo sapiens
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 107 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 108 - protein, 208 aa, Homo sapiens [sequence omitted]

SEQ ID NO: 109 - protein, 104 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSV

SEQ ID NO: 110 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 111 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 112 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 113 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 114 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 115 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 116 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 117 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 118 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 119 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 120 - protein, 5 aa, Artificial Sequence
  Peptide suitable for use as a linker.
GSGGS

SEQ ID NO: 121 - protein, 4 aa, Artificial Sequence
  Peptide suitable for use as a linker.
GGGS

SEQ ID NO: 122 - protein, 4 aa, Artificial Sequence
  Peptide suitable for use as a linker.
GGSG

SEQ ID NO: 123 - protein, 5 aa, Artificial Sequence
  Peptide suitable for use as a linker.
GGSGG

SEQ ID NO: 124 - protein, 5 aa, Artificial Sequence
  Peptide suitable for use as a linker.
GSGSG

SEQ ID NO: 125 - protein, 5 aa, Artificial Sequence
  Peptide suitable for use as a linker.
GSGGG

SEQ ID NO: 126 - protein, 5 aa, Artificial Sequence
  Peptide suitable for use as a linker.
GGGSG

SEQ ID NO: 127 - protein, 5 aa, Artificial Sequence
  Peptide suitable for use as a linker.
GSSSG

SEQ ID NO: 128 - protein, 5 aa, Artificial Sequence
  Peptide suitable for use as a linker.
GSSSS

SEQ ID NO: 129 - protein, 5 aa, Artificial Sequence
  Peptide suitable for use as a linker.
GGGGS

SEQ ID NO: 130 - protein, 5 aa, Artificial Sequence
  Peptide suitable for use as a linker.
AAAGG

SEQ ID NO: 131 - protein, 15 aa, Artificial Sequence
  Linker
GCGASGGGGSGGGGS

SEQ ID NO: 132 - protein, 20 aa, Artificial Sequence
  Linker
GCGGSGGGGSGGGGSGGGGS

SEQ ID NO: 133 - protein, 15 aa, Artificial Sequence
  Linker
GCGGSGGGGSGGGGS

SEQ ID NO: 134 - protein, 9 aa, Artificial Sequence
  Alpha Feto Protein (AFP) Peptide
AITRKMAAT

SEQ ID NO: 135 - protein, 9 aa, Artificial Sequence
  Alpha Feto Protein (AFP) Peptide
AYTKKAPQL

SEQ ID NO: 136 - protein, 9 aa, Artificial Sequence
  Alpha Feto Protein (AFP) Peptide
LLNQHACAV

SEQ ID NO: 137 - protein, 9 aa, Artificial Sequence
  Alpha Feto Protein (AFP) Peptide
KLVLDVAHV

SEQ ID NO: 138 - protein, 9 aa, Artificial Sequence
  Alpha Feto Protein (AFP) Peptide
FMNKFIYEI

SEQ ID NO: 139 - protein, 9 aa, Artificial Sequence
  Alpha Feto Protein (AFP) Peptide
SIPLFQVPE

SEQ ID NO: 140 - protein, 9 aa, Artificial Sequence
  Alpha Feto Protein (AFP) Peptide
LLNFTESRT

SEQ ID NO: 141 - protein, 9 aa, Artificial Sequence
  Alpha Feto Protein (AFP) Peptide
FVQEATYKF

SEQ ID NO: 142 - protein, 9 aa, Artificial Sequence
  Alpha Feto Protein (AFP) Peptide
ATYKEVSKM

SEQ ID NO: 143 - protein, 9 aa, Artificial Sequence
  Alpha Feto Protein (AFP) Peptide
KEVSKMVKD

SEQ ID NO: 144 - protein, 9 aa, Artificial Sequence
  Alpha Feto Protein (AFP) Peptide
RHNCFLAHK

SEQ ID NO: 145 - protein, 9 aa, Artificial Sequence
  Alpha Feto Protein (AFP) Peptide
ATAATCCQL

SEQ ID NO: 146 - protein, 9 aa, Artificial Sequence
  Alpha Feto Protein (AFP) Peptide
YIQESQALA

SEQ ID NO: 147 - protein, 10 aa, Artificial Sequence
  Alpha Feto Protein (AFP) Peptide
QLTSSELMAI

SEQ ID NO: 148 - protein, 9 aa, Artificial Sequence
  Alpha Feto Protein (AFP) Peptide
KLSQKFTKV

SEQ ID NO: 149 - protein, 9 aa, Artificial Sequence
  Alpha Feto Protein (AFP) Peptide
KELRESSLL

SEQ ID NO: 150 - protein, 9 aa, Artificial Sequence
  Alpha Feto Protein (AFP) Peptide
SLVVDETYV

SEQ ID NO: 151 - protein, 9 aa, Artificial Sequence
  Alpha Feto Protein (AFP) Peptide
ILLWAARYD

SEQ ID NO: 152 - protein, 9 aa, Artificial Sequence
  Alpha Feto Protein (AFP) Peptide
KIIPSCCKA

SEQ ID NO: 153 - protein, 9 aa, Artificial Sequence
  Alpha Feto Protein (AFP) Peptide
CRGDVLDCL

SEQ ID NO: 154 - protein, 9 aa, Artificial Sequence
  Alpha Feto Protein (AFP) Peptide
QQDTLSNKI

SEQ ID NO: 155 - protein, 10 aa, Artificial Sequence
  Alpha Feto Protein (AFP) Peptide
TMKQEFLINL

SEQ ID NO: 156 - protein, 9 aa, Artificial Sequence
  Alpha Feto Protein (AFP) Peptide
NLVKQKPQI

SEQ ID NO: 157 - protein, 9 aa, Artificial Sequence
  Alpha Feto Protein (AFP) Peptide
AVIADFSGL

SEQ ID NO: 158 - protein, 9 aa, Artificial Sequence
  Alpha Feto Protein (AFP) Peptide
LLACGEGAA

SEQ ID NO: 159 - protein, 9 aa, Artificial Sequence
  Alpha Feto Protein (AFP) Peptide
LACGEGAAD

SEQ ID NO: 160 - protein, 10 aa, Artificial Sequence
  Alpha Feto Protein (AFP) Peptide
KAPQLTSSEL

SEQ ID NO: 161 - protein, 9 aa, Artificial Sequence
  Alpha Feto Protein (AFP) Peptide
YICSQQDTL

SEQ ID NO: 162 - protein, 9 aa, Artificial Sequence
  Alpha Feto Protein (AFP) Peptide
TECCKLTTL

SEQ ID NO: 163 - protein, 10 aa, Artificial Sequence
  Alpha Feto Protein (AFP) Peptide
CTAEISLADL

SEQ ID NO: 164 - protein, 10 aa, Artificial Sequence
  Alpha Feto Protein (AFP) Peptide
VTKELRESSL

SEQ ID NO: 165 - protein, 10 aa, Artificial Sequence
  Alpha Feto Protein (AFP) Peptide
IMSYICSQQD

SEQ ID NO: 166 - protein, 9 aa, Artificial Sequence
  Alpha Feto Protein (AFP) Peptide
TRTFQAITV

SEQ ID NO: 167 - protein, 9 aa, Artificial Sequence
  Alpha Feto Protein (AFP) Peptide
FQKLGEYYL

SEQ ID NO: 168 - protein, 9 aa, Artificial Sequence
  Alpha Feto Protein (AFP) Peptide
RVAKGYQEL

SEQ ID NO: 169 - protein, 10 aa, Artificial Sequence
  Alpha Feto Protein (AFP) Peptide
SYQCTAEISL

SEQ ID NO: 170 - protein, 9 aa, Artificial Sequence
  Alpha Feto Protein (AFP) Peptide
KQEFLINLV

SEQ ID NO: 171 - protein, 9 aa, Artificial Sequence
  Alpha Feto Protein (AFP) Peptide
MKWVESIFL

SEQ ID NO: 172 - protein, 9 aa, Artificial Sequence
  Alpha Feto Protein (AFP) Peptide
PVNPGVGQC

SEQ ID NO: 173 - protein, 9 aa, Artificial Sequence
  Alpha Feto Protein (AFP) Peptide
AADIIIGHL

SEQ ID NO: 174 - protein, 9 aa, Artificial Sequence
  Alpha Feto Protein (AFP) Peptide
QVPEPVTSC

SEQ ID NO: 175 - protein, 10 aa, Artificial Sequence
  Alpha Feto Protein (AFP) Peptide
TTLERGQCII

SEQ ID NO: 176 - protein, 9 aa, Artificial Sequence
  Alpha Feto Protein (AFP) Peptide
KMAATAATC

SEQ ID NO: 177 - protein, 10 aa, Artificial Sequence
  Alpha Feto Protein (AFP) Peptide
QAQGVALQTM

SEQ ID NO: 178 - protein, 9 aa, Artificial Sequence
  Alpha Feto Protein (AFP) Peptide
FQAITVTKL

SEQ ID NO: 179 - protein, 9 aa, Artificial Sequence
  Alpha Feto Protein (AFP) Peptide
LLEKCFQTE

SEQ ID NO: 180 - protein, 9 aa, Artificial Sequence
  Alpha Feto Protein (AFP) Peptide
VAYTKKAPQ

SEQ ID NO: 181 - protein, 9 aa, Artificial Sequence
  Alpha Feto Protein (AFP) Peptide
KYIQESQAL

SEQ ID NO: 182 - protein, 9 aa, Artificial Sequence
  Alpha Feto Protein (AFP) Peptide
GVALQTMKQ

SEQ ID NO: 183 - protein, 9 aa, Artificial Sequence
  Alpha Feto Protein (AFP) Peptide
GQEQEVCFA

SEQ ID NO: 184 - protein, 10 aa, Artificial Sequence
  Alpha Feto Protein (AFP) Peptide
SEEGRHNCFL

SEQ ID NO: 185 - protein, 10 aa, Artificial Sequence
  Alpha Feto Protein (AFP) Peptide
RHPFLYAPTI

SEQ ID NO: 186 - protein, 10 aa, Artificial Sequence
  Alpha Feto Protein (AFP) Peptide
TEIQKLVLDV

SEQ ID NO: 187 - protein, 10 aa, Artificial Sequence
  Alpha Feto Protein (AFP) Peptide
RRHPQLAVSV

SEQ ID NO: 188 - protein, 10 aa, Artificial Sequence
  Alpha Feto Protein (AFP) Peptide
GEYYLQNAFL

SEQ ID NO: 189 - protein, 10 aa, Artificial Sequence
  Alpha Feto Protein (AFP) Peptide
NRRPCFSSLV

SEQ ID NO: 190 - protein, 10 aa, Artificial Sequence
  Alpha Feto Protein (AFP) Peptide
LQTMKQEFLI

SEQ ID NO: 191 - protein, 10 aa, Artificial Sequence
  Alpha Feto Protein (AFP) Peptide
IADFSGLLEK

SEQ ID NO: 192 - protein, 10 aa, Artificial Sequence
  Alpha Feto Protein (AFP) Peptide
GLLEKCCQGQ

SEQ ID NO: 193 - protein, 9 aa, Artificial Sequence
  Alpha Feto Protein (AFP) Peptide
TLSNKITEC

SEQ ID NO: 194 - protein, 10 aa, Artificial Sequence
  Alpha Feto Protein (AFP) Peptide
LQDGEKIMSY

SEQ ID NO: 195 - protein, 9 aa, Artificial Sequence
  Alpha Feto Protein (AFP) Peptide
GLFQKLGBY

SEQ ID NO: 196 - protein, 10 aa, Artificial Sequence
  Alpha Feto Protein (AFP) Peptide
NEYGIASILD

SEQ ID NO: 197 - protein, 10 aa, Artificial Sequence
  Alpha Feto Protein (AFP) Peptide
KMVKDALTAI

SEQ ID NO: 198 - protein, 9 aa, Artificial Sequence
  Alpha Feto Protein (AFP) Peptide
FLASFVHEY

SEQ ID NO: 199 - protein, 9 aa, Artificial Sequence
  Alpha Feto Protein (AFP) Peptide
AQFVQEATY

SEQ ID NO: 200 - protein, 9 aa, Artificial Sequence
  Wilms Tumor Antigen (WT-1) Peptide
NLMNLGATL

SEQ ID NO: 201 - protein, 9 aa, Artificial Sequence
  Wilms Tumor Antigen (WT-1) Peptide
NYMNLGATL

SEQ ID NO: 202 - protein, 15 aa, Artificial Sequence
  Wilms Tumor Antigen (WT-1) Peptide
CMTWNQMNLGATLKG

SEQ ID NO: 203 - protein, 15 aa, Artificial Sequence
  Wilms Tumor Antigen (WT-1) Peptide
WNQMNLGATLKGVAA

SEQ ID NO: 204 - protein, 15 aa, Artificial Sequence
  Wilms Tumor Antigen (WT-1) Peptide
CMTWNYMNLGATLKG

SEQ ID NO: 205 - protein, 15 aa, Artificial Sequence
  Wilms Tumor Antigen (WT-1) Peptide
WNYMNLGATLKGVAA

SEQ ID NO: 206 - protein, 14 aa, Artificial Sequence
  Wilms Tumor Antigen (WT-1) Peptide
TWNQMNLGATLKGV

SEQ ID NO: 207 - protein, 15 aa, Artificial Sequence
  Wilms Tumor Antigen (WT-1) Peptide
TWNQMNLGATLKGVA

SEQ ID NO: 208 - protein, 15 aa, Artificial Sequence
  Wilms Tumor Antigen (WT-1) Peptide
CMTWNLMNLGATLKG

SEQ ID NO: 209 - protein, 15 aa, Artificial Sequence
  Wilms Tumor Antigen (WT-1) Peptide
MTWNLMNLGATLKGV

SEQ ID NO: 210 - protein, 15 aa, Artificial Sequence
  Wilms Tumor Antigen (WT-1) Peptide
TWNLMNLGATLKGVA

SEQ ID NO: 211 - protein, 15 aa, Artificial Sequence
  Wilms Tumor Antigen (WT-1) Peptide
WNLMNLGATLKGVAA

SEQ ID NO: 212 - protein, 8 aa, Artificial Sequence
  Wilms Tumor Antigen (WT-1) Peptide
MNLGATLK

SEQ ID NO: 213 - protein, 15 aa, Artificial Sequence
  Wilms Tumor Antigen (WT-1) Peptide
MTWNYMNLGATLKGV

SEQ ID NO: 214 - protein, 15 aa, Artificial Sequence
  Wilms Tumor Antigen (WT-1) Peptide
TWNYMNLGATLKGVA

SEQ ID NO: 215 - protein, 17 aa, Artificial Sequence
  Wilms Tumor Antigen (WT-1) Peptide
CMTWNQMNLGATLKGVA

SEQ ID NO: 216 - protein, 17 aa, Artificial Sequence
  Wilms Tumor Antigen (WT-1) Peptide
CMTWNLMNLGATLKGVA

SEQ ID NO: 217 - protein, 17 aa, Artificial Sequence
  Wilms Tumor Antigen (WT-1) Peptide
CMTWNYMNLGATLKGVA

SEQ ID NO: 218 - protein, 9 aa, Artificial Sequence
  Wilms Tumor Antigen (WT-1) Peptide
GYLRNPTAC

SEQ ID NO: 219 - protein, 9 aa, Artificial Sequence
  Wilms Tumor Antigen (WT-1) Peptide
GALRNPTAL

SEQ ID NO: 220 - protein, 9 aa, Artificial Sequence
  Wilms Tumor Antigen (WT-1) Peptide
YALRNPTAC

SEQ ID NO: 221 - protein, 9 aa, Artificial Sequence
  Wilms Tumor Antigen (WT-1) Peptide
GLLRNPTAC

SEQ ID NO: 222 - protein, 9 aa, Artificial Sequence
  Wilms Tumor Antigen (WT-1) Peptide
RYRPHPGAL

SEQ ID NO: 223 - protein, 9 aa, Artificial Sequence
  Wilms Tumor Antigen (WT-1) Peptide
YQRPHPGAL

SEQ ID NO: 224 - protein, 9 aa, Artificial Sequence
  Wilms Tumor Antigen (WT-1) Peptide
RLRPHPGAL

SEQ ID NO: 225 - protein, 9 aa, Artificial Sequence
  Wilms Tumor Antigen (WT-1) Peptide
RIRPHPGAL

SEQ ID NO: 226 - protein, 15 aa, Artificial Sequence
  Wilms Tumor Antigen (WT-1) Peptide
QFPNHSFKHEDPMGQ

SEQ ID NO: 227 - protein, 9 aa, Artificial Sequence
  Wilms Tumor Antigen (WT-1) Peptide
HSFKHEDPY

SEQ ID NO: 228 - protein, 13 aa, Artificial Sequence
  Wilms Tumor Antigen (WT-1) Peptide
QFPNHSFKHEDPM

SEQ ID NO: 229 - protein, 13 aa, Artificial Sequence
  Wilms Tumor Antigen (WT-1) Peptide
QFPNHSFKHEDPY

SEQ ID NO: 230 - protein, 13 aa, Artificial Sequence
  Wilms Tumor Antigen (WT-1) Peptide
KRPFMCAYPGCNK

SEQ ID NO: 231 - protein, 13 aa, Artificial Sequence
  Wilms Tumor Antigen (WT-1) Peptide
KRPFMCAYPGCYK

SEQ ID NO: 232 - protein, 9 aa, Artificial Sequence
  Wilms Tumor Antigen (WT-1) Peptide
FMCAYPGCY

SEQ ID NO: 233 - protein, 9 aa, Artificial Sequence
  Wilms Tumor Antigen (WT-1) Peptide
FMCAYPGCK

SEQ ID NO: 234 - protein, 15 aa, Artificial Sequence
  Wilms Tumor Antigen (WT-1) Peptide
KRPFMCAYPGCNKRY

SEQ ID NO: 235 - protein, 15 aa, Artificial Sequence
  Wilms Tumor Antigen (WT-1) Peptide
SEKRPFMCAYPGCNK

SEQ ID NO: 236 - protein, 15 aa, Artificial Sequence
  Wilms Tumor Antigen (WT-1) Peptide
KRPFMCAYPGCYKRY

SEQ ID NO: 237 - protein, 9 aa, Artificial Sequence
  Wilms Tumor Antigen (WT-1) Peptide
NLMNLGATL

SEQ ID NO: 238 - protein, 9 aa, Artificial Sequence
  Wilms Tumor Antigen (WT-1) Peptide
NYMNLGATL

SEQ ID NO: 239 - protein, 9 aa, Human papillomavirus type 1a
KLPQLCTEL

SEQ ID NO: 240 - protein, 9 aa, Human papillomavirus type 1a
LQTTIHDII

SEQ ID NO: 241 - protein, 9 aa, Human papillomavirus type 1a
VYDFAFRDL

SEQ ID NO: 242 - protein, 9 aa, Human papillomavirus type 1a
FAFRDLCIV

SEQ ID NO: 243 - protein, 9 aa, Human papillomavirus type 1a
KFYSKISEY

SEQ ID NO: 244 - protein, 9 aa, Human papillomavirus type 1a
ISEYRHYCY

SEQ ID NO: 245 - protein, 9 aa, Human papillomavirus type 1a
TLHEYMLDL

SEQ ID NO: 246 - protein, 9 aa, Human papillomavirus type 1a
YMLDLQPET

SEQ ID NO: 247 - protein, 9 aa, Human papillomavirus type 1a
QAEPDRAHY

SEQ ID NO: 248 - protein, 9 aa, Human papillomavirus type 1a
RAHYNIVTF

SEQ ID NO: 249 - protein, 9 aa, Human papillomavirus type 1a
CDSTLRLCV

SEQ ID NO: 250 - protein, 10 aa, Human papillomavirus type 1a
LCVQSTHVDI

SEQ ID NO: 251 - protein, 9 aa, Human papillomavirus type 1a
LLMGTLGIV

SEQ ID NO: 252 - protein, 8 aa, Human papillomavirus type 1a
TLGIVCPI

SEQ ID NO: 253 - protein, 12 aa, Human papillomavirus type 1a
LLMGTLGIVCPI

SEQ ID NO: 254 - protein, 10 aa, Human papillomavirus type 1a
YMLDLQPETT

SEQ ID NO: 255 - protein, 9 aa, Human papillomavirus type 1a
YMLDLQPET

SEQ ID NO: 256 - protein, 10 aa, Hepatitis B virus
FLPSDFFPSV

SEQ ID NO: 257 - protein, 10 aa, Hepatitis B virus
GLSRYVARLG

SEQ ID NO: 258 - protein, 9 aa, Hepatitis B virus
KLHLYSHPI

SEQ ID NO: 259 - protein, 9 aa, Hepatitis B virus
FLLSLGIHL

SEQ ID NO: 260 - protein, 9 aa, Hepatitis B virus
ALMPLYACI

SEQ ID NO: 261 - protein, 9 aa, Hepatitis B virus
SLYADSPSV

SEQ ID NO: 262 - protein, 9 aa, Hepatitis B virus
STLPETTVV

SEQ ID NO: 263 - protein, 10 aa, Hepatitis B virus
LIMPARFYPK

SEQ ID NO: 264 - protein, 10 aa, Hepatitis B virus
AIMPARFYPK

SEQ ID NO: 265 - protein, 9 aa, Hepatitis B virus
YVNVNMGLK

SEQ ID NO: 266 - protein, 9 aa, Hepatitis B virus
CYSLYGTTL

SEQ ID NO: 267 - protein, 15 aa, Hepatitis B virus
MQWNSTALHQALQDP

SEQ ID NO: 268 - protein, 9 aa, Hepatitis B virus
LLDPRVRGL

SEQ ID NO: 269 - protein, 10 aa, Hepatitis B virus
SILSKTGDPV

SEQ ID NO: 270 - protein, 9 aa, Hepatitis B virus
VLQAGFFLL

SEQ ID NO: 271 - protein, 9 aa, Hepatitis B virus
FLLTRILTI

SEQ ID NO: 272 - protein, 9 aa, Hepatitis B virus
FLGGTPVCL

SEQ ID NO: 273 - protein, 9 aa, Hepatitis B virus
LLCLIFLLV

SEQ ID NO: 274 - protein, 10 aa, Hepatitis B virus
LVLLDYQGML

SEQ ID NO: 275 - protein, 10 aa, Hepatitis B virus
LLDYQGMLPV

SEQ ID NO: 276 - protein, 9 aa, Hepatitis B virus
IPIPSSWAF

SEQ ID NO: 277 - protein, 9 aa, Hepatitis B virus
WLSLLVPFV

SEQ ID NO: 278 - protein, 10 aa, Hepatitis B virus
GLSPTVWLSV

SEQ ID NO: 279 - protein, 10 aa, Hepatitis B virus
SIVSPFIPLL

SEQ ID NO: 280 - protein, 9 aa, Hepatitis B virus
ILSPFLPLL

SEQ ID NO: 281 - protein, 17 aa, Hepatitis B virus
ATVELLSFLPSDFFPSV

SEQ ID NO: 282 - protein, 9 aa, Hepatitis B virus
LPSDFFPSV

SEQ ID NO: 283 - protein, 9 aa, Hepatitis B virus
CLTFGRETV

SEQ ID NO: 284 - protein, 10 aa, Hepatitis B virus
VLEYLVSFGV

SEQ ID NO: 285 - protein, 9 aa, Hepatitis B virus
EYLVSFGVW

SEQ ID NO: 286 - protein, 10 aa, Hepatitis B virus
ILSTLPETTV

SEQ ID NO: 287 - protein, 11 aa, Hepatitis B virus
STLPETTVVRR

SEQ ID NO: 288 - protein, 9 aa, Hepatitis B virus
NVSIPWTHK

SEQ ID NO: 289 - protein, 9 aa, Hepatitis B virus
KVGNFTGLY

SEQ ID NO: 290 - protein, 9 aa, Hepatitis B virus
GLYSSTVPV

SEQ ID NO: 291 - protein, 10 aa, Hepatitis B virus
TLWKAGILYK

SEQ ID NO: 292 - protein, 10 aa, Hepatitis B virus
TPARVTGGVF

SEQ ID NO: 293 - protein, 10 aa, Hepatitis B virus
LVVDFSQFSR

SEQ ID NO: 294 - protein, 9 aa, Hepatitis B virus
GLSRYVARL

SEQ ID NO: 295 - protein, 9 aa, Hepatitis B virus
SAICSVVRR

SEQ ID NO: 296 - protein, 9 aa, Hepatitis B virus
YMDDVVLGA

SEQ ID NO: 297 - protein, 8 aa, Hepatitis B virus
PLGFFPDH

SEQ ID NO: 298 - protein, 10 aa, Hepatitis B virus
QAFTFSPTYK

SEQ ID NO: 299 - protein, 9 aa, Hepatitis B virus
KYTSFPWLL

SEQ ID NO: 300 - protein, 10 aa, Hepatitis B virus
ILRGTSFVYV

SEQ ID NO: 301 - protein, 9 aa, Hepatitis B virus
HLSLRGLFV

SEQ ID NO: 302 - protein, 9 aa, Hepatitis B virus
VLHKRTLGL

SEQ ID NO: 303 - protein, 10 aa, Hepatitis B virus
GLSAMSTTDL

SEQ ID NO: 304 - protein, 9 aa, Hepatitis B virus
CLFKDWEEL

SEQ ID NO: 305 - protein, 9 aa, Hepatitis B virus
VLGGCRHKL

SEQ ID NO: 306 - protein, 9 aa, Artificial Sequence
  Epitope tag hemagglutinin
YPYDVPDYA

SEQ ID NO: 307 - protein, 8 aa, Artificial Sequence
  Epitope tag FLAG
DYKDDDDK

SEQ ID NO: 308 - protein, 10 aa, Artificial Sequence
  Epitope tag  c-myc
EQKLISEEDL

SEQ ID NO: 309 - protein, 5 aa, Artificial Sequence
  Affinity domain
HHHHH

SEQ ID NO: 310 - protein, 6 aa, Artificial Sequence
  Affinity domain
HHHHHH

SEQ ID NO: 311 - protein, 10 aa, Artificial Sequence
  Affinity domain
EQKLISEEDL

SEQ ID NO: 312 - protein, 8 aa, Artificial Sequence
  Affinity domain
DYKDDDDK

SEQ ID NO: 313 - protein, 8 aa, Artificial Sequence
  Affinity domain
WSHPQFEK

SEQ ID NO: 314 - protein, 9 aa, Artificial Sequence
  HA Tag Affinity domain
YPYDVPDYA

SEQ ID NO: 315 - protein, 5 aa, Artificial Sequence
  Affinity domain
RYIRS

SEQ ID NO: 316 - protein, 4 aa, Artificial Sequence
  Affinity domain
FHHT

SEQ ID NO: 317 - protein, 17 aa, Artificial Sequence
  Affinity domain
WEAAAREACCRECCARA

SEQ ID NO: 318 - protein, 586 aa, Homo sapiens
LAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 319 - protein, 10 aa, Artificial Sequence
  Mesothelin peptide
KLLGPHVEGL

SEQ ID NO: 320 - protein, 10 aa, Artificial Sequence
  Mesothelin peptide
AFYPGYLCSL

SEQ ID NO: 321 - protein, 9 aa, Artificial Sequence
  Mesothelin peptide
VLPLTVAEV

SEQ ID NO: 322 - protein, 9 aa, Artificial Sequence
  Mesothelin peptide
ELAVALAQK

SEQ ID NO: 323 - protein, 9 aa, Artificial Sequence
  Mesothelin peptide
ALQGGGPPY

SEQ ID NO: 324 - protein, 9 aa, Artificial Sequence
  Mesothelin peptide
FYPGYLCSL

SEQ ID NO: 325 - protein, 9 aa, Artificial Sequence
  Mesothelin peptide
LYPKARLAF

SEQ ID NO: 326 - protein, 15 aa, Artificial Sequence
  Mesothelin peptide
LLFLLFSLGWVGPSR

SEQ ID NO: 327 - protein, 15 aa, Artificial Sequence
  Mesothelin peptide
VNKGHEMSPQAPRRP

SEQ ID NO: 328 - protein, 15 aa, Artificial Sequence
  Mesothelin peptide
FMKLRTDAVLPLTVA

SEQ ID NO: 329 - protein, 10 aa, Artificial Sequence
  Mesothelin peptide
DAALLATQMD

SEQ ID NO: 330 - protein, 1055 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRNM

SEQ ID NO: 331 - protein, 184 aa, Homo sapiens [sequence omitted]

SEQ ID NO: 332 - protein, 502 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 333 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 107 aa, Homo sapiens [sequence omitted]

SEQ ID NO: 336 - protein, 120 aa, Homo sapiens [sequence omitted]

SEQ ID NO: 337 - protein, 120 aa, Homo sapiens [sequence omitted]

SEQ ID NO: 338 - protein, 107 aa, Homo sapiens [sequence omitted]

SEQ ID NO: 339 - protein, 5 aa, Artificial Sequence
  Linker
GGGGS

SEQ ID NO: 340 - protein, 11 aa, Homo sapiens
RASQDVNTAVA

SEQ ID NO: 341 - protein, 6 aa, Homo sapiens
SASFLY

SEQ ID NO: 342 - protein, 8 aa, Homo sapiens
QQHYTTPP

SEQ ID NO: 343 - protein, 8 aa, Homo sapiens
GFNIKDTY

SEQ ID NO: 344 - protein, 8 aa, Homo sapiens
IYPTNGYT

SEQ ID NO: 345 - protein, 13 aa, Homo sapiens
SRWGGDGFYAMDY

SEQ ID NO: 346 - protein, 242 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 347 - protein, 214 aa, Homo sapiens
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 448 aa, Homo sapiens
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 349 - protein, 107 aa, Homo sapiens [sequence omitted]

SEQ ID NO: 350 - protein, 119 aa, Homo sapiens [sequence omitted]

SEQ ID NO: 351 - protein, 11 aa, Homo sapiens
KASQDVSIGVA

SEQ ID NO: 352 - protein, 6 aa, Homo sapiens
SASYRY

SEQ ID NO: 353 - protein, 8 aa, Homo sapiens
QQYYIYPY

SEQ ID NO: 354 - protein, 10 aa, Homo sapiens
GFTFTDYTMD

SEQ ID NO: 355 - protein, 18 aa, Homo sapiens
ADVNPNSGGSIYNQRFKG

SEQ ID NO: 356 - protein, 12 aa, Homo sapiens
ARNLGPSFYFDY

SEQ ID NO: 357 - protein, 242 aa, Homo sapiens; identical to SEQ ID NO: 346

SEQ ID NO: 358 - protein, 15 aa, Homo sapiens
KASQSVDYDGDSYLN

SEQ ID NO: 359 - protein, 7 aa, Homo sapiens
DASNLVS

SEQ ID NO: 360 - protein, 9 aa, Homo sapiens
QQSTEDPWT

SEQ ID NO: 361 - protein, 5 aa, Homo sapiens
SYWMN

SEQ ID NO: 362 - protein, 17 aa, Homo sapiens
QIWPGDGDTNYNGKFKG

SEQ ID NO: 363 - protein, 15 aa, Homo sapiens
RETTTVGRYYYAMDY

SEQ ID NO: 364 - protein, 15 aa, Homo sapiens
KASQSVDYDGDSYLN

SEQ ID NO: 365 - protein, 7 aa, Homo sapiens
DASNLVS

SEQ ID NO: 366 - protein, 9 aa, Homo sapiens
QQSTEDPWT

SEQ ID NO: 367 - protein, 5 aa, Homo sapiens
SYWMN

SEQ ID NO: 368 - protein, 17 aa, Homo sapiens
QIWPGDGDTNYNGKFKG

SEQ ID NO: 369 - protein, 15 aa, Homo sapiens
RETTTVGRYYYAMDY

SEQ ID NO: 370 - protein, 249 aa, Homo sapiens
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 371 - protein, 217 aa, Homo sapiens
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 372 - protein, 450 aa, Homo sapiens
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 107 aa, Homo sapiens
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 374 - protein, 120 aa, Homo sapiens
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 375 - protein, 14 aa, Homo sapiens
TGTSSDIGGYNSVS

SEQ ID NO: 376 - protein, 11 aa, Homo sapiens
LMIYGVNNRPS

SEQ ID NO: 377 - protein, 10 aa, Homo sapiens
SSYDIESATP

SEQ ID NO: 378 - protein, 10 aa, Homo sapiens
GYSFTSYWIG

SEQ ID NO: 379 - protein, 16 aa, Homo sapiens
WMGIIDPGDSRTRYSP

SEQ ID NO: 380 - protein, 11 aa, Homo sapiens
GQLYGGTYMDG

SEQ ID NO: 381 - protein, 244 aa, Homo sapiens
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 382 - protein, 253 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 383 - protein, 217 aa, Homo sapiens
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 384 - protein, 453 aa, Homo sapiens
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 111 aa, Homo sapiens
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 386 - protein, 131 aa, Homo sapiens
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 387 - protein, 8 aa, Homo sapiens
SSNIGSNT

SEQ ID NO: 388 - protein, 11 aa, Homo sapiens
AAWDDSLNGWV

SEQ ID NO: 389 - protein, 8 aa, Homo sapiens
GFTFGDYA

SEQ ID NO: 390 - protein, 10 aa, Homo sapiens
SRSKAYGGTT

SEQ ID NO: 391 - protein, 14 aa, Homo sapiens
ASSGYSSGWTPFDY

SEQ ID NO: 392 - protein, 249 aa, Homo sapiens
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 393 - protein, 249 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 394 - protein, 115 aa, Homo sapiens
MQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 395 - protein, 112 aa, Homo sapiens
MGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 396 - protein, 115 aa, Homo sapiens
MQEVTQIPAALSVPEGENLVLNCSFTDSAIYNLQWFRQDPGKGLTSLLLIMSHQREQTSGRLNASLDKSSGRSTLYIAASQPGDSATYLCAVRPTSGGSYIPTFGRGTSLIVHPY

SEQ ID NO: 397 - protein, 112 aa, Homo sapiens
MGVTQTPKFQVLKTGQSMTLQCAQDMNHEYMSWYRQDPGMGLRLIHYSVSAGITDQGEVPNGYNVSRSTTEDFPLRLLSAAPSQTSVYFCASSYVGNTGELFFGEGSRLTVL

SEQ ID NO: 398 - protein, 9 aa, Human papillomavirus type 1a
YIIFVYIPL

SEQ ID NO: 399 - protein, 9 aa, Human papillomavirus type 1a
DPQERPRKL

SEQ ID NO: 400 - protein, 10 aa, Human papillomavirus type 1a
TIHEIILECV

SEQ ID NO: 401 - protein, 9 aa, Human papillomavirus type 1a
YMLDLQPET

SEQ ID NO: 402 - protein, 9 aa, Human papillomavirus type 1a
KCIDFYSRI

SEQ ID NO: 403 - protein, 9 aa, Human papillomavirus type 1a
FQQLFLNTL

SEQ ID NO: 404 - protein, 132 aa, Homo sapiens
METLLGLLILQLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRETSGSRLTFGEGTQLTVNPD

SEQ ID NO: 405 - protein, 133 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFWGRSTDTQYFGPGTRLTVL

SEQ ID NO: 406 - protein, 635 aa, Artificial Sequence
  MAPP construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 407 - protein, 535 aa, Artificial Sequence
  MAPP heterodimer construct
ELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEGGGGSGGGGSGGGGSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 408 - protein, 535 aa, Artificial Sequence
  MAPP heterodimer construct
ELAGIGILTVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGGGGSGGGGSGGGGSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 409 - protein, 535 aa, Artificial Sequence
  MAPP heterodimer construct
ELAGIGILTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEGGGGSGGGGSGGGGSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 410 - protein, 31 aa, Artificial Sequence
  Coiled-coil polypeptide
LARIEEKLKTIKAQLSEIASTLNMIREQLAQ

SEQ ID NO: 411 - protein, 9 aa, Artificial Sequence
  Alpha Feto Protein (AFP) Peptide
EYSRRHPQL

SEQ ID NO: 412 - protein, 9 aa, Artificial Sequence
  Alpha Feto Protein (AFP) Peptide
AYEEDRETF

SEQ ID NO: 413 - protein, 9 aa, Artificial Sequence
  Alpha Feto Protein (AFP) Peptide
SYANRRPCF

SEQ ID NO: 414 - protein, 9 aa, Artificial Sequence
  Alpha Feto Protein (AFP) Peptide
CFAEEGQKL

SEQ ID NO: 415 - protein, 9 aa, Artificial Sequence
  Alpha Feto Protein (AFP) Peptide
RSCGLFQKL

SEQ ID NO: 416 - protein, 9 aa, Artificial Sequence
  Alpha Feto Protein (AFP) Peptide
IFLIFLLNF

SEQ ID NO: 417 - protein, 9 aa, Artificial Sequence
  Alpha Feto Protein (AFP) Peptide
KPEGLSPNL

SEQ ID NO: 418 - protein, 10 aa, Artificial Sequence
  Alpha Feto Protein (AFP) Peptide
GLSPNLNRFL

SEQ ID NO: 419 - protein, 9 aa, Artificial Sequence
  Alpha Feto Protein (AFP) Peptide
EYYLQNAFL

SEQ ID NO: 420 - protein, 9 aa, Human papillomavirus type 1a
EYRHYCYSL

SEQ ID NO: 421 - protein, 9 aa, Human papillomavirus type 1a
HYCYSLYGT

SEQ ID NO: 422 - protein, 9 aa, Human papillomavirus type 1a
DFAFRDLCI

SEQ ID NO: 423 - protein, 10 aa, Human papillomavirus type 1a
LYGTTLEQQY

SEQ ID NO: 424 - protein, 10 aa, Human papillomavirus type 1a
HYCYSLYGTT

SEQ ID NO: 425 - protein, 10 aa, Human papillomavirus type 1a
EVYDFAFRDL

SEQ ID NO: 426 - protein, 10 aa, Human papillomavirus type 1a
EYRHYCYSLY

SEQ ID NO: 427 - protein, 10 aa, Human papillomavirus type 1a
VYDFAFRDLC

SEQ ID NO: 428 - protein, 10 aa, Human papillomavirus type 1a
YCYSIYGTTL

SEQ ID NO: 429 - protein, 9 aa, Human papillomavirus type 1a
VYCKTVLEL

SEQ ID NO: 430 - protein, 9 aa, Human papillomavirus type 1a
VYGDTLEKL

SEQ ID NO: 431 - protein, 10 aa, Human papillomavirus type 1a
LTNTGLYNLL

SEQ ID NO: 432 - protein, 9 aa, Human papillomavirus type 1a
DLQPETTDL

SEQ ID NO: 433 - protein, 9 aa, Human papillomavirus type 1a
TPTLHEYML

SEQ ID NO: 434 - protein, 9 aa, Human papillomavirus type 1a
GTLGIVCPI

SEQ ID NO: 435 - protein, 9 aa, Human papillomavirus type 1a
EPDRAHYNI

SEQ ID NO: 436 - protein, 9 aa, Human papillomavirus type 1a
QLFLNTLSF

SEQ ID NO: 437 - protein, 9 aa, Human papillomavirus type 1a
FQQLFLNTL

SEQ ID NO: 438 - protein, 10 aa, Human papillomavirus type 1a
AFQQLFLNTL

SEQ ID NO: 439 - protein, 9 aa, Artificial Sequence
  Wilms Tumor (WT-1) Peptide
VLDFAPPGA

SEQ ID NO: 440 - protein, 9 aa, Artificial Sequence
  Wilms Tumor (WT-1) Peptide
RMFPNAPYL

SEQ ID NO: 441 - protein, 9 aa, Artificial Sequence
  Wilms Tumor (WT-1) Peptide
YMFPNAPYL

SEQ ID NO: 442 - protein, 9 aa, Artificial Sequence
  Wilms Tumor (WT-1) Peptide
SLGEQQYSV

SEQ ID NO: 443 - protein, 9 aa, Artificial Sequence
  Wilms Tumor (WT-1) Peptide
CYTWNQMNL

SEQ ID NO: 444 - protein, 9 aa, Artificial Sequence
  Wilms Tumor (WT-1) Peptide
CMTWNQMNL

SEQ ID NO: 445 - protein, 9 aa, Artificial Sequence
  Wilms Tumor (WT-1) Peptide
NQMNLGATL

SEQ ID NO: 446 - protein, 8 aa, Artificial Sequence
  Wilms Tumor (WT-1) Peptide
CMTWNQMN

SEQ ID NO: 447 - protein, 15 aa, Artificial Sequence
  WT-1 peptide
MTWNQMNLGATLKGV

SEQ ID NO: 448 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 449 - protein, 15 aa, Artificial Sequence
  CAA peptide
TEDEVQSYPSKHDYV

SEQ ID NO: 450 - protein, 12 aa, Artificial Sequence
  CAA peptide
EVQSYPSKHDYV

SEQ ID NO: 451 - protein, 293 aa, Homo sapiens
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 452 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIK

SEQ ID NO: 453 - protein, 124 aa, Homo sapiens
QMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSS

SEQ ID NO: 454 - protein, 15 aa, Artificial Sequence
  Linker
GGGGSGGGGSGGGGS

SEQ ID NO: 455 - protein, 106 aa, Homo sapiens
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 456 - protein, 119 aa, Homo sapiens
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 457 - protein, 112 aa, Homo sapiens
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 458 - protein, 120 aa, Homo sapiens; identical to SEQ ID NO: 374

SEQ ID NO: 459 - protein, 106 aa, Homo sapiens
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 460 - protein, 119 aa, Homo sapiens
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 461 - protein, 11 aa, Homo sapiens
KASQDVSIAVA

SEQ ID NO: 462 - protein, 7 aa, Homo sapiens
SASYRYT

SEQ ID NO: 463 - protein, 9 aa, Homo sapiens
QQHYITPLT

SEQ ID NO: 464 - protein, 5 aa, Homo sapiens
NYGMN

SEQ ID NO: 465 - protein, 17 aa, Homo sapiens
WINTYTGEPTYTDDFKG

SEQ ID NO: 466 - protein, 12 aa, Homo sapiens
GGFGSSYWYFDV

SEQ ID NO: 467 - protein, 5 aa, Homo sapiens
TAGMQ

SEQ ID NO: 468 - protein, 17 aa, Homo sapiens
WINTHSGVPKYAEDFKG

SEQ ID NO: 469 - protein, 12 aa, Homo sapiens
SGFGSSYWYFDV

SEQ ID NO: 470 - protein, 11 aa, Homo sapiens
KASQDVSTAVA

SEQ ID NO: 471 - protein, 107 aa, Homo sapiens
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 472 - protein, 121 aa, Homo sapiens
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 473 - protein, 107 aa, Homo sapiens
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 474 - protein, 121 aa, Homo sapiens
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 475 - protein, 75 aa, Homo sapiens
QWYQQKPGQPPKLLIFAASNVESGVPARFSGSGSGTNFSLNIHPVDEDDVAMYFCQQSRKVPYTFGGGTKLEIKR

SEQ ID NO: 476 - protein, 120 aa, Homo sapiens
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 477 - protein, 5 aa, Homo sapiens
DYGVH

SEQ ID NO: 478 - protein, 16 aa, Homo sapiens
VIWAGGGTNYNSALMS

SEQ ID NO: 479 - protein, 12 aa, Homo sapiens
DKGYSYYYSMDY

SEQ ID NO: 480 - protein, 249 aa, Homo sapiens; identical to SEQ ID NO: 392

SEQ ID NO: 481 - protein, 11 aa, Homo sapiens
SASQDISNYLN

SEQ ID NO: 482 - protein, 7 aa, Homo sapiens
YTSNLHS

SEQ ID NO: 483 - protein, 9 aa, Homo sapiens
QQYRKLPWT

SEQ ID NO: 484 - protein, 5 aa, Homo sapiens
NYWMH

SEQ ID NO: 485 - protein, 17 aa, Homo sapiens
ATYRGHSDTYYNQKFKG

SEQ ID NO: 486 - protein, 12 aa, Homo sapiens
GAIYNGYDVLDN

SEQ ID NO: 487 - protein, 108 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 488 - protein, 121 aa, Homo sapiens
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 489 - protein, 20 aa, Homo sapiens
VTSAPDTRPAPGSTAPPAHG

SEQ ID NO: 490 - protein, 43 aa, Homo sapiens
SNIKFRPGSVVVQLTLAFREGTINVHDVETQFNQYKTEAASRY

SEQ ID NO: 491 - protein, 35 aa, Homo sapiens
SVVVQLTLAFREGTINVHDVETQFNQYKTEAASRY

SEQ ID NO: 492 - protein, 20 aa, Homo sapiens
LAFREGTINVHDVETQFNQY

SEQ ID NO: 493 - protein, 25 aa, Homo sapiens
SNIKFRPGSVVVQLTLAAFREGTIN

SEQ ID NO: 494 - protein, 5 aa, Homo sapiens
RYGMS

SEQ ID NO: 495 - protein, 17 aa, Homo sapiens
TISGGGTYIYYPDSVKG

SEQ ID NO: 496 - protein, 13 aa, Homo sapiens
DNYGRNYDYGMDY

SEQ ID NO: 497 - protein, 10 aa, Homo sapiens
SATSSVSYIH

SEQ ID NO: 498 - protein, 7 aa, Homo sapiens
STSNLAS

SEQ ID NO: 499 - protein, 9 aa, Homo sapiens
QQRSSSPFT

SEQ ID NO: 500 - protein, 5 aa, Homo sapiens
GYAMS

SEQ ID NO: 501 - protein, 17 aa, Homo sapiens
TISSGGTYIYYPDSVKG

SEQ ID NO: 502 - protein, 12 aa, Homo sapiens
LGGDNYYEYFDV

SEQ ID NO: 503 - protein, 15 aa, Homo sapiens
RASKSVSTSGYSYMH

SEQ ID NO: 504 - protein, 7 aa, Homo sapiens
LASNLES

SEQ ID NO: 505 - protein, 9 aa, Homo sapiens
QHSRELPFT

SEQ ID NO: 506 - protein, 5 aa, Homo sapiens
DYAMN

SEQ ID NO: 507 - protein, 17 aa, Homo sapiens
VISTFSGNINFNQKFKG

SEQ ID NO: 508 - protein, 10 aa, Homo sapiens
SDYYGPYFDY

SEQ ID NO: 509 - protein, 16 aa, Homo sapiens
RSSQTIVHSNGNTYLE

SEQ ID NO: 510 - protein, 7 aa, Homo sapiens
KVSNRFS

SEQ ID NO: 511 - protein, 9 aa, Homo sapiens
FQGSHVPFT

SEQ ID NO: 512 - protein, 17 aa, Homo sapiens
TISSGGTYIYYPDSVKG

SEQ ID NO: 513 - protein, 9 aa, Homo sapiens
LGGDNYYEY

SEQ ID NO: 514 - protein, 15 aa, Homo sapiens
TASKSVSTSGYSYMH

SEQ ID NO: 515 - protein, 7 aa, Homo sapiens
LVSNLES

SEQ ID NO: 516 - protein, 10 aa, Homo sapiens
QHIRELTRSE

SEQ ID NO: 517 - protein, 8 aa, Homo sapiens
GFTFSNYY

SEQ ID NO: 518 - protein, 8 aa, Homo sapiens
ISGRGSTI

SEQ ID NO: 519 - protein, 10 aa, Homo sapiens
VKDRGGYSPY

SEQ ID NO: 520 - protein, 6 aa, Homo sapiens
QSISTY

SEQ ID NO: 521 - protein, 10 aa, Homo sapiens
QQSYSTPPIT

SEQ ID NO: 522 - protein, 8 aa, Homo sapiens
GYTFTDYS

SEQ ID NO: 523 - protein, 8 aa, Homo sapiens
INTETGVP

SEQ ID NO: 524 - protein, 8 aa, Homo sapiens
ARRTGFDY

SEQ ID NO: 525 - protein, 11 aa, Homo sapiens
KNLLHSDGITY

SEQ ID NO: 526 - protein, 9 aa, Homo sapiens
VQVLELPFT

SEQ ID NO: 527 - protein, 8 aa, Homo sapiens
GFTFSSYA

SEQ ID NO: 528 - protein, 8 aa, Homo sapiens
ISDGGSYS

SEQ ID NO: 529 - protein, 14 aa, Homo sapiens
ARDSYYDNSYVRDY

SEQ ID NO: 530 - protein, 6 aa, Homo sapiens
QDINTF

SEQ ID NO: 531 - protein, 9 aa, Homo sapiens
LQYDEFPLT

SEQ ID NO: 532 - protein, 757 aa, Artificial Sequence
  MAPP construct
NLVPMVATVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 533 - protein, 594 aa, Artificial Sequence
  MAPP construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 534 - protein, 586 aa, Artificial Sequence
  MAPP construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 535 - protein, 895 aa, Artificial Sequence
  MAPP construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 536 - protein, 1058 aa, Artificial Sequence
  MAPP construct
NLVPMVATVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 537 - protein, 887 aa, Artificial Sequence
  MAPP construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 538 - protein, 376 aa, Artificial Sequence
  MAPP construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 539 - protein, 368 aa, Artificial Sequence
  MAPP construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 540 - protein, 539 aa, Artificial Sequence
  MAPP construct
NLVPMVATVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 541 - protein, 677 aa, Artificial Sequence
  MAPP construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGECGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 542 - protein, 669 aa, Artificial Sequence
  MAPP construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGECGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 543 - protein, 840 aa, Artificial Sequence
  MAPP construct
NLVPMVATVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGECGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 544 - protein, 9 aa, Artificial Sequence
  CMV peptide
NLVPMVATV

SEQ ID NO: 545 - protein, 9 aa, Artificial Sequence
  NY-ESO epitope peptide
SLLMWITQC

SEQ ID NO: 546 - protein, 10 aa, Artificial Sequence
  MART peptide
ELAGIGILTV

SEQ ID NO: 547 - protein, 5 aa, Artificial Sequence
  Peptide suitable for use as a linker. The EAAAK may be repeated 2 to 20 times.
EAAAK

SEQ ID NO: 548 - protein, 7 aa, Artificial Sequence
  Peptide suitable for use as a linker. The EAAAK subset may be repeated 2 to 20 times.
AEAAAKA

SEQ ID NO: 549 - protein, 16 aa, Artificial Sequence
  Peptide suitable for use as a linker. The EAAAK subset may be repeated independently 2 to 20 times.
AEAAAKALEAEAAAKA

SEQ ID NO: 550 - protein, 5 aa, Artificial Sequence
  Peptide suitable for use as a linker
EAAAK

SEQ ID NO: 551 - protein, 10 aa, Artificial Sequence
  Peptide suitable for use as a linker.
EAAAKEAAAK

SEQ ID NO: 552 - protein, 15 aa, Artificial Sequence
  Peptide suitable for use as a linker.
EAAAKEAAAKEAAAK

SEQ ID NO: 553 - protein, 46 aa, Artificial Sequence
  Peptide suitable for use as a linker.
AEAAAKEAAAKEAAAKEAAAKALEAEAAAKEAAAKEAAAKEAAAKA

SEQ ID NO: 554 - protein, 12 aa, Artificial Sequence
  Peptide suitable for use as a linker.
AEAAAKEAAAKA

SEQ ID NO: 555 - protein, 5 aa, Artificial Sequence
  Peptide suitable for use as a linker.
PAPAP

SEQ ID NO: 556 - protein, 8 aa, Artificial Sequence
  Peptide suitable for use as a linker.
APAPAPAP

SEQ ID NO: 557 - protein, 12 aa, Artificial Sequence
  Peptide suitable for use as a linker.
APAPAPAPAPAP

SEQ ID NO: 558 - protein, 16 aa, Artificial Sequence
  Peptide suitable for use as a linker.
APAPAPAPAPAPAPAP

SEQ ID NO: 559 - protein, 20 aa, Artificial Sequence
  Peptide suitable for use as a linker.
APAPAPAPAPAPAPAPAPAP

SEQ ID NO: 560 - protein, 4 aa, Artificial Sequence
  Peptide suitable for use as a linker.
KPKP

SEQ ID NO: 561 - protein, 8 aa, Artificial Sequence
  Peptide suitable for use as a linker.
KPKPKPKP

SEQ ID NO: 562 - protein, 12 aa, Artificial Sequence
  Peptide suitable for use as a linker.
KPKPKPKPKPKP

SEQ ID NO: 563 - protein, 16 aa, Artificial Sequence
  Peptide suitable for use as a linker.
KPKPKPKPKPKPKPKP

SEQ ID NO: 564 - protein, 20 aa, Artificial Sequence
  Peptide suitable for use as a linker.
KPKPKPKPKPKPKPKPKPKP

SEQ ID NO: 565 - protein, 4 aa, Artificial Sequence
  Peptide suitable for use as a linker.
EPEP

SEQ ID NO: 566 - protein, 8 aa, Artificial Sequence
  Peptide suitable for use as a linker.
EPEPEPEP

SEQ ID NO: 567 - protein, 12 aa, Artificial Sequence
  Peptide suitable for use as a linker.
EPEPEPEPEPEP

SEQ ID NO: 568 - protein, 16 aa, Artificial Sequence
  Peptide suitable for use as a linker.
EPEPEPEPEPEPEPEP

SEQ ID NO: 569 - protein, 20 aa, Artificial Sequence
  Peptide suitable for use as a linker.
EPEPEPEPEPEPEPEPEPEP

SEQ ID NO: 570 - protein, 5 aa, Artificial Sequence
  Peptide suitable for use as a linker.
CGGGS

SEQ ID NO: 571 - protein, 5 aa, Artificial Sequence
  Peptide suitable for use as a linker.
GCGGS

SEQ ID NO: 572 - protein, 5 aa, Artificial Sequence
  Peptide suitable for use as a linker.
GGCGS

SEQ ID NO: 573 - protein, 5 aa, Artificial Sequence
  Peptide suitable for use as a linker.
GGGCS

SEQ ID NO: 574 - protein, 5 aa, Artificial Sequence
  Peptide suitable for use as a linker.
GGGGC